(19) United States
(12) Patent Application Publication
(10) Pub. No.: US 2025/0281408 A1
(43) Pub. Date: Sep. 11, 2025

(54) AMORPHOUS SOLID DISPERSIONS AND PHARMACEUTICAL COMPOSITIONS COMPRISING THE SAME

(71) Applicant: Shenzhen Pharmacin Co., Ltd., Shenzhen (CN)

(72) Inventors: Zeren WANG, Shenzhen (CN); Shun CHEN, Shenzhen (CN); Dailong FANG, Shenzhen (CN); Jiqian PENG, Shenzhen (CN); Yanxin ZHAO, Shenzhen (CN); Yingfen LIAO, Shenzhen (CN)

(21) Appl. No.: 18/850,375

(22) PCT Filed: Mar. 24, 2023
(86) PCT No.: PCT/CN2023/083771

§ 371 Date: Sep. 24, 2024

(30) Foreign Application Priority Data
Mar. 25, 2022 (WO) PCT/CN2022/083103
Mar. 8, 2023 (WO) PCT/CN2023/080338

(51) Int. Cl. A61K 9/14; A61K 31/47; A61K 31/496; A61K 31/506; A61K 31/5377; A61K 31/58; A61K 31/635

(52) U.S. Cl. CPC A61K 9/145; A61K 9/143; A61K 9/146; A61K 31/47; A61K 31/496; A61K 31/506; A61K 31/5377; A61K 31/58; A61K 31/635

(57) ABSTRACT

Provided are pharmaceutical compositions which include an amorphous solid dispersion comprising i) a lipophilic active pharmaceutical ingredient such as abiraterone, alectinib, pazopanib, cabozantinib, venetoclax, lurasidone, or vilazodone or a salt thereof, ii) a hydrophilic polymer, iii) optionally a surfactant, iv) optionally an adsorbent, and v) optionally an acid. Also described are methods for preparing such pharmaceutical compositions and treating a subject in need thereof. In one aspect, disclosed herein is an amorphous solid dispersion comprising an active pharmaceutical ingredient.

### CROSS-REFERENCE TO RELATED APPLICATION

This application is a U.S. National Stage entry of International Application no. PCT/CN2023/083771, filed on Mar. 24, 2023, which claims the benefit of patent application Nos. PCT/CN2022/083103, filed on Mar. 25, 2022, and PCT/CN2023/080338, filed on Mar. 8, 2023, each of which is hereby incorporated by reference in its entirety.

### TECHNICAL FIELD

The present invention belongs to the pharmaceutical field, and specifically relates to pharmaceutical compositions, and their preparation method and use.

### BACKGROUND

Many active pharmaceutical ingredients (APIs) are compounds with poor aqueous solubility, low oral bioavailability, and food-effect when administered orally. Some examples of these APIs are abiraterone acetate, alectinib, pazopanib, cabozantinib, venetoclax, lurasidone, and vilazodone. Thus, there is a need for improved compositions for such APIs to provide better oral bioavailability, permit administering lower doses, and reduce absorption variations caused by food intake and in vivo inter-subject absorption variations.

### INCORPORATION BY REFERENCE

All publications and patent applications mentioned in this specification are herein incorporated by reference to the same extent as if each individual publication or patent application was specifically and individually indicated to be incorporated by reference.

### BRIEF SUMMARY

Disclosed herein is a pharmaceutical composition, wherein the pharmaceutical composition comprises an amorphous solid dispersion (ASD) that comprises: a) an active pharmaceutical ingredient (API) in an amount of about 5% to about 60% by weight of the ASD, wherein the API is cabozantinib or a pharmaceutically acceptable salt thereof; b) a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in an amount of about 5% to about 60% by weight of the ASD, wherein the API is venetoclax or a pharmaceutically acceptable salt thereof; b) a surfactant in an amount of about 5% to about 50% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is abiraterone or abiraterone acetate; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is abiraterone or abiraterone acetate; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 95% by weight of the ASD; d) optionally an acid in an amount of about 5% to 60% by weight of the ASD; and e) optionally an adsorbent in an amount of about 1% to 60% by weight of the ASD.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is alectinib or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is alectinib or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 95% by weight of the ASD; d) optionally an acid in an amount of about 5% to 40% by weight of the ASD; and e) optionally an adsorbent in an amount of about 1% to 50% by weight of the ASD.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is pazopanib or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is pazopanib or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 95% by weight of the ASD; d) optionally an acid in an amount of about 5% to 60% by weight of the ASD; and e) optionally an adsorbent in an amount of about 1% to 60% by weight of the ASD.

Disclosed herein is a pharmaceutical composition, wherein the pharmaceutical composition comprises an amorphous solid dispersion (ASD) that comprises: a) an active pharmaceutical ingredient (API) in amount of about 3% to about 60% by weight of the ASD, wherein the API is lurasidone or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is lurasidone or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 95% by weight of the ASD; d) optionally an acid in an amount of about 5% to 60% by weight of the ASD; and e) optionally an adsorbent in an amount of about 1% to 60% by weight of the ASD.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is vilazodone or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 80% by weight of the ASD; d) optionally an acid; and e) optionally an adsorbent.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is vilazodone or a pharmaceutically acceptable salts thereof; b) optionally, a surfactant in an amount of about 5% to about 60% by weight of the ASD, wherein the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof; c) a hydrophilic polymer in an amount of about 1% to about 95% by weight of the ASD; d) optionally an acid in an amount of about 5% to 60% by weight of the ASD; and e) optionally an adsorbent in an amount of about 1% to 60% by weight of the ASD.

Disclosed herein is a pharmaceutical composition, wherein the pharmaceutical composition comprises an amorphous solid dispersion (ASD) that comprises: a) an active pharmaceutical ingredient (API), wherein the API is selected from abiraterone, alectinib, pazopanib, cabozantinib, venetoclax, lurasidone, vilazodone and pharmaceutically acceptable salts thereof; b) a surfactant, wherein the surfactant comprises phospholipids or their derivatives; c) a hydrophilic polymer; and d) optionally an adsorbent.

Disclosed herein is a pharmaceutical composition, wherein the pharmaceutical composition comprises an amorphous solid dispersion (ASD) that comprises: a) an active pharmaceutical ingredient (API) in an amount of about 5% to about 35% by weight of the ASD, wherein the API is cabozantinib or a pharmaceutically acceptable salt thereof; b) a surfactant in an amount of about 5% to about 60% by weight of the ASD, wherein the surfactant comprises phospholipids or their derivatives or lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof; c) a hydrophilic polymer in an amount of about 1% to about 80% by weight of the ASD; and d) optionally an adsorbent in an amount of about 1% to 40% by weight of the ASD, wherein the adsorbent is silicone dioxide.

Disclosed herein is a pharmaceutical composition, wherein the pharmaceutical composition comprises an amorphous solid dispersion (ASD) that comprises: a) an active pharmaceutical ingredient (API) in an amount of about 5% to about 45% by weight of the ASD, wherein the API is venetoclax or a pharmaceutically acceptable salt thereof; b) a surfactant in an amount of about 5% to about 50% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof; c) a non-ionic hydrophilic polymer in an amount of about 1% to about 80% by weight of the ASD; d) optionally an inorganic acid or organic acid in an amount of about 1% to 20% by weight of the ASD; and e) optionally an adsorbent in an amount of about 1% to 40% by weight of the ASD, wherein the adsorbent is silicone dioxide.

Disclosed herein is a pharmaceutical composition, wherein the pharmaceutical composition comprises an amorphous solid dispersion (ASD) that comprises: a) an active pharmaceutical ingredient (API) in amount of about 5% to about 60% by weight of the ASD, wherein the API is selected from abiraterone acetate, alectinib hydrochloride, pazopanib hydrochloride, lurasidone, vilazodone, and pharmaceutically acceptable salts thereof; b) a surfactant in an amount of about 5% to about 60% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof; c) a hydrophilic polymer in an amount of about 1% to about 90% by weight of the ASD; d) optionally an inorganic acid or organic acid in an amount of about 5% to 40% by weight of the ASD; and e) optionally an adsorbent in an amount of about 1% to 40% by weight of the ASD.

Disclosed herein is an amorphous solid dispersion (ASD), wherein the amorphous solid dispersion comprises: a) cabozantinib free base or cabozantinib malate; b) a surfactant; c) a hydrophilic polymer; d) optionally an adsorbent; and e) optionally an organic acid.

Disclosed herein is a method of treating a disease or condition, comprising administering to a subject in need thereof the pharmaceutical composition or the amorphous solid dispersion described herein.

Disclosed herein is a method of treating cancer, comprising administering to a subject in need thereof the pharmaceutical composition or the amorphous solid dispersion described herein. In some embodiments, the cancer comprises kidney cancer, liver cancer, and thyroid cancer. In some embodiments, the kidney cancer is advanced renal cell carcinoma. In some embodiments, the liver cancer is hepatocellular carcinoma. In some embodiments, the thyroid cancer is locally advanced or metastatic differentiated thyroid cancer or medullary thyroid cancer.

Disclosed herein is a method of inhibiting multiple tyrosine-kinases, comprising administering to a subject in need thereof the pharmaceutical composition or the amorphous solid dispersion described herein. In some embodiments, the multiple tyrosine-kinases comprise VEGFR2, c-MET or RET. In some embodiments, the method further comprising administering an immunotherapeutic agent. In some embodiments, the immunotherapeutic agent is nivolumab. In some embodiments, the subject was previously treated with sorafenib. In some embodiments, the subject a) is 12 years of age or older, b) progressed following prior VEGFR-targeted therapy, and c) is radioactive iodine-refractory or ineligible.

Disclosed herein is an amorphous solid dispersion (ASD), wherein the amorphous solid dispersion comprises: a) venetoclax or a pharmaceutically acceptable salt thereof; b) a surfactant; c) a hydrophilic polymer; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating cancer comprising administering to a subject in need thereof the pharmaceutical composition or the amorphous solid dispersion described herein. In some embodiments, the caner is a blood cancer. In some embodiments, the blood cancer is chronic lymphocytic leukemia. In some embodiments, the blood cancer is acute myeloid leukemia. In some embodiments, the caner is a solid tumor. In some embodiments, the solid tumor is lymphoma. In some embodiments, the lymphoma is small lymphocytic lymphoma.

Disclosed herein is a method of inhibiting B-cell lymphoma-2 (Bel-2) protein, comprising administering to a subject in need thereof the pharmaceutical composition or the amorphous solid dispersion described herein. In some embodiments, the method further comprising administering an immunotherapeutic agent. In some embodiments, the immunotherapeutic agent is obinutuzumab or rituximab. In some embodiments, the method further comprising administering a chemotherapeutic agent. In some embodiments, the chemotherapeutic agent is azacitidine, or decitabine, or low-dose cytarabine. In some embodiments, the subject is previously untreated. In some embodiments, the subject is previously treated. In some embodiments, the subject a) is newly diagnosed of acute myeloid leukemia; and b) is 75 years of age or older; or c) has other medical conditions that prevent the use of standard chemotherapy. In some embodiments, the subject is an adult.

Disclosed herein is an amorphous solid dispersion (ASD), wherein the amorphous solid dispersion comprises: a) abiraterone free base or abiraterone acetate; b) a hydrophilic polymer; c) optionally a surfactant; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating cancer comprising administering to a subject in need thereof the pharmaceutical composition or the amorphous solid dispersion described herein. In some embodiments, the caner is a solid tumor. In some embodiments, the solid tumor is prostate cancer. In some embodiments, the prostate cancer is metastatic castration-resistant prostate cancer. In some embodiments, the prostate cancer is metastatic high-risk castration-sensitive prostate cancer.

Disclosed herein is a method of inhibiting 17 alpha-hydroxylase/C17, 20-lyase (CYP17), comprising administering to a subject in need thereof the pharmaceutical composition or the amorphous solid dispersion described herein. In some embodiments, the method further comprising administering a corticosteroid. In some embodiments, the corticosteroid is prednisone or methylprednisolone. In some embodiments, the method further comprising administering a corticosteroid. In some embodiments, the corticosteroid is methylprednisolone. In some embodiments, the subject is a male adult.

Disclosed herein is an amorphous solid dispersion (ASD), wherein the amorphous solid dispersion comprises: a) alectinib free base or alectinib hydrochloride; b) a hydrophilic polymer; c) a surfactant; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating cancer comprising administering to a subject in need thereof the pharmaceutical composition or the amorphous solid dispersion described herein. In some embodiments, the caner is a solid tumor. In some embodiments, the solid tumor is lung cancer. In some embodiments, the lung cancer is non-small cell lung cancer. In some embodiments, the non-small cell lung cancer is anaplastic lymphoma kinase (ALK)-positive, metastatic high-risk castration-sensitive prostate cancer.

Disclosed herein is a method of inhibiting ALK and/or RET tyrosine kinases, comprising administering to a subject in need thereof the pharmaceutical composition or the amorphous solid dispersion described herein. In some embodiments, the subject is an adult. In some embodiments, the subject is intolerant to crizotinib.

Disclosed herein is an amorphous solid dispersion (ASD), wherein the amorphous solid dispersion comprises: a) pazopanib free base or pazopanib hydrochloride; b) a hydrophilic polymer; c) a surfactant; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating cancer comprising administering to a subject in need thereof the pharmaceutical composition or the amorphous solid dispersion described herein. In some embodiments, the caner is a solid tumor. the solid tumor is soft tissue sarcoma. In some embodiments, the soft tissue sarcoma is advanced soft tissue sarcoma. In some embodiments, the cancer is kidney cancer. In some embodiments, the kidney cancer is advanced renal cell cancer.

Disclosed herein is a method of inhibiting tyrosine kinases of VEGF receptor (VEGFR) and/or PDGF receptor (PDGFR), comprising administering to a subject in need thereof the pharmaceutical composition or the amorphous solid dispersion described herein. In some embodiments, the subject is an adult. In some embodiments, the subject previously received chemotherapy.

Disclosed herein is an amorphous solid dispersion (ASD), wherein the amorphous solid dispersion comprises: a) lurasidone free base or lurasidone hydrochloride; b) a hydrophilic polymer; c) a surfactant; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating a mental disorder comprising administering to a subject in need thereof the pharmaceutical composition or the amorphous solid dispersion described herein. In some embodiments, the mental disorder is schizophrenia. In some embodiments, the mental disorder is depression. In some embodiments, the depression is associated with bipolar I disorder. In some embodiments, the depression is bipolar depression.

Disclosed herein is a method of inhibiting central dopamine D2 and serotonin Type 2 (5HT2A) receptor, comprising administering to a subject in need thereof the pharmaceutical composition or the amorphous solid dispersion described herein. In some embodiments, the subject is an adult. In some embodiments, the adolescent is 13 to 17 years old. In some embodiments, the method further comprising administering an anticonvulsant. In some embodiments, the anticonvulsant is lithium or valproate.

Disclosed herein is an amorphous solid dispersion (ASD), wherein the amorphous solid dispersion comprises: a) vilazodone free base or vilazodone hydrochloride; b) a hydrophilic polymer; c) a surfactant; d) optionally an organic acid; and e) optionally an adsorbent.

Disclosed herein is a method of treating a disease or condition, comprising administering to a subject in need thereof the pharmaceutical composition or the amorphous solid dispersion described herein.

Disclosed herein is a method of treating a mental disorder comprising administering to a subject in need thereof the pharmaceutical composition or the amorphous solid dispersion described herein. In some embodiments, the mental disorder major depressive disorder.

Disclosed herein is a method of inhibiting serotonin (5-HT1A) receptors, comprising administering to a subject in need thereof the pharmaceutical composition or the amorphous solid dispersion described herein. In some embodiments, the subject is an adult.

Disclosed herein is a method of stimulating serotonin transporter, comprising administering to a subject in need thereof the pharmaceutical composition or the amorphous solid dispersion described herein. In some embodiments, the stimulation is via partial agonism. In some embodiments, the subject is an adult.

### BRIEF DESCRIPTION OF THE DRAWINGS

Provided herein are pharmaceutical compositions comprising amorphous solid dispersions that comprise such APIs and suitable excipients or carriers provide improved bioavailability and eliminate or reduce food-effect compared to the existing compositions comprising crystalline form of such APIs. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows X-ray powder diffraction studies for two amorphous solid dispersions (ASD) batches P211115-2 and P211115-1 for abiraterone acetate, both of which are in amorphous state.

FIG. 1B shows a comparison of X-ray powder diffraction for abiraterone acetate API and two amorphous solid dispersions (ASD) batches P211115-2 and P211115-1 for abiraterone acetate, both of ASD are in amorphous state whereas abiraterone acetate API is not in amorphous state.

FIG. 1C shows a comparison of plasma concentrations of API (Abiraterone) in dog model when reference product ZYTIGA (250 mg) and two ASD compositions of Abiraterone (Batch No. P211115-1 and P211115-2) were given orally at the dose of 50 mg API in fasted condition.

FIG. 2A shows X-ray powder diffraction studies for crystalline alectinib hydrochloride and four solid dispersion batches I-M211221-1, I-M211221-3, I-M211202-3, and I-M211229-1 for alectinib hydrochloride, batch I-M211202-3 is mostly amorphous, and all other batches are in amorphous state.

FIG. 2B shows X-ray powder diffraction studies for six solid dispersion batches I-M221214-1, I-M221214-2, I-M221214-3, I-M221214-4, I-M221214-5, and I-M221214-6 for alectinib hydrochloride, all batches are in amorphous state.

FIG. 3 shows the kinetic solubilities of four ASD batches I-M211221-1, I-M211221-3, I-M211202-3, and I-M211229-1 for alectinib hydrochloride, measured by HPLC assay.

FIG. 4A shows X-ray powder diffraction studies for three ASD batches M210706-1, M210706-2, and M211214-2 for pazopanib hydrochloride, all of which are in amorphous state.

FIG. 4B shows X-ray powder diffraction studies for five ASD batches M221222-2, M221222-3, M221222-4, M230104-2, and M230104-3 for pazopanib hydrochloride, all of which are in amorphous state.

FIG. 5A shows X-ray powder diffraction studies for three ASD batches M210702-1-I, M210702-2-I, and M210702-3-I for cabozantinib malate, all of which are in amorphous state.

FIG. 5B shows X-ray powder diffraction studies for five ASD batches M220701-1, M220701-2, M220622-3, M220622-4, and M220714 for cabozantinib malate, all of which are in amorphous state.

FIG. 6 shows the kinetic solubilities of three ASD batches M210702-1-I, M210702-2-I, and M210702-3-I for cabozantinib malate, measured by HPLC assay.

FIG. 7A shows X-ray powder diffraction studies for two ASD batches M210618-1-I and M210618-2-I for venetoclax, both of which are in amorphous state.

FIG. 7B shows X-ray powder diffraction studies for four ASD batches I-M220808-3, I-M220630-2, I-M220727-2 and I-M221010-1 for venetoclax, all of which are in amorphous state.

FIG. 7C shows X-ray powder diffraction studies for ASD batches I-M221219-1, I-M221219-2, I-M221219-3, I-M221221-1, I-M221221-2, I-M221221-3 and I-M221221-4 for venetoclax, all of which are in amorphous state.

FIG. 8A shows the kinetic solubilities of two ASD batches M210618-1-I and M210618-2-I for venetoclax, measured by HPLC assay.

FIG. 8B shows the comparison of mean PK profile in rat model for venetoclax RLD tablet milled into powder and venetoclax ASD powder from batches I-M221201-2, I-M221118, I-M221219-1, and I-M221219-2, all of which were then suspended in 0.5% CMC-Na solution before administration.

FIG. 9A shows X-ray powder diffraction studies for three ASD batches I-M211207-2, I-M211209-4, and M211028-1 for lurasidone, all of which are in amorphous state.

FIG. 9B shows X-ray powder diffraction studies for five ASD batches I-M211207-2, I-M211209-4, M211028-1, M211101-1, and M211101-2 for lurasidone, all of which are in amorphous state.

FIG. 9C shows X-ray powder diffraction studies for three ASD batches I-M211207-3, I-M211207-4, and I-M220712-1 for lurasidone, all of which are in amorphous state.

FIG. 10 shows the kinetic solubilities of three ASD batches I-M211207-2, I-M211209-4, and M211028-1 for lurasidone, measured by HPLC assay.

FIG. 11 shows X-ray powder diffraction study for an ASD batch P210324-1 for lurasidone, which is in amorphous state.

FIG. 12A shows the kinetic solubilities of the crystalline lurasidone (API) and an ASD batch P210324-1 for lurasidone in FeSSIF medium, measured by HPLC assay.

FIG. 12B shows the kinetic solubilities of the crystalline lurasidone (API) and an ASD batch P210324-1 for lurasidone in FaSSIF medium, measured by HPLC assay.

FIG. 13A shows X-ray powder diffraction study for an ASD batch P210324-1 vilazodone, which is in amorphous state.

FIG. 13B shows X-ray powder diffraction studies for vilazodone API in crystalline form, and an ASD batch P210324-1 vilazodone in amorphous state.

FIG. 14 shows the kinetic solubilities of crystalline vilazodone and an ASD batch P210324-1 for vilazodone in FaSSIF and FeSSIF medium, measured by HPLC assay.

### DETAILED DESCRIPTION

The present invention is generally directed to compositions comprising pharmaceutically active agents that are useful as therapeutics that alleviate, abate or eliminate one or more conditions in a subject in need thereof, as further described herein. In particular, described herein are pharmaceutical compositions, their synthesis and use, where the pharmaceutical compositions comprise a lipophilic API, a hydrophilic polymer, and a surfactant in a combination such that the API has improved bioavailability compared to the API alone. In some embodiments, the lipophilic API, hydrophilic polymer, and the surfactant is in an amorphous solid dispersion. In some embodiments, the pharmaceutical compositions optionally comprise one or more adsorbent. In some embodiments, the pharmaceutical compositions optionally comprise one or more organic or inorganic acid.

### Definitions

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The singular forms “a,” “an,” and, “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“AUC” or “AUC_{inf}” as used herein refers to the area under the plasma drug concentration-versus-time curve extrapolated from zero time to infinity. “AUCₗₐₛₜ” as used herein refers to the area under the curve from the time of dosing to the time of the last measurable concentration. “Cₘₐₓ” as used herein refers to the highest drug concentration observed in plasma following an extravascular dose of drug. “Tₘₐₓ” as used herein refers to the time after administration of a drug when the maximum plasma concentration is reached.

“D10,” “D50,” and “D90” as used herein to describe a particle size distribution. The “D10” as used herein refers to the diameter that has ten percent of the total mass of particles smaller and ninety percent larger. The “D50” as used herein refers to the median diameter where fifty percent of the total mass of particles are larger and 50% are smaller. The “D90” defines the diameter where ninety percent of the mass distribution has a smaller particle diameter and ten percent has a larger particle diameter.

In some embodiments, an error-band is included. The term “total error band” is used herein to specify all sources of including sampling and sample preparation calculated at a 95% confidence level. An example is: D50 100 μm with a total error band of +/−5% on size. Other statistics are sometimes used to describe a particle size distribution. The most common calculations are standard deviation and variance. The standard deviation (St Dev.). The standard deviation specification defines the diameter where approximately 68.27% of the total population lies within +/−1 St Dev, and 95.45% lies within +/−2 St Dev.

“Effective amount,” and “sufficient amount” may be used interchangeably, and refer to an amount of a substance that is sufficient to achieve an intended purpose or objective.

A “therapeutically effective amount” when used in connection with a pharmaceutical composition described herein is an amount of one or more pharmaceutically active agent(s) sufficient to produce a therapeutic result in a subject in need thereof.

“Therapeutically equivalent” when used in connection with a pharmaceutical composition described herein refers to an amount or quantity of a pharmaceutically acceptable salt or ester of a pharmaceutically active agent that is equivalent to the therapeutically effective amount of the free base or alcohol of the pharmaceutically active agent.

### Active Pharmaceutical Ingredient (API)

In one aspect, disclosed herein are pharmaceutical compositions comprising an ASD, wherein the ASD comprises an active pharmaceutical agent. Various embodiments described herein are directed to compositions comprising an effective amount of an active pharmaceutical agent (API). “Active pharmaceutical agent,” “API,” “APIs,” “drug,” “pharmaceutically active agent,” “bioactive agent,” “therapeutic agent,” and “active agent” and the like may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

The partition-coefficient (P) as referenced herein is a ratio of concentrations of a compound between two immiscible solvent phases at equilibrium. Most commonly, one of the solvents is water and the other is hydrophobic, typically 1-octanol. The logarithm of the ratio is log P, as shown below, (conventionally the lipophilic phase is the numerator and hydrophilic phase is the denominator.)

$$log {P}_{octanol / water} = log ( \frac{{[ solute ]}_{octanol}}{{[ solute ]}_{water}} )$$

log P is a measure of lipophilicity or hydrophobicity. Hydrophobicity affects drug absorption, bioavailability, hydrophobic drug-receptor interactions, metabolism of molecules, and toxicity. Hydrophilic compounds are soluble in water (“water-loving”) and polar solvents. Lipophilic compounds are less soluble in water (“water-fearing” or hydrophobic) and polar solvents, but are more soluble in organic solvents. Thus:
Low hydrophilicity=high lipophilicity=high log P=poor aqueous solubility=poor absorption.
High hydrophilicity=low lipophilicity=low log P=good aqueous solubility=good absorption.

Partition coefficients can be measured experimentally or estimated via calculation. Various methods for calculating (or predicting) log P have been developed, typically by fitting calculated log P values with experimentally measured log P values for training sets of thousands of molecules, mostly drug-like. Log P calculations are considered very robust and accurately process many organic molecules. For example, over 50% of molecules log P is predicted with error of less than 0.25, while over 80% with error of less than 0.5. Less than 3.5% of structures are predicted with an error greater 1.0. To distinguish from a measured log P, a calculated log P is sometimes written as clog P. Unless otherwise indicated, “log P” as used herein refers to an experimental log P value.

In some embodiments, the API is lipophilic. An API is considered lipophilic if its log P or calculated log P is 2.0 or higher. A log P of 2.0 or higher denotes that the solubility of the API is 100-fold or higher in a lipophilic solvent than in water. In some embodiments, the API is insoluble in polar solvents. In some embodiments, the API is insoluble in aqueous media. In some embodiments, the API is insoluble in water.

In some embodiments, the lipophilic API has a log P of at least 2.0, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. Exemplary small molecule lipophilic API's include, without limitation, those listed in Table 1.

TABLE 1

| | Calculated | Experimental |
|---|---|---|
| Name | log P | log P |
| Abiraterone Acetate | 5.1¹ | 5.12² |
| pazopanib | 3.59³ | Not Available |
| alectinib | 5.59¹ | Not Available |
| Cabozantinib malate | 4.01¹ | — |
| Venetoclax | 6.92¹ | |
| lurasidone | 5.4¹ | — |
| vilazodone | 3.72³ | — |

¹Calculation source - ALOGPS listed on www.drugbank.ca
²Experimental values listed on drug label
³Calculation source - ChemAxon listed on www.drugbank.ca

An acid dissociation constant, Ka, (or acidity constant) is a measure of the strength of an acid or base in solution, typically water. It is the equilibrium constant for the chemical dissociation of acids and bases. In aqueous solution, the equilibrium of acid dissociation is written:

HA+H₂O [symbol] A⁻+H₃O⁺

where HA is an acid that dissociates into A⁻, (the conjugate base of the acid) and a hydrogen ion (which combines with a water molecule to make a hydronium ion, H₃O⁺). The dissociation constant can also be written with the H₂O removed:

$$HA ⇋ {A}^{-} + {H}^{+} \\ {K}_{a} = \frac{[ {A}^{-} ] [ {H}^{+} ]}{[ HA ]}$$

The equilibrium of the dissociation of the conjugate acid of a base is written:

BH⁺+H₂O [symbol] B+H₃O⁺

where BH⁺ (the conjugate acid of the base) dissociates into B (the free base), and a hydrogen ion, which combines with a water molecule to form a hydronium ion, H₃O⁺.
The dissociation constant can also be written with the H₂O removed:

$${BH}^{+} ⇋ B + {H}^{+} \\ {K}_{b} = \frac{[ B ] [ {H}^{+} ]}{[ {BH}^{+} ]}$$

pKₐ, the logarithmic value of Kₐ, is more often used to express acid the strength/weakness of acids or the conjugate acid of bases:

$${pK}_{a} = - {log}_{10} ( {K}_{a} )$$

The more positive the value of pKₐ, the smaller the extent of dissociation, and the weaker the acid. In general, for acids:
pKₐ=−2 to 12→weak acid (little or only partial dissociation in water)
pKₐ<−2→strong acid (completely or mostly dissociated in water)
while for bases:
pKₐ<12→weak base (little or only partial dissociation in water)
pKₐ≥12→strong base (completely or mostly dissociated in water)

In some embodiments, the API is a weak base.

In some embodiments, the API comprises a weak base functional group.

In some embodiments, the API has a pKa of equal or greater than 3.0. In some embodiments, the API has a pKa of equal or greater than 3.5. In some embodiments, the API has a pKa of equal or greater than 4.0. In some embodiments, the API has a pKa of equal or greater than 4.5. In some embodiments, the API has a pKa of equal or greater than 5.0.

In some embodiments, the API is present in the form of a free base. In some embodiments, the API is present in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. In some embodiments the API is abiraterone acetate, alectinib, pazopanib, cabozantinib, venetoclax, lurasidone, vilazodone or a pharmaceutically acceptable salt or ester thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, vilazodone. In some embodiments, the API is a lipophilic API. In some embodiments, the lipophilic API is one listed in Table 1, or a pharmaceutically acceptable salt thereof. In some embodiments, the API is one selected from Table 1. In some embodiments, the API has a log P 2.0 or higher. In some embodiments, the API has a log P 3.0 or higher. In some embodiments, the API has a log P 3.5 or higher. In some embodiments, the API has a log P 4.0 or higher. In some embodiments, the API has a log P 4.5 or higher. In some embodiments, the lipophilic API, hydrophilic polymer, and the surfactant is formulated as an amorphous solid dispersion. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

Pharmaceutically acceptable salts include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentaflu oropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, 19ydroxyapat, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt. In some embodiments the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound disclosed herein may also form internal salts. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, the lipophilic API has a log P 3.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, the API has a low solubility at a pH of about 6-8. In some embodiments, the API has a solubility of less than 10 mg/ml in a solution with a pH of between about 6-8. In some embodiments, the API has a solubility of less than 1.0 mg/ml in a solution with a pH of between about 6-8. In some embodiments, the API has a solubility of less than 0.5 mg/ml in a solution with a pH of between about 6-8. In some embodiments, the API has a solubility of less than 0.1 mg/ml in a solution with a pH of between about 6-8. In some embodiments, the API has a solubility of less than 0.05 mg/ml in a solution with a pH of between about 6-8. In some embodiments, the API has a solubility of less than 0.04 mg/ml in a solution with a pH of between about 6-8. In some embodiments, the API has a solubility of less than 0.03 mg/ml in a solution with a pH of between about 6-8. In some embodiments, the API has a solubility of less than 0.02 mg/ml in a solution with a pH of between about 6-8. In some embodiments, the API has a solubility of less than 0.01 mg/ml in a solution with a pH of between about 6-8. In some embodiments, the API has a solubility of less than 0.001 mg/ml in a solution with a pH of between about 6-8. In some embodiments, the API has a low solubility at a pH of about 4-8. In some embodiments, the API has a solubility of less than 10 mg/ml in a solution with a pH of between about 4-8. In some embodiments, the API has a solubility of less than 1.0 mg/ml in a solution with a pH of between about 4-8. In some embodiments, the API has a solubility of less than 0.5 mg/ml in a solution with a pH of between about 4-8. In some embodiments, the API has a solubility of less than 0.1 mg/ml in a solution with a pH of between about 4-8. In some embodiments, the API has a solubility of less than 0.05 mg/ml in a solution with a pH of between about 4-8. In some embodiments, the API has a solubility of less than 0.04 mg/ml in a solution with a pH of between about 4-8. In some embodiments, the API has a solubility of less than 0.03 mg/ml in a solution with a pH of between about 4-8. In some embodiments, the API has a solubility of less than 0.02 mg/ml in a solution with a pH of between about 4-8. In some embodiments, the API has a solubility of less than 0.01 mg/ml in a solution with a pH of between about 4-8. In some embodiments, the API has a solubility of less than 0.001 mg/ml in a solution with a pH of between about 4-8. In some embodiments, the API has a low solubility at a pH of about 6-10. In some embodiments, the API has a solubility of less than 10 mg/ml in a solution with a pH of between about 6-10. In some embodiments, the API has a solubility of less than 1.0 mg/ml in a solution with a pH of between about 6-10. In some embodiments, the API has a solubility of less than 0.5 mg/ml in a solution with a pH of between about 6-10. In some embodiments, the API has a solubility of less than 0.1 mg/ml in a solution with a pH of between about 6-10. In some embodiments, the API has a solubility of less than 0.05 mg/ml in a solution with a pH of between about 6-10. In some embodiments, the API has a solubility of less than 0.04 mg/ml in a solution with a pH of between about 6-10. In some embodiments, the API has a solubility of less than 0.03 mg/ml in a solution with a pH of between about 6-10. In some embodiments, the API has a solubility of less than 0.02 mg/ml in a solution with a pH of between about 6-10. In some embodiments, the API has a solubility of less than 0.01 mg/ml in a solution with a pH of between about 6-10. In some embodiments, the API has a solubility of less than 0.001 mg/ml in a solution with a pH of between about 6-10. In some embodiments, the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

Disclosed herein are pharmaceutical compositions comprising an ASD that comprises an API, a hydrophilic polymer, optionally a surfactant, and optionally an adsorbent. In some embodiments, the ASD is formulated in a unit dosage form as a part of the pharmaceutical compositions, such as a capsule or a tablet. In some embodiments, the API is present in the ASD in an amount of at least 10 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, or 200 mg. In some embodiments, the API is present in the ASD in an amount of about 10 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, or 200 mg. In some embodiments, the API is present in the ASD in an amount of no more than 1000 mg, 750 mg, 500 mg, 400 mg, 300 mg, 250 mg, 225 mg, 200 mg, 175 mg, 150 mg, 125 mg, 100 mg, 90 mg, 80 mg, 75 mg, 60 mg, 55 mg, or 50 mg. In some embodiments, the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, the lipophilic API has a log P 3.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

Disclosed herein are pharmaceutical compositions comprising an API. In some embodiments, the pharmaceutical compositions are formulated in a unit dosage form, such as a capsule or a tablet. In some embodiments, the API is present in the pharmaceutical composition in an amount of 10 mg to 1000 mg. In some embodiments, the API is present in an amount of 20 mg to 500 mg. In some embodiments, the API is present in an amount of 20 mg to 400 mg. In some embodiments, the API is present in an amount of 20 mg to 300 mg. In some embodiments, the API is present in an amount of 25 mg to 250 mg. In some embodiments, the API is present in an amount of 30 mg to 200 mg. In some embodiments, the API is present in an amount of about 50 mg, about 100 mg or about 150 mg. In some embodiments, the API is present in an amount of 50 mg, 100 mg or 150 mg. In some embodiments, the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, a pharmaceutical composition provided comprises an API selected from abiraterone acetate, alectinib, pazopanib, cabozantinib, venetoclax, lurasidone, and vilazodone, or a pharmaceutically acceptable salt thereof, that is present at a dose from about 1.0 mg to about 1000 mg, including but not limited to about 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg. In some embodiments, the pharmaceutical composition is formulated in a unit dosage form, such as a capsule or a tablet. In some embodiments, the pharmaceutical composition is an ASD. In some embodiments, the ASD includes an API that is selected from abiraterone acetate, alectinib, pazopanib, cabozantinib, venetoclax, lurasidone, and vilazodone, or a pharmaceutically acceptable salt thereof, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, the ASD comprises from about 1 mg to about 500 mg of the API. In some embodiments, the ASD comprises from about 10 mg to about 400 mg of the API. In some embodiments, the ASD comprises from about 25 mg to about 200 mg of the API. In some embodiments, the ASD comprises from about 50 mg to about 150 mg of the API. In some embodiments, the ASD comprises about 75 mg to about 125 mg of the API. In some embodiments, the ASD comprises from about 75 mg to about 100 mg of the API. In some embodiments, the ASD comprises from about 100 mg to about 125 mg of the API. In some embodiments, the ASD is formulated in a unit dosage form as a part of the pharmaceutical composition, such as a capsule or a tablet. In some embodiments the API is selected from abiraterone acetate, alectinib, pazopanib, cabozantinib, venetoclax, lurasidone, and vilazodone or a pharmaceutically acceptable salt thereof. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, the API comprises about 5%, 10%, 15%, 20%, 25%, 30%, 40%, or 50% of the total weight of the composition. In some embodiments, the API is present in an amount of about 5% to about 70% of the total weight of an ASD or a pharmaceutical composition described herein. In some embodiments, the API is present in an amount of about 10% to about 60% of the total weight of an ASD or a pharmaceutical composition described herein. In some embodiments, the API is present in an amount of about 10% to about 20% of the total weight of an ASD or a pharmaceutical composition described herein. In some embodiments, the API is present in an amount of about 15% to about 25% of the total weight of an ASD or a pharmaceutical composition described herein. In some embodiments, the API is present in an amount of about 20% to about 30% of the total weight of an ASD or a pharmaceutical composition described herein. In some embodiments, the API is present in an amount of about 25% to about 40% of the total weight of an ASD or a pharmaceutical composition described herein. In some embodiments, the API is present in an amount of about 40% to about 50% of the total weight of an ASD or a pharmaceutical composition described herein. In some embodiments, the API is present in an amount of about 50% to about 70% of the total weight of an ASD or a pharmaceutical composition described herein. In some embodiments the API is abiraterone acetate, alectinib, pazopanib, cabozantinib, venetoclax, lurasidone, vilazodone, or a pharmaceutically acceptable salt thereof. In some embodiments, the API is a lipophilic API. In some embodiments the API is abiraterone acetate, alectinib, pazopanib, cabozantinib, venetoclax, lurasidone, vilazodone, or a pharmaceutically acceptable salt thereof. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, the API is present in the ASD in an amount of about 0.1% to about 99% by weight. In some embodiments, the API is present in the ASD in an amount of about 0.1% to about 1%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%. In some embodiments, the API is present in the ASD by weight of about 0.1%, about 1%, about 10%, about 20%, about 30%, about 40%, about 50%, about 60%, about 70%, about 80%, about 90%, or about 99% by weight. In some embodiments, the API is present in the ASD in an amount of at least about 0.1%, about 1%, about 10%, about 20%, about 30%, about 40%, about 50%, about 60%, about 70%, about 80%, or about 90% by weight. In some embodiments, the API is present in the ASD in an amount of at most about 1%, about 10%, about 20%, about 30%, about 40%, about 50%, about 60%, about 70%, about 80%, about 90%, or about 99% by weight. In some embodiments the API is abiraterone acetate, alectinib, pazopanib, cabozantinib, venetoclax, lurasidone, vilazodone, or a pharmaceutically acceptable salt thereof. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, described herein is an amorphous solid dispersion that comprises an API such as abiraterone acetate, alectinib, pazopanib, cabozantinib, venetoclax, lurasidone, or vilazodone, or a pharmaceutically acceptable salt thereof. In some embodiments, the amorphous solid dispersion is characterized by providing an amorphous powder X-ray diffraction pattern. In some embodiments, an API selected from abiraterone acetate, alectinib, pazopanib, cabozantinib, venetoclax, lurasidone, and vilazodone, or a pharmaceutically acceptable salt thereof, is present in the amorphous solid dispersion in an amount of about 5 wt % to about 70 wt % based on solids. In some embodiments, an API selected from abiraterone acetate, alectinib, pazopanib, cabozantinib, venetoclax, lurasidone, and vilazodone, or a pharmaceutically acceptable salt thereof is present in the amorphous solid dispersion in an amount of about 5 wt % to about 60 wt % based on solids. In some embodiments, an API selected from abiraterone acetate, alectinib, pazopanib, cabozantinib, venetoclax, lurasidone, and vilazodone, or a pharmaceutically acceptable salt thereof is present in the amorphous solid dispersion in an amount of about 10 wt % to about 50 wt % based on solids. In some embodiments, an API selected from abiraterone acetate, alectinib, pazopanib, cabozantinib, venetoclax, lurasidone, and vilazodone, or a pharmaceutically acceptable salt thereof is present in the amorphous solid dispersion in an amount of about 20 wt % to about 40 wt % based on solids. In some embodiments, an API selected from abiraterone acetate, alectinib, pazopanib, cabozantinib, venetoclax, lurasidone, and vilazodone, or a pharmaceutically acceptable salt thereof is present in the amorphous solid dispersion in an amount of about 5 wt % to about 30 wt % based on solids.

In some embodiments, an amorphous solid dispersion described herein comprises a surfactant. In some embodiments, the surfactant is selected from polymeric surfactants and phospholipids. In some embodiments, the surfactant is a polymeric non-ionic surfactant. In some embodiments, the surfactant is a polymeric ionic surfactant. In some embodiments, the surfactant comprises a block copolymer of polyethylene glycol and polypropylene glycol. In some embodiments, the surfactant comprises phospholipids or their derivatives. In some embodiments, the surfactant comprises lecithin. In some embodiments, the surfactant comprises polyvinylcaprolactame-based graft copolymer (PVAc-PVCap-PEG). In some embodiments, the surfactant comprises lecithin. In some embodiments, the surfactant comprises polyoxyl hydrogenated castor oil. In some embodiments, the surfactant comprises PEG. In some embodiments, the surfactant comprises polyoxylglycerides. In some embodiments, the surfactant comprises TPGS. In some embodiments, the surfactant comprises SLS. In some embodiments, the surfactant comprises polysorbate. In some embodiments, the polymeric non-ionic surfactant has a number average molecular weight of from about 7000 to about 10,000 Da. In some embodiments, an amorphous solid dispersion described herein comprises a surfactant. In some embodiments, an amorphous solid dispersion described herein comprises a surfactant that comprises one or more phospholipids. In some embodiments, the surfactant comprises one or more of phosphatidylcholine, phosphatidylethanolamine, phosphatidylinositol, phosphatidylserine, plasmalogen, sphingomyelin, and phosphatidic acid. In some embodiments, the one or more phospholipids comprise greater than 50%, 60%, 70%, 80%, or 90% phosphatidylcholine by weight. In some embodiments, the surfactant comprises lecithin. In some embodiments, the surfactant is present in the amorphous solid dispersion in an amount of about 5 wt % to about 70 wt % based on solids. In some embodiments, the surfactant is present in the amorphous solid dispersion in an amount of about 20 wt % to about 60 wt % based on solids. In some embodiments, the surfactant is present in the amorphous solid dispersion in an amount of about 10 wt % to about 30 wt % based on solids. In some embodiments, a weight ratio of an API selected from abiraterone acetate, alectinib, pazopanib, cabozantinib, venetoclax, lurasidone, and vilazodone, or a pharmaceutically acceptable salt thereof to the surfactant is from about 10:1 to about 1:10, or any ranges therebetween. In some embodiments, a weight ratio of an API selected from abiraterone acetate, alectinib, pazopanib, cabozantinib, venetoclax, lurasidone, and vilazodone, or a pharmaceutically acceptable salt thereof to the surfactant is from about 5:1 to about 1:4. In some embodiments, a weight ratio of an API selected from abiraterone acetate, alectinib, pazopanib, cabozantinib, venetoclax, lurasidone, and vilazodone, or a pharmaceutically acceptable salt thereof to the surfactant is from about 2:1 to about 1:2. In some embodiments, a weight ratio of the API free base or a pharmaceutically acceptable salt thereof to the surfactant is from about 1:1 to about 1:2. In some embodiments, a weight ratio of the API free base or a pharmaceutically acceptable salt thereof to the surfactant is from about 0.5:1 to about 1:3. In some embodiments, a weight ratio of the API free base or a pharmaceutically acceptable salt thereof to the surfactant is from about 1:1 to about 1:3.

In some embodiments, an ASD comprises i) an API free base or a pharmaceutically acceptable salt thereof, such as abiraterone acetate, alectinib, pazopanib, cabozantinib, venetoclax, lurasidone, or vilazodone, or a pharmaceutically acceptable salt thereof; ii) a surfactant; and iii) a hydrophilic polymer. In some embodiments, the ASD is formulated in a unit dosage form, such as a capsule or a tablet. In some embodiments, the API free base or a pharmaceutically acceptable salt thereof is present in the ASD in an amount of about 10 mg to about 500 mg. In some embodiments, the API free base or a pharmaceutically acceptable salt thereof is present in the ASD in an amount of about 20 mg to about 200 mg. In some embodiments, the API free base or a pharmaceutically acceptable salt thereof is present in the ASD in an amount of about 25 mg, about 50 mg, about 100 mg about 150 mg, or about 200 mg. In some embodiments, the surfactant is present in the ASD in an amount of about 10 mg to about 500 mg. In some embodiments, the surfactant is present in the ASD in an amount of about 20 mg to about 200 mg. In some embodiments, the hydrophilic polymer is present in the ASD in an amount of about 10 mg to about 500 mg. In some embodiments, the hydrophilic polymer is present in the ASD in an amount of about 20 mg to about 200 mg.

In some embodiments, an amorphous solid dispersion described herein comprises a hydrophilic polymer. In some embodiments, an amorphous solid dispersion described herein comprises a non-ionic or ionic hydrophilic polymer. In some embodiments, the hydrophilic polymer comprises enteric polymer. In some embodiments, an enteric polymer comprises methacrylate copolymers, hydroxypropyl methylcellulose acetate succinates or cellulose acetate phthalate. In some embodiments the hydrophilic polymer comprises phospholipids or their derivatives such as lecithin, polyethylene glycol (PEG), a block copolymer of polyethylene glycol and polypropylene glycol (e.g., sold under the trade name Poloxamer or Pluronic F-68), sodium lauryl sulfate (SLS), TPGS, polyvinyl acetate and polyvinylcaprolactame-based graft copolymer, polyoxyl hydrogenated castor oil, polyoxylglycerides (e.g., caprylocaproyl polyoxyl-8 glycerides sold under the trade name Labrasol; Lauroyl Polyoxyl-32 glycerides, sold under the trade name Gelucire), polysorbate, or a combination thereof. In some embodiments the hydrophilic polymer comprises polyvinyl alcohol (PVA), oligosaccharide, polysaccharide, polyvinylpyrrolidone (PVP), hydroxypropyl methylcellulose (HPMC, or hypromellose), hydroxyethyl cellulose (HEC), hydroxypropyl cellulose (HPC), hydropropylmethylcellulose acetate succinate (HPMCAS), polyethylene glycol (PEG), polyvinyl acetate and polyvinylcaprolactame-based graft copolymer (PVAc-PVCap-PEG), or polyvinyl caprolactam-polyvinyl acetate-polyethylene glycol (PCL-PVAc-PEG also termed Soluplus®), polyethylene oxide, cyclodextrin (CD) and its derivatives such as hydroxypropyl beta cyclodextrin (HP-β-CD), polymethacrylates (e.g., Eudragit), or a combination thereof. In some embodiments, the non-ionic hydrophilic polymer is HPMC, PVP, HP-β-CD, or PVA. In some embodiments, the ionic hydrophilic polymer is sulfobutylether-β-cyclodextrin. In some embodiments, the hydrophilic polymer comprises polymethacrylates (e.g., Eudragit). In some embodiments, the hydrophilic polymer comprises polyvinyl caprolactam-polyvinyl acetate-polyethylene glycol.

In some embodiments, a pharmaceutical composition described herein is free of organic acid. In some embodiments, the ASD is free of organic acid. In some embodiments, the pharmaceutical composition is free of any acid. In some embodiments, the ASD is free of any acid.

The formation of the amorphous solid dispersion can lead to a certain particle size for the ASD. In some embodiments, the particle size of the ASD is from about 1 nm to 1 mm. In some embodiments, the particle size of the ASD is from about 0.01 to 1000 micrometers. In some embodiments, the particle size of the ASD from about 0.01 micrometers to about 1,000 micrometers. In some embodiments, the particle size of the ASD is at least about 0.01 micrometers. In some embodiments, the particle size of the ASD is at most about 1,000 micrometers. In some embodiments, the particle size of the ASD is from about 1 micrometer to about 50 micrometers. In some embodiments, the particle size of the ASD is at least about 1 micrometer. In some embodiments, the particle size of the ASD is at most about 50 micrometers. In some embodiments, the particle size of the ASD is about 10 micrometer to about 15 micrometers. In some embodiments, the particle size of the ASD is from about 1 micrometer to about 3 micrometers, about 1 micrometer to about 7 micrometers, about 1 micrometer to about 10 micrometers, about 1 micrometer to about 13 micrometers, about 1 micrometer to about 17 micrometers, about 1 micrometer to about 20 micrometers, about 1 micrometer to about 23 micrometers, about 1 micrometer to about 27 micrometers, about 1 micrometer to about 30 micrometers, about 1 micrometer to about 40 micrometers, about 1 micrometer to about 50 micrometers, about 10 micrometers to about 13 micrometers, about 10 micrometers to about 17 micrometers, about 10 micrometers to about 20 micrometers, about 10 micrometers to about 23 micrometers, about 10 micrometers to about 27 micrometers, about 10 micrometers to about 30 micrometers, about 10 micrometers to about 40 micrometers, about 10 micrometers to about 50 micrometers, about 20 micrometers to about 27 micrometers, about 20 micrometers to about 30 micrometers, about 20 micrometers to about 40 micrometers, about 20 micrometers to about 50 micrometers, about 30 micrometers to about 40 micrometers, about 30 micrometers to about 50 micrometers, or about 40 micrometers to about 50 micrometers. In some embodiments, the particle size of the ASD is from about 1 micrometer to about 100 micrometers. In some embodiments, the particle size of the ASD is from at least about 1 micrometer. In some embodiments, the particle size of the ASD is about 0.1, 1, 3, 5, 7, 10, 13, 17, 20, 23, 25, 27, 30, 33, 35, 37, 40, 43, 45, 47, 50, 60, 70, 80, 90, or 100 micrometers or less. In some embodiments, the particle size of the ASD is about 20 micrometers or less.

In some embodiments, a distribution of amorphous solid dispersion particle sizes is obtained. In some embodiments, the terms D10, D50, and D90 are used to describe a particle size distribution. In some embodiments, the D90 particle size of the ASD is equal to or less than about 1,000 μm, 950 μm, 900 μm, 800 μm, 700 μm, 600 μm, 500 μm, 400 μm, 300 μm, 200 μm, 100 μm, 75 μm, 65 μm, 50 μm, 25 μm, 20 μm, 15 μm, or 10 μm. In some embodiments, the D50 particle size of the ASD is equal to or less than about 500 μm, 400 μm, 300 μm, 200 μm, 100 μm, 50 μm, 35 μm, 25 μm, 20 μm, 15 μm, 10 μm, or 5 μm. In some embodiments, the D10 particle size of the ASD is equal to or less than about 200 μm, 100 μm, 50 μm, 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, or 1 μm.

In some embodiments, a distribution of amorphous solid dispersion particle sizes is obtained. In some embodiments, the terms D10, D50, and D90 are used to describe a particle size distribution. In some embodiments, the D90 particle size of the ASD is about 10 μm to about 1,000 μm. In some embodiments, the D90 particle size of the ASD is about 10 μm to about 20 μm, about 10 μm to about 30 μm, about 10 μm to about 50 μm, about 10 μm to about 100 μm, about 10 μm to about 150 μm, about 10 μm to about 200 μm, about 10 μm to about 500 μm, about 10 μm to about 750 μm, about 10 μm to about 1,000 μm, about 20 μm to about 30 μm, about 20 μm to about 50 μm, about 20 μm to about 100 μm, about 20 μm to about 150 μm, about 20 μm to about 200 μm, about 50 μm to about 100 μm, about 100 μm to about 1,000 μm, about 500 μm to about 1,000 μm, or about 750 μm to about 1,000 μm. In some embodiments, the D90 particle size is at least about 10 μm, about 20 μm, about 30 μm, about 50 μm, about 100 μm, about 150 μm, about 200 μm, about 500 μm, or about 750 μm. In some embodiments, the D90 particle size is at most about 15 μm. In some embodiments, the D90 particle size is at most about 10 μm, 15 μm, 20 μm, about 30 μm, about 50 μm, about 100 μm, about 150 μm, about 200 μm, about 500 μm, or about 1,000 μm.

In some embodiments, a distribution of amorphous solid dispersion particle sizes is obtained. In some embodiments, the terms D10, D50, and D90 are used to describe a particle size distribution. In some embodiments, the D50 value of the ASD is about 1 μm to about 100 μm. In some embodiments, the D50 value of the ASD is about 10 μm to about 15 μm. In some embodiments, the D50 particle size is about 5 μm to about 10 μm, about 5 μm to about 15 μm, about 5 μm to about 20 μm, about 5 μm to about 25 μm, about 5 μm to about 30 μm, about 5 μm to about 40 μm, about 5 μm to about 50 μm, about 5 μm to about 60 μm, about 5 μm to about 75 μm, about 5 μm to about 100 μm, about 10 μm to about 15 μm, about 10 μm to about 20 μm, about 10 μm to about 25 μm, about 10 μm to about 30 μm, about 10 μm to about 40 μm, about 10 μm to about 50 μm, about 10 μm to about 60 μm, about 10 μm to about 75 μm, about 10 μm to about 100 μm, about 15 μm to about 20 μm, about 15 μm to about 25 μm, about 15 μm to about 30 μm, about 15 μm to about 40 μm, about 15 μm to about 50 μm, about 15 μm to about 60 μm, about 15 μm to about 75 μm, about 15 μm to about 100 μm, about 20 μm to about 25 μm, about 20 μm to about 30 μm, about 20 μm to about 40 μm, about 20 μm to about 50 μm, about 20 μm to about 60 μm, about 20 μm to about 75 μm, about 20 μm to about 100 μm, about 25 μm to about 30 μm, about 25 μm to about 40 μm, about 25 μm to about 50 μm, about 25 μm to about 60 μm, about 25 μm to about 75 μm, about 25 μm to about 100 μm, about 30 μm to about 40 μm, about 30 μm to about 50 μm, about 30 μm to about 60 μm, about 30 μm to about 75 μm, about 50 μm to about 100 μm, or about 75 μm to about 100 μm. In some embodiments, the D50 particle size is about 5 μm, about 10 μm, about 15 μm, about 20 μm, about 25 μm, about 30 μm, about 40 μm, about 50 μm, about 60 μm, about 75 μm, or about 100 μm. In some embodiments, the D50 particle size is at least about 0.5 μm, 5 μm, about 10 μm, about 15 μm, or about 20 μm. In some embodiments, the D50 particle size is at most about 10 μm, about 15 μm, about 20 μm, about 25 μm, about 30 μm, about 40 μm, about 50 μm, about 60 μm, about 75 μm, or about 100 μm.

In some embodiments, a distribution of amorphous solid dispersion particle sizes is obtained. In some embodiments, the terms D10, D50, and D90 are used to describe a particle size distribution. In some embodiments, the D10 value of the ASD is about 0.1 μm to about 50 m. In some embodiments, the D10 particle size is about 0.1 μm to about 1 μm, about 0.1 μm to about 2 μm, about 0.1 μm to about 3 μm, about 0.1 μm to about 4 μm, about 0.1 μm to about 5 μm, about 0.1 μm to about 7 μm, about 0.1 μm to about 10 μm, about 0.1 μm to about 20 μm, about 0.1 μm to about 30 μm, about 0.1 μm to about 40 μm, about 0.1 μm to about 50 μm, about 1 μm to about 2 μm, about 1 μm to about 3 μm, about 1 μm to about 4 μm, about 1 μm to about 5 μm, about 1 μm to about 7 μm, about 1 μm to about 10 μm, about 1 μm to about 20 μm, or 1 μm to about 50 m. In some embodiments, the D10 particle size is about 0.1 μm, about 1 μm, about 2 μm, about 3 μm, about 4 μm, about 5 μm, about 7 μm, about 10 μm, about 20 μm, about 30 μm, about 40 μm, or about 50 μm. In some embodiments, the D10 particle size is at least about 0.1 μm, about 1 μm, about 2 μm, about 3 μm, about 4 μm, about 5 μm, about 7 μm, about 10 μm, about 20 μm, about 30 μm, or about 40 m. In some embodiments, the D10 particle size is at most about 1 μm, about 2 μm, about 3 μm, about 4 μm, about 5 μm, about 7 μm, about 10 μm, about 20 μm, about 30 μm, about 40 μm, or about 50 m.

### a) Cabozantinib

In one aspect, disclosed herein are pharmaceutical compositions comprising a ASD that comprises an API, a surfactant, a hydrophilic polymer, and optionally an adsorbent. In one aspect, disclosed herein are pharmaceutical compositions comprising an ASD, wherein the ASD comprises an API. In some embodiments, the API is cabozantinib free base or a pharmaceutically acceptable salt thereof. In some embodiments, the API is cabozantinib malate.

In some embodiments, an ASD comprises cabozantinib free base or a pharmaceutically acceptable salt thereof in an amount of about 3% to about 60% by weight of the ASD. In some embodiments, the ASD comprises cabozantinib free base or a pharmaceutically acceptable salt thereof in an amount of about 10% to about 30% by weight of the ASD. In some embodiments, the ASD comprises cabozantinib free base or a pharmaceutically acceptable salt thereof in an amount of about 12% to about 25% by weight of the ASD. In some embodiments, the ASD comprises cabozantinib free base or a pharmaceutically acceptable salt thereof in an amount of about 15% to about 20% by weight of the ASD. In some embodiments, the ASD comprises cabozantinib free base or a pharmaceutically acceptable salt thereof in an amount of about 15% by weight of the ASD. In some embodiments, the ASD comprises cabozantinib free base or a pharmaceutically acceptable salt thereof in an amount of about 20% by weight of the ASD. In some embodiments, the API comprises cabozantinib malate.

In some embodiments, the cabozantinib free base or a pharmaceutically acceptable salt thereof is present in the amorphous solid dispersion in a weight percent of about 5% to about 60%. In some embodiments, the cabozantinib free base or a pharmaceutically acceptable salt thereof is present in the amorphous solid dispersion in a weight percent of about 10% to about 50%. In some embodiments, the cabozantinib free base or a pharmaceutically acceptable salt thereof is present in the amorphous solid dispersion in a weight percent of about 15% to about 30%. In some embodiments, the cabozantinib free base or a pharmaceutically acceptable salt thereof is present in the amorphous solid dispersion in a weight percent of about 25% to about 40%. In some embodiments, the cabozantinib free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the cabozantinib free base or a pharmaceutically acceptable salt thereof is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the cabozantinib free base or a pharmaceutically acceptable salt thereof is present in the amorphous solid dispersion in a weight percent of at least about 5%, about 10%, about 15%, about 20%, about 25%, about 30%, about 35%, about 40%, about 45%, about 50%, or about 55%. In some embodiments, the cabozantinib free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%. In some embodiments, the salt of cabozantinib is cabozantinib malate.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMC (such as HPMC-E5), copovidone, polymethacrylates, or a combination thereof. In some embodiments, the hydrophilic polymer comprises copovidone, polymethacrylates, polyvinyl acetate and polyvinylcaprolactame-based graft copolymer, HPMC, HPMCAS, or a combination thereof. In some embodiments, the hydrophilic polymer comprises copovidone. In some embodiments, the hydrophilic polymer comprises polymethacrylates (e.g., Eudragit). In some embodiments, the hydrophilic polymer comprises polyvinyl acetate and polyvinylcaprolactame-based graft copolymer. In some embodiments, the hydrophilic polymer comprises HPMCAS. In some embodiments, the hydrophilic polymer comprises HPMC. In some embodiments, the hydrophilic polymer is present in an amount of about 1% to about 80% by weight of the ASD. In some embodiments, the hydrophilic polymer is present in an amount of about 5% to about 70% by weight of the ASD.

In some embodiments, the ASD comprises an adsorbent. In some embodiments, the adsorbent is silicone dioxide. In some embodiments, the adsorbent is present in an amount of about 1% to about 40% by weight of the ASD.

In some embodiments, the ASD optionally comprises a surfactant. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments, the surfactant comprises lecithin, a block copolymer of polyethylene glycol and polypropylene glycol, TPGS, polyoxylglycerides (e.g., Labrasol or Gelucire), polyoxyl hydrogenated castor, or a combination thereof. In some embodiments, the surfactant is present in an amount of about 5% to about 60% by weight of the ASD. In some embodiments, the surfactant is present in an amount of about 10% to about 55% by weight of the ASD.

In some embodiments, the ASD comprises optionally an inorganic acid or organic acid. In some embodiments, the ASD comprises optionally an organic acid. In some embodiments, the organic acid is malic acid. In some embodiments, the ASD comprises an organic acid, wherein the organic acid is malic acid. In some embodiments, the inorganic acid or organic acid is present in an amount of about 1% to about 40% by weight of the ASD.

In some embodiments, an ASD comprises cabozantinib free base or a pharmaceutically acceptable salt thereof in an amount of about 10% to about 30% by weight of the ASD. In some embodiments, the ASD comprises a surfactant in an amount of about 10% to about 55% by weight of the ASD. In some embodiments, the surfactant comprises phospholipids or their derivatives such as lecithin, a block copolymer of polyethylene glycol and polypropylene glycol, polyvinylcaprolactame-based graft copolymer (PVAc-PVCap-PEG), polyoxyl hydrogenated castor oil, TPGS, or a combination thereof. In some embodiments, the ASD comprises a hydrophilic polymer in an amount of about 5% to about 70% by weight of the ASD. In some embodiments, the hydrophilic polymer is HPMC. In some embodiments, the ASD comprises optionally an adsorbent in an amount of about 5% to 40% by weight of the ASD. In some embodiments, the adsorbent is silicone dioxide. In some embodiments, the ASD comprises an organic acid in an amount of about 5% to 40% by weight of the ASD. In some embodiments, the organic acid is malic acid.

In some embodiments, the ASD comprising cabozantinib free base or a pharmaceutically acceptable salt thereof in an amount of about 12% to about 25% by weight of the ASD. In some embodiments, the ASD comprises a surfactant in an amount of about 15% to about 50% by weight of the ASD. In some embodiments, the surfactant comprises lecithin, TPGS, or a combination thereof. In some embodiments, the ASD comprises a hydrophilic polymer in an amount of about 10% to about 50% by weight of the ASD. In some embodiments, the hydrophilic polymer is HPMC. In some embodiments, the ASD comprises optionally an adsorbent in an amount of about 15% to 30% by weight of the ASD. In some embodiments, the adsorbent is silicone dioxide. In some embodiments, the ASD comprises optionally an organic acid in an amount of about 10% to 30% by weight of the ASD. In some embodiments, the organic acid is malic acid.

In some embodiments, the pharmaceutical composition described herein comprises an ASD comprising cabozantinib free base or a pharmaceutically acceptable salt thereof (such as cabozantinib malate) in an amount of about 15% by weight of the ASD. In some embodiments, the ASD comprises a surfactant in an amount of about 45% by weight of the ASD. In some embodiments, the surfactant comprises lecithin and TPGS. In some embodiments, the ASD comprises a hydrophilic polymer in an amount of about 15% by weight of the ASD. In some embodiments, the hydrophilic polymer is HPMC. In some embodiments, the ASD comprises optionally an adsorbent in an amount of about 23% by weight of the ASD. In some embodiments, the adsorbent is silicone dioxide.

In some embodiments, the pharmaceutical composition described herein comprises an ASD comprising cabozantinib free base or a pharmaceutical acceptable salt thereof. In some embodiments, the cabozantinib free base or a pharmaceutical acceptable salt thereof is in an amount of about 10% to about 55% by weight of the ASD. In some embodiments, the cabozantinib free base or a pharmaceutical acceptable salt thereof is in an amount of about 12% to about 25% by weight of the ASD. In some embodiments, the ASD comprises a surfactant. In some embodiments, the surfactant is in an amount of about 10% to about 55% by weight of the ASD. In some embodiments, the surfactant is in an amount of about 15% to about 50% by weight of the ASD. In some embodiments, the surfactant comprises lecithin, a block copolymer of polyethylene glycol and polypropylene glycol, TPGS, polyoxylglycerides, polyoxyl hydrogenated castor, or a combination thereof. In some embodiments, the ASD comprises a hydrophilic polymer. In some embodiments, the hydrophilic polymer is in an amount of about 5% to about 70% by weight of the ASD. In some embodiments, the hydrophilic polymer is in an amount of about 10% to about 50% by weight of the ASD. In some embodiments, the hydrophilic polymer is in an amount of about 15% to 30% by weight of the ASD. In some embodiments, the hydrophilic polymer is copovidone, polymethacrylates, polyvinyl acetate and polyvinylcaprolactame-based graft copolymer, HPMC, HPMCAS, or a combination thereof. In some embodiments, the ASD comprises an adsorbent. In some embodiments, the adsorbent is in an amount of about 5% to 40% by weight of the ASD. In some embodiments, the adsorbent is silicone dioxide. In some embodiments, the ASD comprises an acid, such as an organic acid. In some embodiments, the organic acid is in an amount of about 5% to 40% by weight of the ASD. In some embodiments, the organic acid is in an amount of about 10% to 30% by weight of the ASD. In some embodiments, the organic acid is malic acid.

In some embodiments, the pharmaceutical composition described herein comprises an ASD comprising cabozantinib free base or a pharmaceutically acceptable salt thereof. In some embodiments, the cabozantinib free base or a pharmaceutically acceptable salt thereof is in an amount of about 10-30% by weight of the ASD. In some embodiments, the cabozantinib free base or a pharmaceutically acceptable salt thereof is in an amount of about 10-28% by weight of the ASD. In some embodiments, the ASD comprises a hydrophilic polymer. In some embodiments, the hydrophilic polymer is in an amount of about 10% to about 60% by weight of the ASD. In some embodiments, the hydrophilic polymer is in an amount of about 15% to about 40% by weight of the ASD. In some embodiments, the hydrophilic polymer is in an amount of about 10% by weight of the ASD. In some embodiments, the hydrophilic polymer is VA64. In some embodiments, the hydrophilic polymer is L100-55. In some embodiments, the hydrophilic polymer is HPMC-E5. In some embodiments, the hydrophilic polymer is HPMCAS-LF. In some embodiments, the hydrophilic polymer is soluplus. In some embodiments, the hydrophilic polymer is VA 64, L100-55, VA64, HPMC-E5, HPMCAS-LF, or any combination thereof. In some embodiments, the ASD comprises a surfactant. In some embodiments, the surfactant is in an amount of about 10% to about 40% by weight of the ASD. In some embodiments, the surfactant is in an amount of about 15% to about 40% by weight of the ASD. In some embodiments, the surfactant is in an amount of about 20% to about 25% by weight of the ASD. In some embodiments, the surfactant is TPGS. In some embodiments, TPGS is present in an amount of about 15-30% by weight of the ASD. In some embodiments, the surfactant is lecithin. In some embodiments, lecithin is present in an amount of about 19% by weight of the ASD. In some embodiments, the ASD comprises an organic acid. In some embodiments, the organic acid is malic acid. In some embodiments, the malic acid is present in an amount of about 10% to about 20% by weight of the ASD. In some embodiments, the ASD comprises an adsorbent. In some embodiments, the adsorbent is present in an amount of about 15% to about 30% by weight of the ASD. In some embodiments, the adsorbent is SiO₂. In some embodiments, the SiO₂ is present in an amount of about 15% to 40% by weight of the ASD.

In some embodiments, the pharmaceutical composition described herein comprises an ASD that comprising cabozantinib free base or a pharmaceutically acceptable salt thereof in an amount of about 24% to 30% by weight of the ASD; a hydrophilic polymer in an amount of about 40% to 60% by weight of the ASD, and wherein the hydrophilic polymer is HPMCAS-LF or Soluplus; and a surfactant in an amount of about 15% to about 25% by weight of the ASD, wherein the surfactant is TPGS.

In some embodiments, the pharmaceutical composition described herein comprises an ASD that consists of cabozantinib free base or a pharmaceutically acceptable salt thereof in an amount of about 20% to about 25% by weight of the ASD; a hydrophilic polymer in an amount of about 20% to about 40% by weight of the ASD, wherein the hydrophilic polymer is HPMC-E5; an organic acid present in an amount of about 15% to 20% by weight of the ASD, and wherein the organic acid is malic acid; and an adsorbent in an amount of about 20% by weight of the ASD, wherein the adsorbent is SiO₂.

In some embodiments, the pharmaceutical composition described herein comprises an ASD that consists of cabozantinib free base or a pharmaceutically acceptable salt thereof in an amount of about 24% to 27% by weight of the ASD; a hydrophilic polymer in an amount of about 9% to 10% by weight of the ASD, wherein the hydrophilic polymer is VA64, L100-55, or any combination thereof; a surfactant in an amount of about 15% to 30% by weight of the ASD, wherein the surfactant is TPGS, lecithin, or any combination thereof; and an absorbent in an amount of about 18-21% by weight of the ASD, wherein the adsorbent is SiO₂.

In some embodiments, the pharmaceutical composition described herein comprises an ASD that consists of cabozantinib free base or a pharmaceutically acceptable salt thereof in an amount of about 20% to 25% by weight of the ASD; a hydrophilic polymer in an amount of about 18-20% by weight of the ASD, wherein the hydrophilic polymer is HPMC-E5; and a surfactant in an amount of about 18% to 20% by weight of the ASD, wherein the surfactant is TPGS; an organic acid in an amount of about 18-20% by weight of the ASD in which the organic acid is malic acid; and an adsorbent in an amount of about 20% by weight of the ASD, wherein the adsorbent is SiO₂.

In some embodiments, the pharmaceutical composition described herein comprising an ASD is formulated in a unit dosage form comprising cabozantinib free base or cabozantinib malate. In some embodiments, the ASD comprises cabozantinib free base or a pharmaceutically acceptable salt thereof (such as cabozantinib malate) in an amount of about 20 mg to about 80 mg. In some embodiments, the cabozantinib free base or a pharmaceutical acceptable salt thereof is in an amount of about 20 mg to about 200 mg. In some embodiments, the ASD comprises a surfactant in an amount of about 40 mg to about 220 mg. In some embodiments, the ASD comprise a surfactant in an amount of about 20 mg to about 220 mg. In some embodiments, the the surfactant phospholipids or their derivatives such as lecithin, a block copolymer of polyethylene glycol and polypropylene glycol, polyvinylcaprolactame-based graft copolymer (PVAc-PVCap-PEG), polyoxyl hydrogenated castor oil, TPGS, or a combination thereof. In some embodiments, the surfactant comprises lecithin, a block copolymer of polyethylene glycol and polypropylene glycol, TPGS, polyoxylglycerides, polyoxyl hydrogenated castor, or a combination thereof. In some embodiments, the ASD comprise a hydrophilic polymer in an amount of about 40 mg to about 250 mg. In some embodiments, the ASD comprises a hydrophilic polymer in an amount of about 40 mg to about 150 mg. In some embodiments, the hydrophilic polymer is HPMC. In some embodiments, the hydrophilic polymer is HPMC (such as HPMC-E5), copovidone, polymethacrylates, polyvinyl acetate and polyvinylcaprolactame-based graft copolymer, HPMCAS, or a combination thereof. In some embodiments, the ASD comprise an adsorbent in an amount of about 40 mg to about 200 mg. In some embodiments, the ASD comprises optionally an adsorbent in an amount of about 40 mg to about 120 mg. In some embodiments, the adsorbent is silicone dioxide. In some embodiments, the ASD comprise an organic acid in an amount of about 20 mg to about 200 mg.

In some embodiments, the organic acid is malic acid.

In some embodiments, an ASD comprises cabozantinib free base or a pharmaceutically acceptable salt thereof (such as cabozantinib malate) in an amount of about 20 mg to about 80 mg. In some embodiments, the ASD comprises a surfactant in an amount of about 50 mg to about 200 mg. In some embodiments, the the surfactant lecithin. In some embodiments, the ASD comprises a block copolymer of polyethylene glycol and polypropylene glycol, TPGS, or a combination thereof, a hydrophilic polymer in an amount of about 50 mg to about 130 mg. In some embodiments, the hydrophilic polymer is HPMC. In some embodiments, the ASD comprises an adsorbent in an amount of about 50 mg to about 100 mg. In some embodiments, the adsorbent is silicone dioxide. In some embodiments, the ASD comprises an organic acid in an amount of about 20 mg to about 70 mg. In some embodiments, the organic acid is malic acid.

In some embodiments, pharmaceutical compositions comprising an ASD that comprises cabozantinib free base or a pharmaceutically acceptable salt thereof (such as cabozantinib malate) have acceptable storage stability. In some embodiments, the pharmaceutical composition is chemically stable for at least 2 weeks at 75° C./75% RH, wherein a storage stable pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline cabozantinib or a salt thereof, when measured as AUC, AUC_{inf}, or AUCₗₐₛₜ after oral administration. In some embodiments, the pharmaceutical composition exhibits a bioavailability that is from about 75% to about 200% of a bioavailability of a corresponding reference composition comprising cabozantinib, when measured as Cₘₐₓ or AUCₗₐₛₜ after oral administration under fasted condition, wherein the reference pharmaceutical composition does not comprise an amorphous solid dispersion. In some embodiments, the pharmaceutical composition exhibits a bioavailability that is from about 100% to about 150% of a bioavailability of a corresponding reference composition comprising cabozantinib, when measured as Cₘₐₓ or AUCₗₐₛₜ after oral administration under fasted condition, wherein the reference pharmaceutical composition does not comprise an amorphous solid dispersion. In some embodiments, the pharmaceutical composition exhibits a bioavailability that is from about 150% to about 200% of a bioavailability of a corresponding reference composition comprising cabozantinib, when measured as Cₘₐₓ or AUCₗₐₛₜ after oral administration under fasted condition, wherein the reference pharmaceutical composition does not comprise an amorphous solid dispersion. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline cabozantinib or salt thereof, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline cabozantinib or a salt thereof, when measured as Cₘₐₓ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of COMETRIQ capsule comprising cabozantinib or a salt thereof, when measured as AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of COMETRIQ capsule comprising cabozantinib or a salt thereof, when measured as Cₘₐₓ after oral administration. In some embodiments, the pharmaceutical composition exhibits a bioavailability that is higher than a bioavailability of COMETRIQ by about 1.1 fold to about 10 fold. In some embodiments, the pharmaceutical composition exhibits a bioavailability that is higher than a bioavailability of COMETRIQ by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical compositions comprise an ASD comprising cabozantinib or a pharmaceutically acceptable salt thereof (such as cabozantinib malate). In some embodiment, the bioavailability is measured under fed condition. In some embodiment, the bioavailability is measured under fasted condition.

In some embodiments, the pharmaceutical composition described herein exhibits a bioavailability that is higher than a bioavailability of COMETRIQ by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUC, AUCinf, or AUCₗₐₛₜ or Cₘₐₓ after oral administration. In some embodiments, the pharmaceutical composition exhibits a bioavailability that is higher than a bioavailability of COMETRIQ by at least about 2 fold. In some embodiments, the pharmaceutical composition exhibits a bioavailability that is higher than a bioavailability of COMETRIQ by at least about 4 fold. In some embodiments, the pharmaceutical composition exhibits a bioavailability that is higher than a bioavailability of COMETRIQ by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUC, AUCinf, or AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiments, the pharmaceutical compositions comprise an ASD comprising cabozantinib or a pharmaceutically acceptable salt thereof (such as cabozantinib malate). In some embodiment, the bioavailability is measured under fed condition. In some embodiment, the bioavailability is measured under fasted condition.

In some embodiments, salts of compounds of cabozantinib are formed, for example, as acid addition salts (e.g., with organic or inorganic acids), from compounds of cabozantinib with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### b) Venetoclax

In one aspect, disclosed herein are pharmaceutical compositions comprising an ASD that comprises an API, a surfactant, a hydrophilic polymer, optionally an inorganic acid or organic acid, and optionally an adsorbent. In one aspect, disclosed herein are pharmaceutical compositions comprising an ASD, wherein the ASD comprises an API. In some embodiments, the API is venetoclax free base (i.e., venetoclax) or a pharmaceutically acceptable salt thereof. In some embodiments, the API is a pharmaceutically acceptable salt of venetoclax.

In some embodiments, the ASD comprises venetoclax free base or a pharmaceutically acceptable salt thereof in an amount of about 3% to about 60% by weight of the ASD. In some embodiments, the ASD comprises venetoclax free base or a pharmaceutically acceptable salt thereof in an amount of about 5% to about 45% by weight of the ASD. In some embodiments, the ASD comprises venetoclax free base or a pharmaceutically acceptable salt thereof in an amount of about 10% to about 40% by weight of the ASD. In some embodiments, the ASD comprises venetoclax free base or a pharmaceutically acceptable salt thereof in an amount of about 15% to about 35% by weight of the ASD. In some embodiments, the ASD comprises venetoclax free base or a pharmaceutically acceptable salt thereof in an amount of about 31% by weight of the ASD.

In some embodiments, the venetoclax free base or a pharmaceutically acceptable salt thereof is present in the amorphous solid dispersion in a weight percent of about 5% to about 60%. In some embodiments, the venetoclax free base or a pharmaceutically acceptable salt thereof is present in the amorphous solid dispersion in a weight percent of about 10% to about 50%. In some embodiments, the venetoclax free base or a pharmaceutically acceptable salt thereof is present in the amorphous solid dispersion in a weight percent of about 15% to about 30%. In some embodiments, the venetoclax free base or a pharmaceutically acceptable salt thereof is present in the amorphous solid dispersion in a weight percent of about 25% to about 40%. In some embodiments, the venetoclax free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the venetoclax free base or a pharmaceutically acceptable salt thereof is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the venetoclax free base or a pharmaceutically acceptable salt thereof is present in the amorphous solid dispersion in a weight percent of at least about 5%, about 10%, about 15%, about 20%, about 25%, about 30%, about 35%, about 40%, about 45%, about 50%, or about 55%. In some embodiments, the venetoclax free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a surfactant. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments, the surfactant is present in an amount of about 5% to about 60% by weight of the ASD.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the hydrophilic polymer is copovidone. In some embodiments, the hydrophilic polymer is HPMCAS. In some embodiments, the hydrophilic polymer is polyvinyl acetate and polyvinylcaprolactame-based graft copolymer (PVAc-PVCap-PEG). In some embodiments, the hydrophilic polymer is polymethacrylates (e.g., Eudragit). In some embodiments, the pharmaceutical composition described herein comprises the ASD comprising a hydrophilic polymer, wherein the hydrophilic polymer is VA64. In some embodiments, the hydrophilic polymer is present in an amount of about 1% to about 80% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD comprises an inorganic acid or organic acid. In some embodiments, the ASD comprises an organic acid. In some embodiments, the organic acid is citric acid. In some embodiments, the inorganic acid or organic acid is present in an amount of about 1% to about 40% by weight of the ASD. In some embodiments, the inorganic acid or organic acid is present in an amount of about 1% to about 30% by weight of the ASD. In some embodiments, the inorganic acid or organic acid is present in an amount of about 3% to about 20% by weight of the ASD.

In some embodiments, the ASD optionally comprises an adsorbent. In some embodiments, the adsorbent is in an amount of about 1% to 20% by weight of the ASD. In some embodiments, the adsorbent is in an amount of about 1% to 40% by weight of the ASD. In some embodiments, the adsorbent is in an amount of about 10% to 40% by weight of the ASD. In some embodiments, the adsorbent is silicone dioxide.

In some embodiments, an ASD comprising venetoclax free base or a pharmaceutically acceptable salt thereof. In some embodiments, the venetoclax free base or a pharmaceutically acceptable salt thereof is in an amount of about 10% to about 40% by weight of the ASD. In some embodiments, the ASD comprises a surfactant. In some embodiments, the surfactant is in an amount of about 10% to about 50% by weight of the ASD. In some embodiments, the surfactant comprises lecithin, TPGS, or a combination thereof. In some embodiments, the ASD comprises a hydrophilic polymer. In some embodiments, the hydrophilic polymer is in an amount of about 10% to about 50% by weight of the ASD. In some embodiments, the hydrophilic polymer is copovidone, HPMCAS, or a combination thereof. In some embodiments, the ASD comprises an organic acid. In some embodiments, the organic acid is in an amount of about 1% to 20% by weight of the ASD. In some embodiments, the organic acid is citric acid. In some embodiments, the ASD comprises an adsorbent. In some embodiments, the adsorbent is silicon dioxide.

In some embodiments, the ASD comprising venetoclax free base or a pharmaceutically acceptable salt thereof. In some embodiments, the venetoclax free base or a pharmaceutically acceptable salt thereof is in an amount of about 20% to about 40% by weight of the ASD. In some embodiments, the ASD comprises a surfactant in an amount of about 20% to about 40% by weight of the ASD. In some embodiments, the surfactant comprises lecithin, TPGS, or a combination thereof. In some embodiments, the ASD comprises a hydrophilic polymer in an amount of about 20% to about 40% by weight of the ASD. In some embodiments, the hydrophilic polymer is copovidone, HPMCAS, or a combination thereof. In some embodiments, the ASD comprises an organic acid in an amount of about 3% to 15% by weight of the ASD. In some embodiments, the organic acid is citric acid.

In some embodiments, the pharmaceutical composition described herein comprises an ASD comprising venetoclax free base or a pharmaceutically acceptable salt thereof. In some embodiments, the venetoclax free base or a pharmaceutically acceptable salt thereof is in an amount of about 31% by weight of the ASD. In some embodiments, the ASD comprises a surfactant in an amount of about 31% by weight of the ASD. In some embodiments, the ASD comprises a hydrophilic polymer in an amount of about 31% by weight of the ASD. In some embodiments, the hydrophilic polymer is copovidone. In some embodiments, the ASD comprises an organic acid in an amount of about 7% by weight of the ASD. In some embodiments, the organic acid is citric acid.

In some embodiments, the pharmaceutical composition described herein comprises an ASD comprising venetoclax or a pharmaceutically acceptable salt thereof in an amount of about 10% to about 50% by weight of the ASD. In some embodiments, the venetoclax or a pharmaceutically acceptable salt thereof is in an amount of about 20% to about 50% by weight of the ASD. In some embodiments, the ASD comprises a surfactant. In some embodiments, the surfactant is in an amount of about 10% to about 50% by weight of the ASD. In some embodiments, the surfactant is in an amount of about 20% to about 40% by weight of the ASD. In some embodiments, the surfactant comprises lecithin, a block copolymer of polyethylene glycol and polypropylene glycol, TPGS, polyoxyl hydrogenated castor oil, polyoxylglycerides (e.g., Labrasol or Gelucire), polysorbate, or a combination thereof. In some embodiments, the ASD comprises a hydrophilic polymer. In some embodiments, the hydrophilic polymer is in an amount of about 10% to about 50% by weight of the ASD. In some embodiments, the hydrophilic polymer is in an amount of about 20% to about 40% by weight of the ASD. In some embodiments, the hydrophilic polymer is copovidone, polyvinyl acetate and polyvinylcaprolactame-based graft copolymer (PVAc-PVCap-PEG), polymethacrylates, HPMCAS, or a combination thereof. In some embodiments, the ASD comprises an organic acid. In some embodiments, the organic acid is in an amount of about 1% to 20% by weight of the ASD. In some embodiments, the organic acid is in an amount of about 3% to 20% by weight of the ASD. In some embodiments, the organic acid is citric acid. In some embodiments, the ASD comprises an adsorbent. In some embodiments, the adsorbent is in an amount of about 5% to 40% by weight of the ASD. In some embodiments, the adsorbent is in an amount of about 10% to 40% by weight of the ASD. In some embodiments, the adsorbent is silicone dioxide.

In some embodiments, the pharmaceutical composition described herein comprises an ASD comprising venetoclax free base or a pharmaceutically acceptable salt thereof. In some embodiments, the venetoclax free base or a pharmaceutically acceptable salt thereof is in an amount of about 10-30% by weight of the ASD. In some embodiments, the venetoclax free base or a pharmaceutically acceptable salt thereof is in an amount of about 10-30% by weight of the ASD. In some embodiments, the venetoclax free base or a pharmaceutically acceptable salt thereof is in an amount of about 15-25% by weight of the ASD. In some embodiments, the ASD comprises a hydrophilic polymer. In some embodiments, the hydrophilic polymer is in an amount of about 10% to 60% by weight of the ASD. In some embodiments, the hydrophilic polymer is in an amount of about 18% by weight of the ASD. In some embodiments, the hydrophilic polymer is in an amount of about 20% by weight of the ASD. In some embodiments, the hydrophilic polymer is L100. In some embodiments, the hydrophilic polymer is VA64. In some embodiments, the ASD comprises a surfactant. In some embodiments, the surfactant is in an amount of about 10% to about 40% by weight of the ASD. In some embodiments, the surfactant is in an amount of about 18% by weight of the ASD. In some embodiments, the surfactant is in an amount of about 20% by weight of the ASD. In some embodiments, the surfactant is TPGS. In some embodiments, TPGS is present in an amount of about 18-20% by weight of the ASD. In some embodiments, the ASD comprises an organic acid. In some embodiments, the organic acid is citric acid. In some embodiments, the citric acid is present in an amount of about 10% to 20% by weight of the ASD. In some embodiments, the ASD comprises an adsorbent. In some embodiments, the adsorbent is present in an amount of about 25% to about 40% by weight of the ASD. In some embodiments, the adsorbent is SiO₂. In some embodiments, the SiO₂ is present in an amount of about 27-35% by weight of the ASD.

In some embodiments, the pharmaceutical composition described herein comprises an ASD that comprising venetoclax free base or a pharmaceutically acceptable salt thereof in an amount of about 24% by weight of the ASD; a hydrophilic polymer in an amount of about 24% by weight of the ASD, and wherein the hydrophilic polymer is L100; a surfactant in an amount of about 18% by weight of the ASD, wherein the surfactant is TPGS; and an absorbent in an amount of about 36% by weight of the ASD, wherein the adsorbent is SiO₂.

In some embodiments, the pharmaceutical composition described herein comprises an ASD that consists of venetoclax free base or a pharmaceutically acceptable salt thereof in an amount of about 18% to about 20% by weight of the ASD; a hydrophilic polymer in an amount of about 18% to about 20% by weight of the ASD, wherein the hydrophilic polymer is VA64; a surfactant in an amount of about 18% to 20% by weight of the ASD, wherein the surfactant is TPGS; an organic acid present in an amount of about 10% to 18% by weight of the ASD, and wherein the organic acid is citric acid; and an adsorbent in an amount of about 27% to 30% by weight of the ASD, wherein the adsorbent is SiO₂.

In some embodiments, the pharmaceutical composition described herein comprising an ASD is formulated in a unit dosage form comprising venetoclax or a pharmaceutically acceptable salt thereof in an amount of about 60 mg to about 300 mg. In some embodiments, the venetoclax or a pharmaceutically acceptable salt thereof is in an amount of about 80 mg to about 120 mg. In some embodiments, the ASD comprises a surfactant. In some embodiments, the surfactant is in an amount of about 20 mg to about 450 mg. In some embodiments, the surfactant is in an amount of about 70 mg to about 130 mg. In some embodiments, the surfactant is TPGS, lecithin, polyoxyl hydrogenated castor oil, polyoxylglycerides, polysorbate, or a combination thereof. In some embodiments, the ASD comprises a hydrophilic polymer. In some embodiments, the hydrophilic polymer is in an amount of about 50 mg to about 450 mg. In some embodiments, the hydrophilic polymer is in an amount of about 70 mg to about 130 mg. In some embodiments, the hydrophilic polymer is copovidone, polymethacrylates, polyvinyl acetate and polyvinylcaprolactame-based graft copolymer (PVAc-PVCap-PEG), HPMCAS, or a combination thereof. In some embodiments, the ASD comprises an organic acid. In some embodiments, the organic acid is in an amount of about 5 mg to about 150 mg. In some embodiments, the organic acid is in an amount of about 10 mg to about 40 mg. In some embodiments, the organic acid is citric acid. In some embodiments, the ASD comprises an adsorbent. In some embodiments, the adsorbent is in an amount of about 10 mg to about 300 mg. In some embodiments, the adsorbent is in an amount of about 50 mg to about 180 mg. In some embodiments, the adsorbent is silicone dioxide.

In some embodiments, an ASD comprises venetoclax free base or a pharmaceutically acceptable salt thereof. In some embodiments, the venetoclax free base or a pharmaceutically acceptable salt thereof is in an amount of about 60 mg to about 140 mg. In some embodiments, the ASD comprises a surfactant. In some embodiments, the surfactant is amount of about 50 mg to about 150 mg. In some embodiments, the surfactant is TPGS. In some embodiments, the ASD comprises a hydrophilic polymer in an amount of about 50 mg to about 150 mg. In some embodiments, the hydrophilic polymer is copovidone, HPMCAS, or a combination of both. In some embodiments, the ASD comprises an organic acid in an amount of about 5 mg to about 50 mg. In some embodiments, the organic acid is citric acid.

In some embodiments, an ASD comprises venetoclax free base or a pharmaceutically acceptable salt thereof. In some embodiments, the venetoclax free base or a pharmaceutically acceptable salt thereof is in an amount of about 80 mg to about 120 mg. In some embodiments, the ASD comprises a surfactant. In some embodiments, the surfactant is in an amount of about 70 mg to about 130 mg. In some embodiments, the surfactant is TPGS. In some embodiments, the ASD comprises a hydrophilic polymer in an amount of about 70 mg to about 130 mg. In some embodiments, the hydrophilic polymer is copovidone, HPMCAS, or a combination of both. In some embodiments, the ASD comprises an organic acid in an amount of about 10 mg to about 40 mg. In some embodiments, the organic acid is citric acid.

In some embodiments, pharmaceutical compositions comprising an ASD that comprises venetoclax free base or a pharmaceutically acceptable salt thereof have acceptable storage stability. In some embodiments, the pharmaceutical composition is storage stable chemically for at least 2 weeks at 75° C./75% RH, wherein a storage stable pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline a pharmaceutically acceptable salt thereof, when measured as AUC, AUC_{inf}, or AUCₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline a pharmaceutically acceptable salt thereof, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline a pharmaceutically acceptable salt thereof, when measured as Cₘₐₓ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VENCLEXTA capsule comprising a pharmaceutically acceptable salt thereof, when measured as AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VENCLEXTA capsule comprising a pharmaceutically acceptable salt thereof, when measured as Cₘₐₓ after oral administration. In some embodiments, the pharmaceutical composition exhibits a bioavailability that is higher than a bioavailability of VENCLEXTA by about 1.1 fold to about 10 fold. In some embodiments, the pharmaceutical composition exhibits a bioavailability that is higher than a bioavailability of VENCLEXTA by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical composition comprises an ASD comprising venetoclax or a pharmaceutically acceptable salt thereof. In some embodiment, the bioavailability is measured under fed condition. In some embodiment, the bioavailability is measured under fasted condition.

In some embodiments, the pharmaceutical composition exhibits a bioavailability that is from about 75% to about 200% of a bioavailability of a corresponding reference composition comprising venetoclax, when measured as Cₘₐₓ or AUCₗₐₛₜ after oral administration under fasted condition, wherein the reference pharmaceutical composition does not comprise an amorphous solid dispersion. In some embodiments, the pharmaceutical composition exhibits a bioavailability that is from about 125% to about 200% of a bioavailability of a corresponding reference composition comprising venetoclax, when measured as Cₘₐₓ or AUCₗₐₛₜ after oral administration under fasted condition, wherein the reference pharmaceutical composition does not comprise an amorphous solid dispersion. In some embodiments, the pharmaceutical composition exhibits a bioavailability that is from about 150% to about 200% of a bioavailability of a corresponding reference composition comprising venetoclax, when measured as Cₘₐₓ or AUCₗₐₛₜ after oral administration under fasted condition, wherein the reference pharmaceutical composition does not comprise an amorphous solid dispersion. In some embodiments, the pharmaceutical composition described herein exhibits a bioavailability that is higher than a bioavailability of VENCLEXTA by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUC, AUCinf, or AUCₗₐₛₜ or Cₘₐₓ after oral administration. In some embodiments, the pharmaceutical composition exhibits a bioavailability that is higher than a bioavailability of VENCLEXTA by at least about 2 fold. In some embodiments, the pharmaceutical composition exhibits a bioavailability that is higher than a bioavailability of VENCLEXTA by at least about 4 fold. In some embodiments, the pharmaceutical composition exhibits a bioavailability that is higher than a bioavailability of VENCLEXTA by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUC, AUCinf, or AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiments, the pharmaceutical composition comprises an ASD comprising venetoclax or a pharmaceutically acceptable salt thereof. In some embodiment, the bioavailability is measured under fed condition. In some embodiment, the bioavailability is measured under fasted condition.

In some embodiments, salts of compounds of venetoclax are formed, for example, as acid addition salts (e.g., with organic or inorganic acids), from compounds of venetoclax with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### c) Abiraterone Acetate

In one aspect, disclosed herein are pharmaceutical compositions comprising a ASD that comprises an API, a hydrophilic polymer, optionally a surfactant, optionally an inorganic acid or organic acid, and optionally an adsorbent. In one aspect, disclosed herein are pharmaceutical compositions comprising an ASD, wherein the ASD comprises an API. In some embodiments, the API is abiraterone free base or a pharmaceutically acceptable salt thereof. In some embodiments, the API is abiraterone acetate.

In some embodiments, the ASD comprises abiraterone free base or a pharmaceutically acceptable salt thereof (such as abiraterone acetate) in an amount of about 3% to about 60% by weight of the ASD. In some embodiments, the abiraterone free base or a pharmaceutically acceptable salt thereof is present in the amorphous solid dispersion in a weight percent of about 10% to about 50%. In some embodiments, the abiraterone free base or a pharmaceutically acceptable salt thereof is present in the amorphous solid dispersion in a weight percent of about 15% to about 30%. In some embodiments, the abiraterone free base or a pharmaceutically acceptable salt thereof is present in the amorphous solid dispersion in a weight percent of about 25% to about 40%. In some embodiments, the ASD comprises abiraterone free base or a pharmaceutically acceptable salt thereof (such as abiraterone acetate) in an amount of about 3% to about 30% by weight of the ASD. In some embodiments, the ASD comprises abiraterone free base or a pharmaceutically acceptable salt thereof (such as abiraterone acetate) in an amount of about 5% to about 25% by weight of the ASD. In some embodiments, the ASD comprises abiraterone free base or a pharmaceutically acceptable salt thereof (such as abiraterone acetate) in an amount of about 10% to about 20% by weight of the ASD. In some embodiments, the ASD comprises abiraterone free base or a pharmaceutically acceptable salt thereof (such as abiraterone acetate) in an amount of about 10% by weight of the ASD. In some embodiments, the ASD comprises abiraterone free base or abiraterone acetate in an amount of about 20% by weight of the ASD.

In some embodiments, the abiraterone free base or a pharmaceutically acceptable salt thereof (such as abiraterone acetate) is present in the amorphous solid dispersion in a weight percent of about 5% to about 60%. In some embodiments, the abiraterone free base or abiraterone acetate is present in the amorphous solid dispersion in an amount of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60% by weight. In some embodiments, the abiraterone free base or a pharmaceutically acceptable salt thereof (such as abiraterone acetate) is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the abiraterone free base or abiraterone acetate is present in the amorphous solid dispersion in a weight percent of at least about 5%, about 10%, about 15%, about 20%, about 25%, about 30%, about 35%, about 40%, about 45%, about 50%, or about 55%. In some embodiments, the abiraterone free base or abiraterone acetate is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMCAS. In some embodiments, the hydrophilic polymer is polymethacrylates. In some embodiments, the hydrophilic polymer is copovidone. In some embodiments, the hydrophilic polymer is HPMCAS, polymethacrylates, or copovidone or a combination thereof. In some embodiments, the hydrophilic polymer comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides (e.g., Labrasol or Gelucire), polysorbate, or a combination thereof. In some embodiments, the hydrophilic polymer is present in an amount of about 1% to about 80% by weight of the ASD. In some embodiments, the hydrophilic polymer is present in an amount of about 5% to about 70% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD optionally comprises a surfactant In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments, the surfactant is present in an amount of about 5% to about 50% by weight of the ASD. In some embodiments, the surfactant is present in an amount of about 5% to about 40% by weight of the ASD. In some embodiments, the surfactant is present in an amount of about 5% to about 30% by weight of the ASD.

In some embodiments, the ASD comprises optionally an adsorbent. In some embodiments, the adsorbent is silicone dioxide. In some embodiments, the adsorbent is present in an amount of about 1% to about 50% by weight of the ASD. In some embodiments, the adsorbent is present in an amount of about 1% to about 40% by weight of the ASD.

In some embodiments, the ASD comprises optionally an inorganic acid or organic acid. In some embodiments, the ASD comprises optionally an organic acid. In some embodiments, the ASD comprises an organic acid. In some embodiments, the organic acid is malic acid. In some embodiments, the organic acid is a fatty acid, such as oleic acid. In some embodiments, the organic acid is tartaric acid. In some embodiments, the organic acid is malic acid, tartaric acid, oleic acid, or a combination thereof. In some embodiments, the inorganic acid or organic acid is present in an amount of about 1% to about 40% by weight of the ASD. In some embodiments, the inorganic acid or organic acid is present in an amount of about 1% to about 30% by weight of the ASD. In some embodiments, the inorganic acid or organic acid is present in an amount of about 1% to about 20% by weight of the ASD.

In some embodiments, the ASD comprises an adsorbent. In some embodiments, the adsorbent is SiO₂. In some embodiments, the adsorbent is magnesium aluminum silicate. In some embodiments, the adsorbent is silicon dioxide or magnesium aluminum silicate or a combination thereof. In some embodiments, the adsorbent is present in an amount of about 10% to about 40% by weight of the ASD. In some embodiments, the adsorbent is present in an amount of about 1% to about 30% by weight of the ASD. In some embodiments, the adsorbent is present in an amount of about 1% to about 20% by weight of the ASD.

In some embodiments, an ASD comprises abiraterone free base or a pharmaceutically acceptable salt thereof (such as abiraterone acetate). In some embodiments, the abiraterone free base or a pharmaceutically acceptable salt thereof is in an amount of about 5% to about 40% by weight of the ASD. In some embodiments, the ASD comprises a hydrophilic polymer. In some embodiments, the hydrophilic polymer is in an amount of about 30% to about 95% by weight of the ASD. In some embodiments, the hydrophilic polymer is in an amount of about 30% to about 90% by weight of the ASD. In some embodiments, the hydrophilic polymer is HPMCAS, polymethacrylates, or copovidone or a combination thereof. In some embodiments, the ASD comprises a surfactant. In some embodiments, the surfactant is in an amount of about 10% to about 40% by weight of the ASD. In some embodiments, the surfactant is lecithin or TPGS or a combination thereof. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof.

In some embodiments, the ASD comprising abiraterone free base or a pharmaceutically acceptable salt thereof (such as abiraterone acetate). In some embodiments, the abiraterone free base or a pharmaceutically acceptable salt thereof is in an amount of about 8% to about 30% by weight of the ASD. In some embodiments, the ASD comprises a hydrophilic polymer. In some embodiments, the hydrophilic polymer is in an amount of about 50% to about 91% by weight of the ASD. In some embodiments, the hydrophilic polymer is in an amount of about 30% to about 90% by weight of the ASD. In some embodiments, the hydrophilic polymer is HPMCAS, polymethacrylates, or copovidone or a combination thereof. In some embodiments, the ASD comprises a surfactant. In some embodiments, the surfactant is in an amount of about 15% to about 30% by weight of the ASD. In some embodiments, the surfactant is lecithin or TPGS or a combination thereof. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof.

In some embodiments, the pharmaceutical composition described herein comprises an ASD comprising abiraterone free base or a pharmaceutically acceptable salt thereof (such as abiraterone acetate) in an amount of about 10% by weight of the ASD. In some embodiments, the ASD comprises a hydrophilic polymer in an amount of about 90% by weight of the ASD. In some embodiments, the hydrophilic polymer is HPMCAS, polymethacrylates, or copovidone or a combination thereof. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof.

In some embodiments, the pharmaceutical composition described herein comprises an ASD comprising abiraterone acetate or a pharmaceutically acceptable salt thereof. In some embodiments, the abiraterone acetate or a pharmaceutically acceptable salt thereof is in an amount of about 5% to about 50% by weight of the ASD. In some embodiments, the abiraterone acetate or a pharmaceutically acceptable salt thereof is in an amount of about 10-30% by weight of the ASD. In some embodiments, the abiraterone acetate or a pharmaceutically acceptable salt thereof is in an amount of about 15-20% by weight of the ASD. In some embodiments, the abiraterone acetate or a pharmaceutically acceptable salt thereof is in an amount of about 16% by weight of the ASD. In some embodiments, the ASD comprises a hydrophilic polymer. In some embodiments, the hydrophilic polymer is in an amount of about 5% to about 95% by weight of the ASD. In some embodiments, the hydrophilic polymer is in an amount of about 10% to about 95% by weight of the ASD. In some embodiments, the hydrophilic polymer is in an amount of about 10% to about 95% by weight of the ASD. In some embodiments, the hydrophilic polymer is in an amount of about 30% to about 80% by weight of the ASD. In some embodiments, the hydrophilic polymer is in an amount of about 50% to about 80% by weight of the ASD. In some embodiments, the hydrophilic polymer is in an amount of about 10% to about 60% by weight of the ASD. In some embodiments, the hydrophilic polymer is in an amount of about 38% by weight of the ASD. In some embodiments, the hydrophilic polymer is HPMC. In some embodiments, the hydrophilic polymer is HPMCAS. In some embodiments, the hydrophilic polymer is polyvinyl acetate. In some embodiments, the hydrophilic polymer is polyvinylcaprolactame-based graft copolymer. In some embodiments, the hydrophilic polymer is PVP. In some embodiments, the hydrophilic polymer is copovidone. In some embodiments, the hydrophilic polymer is HPMC, HPMCAS, polyvinyl acetate, polyvinylcaprolactame-based graft copolymer, PVP, copovidone, or a combination thereof. In some embodiments, the hydrophilic polymer is VA64. In some embodiments, the hydrophilic polymer is HPMCAS-LF. In some embodiments, the ASD comprises a surfactant. In some embodiments, the surfactant is in an amount of about 10% to about 40% by weight of the ASD. In some embodiments, the surfactant is in an amount of about 10% to about 30% by weight of the ASD. In some embodiments, the surfactant is in an amount of about 20% by weight of the ASD. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant is TPGS or lecithin or a combination thereof. In some embodiments, TPGS is present in an amount of about 10% by weight of the ASD. In some embodiments, lecithin is present in an amount of about 15% to about 20% by weight of the ASD. In some embodiments, the ASD comprises an organic acid. In some embodiments, the organic acid is present in an amount of about 10% to about 35% by weight of the ASD. In some embodiments, the organic acid is present in an amount of about 10% to about 30% by weight of the ASD. In some embodiments, the organic acid is present in an amount of about 10% to about 20% by weight of the ASD. In some embodiments, the organic acid is tartaric acid. In some embodiments, the tartaric acid is present in an amount of about 19% by weight of the ASD. In some embodiments, the organic acid is oleic acid. In some embodiments, the oleic acid is present in an amount of about 13% by weight of the ASD. In some embodiments, the ASD comprises an adsorbent. In some embodiments, the adsorbent is present in an amount of about 15% to about 40% by weight of the ASD. In some embodiments, the adsorbent is present in an amount of about 20% to about 40% by weight of the ASD. In some embodiments, the adsorbent is SiO₂. In some embodiments, the SiO₂ is present in an amount of about 27% by weight of the ASD. In some embodiments, the adsorbent is magnesium aluminum silicate. In some embodiments, the magnesium aluminum silicate is present in an amount of about 38% by weight of the ASD.

In some embodiments, the pharmaceutical composition described herein comprises an ASD that comprising abiraterone acetate or a pharmaceutically acceptable salt thereof in an amount of about 9% by weight of the ASD; a hydrophilic polymer in an amount of about 91% by weight of the ASD, and wherein the hydrophilic polymer is HPMCAS.

In some embodiments, the pharmaceutical composition described herein comprises an ASD that consists of abiraterone acetate or a pharmaceutically acceptable salt thereof in an amount of about 10% to about 30% by weight of the ASD; a hydrophilic polymer in an amount of about 30% to about 60% by weight of the ASD, wherein the hydrophilic polymer is HPMCAS; and a surfactant in an amount of about 10% to about 30% by weight of the ASD, wherein the surfactant is lecithin.

In some embodiments, the pharmaceutical composition described herein comprises an ASD that consists of abiraterone acetate or a pharmaceutically acceptable salt thereof in an amount of about 13% by weight of the ASD; a hydrophilic polymer in an amount of about 38% by weight of the ASD, wherein the hydrophilic polymer is HPMCAS; oleic acid in an amount of about 13% by weight of the ASD; and an adsorbent in an amount of about 38% by weight of the ASD, wherein the adsorbent is magnesium aluminum silicate.

In some embodiments, the pharmaceutical composition described herein comprises an ASD that consists of abiraterone acetate or a pharmaceutically acceptable salt thereof in an amount of about 18% by weight of the ASD; a hydrophilic polymer in an amount of about 18% by weight of the ASD, wherein the hydrophilic polymer is VA64; and a surfactant in an amount of about 9% by weight of the ASD, wherein the surfactant is lecithin, TPGS, or a combination thereof; tartaric acid in an amount of about 18% by weight of the ASD; and an adsorbent in an amount of about 27% by weight of the ASD, wherein the adsorbent is SiO₂.

In some embodiments, the pharmaceutical composition described herein comprises an ASD comprising abiraterone free base or a pharmaceutically acceptable salt thereof (such as abiraterone acetate). in an amount of about 20% by weight of the ASD. In some embodiments, the ASD comprises a hydrophilic polymer in an amount of about 60% by weight of the ASD. In some embodiments, the hydrophilic polymer is HPMCAS, polymethacrylates, or copovidone or a combination thereof. In some embodiments, the ASD comprises a surfactant in an amount of about 20% by weight of the ASD. In some embodiments, the surfactant is lecithin or TPGS or a combination thereof. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof. In some embodiments, the pharmaceutical composition described herein comprising an ASD is formulated in a unit dosage form.

In some embodiments, the ASD comprises abiraterone free base or abiraterone acetate. in an amount of about 20 mg to about 200 mg. In some embodiments, the ASD comprises a hydrophilic polymer in an amount of about 80 mg to about 700 mg. In some embodiments, the hydrophilic polymer is HPMCAS, polymethacrylates, or copovidone or a combination thereof. In some embodiments, the ASD comprises a surfactant in an amount of about 10 mg to about 80 mg. In some embodiments, the surfactant is lecithin or TPGS or a combination thereof. In some embodiments, the ASD comprises an organic acid. In some embodiments, the organic acid is in an amount of about 10 mg to about 250 mg. In some embodiments, the organic acid is tartaric acid, oleic acid, or a combination thereof. In some embodiments, the ASD comprises an adsorbent. In some embodiments, the adsorbent is in an amount of about 10 mg to about 400 mg. In some embodiments, the adsorbent is silicon dioxide or magnesium aluminum silicate or a combination thereof. In some embodiments, the pharmaceutical composition described herein comprising an ASD is formulated in a unit dosage form.

In some embodiments, the ASD comprises abiraterone free base or abiraterone acetate. in an amount of about 30 mg to about 150 mg. In some embodiments, the ASD comprises a hydrophilic polymer in an amount of about 100 mg to about 600 mg. In some embodiments, the hydrophilic polymer is HPMCAS, polymethacrylates, or copovidone or a combination thereof. In some embodiments, the ASD comprises a surfactant in an amount of about 20 mg to about 60 mg. In some embodiments, the surfactant is lecithin or TPGS or a combination thereof. In some embodiments, the ASD comprises an organic acid. In some embodiments, the organic acid is in an amount of about 10 mg to about 150 mg. In some embodiments, the organic acid is tartaric acid, oleic acid, or a combination thereof. In some embodiments, the ASD comprises an adsorbent. In some embodiments, the adsorbent is in an amount of about 20 mg to about 180 mg. In some embodiments, the adsorbent is silicon dioxide or magnesium aluminum silicate or a combination thereof. In some embodiments, the pharmaceutical composition described herein comprising an ASD is formulated in a unit dosage form.

In some embodiments, the pharmaceutical compositions described herein have a superior bioavailability than a bioavailability of a corresponding reference composition comprising crystalline abiraterone acetate, when measured as AUC, AUCₘf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline abiraterone acetate, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline abiraterone acetate, when measured as Cₘₐₓ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of ZYTIGA tablet comprising abiraterone acetate, when measured as AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of ZYTIGA capsule comprising abiraterone acetate, when measured as Cₘₐₓ after oral administration. In some embodiments, the pharmaceutical composition exhibits a bioavailability that is higher than a bioavailability of ZYTIGA by about 1.1 fold to about 10 fold. In some embodiments, the pharmaceutical composition exhibits a bioavailability that is higher than a bioavailability of ZYTIGA by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical compositions comprise an ASD comprising abiraterone or abiraterone acetate. In some embodiment, the bioavailability is measured in fasted condition.

In some embodiments, the pharmaceutical composition described herein exhibits a bioavailability that is higher than a bioavailability of ZYTIGA by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUC, AUCinf, or AUCₗₐₛₜ or Cₘₐₓ after oral administration. In some embodiments, the pharmaceutical composition exhibits a bioavailability that is higher than a bioavailability of ZYTIGA by at least about 2 fold. In some embodiments, the pharmaceutical composition exhibits a bioavailability that is higher than a bioavailability of ZYTIGA by at least about 4 fold. In some embodiments, the pharmaceutical composition exhibits a bioavailability that is higher than a bioavailability of ZYTIGA by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUC, AUCinf, or AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiment, the bioavailability is measured under fasted condition. In some embodiments, the pharmaceutical compositions comprise an ASD comprising abiraterone or abiraterone acetate.

In some embodiments, the pharmaceutical compositions described herein exhibits a bioavailability that is from about 30% to about 1500%, 40% to about 1000%, 50% to about 500%, about 70% to about 300%, 75% to about 200%, or about 100% to about 200% of a bioavailability of a corresponding reference composition comprising abiraterone acetate, when measured as AUCₗₐₛₜ or Cₘₐₓ after oral administration, wherein the corresponding reference pharmaceutical composition does not comprise an amorphous solid dispersion. In some embodiments, the reference composition is at least about 1.1 times the dosage of the pharmaceutical compositions. In some embodiments, the reference composition is at least about 1.1 times, about 1.5 times, about 2 times, about 2.5 times, about 3 times, about 4 times, about 5 times, about 6 times, about 7 times, about 8 times, about 9 times, or about 10 times the dosage of the pharmaceutical composition. In some embodiments, the pharmaceutical composition comprises an ASD comprising abiraterone free base or abiraterone acetate. In some embodiments, the reference composition comprises abiraterone free base or abiraterone acetate, wherein the reference composition does not comprise an ASD. In some embodiments, the reference composition is ZYTIGA. In some embodiment, the bioavailability is measured under fasted condition.

In some embodiments, the pharmaceutical compositions described herein exhibits a bioavailability that is from about 75% to about 200% of a bioavailability of a corresponding reference composition comprising abiraterone acetate, when measured as AUCₗₐₛₜ after oral administration, wherein the corresponding reference pharmaceutical composition does not comprise an amorphous solid dispersion, and wherein the reference composition is at least 3 times the dosage of the pharmaceutical composition. In some embodiments, the reference composition is ZYTIGA. In some embodiment, the bioavailability is measured under fasted condition.

In some embodiments, the pharmaceutical compositions described herein exhibits a bioavailability that is from about 100% to about 200% of a bioavailability of a corresponding reference composition comprising abiraterone acetate, when measured as Cₘₐₓ after oral administration, wherein the corresponding reference pharmaceutical composition does not comprise an amorphous solid dispersion, and wherein the reference composition is at least 3 times the dosage of the pharmaceutical composition. In some embodiments, the reference composition is ZYTIGA. In some embodiment, the bioavailability is measured under fasted condition.

In some embodiments, the pharmaceutical compositions described herein exhibits a bioavailability that is from about 90% to about 110% of a bioavailability of a corresponding reference composition comprising abiraterone acetate, when measured as AUCₗₐₛₜ after oral administration, wherein the corresponding reference pharmaceutical composition does not comprise an amorphous solid dispersion, and wherein the reference composition is about 5 times the dosage of the pharmaceutical composition. In some embodiments, the reference composition is ZYTIGA. In some embodiment, the bioavailability is measured under fasted condition.

In some embodiments, the pharmaceutical compositions described herein exhibits a bioavailability that is from about 100% to about 175% of a bioavailability of a corresponding reference composition comprising abiraterone acetate, when measured as Cₘₐₓ after oral administration, wherein the corresponding reference pharmaceutical composition does not comprise an amorphous solid dispersion, and wherein the reference composition is about 5 times the dosage of the pharmaceutical composition. In some embodiments, the reference composition is ZYTIGA. In some embodiment, the bioavailability is measured under fasted condition.

In some embodiments, salts of compounds of abiraterone are formed, for example, as acid addition salts (e.g., with organic or inorganic acids), from compounds of abiraterone with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### d) Alectinib Hydrochloride

In one aspect, disclosed herein are pharmaceutical compositions comprising a ASD that comprises an API, a hydrophilic polymer, a surfactant, optionally an organic acid, and optionally an adsorbent. In one aspect, disclosed herein are pharmaceutical compositions comprising an ASD, wherein the ASD comprises an API. In some embodiments, the API is alectinib free base or a pharmaceutically acceptable salt thereof. In some embodiments, the API is alectinib hydrochloride.

In some embodiments, the ASD comprises alectinib free base or a pharmaceutically acceptable salt thereof (such as alectinib hydrochloride) in an amount of about 3% to about 60% by weight of the ASD. In some embodiments, the alectinib free base or a pharmaceutically acceptable salt thereof is present in the amorphous solid dispersion in a weight percent of about 10% to about 50%. In some embodiments, the alectinib free base or a pharmaceutically acceptable salt thereof is present in the amorphous solid dispersion in a weight percent of about 15% to about 30%. In some embodiments, the alectinib free base or a pharmaceutically acceptable salt thereof is present in the amorphous solid dispersion in a weight percent of about 25% to about 40%. In some embodiments, the ASD comprises alectinib free base or a pharmaceutically acceptable salt thereof in an amount of about 3% to about 30% by weight of the ASD. In some embodiments, the ASD comprises alectinib free base or a pharmaceutically acceptable salt thereof in an amount of about 5% to about 25% by weight of the ASD. In some embodiments, the ASD comprises alectinib free base or a pharmaceutically acceptable salt thereof in an amount of about 10% to about 20% by weight of the ASD. In some embodiments, the ASD comprises alectinib free base or a pharmaceutically acceptable salt thereof in an amount of about 10% by weight of the ASD. In some embodiments, the ASD comprises alectinib free base or a pharmaceutically acceptable salt thereof in an amount of about 20% by weight of the ASD.

In some embodiments, the alectinib free base or a pharmaceutically acceptable salt thereof (such as alectinib hydrochloride) is present in the amorphous solid dispersion in a weight percent of about 5% to about 60%. In some embodiments, the alectinib free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the alectinib free base or a pharmaceutically acceptable salt thereof is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the alectinib free base or a pharmaceutically acceptable salt thereof is present in the amorphous solid dispersion in a weight percent of at least about 5%, about 10%, about 15%, about 20%, about 25%, about 30%, about 35%, about 40%, about 45%, about 50%, or about 55%. In some embodiments, the alectinib free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMC. In some embodiments, the hydrophilic polymer is polymethacrylates. In some embodiments, the hydrophilic polymer HPMCAS HPMC. In some embodiments, the hydrophilic polymer is Soluplus. In some embodiments, the hydrophilic polymer is HPMC, polymethacrylates, HPMCAS or Soluplus or a combination thereof. In some embodiments, the hydrophilic polymer comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides (e.g., Labrasol or Gelucire), polysorbate, or a combination thereof. In some embodiments, the hydrophilic polymer is present in an amount of about 1% to about 80% by weight of the ASD. In some embodiments, the hydrophilic polymer is present in an amount of about 5% to about 70% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD optionally comprises a surfactant. In some embodiments, the ASD optionally comprises a surfactant. In some embodiments, the surfactant is TPGS. In some embodiments, the surfactant is polyoxylglycerides. In some embodiments, the surfactant is SLS. In some embodiments, the surfactant is polysorbate. In some embodiments, the surfactant is polyoxyl hydrogenated castor oil. In some embodiments, the surfactant is TPGS, polyoxylglycerides, polysorbate, polyoxyl hydrogenated castor oil, or SLS or a combination thereof. In some embodiments the surfactant is present in an amount of about 5% to about 60% by weight of the ASD. In some embodiments, the surfactant is present in an amount of about 5% to about 40% by weight of the ASD. In some embodiments, the surfactant is present in an amount of about 5% to about 30% by weight of the ASD.

In some embodiments, the ASD comprises optionally an adsorbent. In some embodiments, the adsorbent is silicone dioxide. In some embodiments, the adsorbent is present in an amount of about 1% to about 40% by weight of the ASD.

In some embodiments, the ASD comprises optionally an inorganic acid or organic acid. In some embodiments, the ASD comprises optionally an organic acid. In some embodiments, the ASD comprises an organic acid. In some embodiments, the organic acid is tartaric acid. In some embodiments, the organic acid is citric acid. In some embodiments, the organic acid is malic acid. In some embodiments, the organic acid is tartaric acid, citric acid, or malic acid, or a combination thereof. In some embodiments, the inorganic acid or organic acid is present in an amount of about 1% to about 40% by weight of the ASD. In some embodiments, the inorganic acid or organic acid is present in an amount of about 10% to about 50% by weight of the ASD. In some embodiments, the inorganic acid or organic acid is present in an amount of about 1% to about 30% by weight of the ASD. In some embodiments, the inorganic acid or organic acid is present in an amount of about 1% to about 20% by weight of the ASD.

In some embodiments, an ASD comprises alectinib free base or a pharmaceutically acceptable salt thereof in an amount of about 5% to about 60% by weight of the ASD. In some embodiments, the ASD comprises a hydrophilic polymer. In some embodiments, the hydrophilic polymer is in an amount of about 15% to about 80% by weight of the ASD. In some embodiments, the hydrophilic polymer is HPMC, polymethacrylates HPMCAS or Soluplus or a combination thereof. In some embodiments, the ASD comprises a surfactant. In some embodiments, the surfactant is in an amount of about 10% to about 40% by weight of the ASD. In some embodiments, the surfactant is TPGS or SLS or a combination thereof. In some embodiments, the ASD comprises an organic acid. In some embodiments, the organic acid is in an amount of about 10% to about 50% by weight of the ASD. In some embodiments, the organic acid is tartaric acid.

In some embodiments, the ASD comprises alectinib free base or a pharmaceutically acceptable salt thereof in an amount of about 15% to about 55% by weight of the ASD. In some embodiments, the ASD comprises a hydrophilic polymer. In some embodiments, the hydrophilic polymer is in an amount of about 15% to about 70% by weight of the ASD. In some embodiments, the hydrophilic polymer is HPMC, polymethacrylates, HPMCAS or Soluplus or a combination thereof. In some embodiments, the ASD comprises a surfactant. In some embodiments, the surfactant is in an amount of about 15% to about 30% by weight of the ASD. In some embodiments, the surfactant is TPGS, polyoxylglycerides, polysorbate, polyoxyl hydrogenated castor oil, or SLS or a combination thereof. In some embodiments, the ASD comprises an organic acid. In some embodiments, the organic acid is in an amount of about 20% to about 40% by weight of the ASD. In some embodiments, the organic acid is tartaric acid. In some embodiments, the ASD comprises an adsorbent in an amount of about 1% to 40% by weight of the ASD. In some embodiments, the adsorbent is silicon dioxide.

In some embodiments, the pharmaceutical composition described herein comprises an ASD comprising alectinib free base or a pharmaceutically acceptable salt thereof (such as alectinib hydrochloride) in an amount of about 25% by weight of the ASD. In some embodiments, the ASD comprises a hydrophilic polymer. In some embodiments, the hydrophilic polymer is in an amount of about 50% by weight of the ASD. In some embodiments, the hydrophilic polymer is HPMCAS. In some embodiments, the ASD comprises a surfactant. In some embodiments, the surfactant is in an amount of about 25% by weight of the ASD. In some embodiments, the surfactant is SLS. In some embodiments, the ASD comprises an adsorbent. In some embodiments, the adsorbent is in an amount of about 1% to 30% by weight of the ASD. In some embodiments, the adsorbent is silicon dioxide.

In some embodiments, the pharmaceutical composition described herein comprises an ASD comprising alectinib free base or a pharmaceutically acceptable salt thereof. In some embodiments, the alectinib free base or a pharmaceutically acceptable salt thereof is in an amount of about 5% to about 50% by weight of the ASD. In some embodiments, the alectinib free base or a pharmaceutically acceptable salt thereof is in an amount of about 10-30% by weight of the ASD. In some embodiments, the alectinib free base or a pharmaceutically acceptable salt thereof is in an amount of about 10-25% by weight of the ASD. In some embodiments, the alectinib free base or a pharmaceutically acceptable salt thereof is in an amount of about 15% by weight of the ASD. In some embodiments, the ASD comprises a hydrophilic polymer. In some embodiments, the hydrophilic polymer is in an amount of about 10% to about 80% by weight of the ASD. In some embodiments, the hydrophilic polymer is in an amount of about 5% to about 70% by weight of the ASD. In some embodiments, the hydrophilic polymer is in an amount of about 15% to about 60% by weight of the ASD. In some embodiments, the hydrophilic polymer is in an amount of about 25% by weight of the ASD. In some embodiments, the hydrophilic polymer is HPMC, HPMCAS, polyvinyl acetate, polyvinylcaprolactame-based graft copolymer, PVP, copovidone, polymethacrylates, or a combination thereof. In some embodiments, the hydrophilic polymer is eudragit. In some embodiments, the hydrophilic polymer is lauroyl polyoxyl-32 gylcerides. In some embodiments, the hydrophilic polymer is HPMC-E5. In some embodiments, the hydrophilic polymer is HPMCAS-LF. In some embodiments, the ASD comprises a surfactant. In some embodiments, the surfactant is in an amount of about 10% to about 50% by weight of the ASD. In some embodiments, the surfactant is in an amount of about 10% to about 40% by weight of the ASD. In some embodiments, the surfactant is in an amount of about 10% to about 30% by weight of the ASD. In some embodiments, the surfactant is in an amount of about 20% to about 25% by weight of the ASD. In some embodiments, the surfactant is TPGS. In some embodiments, TPGS is present in an amount of about 20-25% by weight of the ASD. In some embodiments, the surfactant is SDS. In some embodiments, SDS is present in an amount of about 15% to about 40% by weight of the ASD. In some embodiments, the surfactant is RH40. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, RH40 is present in an amount of about 20% by weight of the ASD. In some embodiments, the surfactant is Tween-20. In some embodiments, Tween-20 is present in an amount of about 20% by weight of the ASD. In some embodiments, the surfactant is Gelucire44/14. In some embodiments, Gelucire44/14 is present in an amount of about 20% by weight of the ASD. In some embodiments, the ASD comprises an organic acid. In some embodiments, the organic acid is present in an amount of about 10% to about 35% by weight of the ASD. In some embodiments, the organic acid is present in an amount of about 10% to about 30% by weight of the ASD. In some embodiments, the organic acid is tartaric acid. In some embodiments, the tartaric acid is present in an amount of about 20% to 25% by weight of the ASD. In some embodiments, the ASD comprises an adsorbent. In some embodiments, the adsorbent is present in an amount of about 15% to about 40% by weight of the ASD. In some embodiments, the adsorbent is SiO₂. In some embodiments, the SiO₂ is present in an amount of about 20% by weight of the ASD.

In some embodiments, the pharmaceutical composition described herein comprises an ASD that comprising alectinib free base or a pharmaceutically acceptable salt thereof in an amount of about 25% by weight of the ASD; a hydrophilic polymer in an amount of about 75% by weight of the ASD, and wherein the hydrophilic polymer is Eudagrit L100-55 or HPMC-E5.

In some embodiments, the pharmaceutical composition described herein comprises an ASD that consists of alectinib free base or a pharmaceutically acceptable salt thereof in an amount of about 10% to about 30% by weight of the ASD; a hydrophilic polymer in an amount of about 20% to about 75% by weight of the ASD, wherein the hydrophilic polymer is HPMCAS, Soluplus, or HPMC-E5; and a surfactant in an amount of about 10% to about 25% by weight of the ASD, wherein the surfactant is TPGS, SDS, or lecithin.

In some embodiments, the pharmaceutical composition described herein comprises an ASD that consists of alectinib free base or a pharmaceutically acceptable salt thereof in an amount of about 20% to 25% by weight of the ASD; a hydrophilic polymer in an amount of about 15% to 25% by weight of the ASD, wherein the hydrophilic polymer is HPMCAS, HPMC-E5, or Eudragit L100; and tartaric acid in an amount of about 25% to 30% by weight of the ASD.

In some embodiments, the pharmaceutical composition described herein comprises an ASD that consists of alectinib free base or a pharmaceutically acceptable salt thereof in an amount of about 20% by weight of the ASD; a hydrophilic polymer in an amount of about 20% by weight of the ASD, wherein the hydrophilic polymer is HPMCAS-LF; and a surfactant in an amount of about 10% to 25% by weight of the ASD, wherein the surfactant is TPGS, Tween-20, Gelcure44/14, RH40, SDS or any combination thereof; tartaric acid in an amount of about 20% by weight of the ASD; and an adsorbent in an amount of about 20% by weight of the ASD, wherein the adsorbent is SiO₂.

In some embodiments, the ASD comprises alectinib free base or a pharmaceutically acceptable salt thereof in an amount of about 50 mg to about 200 mg. In some embodiments, the ASD comprises a hydrophilic polymer in an amount of about 100 mg to about 500 mg. In some embodiments, the hydrophilic polymer is HPMC, HPMCAS or Soluplus or a combination thereof. In some embodiments, the ASD comprises a surfactant in an amount of about 40 mg to about 250 mg. In some embodiments, the surfactant is TPGS or SLS or a combination thereof. In some embodiments, the ASD comprises an organic acid in an amount of about 70 mg to 250 mg. In some embodiments, the organic acid is tartaric acid. In some embodiments, the ASD comprises an adsorbent in an amount of about 20 mg to 300 mg by weight of the ASD. In some embodiments, the adsorbent is silicon dioxide. In some embodiments, the pharmaceutical composition described herein comprising an ASD is formulated in a unit dosage form.

In some embodiments, the ASD comprises alectinib free base or a pharmaceutically acceptable salt thereof in an amount of about 60 mg to about 180 mg, a hydrophilic polymer in an amount of about 100 mg to about 400 mg. In some embodiments, the hydrophilic polymer is HPMC, HPMCAS or Soluplus or a combination thereof, a surfactant in an amount of about 50 mg to about 200 mg. In some embodiments, the surfactant is TPGS or SLS or a combination thereof, and optionally an organic acid in an amount of about 130 mg to 180 mg. In some embodiments, the organic acid is tartaric acid. In some embodiments, the ASD comprises an adsorbent in an amount of about 50 mg to 200 mg by weight of the ASD. In some embodiments, the adsorbent is silicon dioxide. In some embodiments, the pharmaceutical composition described herein comprising an ASD is formulated in a unit dosage form.

In some embodiments, pharmaceutical compositions described herein comprising an ASD that comprises alectinib free base or a pharmaceutically acceptable salt thereof have acceptable storage stability. In some embodiments, the pharmaceutical composition is storage stable for at least 2 weeks at 75° C./75% RH, wherein a storage stable chemically pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline alectinib hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline alectinib hydrochloride, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline alectinib hydrochloride, when measured as Cₘₐₓ after oral administration.

In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of ALECENSA capsule comprising alectinib hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of ALECENSA capsule comprising alectinib hydrochloride, when measured as Cₘₐₓ after oral administration. In some embodiments, the pharmaceutical composition exhibits a bioavailability that is higher than a bioavailability of ALECENSA by about 1.1 fold to about 10 fold. In some embodiments, the pharmaceutical composition exhibits a bioavailability that is higher than a bioavailability of ALECENSA by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical compositions comprise an ASD comprising alectinib or a salt thereof (such as alectinib hydrochloride). In some embodiment, the bioavailability is measured in fasted condition.

In some embodiments, the pharmaceutical composition described herein exhibits a bioavailability that is higher than a bioavailability of ALECENSA by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUCₗₐₛₜ or Cₘₐₓ after oral administration. In some embodiments, the pharmaceutical composition exhibits a bioavailability that is higher than a bioavailability of ALECENSA by at least about 2 fold. In some embodiments, the pharmaceutical composition exhibits a bioavailability that is higher than a bioavailability of ALECENSA by at least about 4 fold. In some embodiments, the pharmaceutical composition exhibits a bioavailability that is higher than a bioavailability of ALECENSA by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiment, the bioavailability is measured under fasted condition. In some embodiment, the bioavailability is measured under fed condition. In some embodiments, the pharmaceutical compositions comprise an ASD comprising alectinib free base or a pharmaceutically acceptable salt thereof (such as alectinib hydrochloride).

In some embodiments, the pharmaceutical compositions described herein exhibits a bioavailability that is from about 30% to about 1500%, 40% to about 1000%, 50% to about 500%, about 70% to about 300%, 75% to about 200%, or about 80% to about 150% of a bioavailability of a corresponding reference composition comprising alectinib hydrochloride when measured as AUCₗₐₛₜ or Cₘₐₓ after oral administration, wherein the corresponding reference pharmaceutical composition does not comprise an amorphous solid dispersion. In some embodiments, the reference composition is at least about 1.1 times the dosage of the pharmaceutical compositions. In some embodiments, the reference composition is at least about 1.1 times, about 1.5 times, about 2 times, about 2.5 times, about 3 times, about 4 times, about 5 times, about 6 times, about 7 times, about 8 times, about 9 times, or about 10 times the dosage of the pharmaceutical composition. In some embodiments, the pharmaceutical composition comprises an ASD comprising alectinib free base or a pharmaceutically acceptable salt thereof (such as alectinib hydrochloride). In some embodiments, the reference composition comprises alectinib free base or a pharmaceutically acceptable salt thereof, wherein the reference composition does not comprise an ASD. In some embodiments, the reference composition is ALECENSA. In some embodiment, the bioavailability is measured under fasted condition. In some embodiment, the bioavailability is measured under fed condition. In some embodiment, the bioavailability is measured under fed condition.

In some embodiments, the pharmaceutical compositions described herein exhibits a bioavailability that is from about 75% to about 200% of a bioavailability of a corresponding reference composition comprising alectinib hydrochloride, when measured as AUCₗₐₛₜ after oral administration, wherein the corresponding reference pharmaceutical composition does not comprise an amorphous solid dispersion, and wherein the reference composition is at least 1.75 times the dosage of the pharmaceutical composition. In some embodiments, the reference composition is ALECENSA. In some embodiment, the bioavailability is measured under fasted condition.

In some embodiments, the pharmaceutical compositions described herein exhibits a bioavailability that is from about 75% to about 200% of a bioavailability of a corresponding reference composition comprising alectinib hydrochloride, when measured as Cₘₐₓ after oral administration, wherein the corresponding reference pharmaceutical composition does not comprise an amorphous solid dispersion, and wherein the reference composition is at least 1.75 times the dosage of the pharmaceutical composition. In some embodiments, the reference composition is ALECENSA. In some embodiment, the bioavailability is measured under fasted condition. In some embodiment, the bioavailability is measured under fed condition.

In some embodiments, the pharmaceutical compositions described herein exhibits a bioavailability that is at least about 110% of a bioavailability of a corresponding reference composition comprising alectinib hydrochloride, when measured as AUCₗₐₛₜ after oral administration under fasted condition, wherein the corresponding reference pharmaceutical composition does not comprise an amorphous solid dispersion, and wherein the reference composition is about 2 times the dosage of the pharmaceutical composition. In some embodiments, the pharmaceutical compositions described herein exhibits a bioavailability that is at least about 80% of a bioavailability of a corresponding reference composition comprising alectinib hydrochloride, when measured as AUCₗₐₛₜ after oral administration under fasted condition, wherein the corresponding reference pharmaceutical composition does not comprise an amorphous solid dispersion, and wherein the reference composition is about 2 times the dosage of the pharmaceutical composition. In some embodiments, the reference composition is ALECENSA.

In some embodiments, the pharmaceutical compositions described herein exhibits a bioavailability that is at least about 110% of a bioavailability of a corresponding reference composition comprising alectinib hydrochloride, when measured as Cₘₐₓ after oral administration under fasted condition, wherein the corresponding reference pharmaceutical composition does not comprise an amorphous solid dispersion, and wherein the reference composition is about 2 times the dosage of the pharmaceutical composition. In some embodiments, the pharmaceutical compositions described herein exhibits a bioavailability that is about at least about 80% of a bioavailability of a corresponding reference composition comprising alectinib hydrochloride, when measured as Cₘₐₓ after oral administration under fed condition, wherein the corresponding reference pharmaceutical composition does not comprise an amorphous solid dispersion, and wherein the reference composition is about 2 times the dosage of the pharmaceutical composition. In some embodiments, the reference composition is ALECENSA.

In some embodiments, salts of compounds of alectinib are formed, for example, as acid addition salts (e.g., with organic or inorganic acids), from compounds of alectinib with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### e) Pazopanib Hydrochloride

In one aspect, disclosed herein are pharmaceutical compositions comprising a ASD that comprises an API, a hydrophilic polymer, a surfactant, optionally an organic acid, and optionally an adsorbent. In one aspect, disclosed herein are pharmaceutical compositions comprising an ASD, wherein the ASD comprises an API. In some embodiments, the API is pazopanib free base or a pharmaceutically acceptable salt thereof. In some embodiments, the API is pazopanib hydrochloride.

In some embodiments, the ASD comprises pazopanib free base or a pharmaceutically acceptable salt thereof (such as pazopanib hydrochloride) in an amount of about 3% to about 60% by weight of the ASD. In some embodiments, the pazopanib free base or a pharmaceutically acceptable salt thereof is present in the amorphous solid dispersion in a weight percent of about 10% to about 50%. In some embodiments, the pazopanib free base or a pharmaceutically acceptable salt thereof is present in the amorphous solid dispersion in a weight percent of about 15% to about 30%. In some embodiments, the pazopanib free base or a pharmaceutically acceptable salt thereof is present in the amorphous solid dispersion in a weight percent of about 25% to about 40%. In some embodiments, the ASD comprises pazopanib free base or a pharmaceutically acceptable salt thereof in an amount of about 3% to about 50% by weight of the ASD. In some embodiments, the ASD comprises pazopanib free base or a pharmaceutically acceptable salt thereof in an amount of about 5% to about 40% by weight of the ASD. In some embodiments, the ASD comprises pazopanib free base or a pharmaceutically acceptable salt thereof in an amount of about 10% to about 30% by weight of the ASD. In some embodiments, the ASD comprises pazopanib free base or a pharmaceutically acceptable salt thereof in an amount of about 18.2% by weight of the ASD. In some embodiments, the ASD comprises pazopanib free base or a pharmaceutically acceptable salt thereof in an amount of about 20% by weight of the ASD. In some embodiments, the ASD comprises pazopanib free base or a pharmaceutically acceptable salt thereof in an amount of about 25% by weight of the ASD.

In some embodiments, the pazopanib free base or a pharmaceutically acceptable salt thereof (such as pazopanib hydrochloride) is present in the amorphous solid dispersion in a weight percent of about 5% to about 60%. In some embodiments, the pazopanib free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the pazopanib free base or a pharmaceutically acceptable salt thereof (such as pazopanib hydrochloride) is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the pazopanib free base or a pharmaceutically acceptable salt thereof is present in the amorphous solid dispersion in a weight percent of at least about 5%, about 10%, about 15%, about 20%, about 25%, about 30%, about 35%, about 40%, about 45%, about 50%, or about 55%. In some embodiments, the pazopanib free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMC. In some embodiments, the hydrophilic polymer is polymethacrylates. In some embodiments, the hydrophilic polymer is polyvinyl acetate and polyvinylcaprolactame-based graft copolymer. In some embodiments, the hydrophilic polymer is PVP. In some embodiments, the hydrophilic polymer is copovidone. In some embodiments, the hydrophilic polymer is HPMC. In some embodiments, the hydrophilic polymer is HPMC (such as HPMC-E5), polymethacrylates, HPMCAS, polyvinyl acetate and polyvinylcaprolactame-based graft copolymer, PVP, copovidone, or a combination thereof. In some embodiments, the hydrophilic polymer comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides (e.g., Labrasol or Gelucire), polysorbate, or a combination thereof. In some embodiments, the hydrophilic polymer is present in an amount of about 1% to about 80% by weight of the ASD. In some embodiments, the hydrophilic polymer is present in an amount of about 5% to about 70% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD optionally comprises a surfactant. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments the surfactant is present in an amount of about 5% to about 60% by weight of the ASD. In some embodiments, the surfactant is present in an amount of about 5% to about 40% by weight of the ASD. In some embodiments, the surfactant is present in an amount of about 5% to about 30% by weight of the ASD.

In some embodiments, the ASD comprises optionally an adsorbent. In some embodiments, the adsorbent is silicone dioxide. In some embodiments, the adsorbent is present in an amount of about 15% to about 40% by weight of the ASD.

In some embodiments, the ASD comprises optionally an inorganic acid or organic acid. In some embodiments, the ASD comprises optionally an organic acid. In some embodiments, the ASD comprises an organic acid, wherein the organic acid is tartaric acid. In some embodiments, the inorganic acid or organic acid is present in an amount of about 10% to about 40% by weight of the ASD. In some embodiments, the inorganic acid or organic acid is present in an amount of about 1% to about 30% by weight of the ASD. In some embodiments, the inorganic acid or organic acid is present in an amount of about 1% to about 20% by weight of the ASD.

In some embodiments, the ASD comprises an adsorbent. In some embodiments, the adsorbent is SiO₂. In some embodiments, the adsorbent is present in an amount of about 10% to about 40% by weight of the ASD. In some embodiments, the adsorbent is present in an amount of about 1% to about 30% by weight of the ASD. In some embodiments, the adsorbent is present in an amount of about 1% to about 20% by weight of the ASD.

In some embodiments, an ASD comprises pazopanib free base or a pharmaceutically acceptable salt thereof (such as pazopanib hydrochloride). In some embodiments, pazopanib free base or a pharmaceutically acceptable salt thereof is in an amount of about 5% to about 50% by weight of the ASD. In some embodiments, pazopanib free base or a pharmaceutically acceptable salt thereof is in an amount of about 5% to about 40% by weight of the ASD. In some embodiments, the ASD comprises a hydrophilic polymer. In some embodiments, the hydrophilic polymer is in an amount of about 5% to about 70% by weight of the ASD. In some embodiments, the hydrophilic polymer is in an amount of about 10% to about 70% by weight of the ASD. In some embodiments, the hydrophilic polymer is in an amount of about 10% to about 40% by weight of the ASD. In some embodiments, the hydrophilic polymer is in an amount of about 10% to about 30% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the hydrophilic polymer comprises HPMC, HPMCAS, polyvinyl acetate and polyvinylcaprolactame-based graft copolymer, PVP, copovidone, polymethacrylates, or a combination thereof. In some embodiments, the ASD comprises a surfactant. In some embodiments, the surfactant is in an amount of about 10% to about 50% by weight of the ASD. In some embodiments, the surfactant is in an amount of about 10% to about 40% by weight of the ASD. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant is TPGS or Lecithin or a combination thereof. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the ASD comprises an organic acid. In some embodiments, the organic acid is in an amount of about 10% to about 40% by weight of the ASD. In some embodiments, the organic acid is in an amount of about 10% to about 35% by weight of the ASD. In some embodiments, the organic acid is in an amount of about 10% to about 30% by weight of the ASD. In some embodiments, the organic acid is tartaric acid. In some embodiments, the ASD comprises an adsorbent. In some embodiments, the adsorbent is in an amount of about 15% to about 40% by weight of the ASD. In some embodiments, the adsorbent is SiO₂.

In some embodiments, the ASD comprises pazopanib free base or a pharmaceutically acceptable salt thereof in an amount of about 15% to about 30% by weight of the ASD. In some embodiments, the ASD comprises a hydrophilic polymer. In some embodiments, the hydrophilic polymer is in an amount of about 15% to about 60% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 15% to about 30% by weight of the ASD. In some embodiments, the surfactant is TPGS or Lecithin or a combination thereof. In some embodiments, the ASD comprises an organic acid. In some embodiments, the organic acid is in an amount of about 15% to about 30% by weight of the ASD. In some embodiments, the organic acid is tartaric acid. In some embodiments, the ASD comprises an adsorbent. In some embodiments, the adsorbent is in an amount of about 20% to about 35% by weight of the ASD. In some embodiments, the adsorbent is SiO₂.

In some embodiments, the pharmaceutical composition described herein comprises an ASD comprising pazopanib free base or a pharmaceutically acceptable salt thereof in an amount of about 15-20% by weight of the ASD. In some embodiments, the ASD comprises a hydrophilic polymer. In some embodiments, the hydrophilic polymer is in an amount of about 15-20% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 15-20% by weight of the ASD. In some embodiments, the surfactant is TPGS. In some embodiments, the ASD comprises an organic acid. In some embodiments, the organic acid is in an amount of about 15-20% by weight of the ASD. In some embodiments, the organic acid is tartaric acid. In some embodiments, the ASD comprises an adsorbent. In some embodiments, the adsorbent is in an amount of about 25-30% by weight of the ASD. In some embodiments, the adsorbent is SiO₂.

In some embodiments, the pharmaceutical composition described herein comprises an ASD comprising pazopanib free base or a pharmaceutically acceptable salt thereof. In some embodiments, the pazopanib free base or a pharmaceutically acceptable salt thereof is in an amount of about 10-30% by weight of the ASD. In some embodiments, the pazopanib free base or a pharmaceutically acceptable salt thereof is in an amount of about 15-20% by weight of the ASD. In some embodiments, the pazopanib free base or a pharmaceutically acceptable salt thereof is in an amount of about 16% by weight of the ASD. In some embodiments, the ASD comprises a hydrophilic polymer. In some embodiments, the hydrophilic polymer is in an amount of about 10% to about 30% by weight of the ASD. In some embodiments, the hydrophilic polymer is in an amount of about 10% to about 20% by weight of the ASD. In some embodiments, the hydrophilic polymer is in an amount of about 15% by weight of the ASD. In some embodiments, the hydrophilic polymer is HPMC, HPMCAS, polyvinyl acetate and polyvinylcaprolactame-based graft copolymer, PVP, copovidone, or a combination thereof. In some embodiments, the hydrophilic polymer is HPMC-E5. In some embodiments, the hydrophilic polymer is VA64. In some embodiments, the hydrophilic polymer is VA64. In some embodiments, the ASD comprises a surfactant. In some embodiments, the surfactant is in an amount of about 10% to about 40% by weight of the ASD. In some embodiments, the surfactant is in an amount of about 20% to about 40% by weight of the ASD. In some embodiments, the surfactant is in an amount of about 30% by weight of the ASD. In some embodiments, the surfactant is TPGS or lecithin or a combination thereof. In some embodiments, TPGS is present in an amount of about 15% by weight of the ASD. In some embodiments, lecithin is present in an amount of about 15% by weight of the ASD. In some embodiments, the ASD comprises an organic acid. In some embodiments, the organic acid is present in an amount of about 10% to about 30% by weight of the ASD. In some embodiments, the organic acid is present in an amount of about 10% to about 20% by weight of the ASD. In some embodiments, the organic acid is present in an amount of about 15% by weight of the ASD. In some embodiments, the organic acid is tartaric acid. In some embodiments, the ASD comprises an adsorbent. In some embodiments, the adsorbent is present in an amount of about 15% to about 40% by weight of the ASD. In some embodiments, the adsorbent is present in an amount of about 20% to about 30% by weight of the ASD. In some embodiments, the adsorbent is present in an amount of about 23% by weight of the ASD. In some embodiments, the adsorbent is SiO₂.

In some embodiments, the pharmaceutical composition described herein comprises an ASD that comprising pazopanib free base or a pharmaceutically acceptable salt thereof in an amount of about 16% by weight of the ASD; a hydrophilic polymer in an amount of about 15% by weight of the ASD, wherein the hydrophilic polymer is HPMC-E5; TPGS in an amount of about 15% by weight of the ASD; lecithin in an amount of about 15% by weight of the ASD; tartaric acid in an amount of about 15% by weight of the ASD; and an adsorbent in an amount of about 23% by weight of the ASD, wherein the adsorbent is SiO₂.

In some embodiments, the pharmaceutical composition described herein comprises an ASD that consists of pazopanib free base or a pharmaceutically acceptable salt thereof in an amount of about 10% to about 30% by weight of the ASD; a hydrophilic polymer in an amount of about 10% to about 30% by weight of the ASD, wherein the hydrophilic polymer is HPMC, polyvinyl acetate and polyvinylcaprolactame-based graft copolymer, PVP, copovidone, or a combination thereof; a surfactant in an amount of about 10% to about 40% by weight of the ASD, wherein the surfactant is TPGS or lecithin or a combination thereof; an organic acid in an amount of about 10% to about 30% by weight of the ASD, wherein the organic acid is tartaric acid; and optionally an adsorbent in an amount of about 15% to about 40% by weight of the ASD, wherein the adsorbent is SiO₂.

In some embodiments, the pharmaceutical composition described herein comprises an ASD that consists of pazopanib free base or a pharmaceutically acceptable salt thereof in an amount of about 16% by weight of the ASD; a hydrophilic polymer in an amount of about 15% by weight of the ASD, wherein the hydrophilic polymer is HPMC-E5; TPGS in an amount of about 15% by weight of the ASD; lecithin in an amount of about 15% by weight of the ASD; tartaric acid in an amount of about 15% by weight of the ASD; and an adsorbent in an amount of about 23% by weight of the ASD, wherein the adsorbent is SiO₂.

In some embodiments, the ASD comprises pazopanib free base or a pharmaceutically acceptable salt thereof (e.g., pazopanib hydrochloride) in an amount of about 30 mg to about 200 mg. In some embodiments, the ASD comprises a hydrophilic polymer/In some embodiments, the hydrophilic polymer is in an amount of about 30 mg to about 400 mg. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 30 mg to about 200 mg. In some embodiments, the surfactant is TPGS or Lecithin or a combination thereof. In some embodiments, the ASD comprises an organic acid. In some embodiments, the organic acid is in an amount of about 30 mg to 200 mg. In some embodiments, the organic acid is tartaric acid. In some embodiments, the ASD comprises an adsorbent. In some embodiments, the adsorbent is in an amount of about 30 mg to about 150 mg. In some embodiments, the adsorbent is SiO2. In some embodiments, the pharmaceutical composition described herein comprising an ASD is formulated in a unit dosage form.

In some embodiments, the ASD comprises pazopanib free base or a pharmaceutically acceptable salt thereof (e.g., pazopanib hydrochloride) in an amount of about 30 mg to about 150 mg. In some embodiments, the ASD comprises a hydrophilic polymer. In some embodiments, the hydrophilic polymer is in an amount of about 30 mg to about 300 mg. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 30 mg to about 150 mg. In some embodiments, the surfactant is TPGS or Lecithin or a combination thereof. In some embodiments, the ASD comprises an organic acid. In some embodiments, the organic acid is in an amount of about 30 mg to 150 mg. In some embodiments, the organic acid is tartaric acid. In some embodiments, the ASD comprises an adsorbent. In some embodiments, the adsorbent is in an amount of about 40 mg to about 100 mg. In some embodiments, the adsorbent is SiO2. In some embodiments, the pharmaceutical composition described herein comprising an ASD is formulated in a unit dosage form.

In some embodiments, pharmaceutical compositions described herein comprising an ASD that comprises pazopanib free base or a pharmaceutically acceptable salt thereof (e.g., pazopanib hydrochloride) have acceptable storage stability. In some embodiments, the pharmaceutical composition is storage stable chemically for at least 2 weeks at 75° C./75% RH, wherein a storage stable pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline pazopanib hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline pazopanib hydrochloride, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline pazopanib hydrochloride, when measured as Cₘₐₓ after oral administration.

In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VOTRIENT capsule comprising pazopanib hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VOTRIENT capsule comprising pazopanib hydrochloride, when measured as Cₘₐₓ after oral administration. In some embodiments, the pharmaceutical composition exhibits a bioavailability that is higher than a bioavailability of VOTRIENT by about 1.1 fold to about 10 fold. In some embodiments, the pharmaceutical composition exhibits a bioavailability that is higher than a bioavailability of VOTRIENT by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical compositions comprise an ASD comprising pazopanib or pazopanib hydrochloride. In some embodiment, the bioavailability is measured in fasted condition.

In some embodiments, the pharmaceutical composition described herein exhibits a bioavailability that is higher than a bioavailability of VOTRIENT by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUCₗₐₛₜ or Cₘₐₓ after oral administration. In some embodiments, the pharmaceutical composition exhibits a bioavailability that is higher than a bioavailability of VOTRIENT by at least about 2 fold. In some embodiments, the pharmaceutical composition exhibits a bioavailability that is higher than a bioavailability of VOTRIENT by at least about 4 fold. In some embodiments, the pharmaceutical composition exhibits a bioavailability that is higher than a bioavailability of VOTRIENT by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiment, the bioavailability is measured under fasted condition. In some embodiment, the bioavailability is measured under fed condition. In some embodiments, the pharmaceutical compositions comprise an ASD comprising pazopanib free base or a pharmaceutically acceptable salt thereof (e.g., pazopanib hydrochloride).

In some embodiments, the pharmaceutical compositions described herein exhibits a bioavailability that is from about 25% to about 1500%, 30% to about 1000%, 35% to about 500%, about 40% to about 300%, 40% to about 200%, or about 50% to about 120% of a bioavailability of a corresponding reference composition comprising pazopanib hydrochloride when measured as AUCₗₐₛₜ or Cₘₐₓ after oral administration, wherein the corresponding reference pharmaceutical composition does not comprise an amorphous solid dispersion. In some embodiments, the reference composition is at least about 1.1 times the dosage of the pharmaceutical compositions. In some embodiments, the reference composition is at least about 1.1 times, about 1.5 times, about 2 times, about 2.5 times, about 3 times, about 4 times, about 5 times, about 6 times, about 7 times, about 8 times, about 9 times, or about 10 times the dosage of the pharmaceutical composition. In some embodiments, the pharmaceutical composition comprises an ASD comprising pazopanib free base or a pharmaceutically acceptable salt thereof (e.g, pazopanib hydrochloride). In some embodiments, the reference composition comprises pazopanib free base or a pharmaceutically acceptable salt thereof, wherein the reference composition does not comprise an ASD. In some embodiments, the reference composition is VOTRIENT. In some embodiment, the bioavailability is measured under fasted condition. In some embodiment, the bioavailability is measured under fed condition. In some embodiment, the bioavailability is measured under fed condition.

In some embodiments, the pharmaceutical compositions described herein exhibits a bioavailability that is from about 40% to about 100% of a bioavailability of a corresponding reference composition comprising pazopanib hydrochloride, when measured as AUCₗₐₛₜ after oral administration under fasted condition, wherein the corresponding reference pharmaceutical composition does not comprise an amorphous solid dispersion, and wherein the reference composition is at least 3 times the dosage of the pharmaceutical composition. In some embodiments, the reference composition is VOTRIENT. In some embodiment, the bioavailability is measured under fasted condition.

In some embodiments, the pharmaceutical compositions described herein exhibits a bioavailability that is from about 50% to about 120% of a bioavailability of a corresponding reference composition comprising pazopanib hydrochloride, when measured as Cₘₐₓ after oral administration under fasted condition, wherein the corresponding reference pharmaceutical composition does not comprise an amorphous solid dispersion, and wherein the reference composition is at least 3 times the dosage of the pharmaceutical composition. In some embodiments, the reference composition is VOTRIENT. In some embodiment, the bioavailability is measured under fasted condition.

In some embodiments, the pharmaceutical compositions described herein exhibits a bioavailability that is at least about 50% of a bioavailability of a corresponding reference composition comprising pazopanib hydrochloride, when measured as AUCₗₐₛₜ after oral administration under fasted condition, wherein the corresponding reference pharmaceutical composition does not comprise an amorphous solid dispersion, and wherein the reference composition is about 4 times the dosage of the pharmaceutical composition. In some embodiments, the reference composition is VOTRIENT. In some embodiment, the bioavailability is measured under fasted condition.

In some embodiments, the pharmaceutical compositions described herein exhibits a bioavailability that is at least about 70% of a bioavailability of a corresponding reference composition comprising pazopanib hydrochloride, when measured as Cₘₐₓ after oral administration under fasted condition, wherein the corresponding reference pharmaceutical composition does not comprise an amorphous solid dispersion, and wherein the reference composition is about 4 times the dosage of the pharmaceutical composition. In some embodiments, the reference composition is VOTRIENT. In some embodiment, the bioavailability is measured under fasted condition.

In some embodiments, salts of compounds of pazopanib are formed, for example, as acid addition salts (e.g., with organic or inorganic acids), from compounds of pazopanib with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### f) Lurasidone Hydrochloride

In one aspect, disclosed herein are pharmaceutical compositions comprising a ASD that comprises an API, a hydrophilic polymer, a surfactant, optionally an organic acid, and optionally an adsorbent. In one aspect, disclosed herein are pharmaceutical compositions comprising an ASD, wherein the ASD comprises an API. In some embodiments, the API is lurasidone free base or a pharmaceutically acceptable salt thereof. In some embodiments, the API is lurasidone hydrochloride.

In some embodiments, the ASD comprises lurasidone free base or a pharmaceutically acceptable salt thereof (such as lurasidone hydrochloride) in an amount of about 3% to about 60% by weight of the ASD. In some embodiments, the lurasidone free base or a pharmaceutically acceptable salt thereof is present in the amorphous solid dispersion in a weight percent of about 10% to about 50%. In some embodiments, the lurasidone free base or a pharmaceutically acceptable salt thereof is present in the amorphous solid dispersion in a weight percent of about 15% to about 30%. In some embodiments, the lurasidone free base or a pharmaceutically acceptable salt thereof is present in the amorphous solid dispersion in a weight percent of about 25% to about 40%. In some embodiments, the ASD comprises lurasidone free base or a pharmaceutically acceptable salt thereof in an amount of about 3% to about 50% by weight of the ASD. In some embodiments, the ASD comprises lurasidone free base or a pharmaceutically acceptable salt thereof in an amount of about 5% to about 40% by weight of the ASD. In some embodiments, the ASD comprises lurasidone free base or a pharmaceutically acceptable salt thereof in an amount of about 10% to about 35% by weight of the ASD.

In some embodiments, the lurasidone free base or a pharmaceutically acceptable salt thereof (such as lurasidone hydrochloride) is present in the amorphous solid dispersion in a weight percent of about 5% to about 60%. In some embodiments, the lurasidone free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the lurasidone free base or a pharmaceutically acceptable salt thereof is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the lurasidone free base or a pharmaceutically acceptable salt thereof is present in the amorphous solid dispersion in a weight percent of at least about 5%, about 10%, about 15%, about 20%, about 25%, about 30%, about 35%, about 40%, about 45%, about 50%, or about 55%. In some embodiments, the lurasidone free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMC. In some embodiments, the hydrophilic polymer is polymethacrylates. In some embodiments, the hydrophilic polymer is PVP/VA. In some embodiments, the hydrophilic polymer is HPMC AS. In some embodiments, the hydrophilic polymer is PVP. In some embodiments, the hydrophilic polymer is HPMC, polymethacrylates, PVP/VA, HPMC AS, PVP, or a combination thereof. In some embodiments, the hydrophilic polymer is present in an amount of about 1% to about 80% by weight of the ASD. In some embodiments, the hydrophilic polymer is present in an amount of about 5% to about 70% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD comprises a surfactant. In some embodiments, the ASD optionally comprises a surfactant. In some embodiments, the surfactant is TPGS. In some embodiments, the surfactant is PEG. In some embodiments, the surfactant is block copolymer of polyethylene glycol and polypropylene glycol. In some embodiments, the surfactant is polyoxyl hydrogenated castor oil. In some embodiments, the surfactant is lecithin. In some embodiments, the surfactant is TPGS, PEG, block copolymer of polyethylene glycol and polypropylene glycol, polyoxyl hydrogenated castor oil, lecithin or a combination thereof. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RIH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments the surfactant is present in an amount of about 5% to about 60% by weight of the ASD. In some embodiments, the surfactant is present in an amount of about 5% to about 40% by weight of the ASD. In some embodiments, the surfactant is present in an amount of about 5% to about 30% by weight of the ASD.

In some embodiments, the ASD comprises an inorganic acid or organic acid. In some embodiments, the ASD comprises optionally an organic acid. In some embodiments, the ASD comprises an organic acid, wherein the organic acid is tartaric acid or citric acid or a combination thereof. In some embodiments, the organic acid is tartaric acid. In some embodiments, the organic acid is citric acid. In some embodiments, the organic acid is citric acid. In some embodiments, the inorganic acid or organic acid is present in an amount of about 10% to about 40% by weight of the ASD. In some embodiments, the organic acid is citric acid. In some embodiments, the inorganic acid or organic acid is present in an amount of about 10% to about 30% by weight of the ASD.

In some embodiments, the ASD comprises optionally an adsorbent. In some embodiments, the adsorbent is silicone dioxide. In some embodiments, the adsorbent is present in an amount of about 15% to about 40% by weight of the ASD.

In some embodiments, an ASD comprises lurasidone free base or a pharmaceutically acceptable salt thereof (such as lurasidone hydrochloride). In some embodiments, the lurasidone free base or a pharmaceutically acceptable salt thereof is in an amount of about 5% to about 50% by weight of the ASD. In some embodiments, the ASD comprises a hydrophilic polymer. In some embodiments, the hydrophilic polymer is in an amount of about 10% to about 70% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 40% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 10% to about 40% by weight of the ASD. In some embodiments, the organic acid is tartaric acid or citric acid or a combination thereof, and optionally an adsorbent. In some embodiments, the adsorbent is in an amount of about 15% to about 40% by weight of the ASD. In some embodiments, the adsorbent is SiO₂.

In some embodiments, the ASD comprises lurasidone free base or a pharmaceutically acceptable salt thereof (such as lurasidone hydrochloride). In some embodiments, the lurasidone free base or a pharmaceutically acceptable salt thereof is in an amount of about 15% to about 40% by weight of the ASD. In some embodiments, the ASD comprises a hydrophilic polymer. In some embodiments, the hydrophilic polymer is in an amount of about 15% to about 60% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 30% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 15% to about 40% by weight of the ASD. In some embodiments, the organic acid is tartaric acid or citric acid or a combination thereof. In some embodiments, the ASD comprises an adsorbent. In some embodiments, the adsorbent is in an amount of about 15% to about 30% by weight of the ASD. In some embodiments, the adsorbent is SiO₂.

In some embodiments, the pharmaceutical composition described herein comprises an ASD comprising lurasidone free base or a pharmaceutically acceptable salt thereof. In some embodiments, the lurasidone free base or a pharmaceutically acceptable salt thereof is in an amount of about 25% by weight of the ASD. In some embodiments, the ASD comprises a hydrophilic polymer. In some embodiments, the hydrophilic polymer is in an amount of about 25% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 25% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 25% by weight of the ASD. In some embodiments, the organic acid is tartaric acid.

In some embodiments, the pharmaceutical composition described herein comprises an ASD comprising lurasidone free base or a pharmaceutically acceptable salt thereof. In some embodiments, the lurasidone free base or a pharmaceutically acceptable salt thereof is in an amount of about 25% by weight of the ASD. In some embodiments, the ASD comprises a hydrophilic polymer. In some embodiments, the hydrophilic polymer is in an amount of about 25% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 25% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 25% by weight of the ASD. In some embodiments, the organic acid is citric acid.

In some embodiments, the pharmaceutical composition described herein comprises an ASD comprising lurasidone free base or a pharmaceutically acceptable salt thereof. In some embodiments, the lurasidone free base or a pharmaceutically acceptable salt thereof is in an amount of about 5% to 50% by weight of the ASD. In some embodiments, the lurasidone free base or a pharmaceutically acceptable salt thereof is in an amount of about 10% to 30% by weight of the ASD. In some embodiments, the lurasidone free base or a pharmaceutically acceptable salt thereof is in an amount of about 10-25% by weight of the ASD. In some embodiments, the ASD comprises a hydrophilic polymer. In some embodiments, the hydrophilic polymer is in an amount of about 5% to about 70% by weight of the ASD. In some embodiments, the hydrophilic polymer is in an amount of about 15% to about 65% by weight of the ASD. In some embodiments, the hydrophilic polymer is in an amount of about 15% to about 40% by weight of the ASD. In some embodiments, the hydrophilic polymer is in an amount of about 13% by weight of the ASD. In some embodiments, the hydrophilic polymer is VA64, HPMC-E5, HPMCAS-LF, PVP-K30, or any combination thereof. In some embodiments, the hydrophilic polymer is VA64. In some embodiments, the hydrophilic polymer is HPMC-E5. In some embodiments, the hydrophilic polymer is PVP-K30. In some embodiments, the hydrophilic polymer is HPMCAS-LF. In some embodiments, the hydrophilic polymer comprises HPMC, HPMCAS, polyvinyl acetate and polyvinylcaprolactame-based graft copolymer, PVP, copovidone, polymethacrylates, or a combination thereof. In some embodiments, the ASD comprises a surfactant. In some embodiments, the surfactant is in an amount of about 10% to about 50% by weight of the ASD. In some embodiments, the surfactant is in an amount of about 9% to about 40% by weight of the ASD. In some embodiments, the surfactant is in an amount of about 15% to about 30% by weight of the ASD. In some embodiments, the surfactant is in an amount of about 20% to about 25% by weight of the ASD. In some embodiments, the surfactant is TPGS. In some embodiments, TPGS is present in an amount of about 9-30% by weight of the ASD. In some embodiments, the surfactant is lecithin. In some embodiments, lecithin is present in an amount of about 9-25% by weight of the ASD. In some embodiments, the surfactant is RH40. In some embodiments, RH40 is present in an amount of about 18% by weight of the ASD. In some embodiments, the surfactant is a Pluronic F-68. In some embodiments, Pluronic F-68 is present in an amount of about 18% by weight of the ASD. In some embodiments, the surfactant is PEG6000. In some embodiments, PEG6000 is present in an amount of about 18% by weight of the ASD. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the ASD comprises an organic acid. In some embodiments, the organic acid is present in an amount of about 10% to about 40% by weight of the ASD. In some embodiments, the organic acid is present in an amount of about 10% to about 35% by weight of the ASD. In some embodiments, the organic acid is present in an amount of about 10% to about 30% by weight of the ASD. In some embodiments, the organic acid is tartaric acid. In some embodiments, the tartaric acid is present in an amount of about 10% to about 35% by weight of the ASD. In some embodiments, the organic acid is citric acid. In some embodiments, the citric acid is present in an amount of about 10% to 25% by weight of the ASD. In some embodiments, the ASD comprises an adsorbent. In some embodiments, the adsorbent is present in an amount of about 15% to about 40% by weight of the ASD. In some embodiments, the adsorbent is present in an amount of about 10% to about 30% by weight of the ASD. In some embodiments, the adsorbent is SiO₂.

In some embodiments, the pharmaceutical composition described herein comprises an ASD that comprising lurasidone free base or a pharmaceutically acceptable salt thereof in an amount of about 10% to 30% by weight of the ASD; a hydrophilic polymer in an amount of about 25% to 60% by weight of the ASD, and wherein the hydrophilic polymer is HPMCAS-LF; and a surfactant in an amount of about 9% to about 25% by weight of the ASD, wherein the surfactant is TPGS, lecithin, or any combination thereof.

In some embodiments, the pharmaceutical composition described herein comprises an ASD that consists of lurasidone free base or a pharmaceutically acceptable salt thereof in an amount of about 13% to about 30% by weight of the ASD; a hydrophilic polymer in an amount of about 25% to about 65% by weight of the ASD, wherein the hydrophilic polymer is HPMC-E5, VA64, PVP-K30, or HPMC-E5; a surfactant in an amount of about 13% to 25% by weight of the ASD, wherein the surfactant is TPGS; and an organic acid present in an amount of about 13% to 29% by weight of the ASD, and wherein the organic acid is tartaric acid or citric acid.

In some embodiments, the pharmaceutical composition described herein comprises an ASD that consists of lurasidone free base or a pharmaceutically acceptable salt thereof in an amount of about 13% to 22% by weight of the ASD; a hydrophilic polymer in an amount of about 22% to 46% by weight of the ASD, wherein the hydrophilic polymer is HPMCAS-LF or HPMC-E5; a surfactant in an amount of about 13% to 22% by weight of the ASD, wherein the surfactant is TPGS, lecithin, or any combination thereof; and an absorbent in an amount of about 20-33% by weight of the ASD, wherein the adsorbent is SiO₂.

In some embodiments, the pharmaceutical composition described herein comprises an ASD that consists of lurasidone free base or a pharmaceutically acceptable salt thereof in an amount of about 11% to 20% by weight of the ASD; a hydrophilic polymer in an amount of about 11-30% by weight of the ASD, wherein the hydrophilic polymer is HPMC-E5; a surfactant in an amount of about 11% to 20% by weight of the ASD, wherein the surfactant is TPGS, RH40, Pluronic F-68, PEG6000, or lecithin, or any combination thereof; an organic acid in an amount of about 11-20% by weight of the ASD in which the organic acid is tartaric acid or citric acid; and an adsorbent in an amount of about 10% to 27% by weight of the ASD, wherein the adsorbent is SiO₂.

In some embodiments, the pharmaceutical composition described herein comprises an ASD comprising lurasidone free base or a pharmaceutically acceptable salt thereof. In some embodiments, the lurasidone free base or a pharmaceutically acceptable salt thereof is in an amount of about 10-30% by weight of the ASD. In some embodiments, the lurasidone free base or a pharmaceutically acceptable salt thereof is in an amount of about 15-20% by weight of the ASD. In some embodiments, the lurasidone free base or a pharmaceutically acceptable salt thereof is in an amount of about 15% by weight of the ASD. In some embodiments, the lurasidone free base or a pharmaceutically acceptable salt thereof is in an amount of about 18% by weight of the ASD. In some embodiments, the ASD comprises a hydrophilic polymer. In some embodiments, the hydrophilic polymer is in an amount of about 10% to about 30% by weight of the ASD. In some embodiments, the hydrophilic polymer is in an amount of about 10% to about 20% by weight of the ASD. In some embodiments, the hydrophilic polymer is in an amount of about 15% by weight of the ASD. In some embodiments, the hydrophilic polymer is in an amount of about 18% by weight of the ASD. In some embodiments, the hydrophilic polymer is HPMC-E5, HPMCAS, PVP, or PVP/VA or a combination thereof. In some embodiments, the hydrophilic polymer is HPMC-E5. In some embodiments, the hydrophilic polymer is VA64. In some embodiments, the hydrophilic polymer is VA64. In some embodiments, the ASD comprises a surfactant. In some embodiments, the surfactant is in an amount of about 10% to about 40% by weight of the ASD. In some embodiments, the surfactant is in an amount of about 10% to about 20% by weight of the ASD. In some embodiments, the surfactant is in an amount of about 18% by weight of the ASD. In some embodiments, the surfactant is in an amount of about 20% to about 40% by weight of the ASD. In some embodiments, the surfactant is in an amount of about 31% by weight of the ASD. In some embodiments, the surfactant is TPGS or lecithin or a combination thereof. In some embodiments, TPGS is present in an amount of about 15% by weight of the ASD. In some embodiments, lecithin is present in an amount of about 15% by weight of the ASD. In some embodiments, the ASD comprises an organic acid. In some embodiments, the organic acid is present in an amount of about 10% to about 30% by weight of the ASD. In some embodiments, the organic acid is present in an amount of about 10% to about 20% by weight of the ASD. In some embodiments, the organic acid is present in an amount of about 15% by weight of the ASD. In some embodiments, the organic acid is present in an amount of about 18% by weight of the ASD. In some embodiments, the organic acid is citric acid, or tartaric acid, or a combination thereof. In some embodiments, the organic acid is citric acid. In some embodiments, the organic acid is tartaric acid. In some embodiments, the ASD comprises an adsorbent. In some embodiments, the adsorbent is present in an amount of about 15% to about 40% by weight of the ASD. In some embodiments, the adsorbent is present in an amount of about 20% to about 30% by weight of the ASD. In some embodiments, the adsorbent is present in an amount of about 23% by weight of the ASD. In some embodiments, the adsorbent is present in an amount of about 27% by weight of the ASD. In some embodiments, the adsorbent is SiO₂.

In some embodiments, the pharmaceutical composition described herein comprises an ASD that comprising lurasidone free base or a pharmaceutically acceptable salt thereof in an amount of about 15% by weight of the ASD; a hydrophilic polymer in an amount of about 15% by weight of the ASD, wherein the hydrophilic polymer is HPMC-E5; TPGS in an amount of about 15% by weight of the ASD; lecithin in an amount of about 15% by weight of the ASD; citric acid in an amount of about 15% by weight of the ASD; and an adsorbent in an amount of about 23% by weight of the ASD, wherein the adsorbent is SiO₂.

In some embodiments, the pharmaceutical composition described herein comprises an ASD that comprising lurasidone free base or a pharmaceutically acceptable salt thereof in an amount of about 18% by weight of the ASD; a hydrophilic polymer in an amount of about 18% by weight of the ASD, wherein the hydrophilic polymer is HPMC-E5; TPGS in an amount of about 18% by weight of the ASD; citric acid in an amount of about 18% by weight of the ASD; and an adsorbent in an amount of about 27% by weight of the ASD, wherein the adsorbent is SiO₂.

In some embodiments, the pharmaceutical composition described herein comprises an ASD that consists of lurasidone free base or a pharmaceutically acceptable salt thereof in an amount of about 10% to about 30% by weight of the ASD; a hydrophilic polymer in an amount of about 10% to about 30% by weight of the ASD, wherein the hydrophilic polymer is HPMC-E5, HPMCAS, PVP, or PVP/VA or a combination thereof; a surfactant in an amount of about 10% to about 40% by weight of the ASD, wherein the surfactant is TPGS or lecithin or a combination thereof; an organic acid in an amount of about 10% to about 30% by weight of the ASD, wherein the organic acid is citric acid; and optionally an adsorbent in an amount of about 15% to about 40% by weight of the ASD, wherein the adsorbent is SiO₂.

In some embodiments, the pharmaceutical composition described herein comprises an ASD that consists of lurasidone free base or a pharmaceutically acceptable salt thereof in an amount of about 15% by weight of the ASD; a hydrophilic polymer in an amount of about 15% by weight of the ASD, wherein the hydrophilic polymer is HPMC-E5; TPGS in an amount of about 15% by weight of the ASD; lecithin in an amount of about 15% by weight of the ASD; citric acid in an amount of about 15% by weight of the ASD; and an adsorbent in an amount of about 23% by weight of the ASD, wherein the adsorbent is SiO₂.

In some embodiments, the pharmaceutical composition described herein comprises an ASD that consists of lurasidone free base or a pharmaceutically acceptable salt thereof in an amount of about 18% by weight of the ASD; a hydrophilic polymer in an amount of about 18% by weight of the ASD, wherein the hydrophilic polymer is HPMC-E5; TPGS in an amount of about 18% by weight of the ASD; citric acid in an amount of about 18% by weight of the ASD; and an adsorbent in an amount of about 27% by weight of the ASD, wherein the adsorbent is SiO₂.

In some embodiments, an ASD comprises lurasidone free base or a pharmaceutically acceptable salt thereof. In some embodiments, the lurasidone free base or a pharmaceutically acceptable salt thereof is in an amount of about 20 mg to about 80 mg. In some embodiments, the ASD comprises a hydrophilic polymer. In some embodiments, the hydrophilic polymer is in an amount of about 20 mg to about 180 mg. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 20 mg to about 70 mg. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 20 mg to about 70 mg. In some embodiments, the organic acid is tartaric acid or citric acid or a combination thereof. In some embodiments, the ASD comprises an adsorbent. In some embodiments, the adsorbent is in an amount of about 30 mg to about 100 mg. In some embodiments, the adsorbent is SiO2. In some embodiments, the pharmaceutical composition described herein comprising an ASD is formulated in a unit dosage form.

In some embodiments, an ASD comprises lurasidone free base or a pharmaceutically acceptable salt thereof. In some embodiments, the lurasidone free base or a pharmaceutically acceptable salt thereof is in an amount of about 20 mg to about 60 mg. In some embodiments, the ASD comprises a hydrophilic polymer. In some embodiments, the hydrophilic polymer is in an amount of about 20 mg to about 160 mg. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 20 mg to about 60 mg. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 20 mg to 60 mg. In some embodiments, the organic acid is tartaric acid or citric acid or a combination thereof. In some embodiments, the ASD comprises an adsorbent. In some embodiments, the adsorbent is in an amount of about 30 mg to about 80 mg. In some embodiments, the adsorbent is SiO2. In some embodiments, the pharmaceutical composition described herein comprising an ASD is formulated in a unit dosage form.

In some embodiments, pharmaceutical compositions described herein comprising an ASD that comprises lurasidone free base or a pharmaceutically acceptable salt thereof have acceptable storage stability. In some embodiments, the pharmaceutical composition is storage stable chemically for at least 2 weeks at 75° C./75% RH, wherein a storage stable pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline lurasidone hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline lurasidone hydrochloride, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline lurasidone hydrochloride, when measured as Cₘₐₓ after oral administration.

In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of LATUDA capsule comprising lurasidone hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of LATUDA capsule comprising lurasidone hydrochloride, when measured as Cₘₐₓ after oral administration. In some embodiments, the pharmaceutical composition exhibits a bioavailability that is higher than a bioavailability of LATUDA by about 1.1 fold to about 10 fold. In some embodiments, the pharmaceutical composition exhibits a bioavailability that is higher than a bioavailability of LATUDA by about 1.1 fold to about 3 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, or about 5 fold to about 10 fold. In some embodiments, the pharmaceutical compositions comprise an ASD comprising lurasidone or lurasidone hydrochloride. In some embodiment, the bioavailability is measured in fasted condition.

In some embodiments, the pharmaceutical composition described herein exhibits a bioavailability that is higher than a bioavailability of LATUDA by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUCₗₐₛₜ or Cₘₐₓ after oral administration. In some embodiments, the pharmaceutical composition exhibits a bioavailability that is higher than a bioavailability of LATUDA by at least about 2 fold. In some embodiments, the pharmaceutical composition exhibits a bioavailability that is higher than a bioavailability of LATUDA by at least about 4 fold. In some embodiments, the pharmaceutical composition exhibits a bioavailability that is higher than a bioavailability of LATUDA by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘax after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiment, the bioavailability is measured under fasted condition. In some embodiments, the pharmaceutical compositions comprise an ASD comprising lurasidone free base or a pharmaceutically acceptable salt thereof such as lurasidone hydrochloride.

In some embodiments, the pharmaceutical compositions described herein exhibits a bioavailability that is from about 100% to about 3000%, 200% to about 2000%, 300% to about 1000%, about 400% to about 700%, 500% to about 800%, or about 600% to about 700% of a bioavailability of a corresponding reference composition comprising lurasidone hydrochloride when measured as AUCₗₐₛₜ or Cₘₐₓ after oral administration, wherein the corresponding reference pharmaceutical composition does not comprise an amorphous solid dispersion. In some embodiments, the reference composition is at least about 1.1 times the dosage of the pharmaceutical compositions. In some embodiments, the reference composition is at least about 1.1 times, about 1.5 times, about 2 times, about 2.5 times, about 3 times, about 4 times, about 5 times, about 6 times, about 7 times, about 8 times, about 9 times, or about 10 times the dosage of the pharmaceutical composition. In some embodiments, the pharmaceutical composition comprises an ASD comprising lurasidone free base or a pharmaceutically acceptable salt thereof. In some embodiments, the reference composition comprises lurasidone free base or a pharmaceutically acceptable salt thereof, wherein the reference composition does not comprise an ASD. In some embodiments, the reference composition is LATUDA. In some embodiment, the bioavailability is measured under fasted condition.

In some embodiments, the pharmaceutical compositions described herein exhibits a bioavailability that is from about 300% to about 1000% of a bioavailability of a corresponding reference composition comprising lurasidone hydrochloride, when measured as AUCₗₐₛₜ after oral administration under fasted condition, wherein the corresponding reference pharmaceutical composition does not comprise an amorphous solid dispersion. In some embodiments, the reference composition is LATUDA. In some embodiment, the bioavailability is measured under fasted condition.

In some embodiments, the pharmaceutical compositions described herein exhibits a bioavailability that is from about 500% to about 1500% of a bioavailability of a corresponding reference composition comprising lurasidone hydrochloride, when measured as Cₘₐₓ after oral administration under fasted condition, wherein the corresponding reference pharmaceutical composition does not comprise an amorphous solid dispersion. In some embodiments, the reference composition is LATUDA. In some embodiment, the bioavailability is measured under fasted condition.

In some embodiments, the pharmaceutical compositions described herein exhibits a bioavailability that is at least about 300% of a bioavailability of a corresponding reference composition comprising lurasidone hydrochloride, when measured as AUCₗₐₛₜ after oral administration under fasted condition, wherein the corresponding reference pharmaceutical composition does not comprise an amorphous solid dispersion. In some embodiments, the reference composition is LATUDA. In some embodiment, the bioavailability is measured under fasted condition.

In some embodiments, the pharmaceutical compositions described herein exhibits a bioavailability that is at least about 1000% of a bioavailability of a corresponding reference composition comprising lurasidone hydrochloride, when measured as Cₘₐₓ after oral administration under fasted condition, wherein the corresponding reference pharmaceutical composition does not comprise an amorphous solid dispersion. In some embodiments, the reference composition is LATUDA. In some embodiment, the bioavailability is measured under fasted condition.

In some embodiments, salts of compounds of lurasidone are formed, for example, as acid addition salts (e.g., with organic or inorganic acids), from compounds of lurasidone with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid or citric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### g) Vilazodone Hydrochloride

In one aspect, disclosed herein are pharmaceutical compositions comprising a ASD that comprises an API, a hydrophilic polymer, a surfactant, optionally an organic acid, and optionally an adsorbent. In one aspect, disclosed herein are pharmaceutical compositions comprising an ASD, wherein the ASD comprises an API. In some embodiments, the API is vilazodone free base or a pharmaceutically acceptable salt thereof. In some embodiments, the API is vilazodone hydrochloride.

In some embodiments, the ASD comprises vilazodone free base or a pharmaceutically acceptable salt thereof (such as vilazodone hydrochloride) in an amount of about 3% to about 60% by weight of the ASD. In some embodiments, the vilazodone free base or a pharmaceutically acceptable salt thereof is present in the amorphous solid dispersion in a weight percent of about 10% to about 50%. In some embodiments, the vilazodone free base or a pharmaceutically acceptable salt thereof is present in the amorphous solid dispersion in a weight percent of about 15% to about 30%. In some embodiments, the vilazodone free base or a pharmaceutically acceptable salt thereof is present in the amorphous solid dispersion in a weight percent of about 25% to about 40%. In some embodiments, the ASD comprises vilazodone free base or a pharmaceutically acceptable salt thereof in an amount of about 3% to about 50% by weight of the ASD. In some embodiments, the ASD comprises vilazodone free base or a pharmaceutically acceptable salt thereof in an amount of about 5% to about 40% by weight of the ASD. In some embodiments, the ASD comprises vilazodone free base or a pharmaceutically acceptable salt thereof in an amount of about 10% to about 35% by weight of the ASD. In some embodiments, the ASD comprises vilazodone free base or a pharmaceutically acceptable salt thereof in an amount of about 25% by weight of the ASD.

In some embodiments, the vilazodone free base or a pharmaceutically acceptable salt thereof such as vilazodone hydrochloride is present in the amorphous solid dispersion in a weight percent of about 5% to about 60%. In some embodiments, the vilazodone free base or a pharmaceutically acceptable salt thereof is present in the amorphous solid dispersion in a weight percent of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, or about 55% to about 60%. In some embodiments, the vilazodone free base or a pharmaceutically acceptable salt thereof is present in the amorphous solid dispersion in a weight percent of about 5%, about 10%, about 15%, about 20%, about 25%, about 30%, about 35%, about 40%, about 45%, about 50%, about 55%, or about 60%. In some embodiments, the vilazodone free base or a pharmaceutically acceptable salt thereof e is present in the amorphous solid dispersion in a weight percent of at least about 5%, about 10%, about 15%, about 20%, about 25%, about 30%, about 35%, about 40%, about 45%, about 50%, or about 55%. In some embodiments, the vilazodone free base or a pharmaceutically acceptable salt thereof is present in the amorphous solid dispersion in a weight percent of at most about 10%, about 15%, about 20%, about 25%, about 30%, about 35%, about 40%, about 45%, about 50%, about 55%, or about 60%.

In some embodiments, the ASD comprises a hydrophilic polymer. In some embodiments, the hydrophilic polymer is non-ionic. In some embodiments, the hydrophilic polymer is ionic. In some embodiments, the hydrophilic polymer is enteric polymer. In some embodiments, the pharmaceutical composition described herein comprises the ASD comprising a hydrophilic polymer. In some embodiments, the hydrophilic polymer is HPMC. In some embodiments, the hydrophilic polymer is HPMC. In some embodiments, the hydrophilic polymer is polymethacrylates. In some embodiments, the hydrophilic polymer is PVP/VA. In some embodiments, the hydrophilic polymer is HPMCAS. In some embodiments, the hydrophilic polymer is polyvinyl acetate and polyvinylcaprolactame-based graft copolymer. In some embodiments, the hydrophilic polymer is PVP. In some embodiments, the hydrophilic polymer is HPMC, polymethacrylates, PVP/VA, HPMCAS, polyvinyl acetate and polyvinylcaprolactame-based graft copolymer, PVP, or a combination thereof. In some embodiments, the hydrophilic polymer comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides (e.g., Labrasol or Gelucire), polysorbate, or a combination thereof. In some embodiments, the hydrophilic polymer is present in an amount of about 1% to about 80% by weight of the ASD. In some embodiments, the hydrophilic polymer is present in an amount of about 5% to about 70% by weight of the ASD. In some embodiments, the hydrophilic polymer is present in an amount of about 10% to about 60% by weight of the ASD. In some embodiments, the hydrophilic polymer is present in an amount of about 20% to about 50% by weight of the ASD. In some embodiments, the hydrophilic polymer is present in an amount of about 20% to about 40% by weight of the ASD.

In some embodiments, the ASD comprises a surfactant. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS. In some embodiments, the surfactant is TPGS or lecithin or a combination thereof. In some embodiments, the surfactant is TPGS. In some embodiments, the surfactant is lecithin. In some embodiments the surfactant is present in an amount of about 5% to about 60% by weight of the ASD. In some embodiments, the surfactant is present in an amount of about 5% to about 40% by weight of the ASD. In some embodiments, the surfactant is present in an amount of about 5% to about 30% by weight of the ASD.

In some embodiments, the ASD comprises an inorganic acid or organic acid. In some embodiments, the ASD comprises optionally an organic acid. In some embodiments, the ASD comprises an organic acid, wherein the organic acid is tartaric acid or citric acid. In some embodiments, the organic acid is tartaric acid. In some embodiments, the organic acid is citric acid. In some embodiments, the inorganic acid or organic acid is present in an amount of about 10% to about 40% by weight of the ASD. In some embodiments, the inorganic acid or organic acid is present in an amount of about 10% to about 30% by weight of the ASD.

In some embodiments, the ASD comprises optionally an adsorbent. In some embodiments, the adsorbent is silicone dioxide. In some embodiments, the adsorbent is present in an amount of about 15% to about 40% by weight of the ASD.

In some embodiments, an ASD comprises vilazodone free base or a pharmaceutically acceptable salt thereof such as vilazodone hydrochloride. In some embodiments, vilazodone free base or a pharmaceutically acceptable salt thereof is in an amount of about 5% to about 40% by weight of the ASD. In some embodiments, the ASD comprises a hydrophilic polymer. In some embodiments, the hydrophilic polymer is in an amount of about 10% to about 50% by weight of the ASD. In some embodiments, the hydrophilic polymer is HPMC, polymethacrylates, PVP/VA, HPMCAS, polyvinyl acetate and polyvinylcaprolactame-based graft copolymer, PVP, or a combination thereof. In some embodiments, the ASD comprises a surfactant. In some embodiments, the surfactant is in an amount of about 10% to about 40% by weight of the ASD. In some embodiments, the surfactant is TPGS or lecithin or a combination thereof. In some embodiments, the ASD comprises an organic acid. In some embodiments, the organic acid is in an amount of about 10% to about 40% by weight of the ASD. In some embodiments, the organic acid is citric acid.

In some embodiments, an ASD comprises vilazodone free base or a pharmaceutically acceptable salt thereof. In some embodiments, vilazodone free base or a pharmaceutically acceptable salt thereof is in an amount of about 15% to about 30% by weight of the ASD. In some embodiments, the ASD comprises a hydrophilic polymer. In some embodiments, the hydrophilic polymer is in an amount of about 15% to about 30% by weight of the ASD. In some embodiments, the hydrophilic polymer is HPMC, polymethacrylates, PVP/VA, HPMCAS, polyvinyl acetate and polyvinylcaprolactame-based graft copolymer, PVP, or a combination thereof. In some embodiments, the ASD comprises a surfactant. In some embodiments, the surfactant is in an amount of about 15% to about 30% by weight of the ASD. In some embodiments, the surfactant is TPGS or lecithin or a combination thereof. In some embodiments, the ASD comprises an organic acid. In some embodiments, the organic acid is in an amount of about 15% to about 30% by weight of the ASD. In some embodiments, the organic acid is citric acid.

In some embodiments, the pharmaceutical composition described herein comprises an ASD comprising vilazodone free base or a pharmaceutically acceptable salt thereof. In some embodiments, vilazodone free base or a pharmaceutically acceptable salt thereof is in an amount of about 15% to about 25% by weight of the ASD. In some embodiments, the ASD comprises a hydrophilic polymer. In some embodiments, the hydrophilic polymer is in an amount of about 25% to about 35% by weight of the ASD. In some embodiments, the hydrophilic polymer is HPMC, polymethacrylates, PVP/VA, HPMCAS, polyvinyl acetate and polyvinylcaprolactame-based graft copolymer, PVP, or a combination thereof. In some embodiments, the ASD comprises a surfactant. In some embodiments, the surfactant is in an amount of about 15% to about 25% by weight of the ASD. In some embodiments, the surfactant is TPGS or lecithin or a combination thereof. In some embodiments, the ASD comprises an organic acid. In some embodiments, the organic acid is in an amount of about 15% to about 25% by weight of the ASD. In some embodiments, the organic acid is citric acid.

In some embodiments, the pharmaceutical composition described herein comprising an ASD is formulated in a unit dosage form. In some embodiments, the ASD comprises vilazodone free base or a pharmaceutically acceptable salt thereof. In some embodiments, the vilazodone free base or a pharmaceutically acceptable salt thereof is in an amount of about 5 mg to about 100 mg. In some embodiments, the vilazodone free base or a pharmaceutically acceptable salt thereof is in an amount of about 5 mg to about 50 mg. In some embodiments, the ASD comprises a hydrophilic polymer. In some embodiments, the hydrophilic polymer is in an amount of about 10 mg to about 300 mg. In some embodiments, the hydrophilic polymer is in an amount of about 10 mg to about 200 mg. In some embodiments, the hydrophilic polymer is in an amount of about 10 mg to about 100 mg. In some embodiments, the hydrophilic polymer is HPMC, polymethacrylates, PVP/VA, HPMCAS, polyvinyl acetate and polyvinylcaprolactame-based graft copolymer, PVP, or a combination thereof. In some embodiments, the ASD comprises a surfactant. In some embodiments, the surfactant is in an amount of about 5 mg to about 100 mg. In some embodiments, the surfactant is in an amount of about 5 mg to about 50 mg. In some embodiments, the surfactant is TPGS or lecithin or a combination thereof. In some embodiments, the ASD comprises an organic acid. In some embodiments, the organic acid is in an amount of about 5 mg to about 100 mg. In some embodiments, the organic acid is in an amount of about 5 mg to about 50 mg. In some embodiments, the organic acid is citric acid.

In some embodiments, the pharmaceutical composition described herein comprises an ASD comprising vilazodone free base or a pharmaceutically acceptable salt thereof. In some embodiments, the vilazodone free base or a pharmaceutically acceptable salt thereof is in an amount of about 5-50% by weight of the ASD. In some embodiments, the vilazodone free base or a pharmaceutically acceptable salt thereof is in an amount of about 10-30% by weight of the ASD. In some embodiments, the vilazodone free base or a pharmaceutically acceptable salt thereof is in an amount of about 10-25% by weight of the ASD. In some embodiments, the ASD comprises a hydrophilic polymer. In some embodiments, the hydrophilic polymer is in an amount of about 5% to about 70% by weight of the ASD. In some embodiments, the hydrophilic polymer is in an amount of about 15% to about 60% by weight of the ASD. In some embodiments, the hydrophilic polymer is in an amount of about 25% to about 50% by weight of the ASD. In some embodiments, the hydrophilic polymer is in an amount of about 25% by weight of the ASD. In some embodiments, the hydrophilic polymer is in an amount of about 50% by weight of the ASD. In some embodiments, the hydrophilic polymer is HPMC-E5, HPMCAS-LF, PVP-K30, PVP/VA, or soluplus. In some embodiments, the hydrophilic polymer comprises HPMC, HPMCAS, polyvinyl acetate and polyvinylcaprolactame-based graft copolymer, PVP, copovidone, polymethacrylates, or a combination thereof. In some embodiments, the hydrophilic polymer is HPMC-E5. In some embodiments, the hydrophilic polymer is PVP-K30. In some embodiments, the hydrophilic polymer is PVP/VA. In some embodiments, the hydrophilic polymer is HPMCAS-LF. In some embodiments, the hydrophilic polymer is soluplus. In some embodiments, the ASD comprises a surfactant. In some embodiments, the surfactant is in an amount of about 10% to about 50% by weight of the ASD. In some embodiments, the surfactant is in an amount of about 10% to about 40% by weight of the ASD. In some embodiments, the surfactant is in an amount of about 10% to about 30% by weight of the ASD. In some embodiments, the surfactant is in an amount of about 25% by weight of the ASD. In some embodiments, the surfactant is in an amount of about 29% by weight of the ASD. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant is TPGS. In some embodiments, TPGS is present in an amount of about 25% by weight of the ASD. In some embodiments, the surfactant is lecithin. In some embodiments, lecithin is present in an amount of about 29% by weight of the ASD. In some embodiments, the ASD comprises an organic acid. In some embodiments, the organic acid is present in an amount of about 10% to about 35% by weight of the ASD. In some embodiments, the organic acid is present in an amount of about 15% to about 30% by weight of the ASD. In some embodiments, the organic acid is citric acid. In some embodiments, the citric acid is present in an amount of about 25% to about 29% by weight of the ASD.

In some embodiments, the pharmaceutical composition described herein comprises an ASD that consists of vilazodone free base or a pharmaceutically acceptable salt thereof in an amount of about 25% by weight of the ASD; a hydrophilic polymer in an amount of about 50% by weight of the ASD, wherein the hydrophilic polymer is HPMC-E5, PVP/VA, PVP-K30, soluplus, or HPMCAS-LF; and a surfactant in an amount of about 25% by weight of the ASD, wherein the surfactant is TPGS.

In some embodiments, the pharmaceutical composition described herein comprises an ASD that consists of vilazodone free base or a pharmaceutically acceptable salt thereof in an amount of about 13% to 22% by weight of the ASD; a hydrophilic polymer in an amount of about 22% to 46% by weight of the ASD, wherein the hydrophilic polymer is HPMCAS-LF or HPMC-E5; a surfactant in an amount of about 13% to 22% by weight of the ASD, wherein the surfactant is TPGS, lecithin, or any combination thereof; and an absorbent in an amount of about 20-33% by weight of the ASD, wherein the adsorbent is SiO₂.

In some embodiments, the pharmaceutical composition described herein comprises an ASD that consists of vilazodone free base or a pharmaceutically acceptable salt thereof in an amount of about 14% to 25% by weight of the ASD; a hydrophilic polymer in an amount of about 25-29% by weight of the ASD, wherein the hydrophilic polymer is HPMC-E5; a surfactant in an amount of about 25% to 29% by weight of the ASD, wherein the surfactant is TPGS or lecithin; and an organic acid in an amount of about 25-29% by weight of the ASD in which the organic acid is citric acid.

In some embodiments, pharmaceutical compositions described herein comprising an ASD that comprises vilazodone free base or a pharmaceutically acceptable salt thereof such as vilazodone hydrochloride have acceptable storage stability. In some embodiments, the pharmaceutical composition is storage stable chemically for at least 2 weeks at 75° C./75% RH, wherein a storage stable pharmaceutical composition has less than 5% degradation of the API at the end of the storage period. In some embodiments, the pharmaceutical composition is storage stable for at least 6 months at 40° C./75% RH, wherein a storage stable pharmaceutical composition has less than 0.5% of any impurity at the end of the storage period. In some embodiments, the pharmaceutical composition is storage stable for at least 2 weeks, 1 month, 2 months, 3 months, 6 months, 12 months, or 24 months at 25° C./60% RH, wherein a storage stable pharmaceutical composition has less than 0.5% of any impurity at the end of the storage period.

In some embodiments, the pharmaceutical compositions described herein have a superior bioavailability than a bioavailability of a corresponding reference composition comprising crystalline vilazodone hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline vilazodone hydrochloride, when measured as the AUC, AUCinf, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of a corresponding composition comprising crystalline vilazodone hydrochloride, when measured as Cₘₐₓ after oral administration.

In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VIIBRYD capsule comprising vilazodone hydrochloride, when measured as AUC, AUC_{inf}, or AUCₗₐₛₜ after oral administration. In some embodiments, the pharmaceutical composition exhibits a bioavailability that is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 5.5-fold, 6-fold, 7-fold, 8-fold, 9-fold, or 10-fold higher than a bioavailability of VIIBRYD capsule comprising vilazodone hydrochloride, when measured as Cₘₐₓ after oral administration. In some embodiments, the pharmaceutical composition exhibits a bioavailability that is higher than a bioavailability of VIIBRYD by about 1.1 fold to about 10 fold. In some embodiments, the pharmaceutical composition exhibits a bioavailability that is higher than a bioavailability of VIIBRYD by about 1.1 fold to about 2 fold, about 1.1 fold to about 3 fold, about 1.1 fold to about 4 fold, about 1.1 fold to about 5 fold, about 1.1 fold to about 6 fold, about 1.1 fold to about 7 fold, about 1.1 fold to about 8 fold, about 1.1 fold to about 10 fold, about 1.5 fold to about 2 fold, about 1.5 fold to about 3 fold, about 1.5 fold to about 4 fold, about 1.5 fold to about 5 fold, about 1.5 fold to about 6 fold, about 1.5 fold to about 7 fold, about 1.5 fold to about 8 fold, about 1.5 fold to about 10 fold, about 2 fold to about 4 fold, about 2 fold to about 5 fold, about 2 fold to about 6 fold, about 2 fold to about 7 fold, about 2 fold to about 8 fold, about 2 fold to about 10 fold, about 3 fold to about 4 fold, about 3 fold to about 5 fold, about 3 fold to about 6 fold, about 3 fold to about 7 fold, about 3 fold to about 8 fold, about 3 fold to about 10 fold, about 4 fold to about 5 fold, about 4 fold to about 6 fold, about 4 fold to about 7 fold, about 4 fold to about 8 fold, about 4 fold to about 10 fold, about 5 fold to about 6 fold, about 5 fold to about 7 fold, about 5 fold to about 8 fold, about 5 fold to about 10 fold, about 6 fold to about 7 fold, about 6 fold to about 8 fold, about 6 fold to about 10 fold, about 7 fold to about 8 fold, about 7 fold to about 10 fold, or about 8 fold to about 10 fold. In some embodiments, the pharmaceutical compositions comprise an ASD comprising vilazodone or vilazodone hydrochloride. In some embodiment, the bioavailability is measured in fasted condition.

In some embodiments, the pharmaceutical composition described herein exhibits a bioavailability that is higher than a bioavailability of VIIBRYD by at least about 1.1 fold, about 1.3 fold, about 1.5 fold, about 1.8 fold, about 2 fold, about 3 fold, about 4 fold, about 5 fold, about 6 fold, about 7 fold, or about 8 fold when measured as AUCₗₐₛₜ or Cₘₐₓ after oral administration. In some embodiments, the pharmaceutical composition exhibits a bioavailability that is higher than a bioavailability of VIIBRYD by at least about 2 fold. In some embodiments, the pharmaceutical composition exhibits a bioavailability that is higher than a bioavailability of VIIBRYD by at least about 4 fold. In some embodiments, the pharmaceutical composition exhibits a bioavailability that is higher than a bioavailability of VIIBRYD by at most about 1.3 fold, about 1.5 fold, about 1.8 fold, about 2 fold, about 3 fold, about 4 fold, about 5 fold, about 6 fold, about 7 fold, about 8 fold, or about 10 fold. In some embodiments, the bioavailability is measured in a dog model in a fasted state. In some embodiments, the bioavailability is measured in a dog model in a fed state. In some embodiments, a bioavailability of the pharmaceutical composition does not vary for more than 50%, 40%, 30%, 20%, 15%, or 10% when orally administered in a fed state compared to administered in a fasted state, when measured AUCₗₐₛₜ after oral administration. In some embodiments, a bioavailability of the pharmaceutical composition does not vary for more than 50%, 40%, 30%, 20%, 15%, or 10% when orally administered in a fed state compared to administered in a fasted state, when measured as Cₘₐₓ after oral administration. In some embodiments, a bioavailability of the pharmaceutical composition does not vary for more than 50% when orally administered in a fed state compared to administered in a fasted state. In some embodiments, a bioavailability of the pharmaceutical composition does not vary for more than 40% when orally administered in a fed state compared to administered in a fasted state. In some embodiments, a bioavailability of the pharmaceutical composition does not vary for more than 20% when orally administered in a fed state compared to administered in a fasted state. In some embodiment, the bioavailability is measured in a dog model. In some embodiment, the dog model is beagle dog. In some embodiment, the bioavailability is measured under fasted condition. In some embodiments, the pharmaceutical compositions comprise an ASD comprising vilazodone free base or a pharmaceutically acceptable salt thereof such as vilazodone hydrochloride.

In some embodiments, the pharmaceutical compositions described herein exhibits a bioavailability that is from about 100% to about 1500%, 120% to about 1000%, 125% to about 500%, about 130% to about 450%, 140% to about 400%, or about 150% to about 300% of a bioavailability of a corresponding reference composition comprising vilazodone hydrochloride when measured as AUCₗₐₛₜ or Cₘax after oral administration, wherein the corresponding reference pharmaceutical composition does not comprise an amorphous solid dispersion. In some embodiments, the reference composition is at least about 1.1 times the dosage of the pharmaceutical compositions. In some embodiments, the reference composition is at least about 1.1 times, about 1.5 times, about 2 times, about 2.5 times, about 3 times, about 4 times, about 5 times, about 6 times, about 7 times, about 8 times, about 9 times, or about 10 times the dosage of the pharmaceutical composition. In some embodiments, the pharmaceutical composition comprises an ASD comprising vilazodone free base or a pharmaceutically acceptable salt thereof such as vilazodone hydrochloride. In some embodiments, the reference composition comprises vilazodone free base or a pharmaceutically acceptable salt thereof, wherein the reference composition does not comprise an ASD. In some embodiments, the reference composition is VIIBRYD. In some embodiment, the bioavailability is measured under fasted condition. In some embodiment, the bioavailability is measured under fed condition.

In some embodiments, the pharmaceutical compositions described herein exhibits a bioavailability that is from about 130% to about 500% of a bioavailability of a corresponding reference composition comprising vilazodone hydrochloride, when measured as AUCₗₐₛₜ after oral administration under fasted condition, wherein the corresponding reference pharmaceutical composition does not comprise an amorphous solid dispersion. In some embodiments, the reference composition is VIIBRYD. In some embodiment, the bioavailability is measured under fasted condition. In some embodiment, the bioavailability is measured under fed condition.

In some embodiments, the pharmaceutical compositions described herein exhibits a bioavailability that is from about 115% to about 600% of a bioavailability of a corresponding reference composition comprising vilazodone hydrochloride, when measured as Cₘₐₓ after oral administration under fasted condition, wherein the corresponding reference pharmaceutical composition does not comprise an amorphous solid dispersion. In some embodiments, the reference composition is VIIBRYD. In some embodiment, the bioavailability is measured under fasted condition. In some embodiment, the bioavailability is measured under fed condition.

In some embodiments, the pharmaceutical compositions described herein exhibits a bioavailability that is at least about 300% of a bioavailability of a corresponding reference composition comprising vilazodone hydrochloride, when measured as AUCₗₐₛₜ after oral administration under fasted condition, wherein the corresponding reference pharmaceutical composition does not comprise an amorphous solid dispersion. In some embodiments, the reference composition is VIIBRYD. In some embodiments, the pharmaceutical compositions described herein exhibits bioavailability that is at least about 100% of a bioavailability of a corresponding reference composition comprising vilazodone hydrochloride, when measured as AUCₗₐₛₜ after oral administration under fed condition, wherein the corresponding reference pharmaceutical composition does not comprise an amorphous solid dispersion. In some embodiments, the reference composition is VIIBRYD. In some embodiment, the bioavailability is measured under fasted condition.

In some embodiments, the pharmaceutical compositions described herein exhibits a bioavailability that is at least about 300% of a bioavailability of a corresponding reference composition comprising vilazodone hydrochloride, when measured as Cₘₐₓ after oral administration under fasted condition, wherein the corresponding reference pharmaceutical composition does not comprise an amorphous solid dispersion. In some embodiments, the reference composition is VIIBRYD. In some embodiments, the pharmaceutical compositions described herein exhibits a bioavailability that is at least about 100% of a bioavailability of a corresponding reference composition comprising vilazodone hydrochloride, when measured as Cₘₐₓ after oral administration under fed condition, wherein the corresponding reference pharmaceutical composition does not comprise an amorphous solid dispersion. In some embodiments, the reference composition is VIIBRYD. In some embodiment, the bioavailability is measured under fasted condition.

In some embodiments, salts of compounds of vilazodone are formed, for example, as acid addition salts (e.g., with organic or inorganic acids), from compounds of vilazodone with a basic nitrogen atom, e.g., the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid or citric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

### Amorphous Solid Dispersion

The present disclosure relates to pharmaceutical compositions, methods of preparing the pharmaceutical composition described thereof, methods of treating a disease or a condition by administering thereof, the pharmaceutical compositions comprising an amorphous solid dispersion further comprising a lipophilic API, a hydrophilic polymer, and optionally a surfactant.

In one aspect, disclosed herein is an amorphous solid dispersion, wherein the amorphous solid dispersion comprises: a) a lipophilic active pharmaceutical ingredient or a pharmaceutically acceptable salt thereof. In some embodiments, the active pharmaceutical ingredient has a log P in octanol-water equal or greater than 2.0. In some embodiments, the ASD comprises a hydrophilic polymer. In some embodiments, the ASD comprises a surfactant. In some embodiments, the surfactant is selected from polymeric surfactants and phospholipids. In some embodiments, the ASD comprises an adsorbent. In some embodiments, the ASD comprises an organic acid or inorganic acid.

In some embodiments, the active pharmaceutical ingredient or a pharmaceutically acceptable salt thereof is selected from abiraterone acetate, alectinib, pazopanib, cabozantinib, venetoclax, lurasidone, vilazodone, the corresponding free base thereof and pharmaceutically acceptable salts thereof. In some embodiments, the active pharmaceutical ingredient or a pharmaceutically acceptable salt thereof is present in the amorphous solid dispersion in an amount of about 5 wt % to about 70 wt % based on solids. In some embodiments, the surfactant comprises a surfactant. In some embodiments, the surfactants are selected from phospholipids, lecithin, polysorbate, TPGS, Kolliphor series (RH40), sorbitan oleate, SDS, polyvinylcaprolactame-based graft copolymer (PVAc-PVCap-PEG), macrogol (15)-hydroxystearate (e.g., sold under the trade name Solutol), and Soluplus or a combination thereof. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides (e.g., caprylocaproyl polyoxyl-8 glycerides), polysorbate, or a combination thereof. In some embodiments, the surfactant comprises lecithin. In some embodiments, the surfactant comprises PEG. In some embodiments, the surfactant comprises block copolymer of polyethylene glycol and polypropylene glycol. In some embodiments, the surfactant comprises SLS. In some embodiments, the surfactant comprises polyvinyl acetate and polyvinylcaprolactame-based graft copolymer (e.g., Soluplus). In some embodiments, the surfactant comprises polyoxyl hydrogenated castor oil (e.g., Kolliphor® RH 40). In some embodiments, the surfactant comprises polyoxylglycerides (e.g., caprylocaproyl polyoxyl-8 glycerides or Labrasol, or Lauroyl Polyoxyl-32 glycerides, or Gelucire). In some embodiments, the surfactant comprises polysorbate. In some embodiments, the surfactant comprises one or more phospholipids. In some embodiments, the surfactant comprises one or more of phosphatidylcholine, phosphatidylethanolamine, phosphatidylinositol, phosphatidylserine, plasmalogen, sphingomyelin, and phosphatidic acid. In some embodiments, the surfactant comprises lecithin. In some embodiments, the surfactant is present in the amorphous solid dispersion in an amount of about 5 wt % to about 70 wt %.

In some embodiments, a weight ratio of the active pharmaceutical ingredient or a pharmaceutically acceptable salt thereof to the surfactant is from about 10:1 to about 1:10. In some embodiments, the hydrophilic polymer is selected from PVA, oligosaccharide, polysaccharide, PVP, HPMC, HEC, HPC, PEO, HP-β-CD, HPMCAS, PEG, HPMCP, Eudragit, and Soluplus, povidone, copovidone, or a combination thereof. In some embodiments, the hydrophilic polymer is non-ionic. In some embodiments, the hydrophilic polymer is ionic. In some embodiments, the hydrophilic polymer comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the non-ionic hydrophilic polymer comprises polyvinyl alcohol (PVA), oligosaccharide, polysaccharide, polyvinylpyrrolidone (PVP), hydroxypropyl methylcellulose (HPMC, or hypromellose), hydroxyethyl cellulose (HEC), hydroxypropyl cellulose (HPC), polyethylene oxide, cyclodextrin (CD) and its derivatives such as hydroxypropyl beta cyclodextrin (HP-β-CD), hydropropylmethylcellulose acetate succinate (HPMCAS), polyethylene glycol (PEG), polyvinyl acetate and polyvinylcaprolactame-based graft copolymer (PVAc-PVCap-PEG), polyvinyl caprolactam-polyvinyl acetate-polyethylene glycol (PCL-PVAc-PEG), or a combination thereof. In some embodiments, the hydrophilic polymer is HPMC, PVP, HP-β-CD, PVA, HPMCAS, or PCL-PVAc-PEG. In some embodiments, the hydrophilic polymer is present in the amorphous solid dispersion in an amount of about 5 wt % to about 70 wt % based on solids. In some embodiments, a weight ratio of the active pharmaceutical ingredient or a pharmaceutically acceptable salt thereof to the hydrophilic polymer is from about 10:1 to about 1:10.

In some embodiments, the adsorbent is selected from silicon dioxide, active carbon, magnesium aluminum silicate, diatomite, microcrystalline cellulose (MCC), silicified microcrystalline cellulose (SMCC), tale, crosslinked povidone, sodium carboxymethylcellulose, sodium carboxymethyl starch, and also sugars or sugar alcohols such as sorbitol, mannitol, lactose, cyclodextrin, and maltodextrin. In some embodiments, the adsorbent is silicon dioxide. In some embodiments, the adsorbent is present in the amorphous solid dispersion in an amount of about 5 to about 80% wt. In some embodiments, the average particle diameter of the amorphous solid dispersion is from 1 μm to 1000 μm. In some embodiments, an average particle size of the amorphous solid dispersion, in terms of particle diameter, is from about 5 μm to about 150 μm.

In some embodiments, the amorphous solid dispersion additionally comprises an inorganic acid or organic acid. In some embodiments, the organic acid is selected from the group consisting of tartaric acid, fumaric acid, succinic acid, citric acid, lactic acid, malic acid, methanesulfonic acid, ethanesulfonic acid, isethionic acid, benzenesulfonic acid, and p-toluenesulfonic acid. In some embodiments, the inorganic acid is selected from the group consisting of hydrochloric acid, sulfuric acid, and phosphoric acid. In some embodiments, the API is an API of Table 1 or a pharmaceutically acceptable salt thereof.

In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, a solid dispersion is a solid state solution wherein an API (or API salt thereof) and hydrophilic polymer act as solute and solvent, respectively. The solid dispersion can form multiple structures depending on the composition and sample processing history. When the API loading is lower than the equilibrium solubility of API in the hydrophilic polymer, the drug is molecularly dispersed within the polymer matrix and forms a thermodynamically stable, homogeneous solution. A homogenous solution is often attainable only at very low API loading and/or high temperature. For higher loadings, the mixture becomes a supersaturated solution and the drug precipitates out. This can result in a dispersion of crystalline API particles in a hydrophilic polymer matrix, in which the drug concentration corresponds to its equilibrium solubility at that temperature. Alternatively, as API crystallization can be a slow process, an intermediate meta-stable structure may form in which amorphous API aggregates are dispersed in a hydrophilic polymer matrix containing the API in a non-crystalline amorphous state. Such amorphous solid dispersions can provide superior dissolution properties, as compared to the crystalline API.

In some embodiments, an amorphous solid dispersion described herein comprises an API, hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants. In some embodiments, the amorphous solid dispersions described here are homogenous amorphous solid dispersions. In some embodiments, the components of the amorphous dispersion are mixed and heated in a solvent, and the solvent is removed to form the amorphous solid dispersion. In some embodiments, the solvent is water. In some embodiments, the solvent is a polar organic solvent. In some embodiments, the solvent is a non-polar organic solvent. In some embodiments, the solvent is selected from water, n-butanol, n-propanol, isopropanol, formic acid, nitromethane, ethanol, methanol, acetic acid, N-methylpyrrolidone, tetrahydrofuran, ethyl acetate, methyl acetate, dimethylformamide, acetonitrile, dimethyl sulfoxide, dichloromethane (DCM), acetone, tetrahydrofuran (THF), and any combination thereof. In some embodiments, the solvent is selected from water, n-butanol, n-propanol, isopropanol, formic acid, nitromethane, ethanol, methanol, acetic acid, and any combination thereof. In some embodiments, the solvent is selected from water, methanol, ethanol and isopropanol. In some embodiments, the solvent is selected from dichloromethane, methanol, THF, and acetone. In some embodiments, the solvent is selected from a mixture of these solvents.

In some embodiments, an amorphous solid dispersion described herein comprises an API, hydrophilic polymer, optionally a surfactant, optionally an acid, and optionally, an adsorbent. In some embodiments, the components of the amorphous dispersion, such as API, hydrophilic polymer and surfactant are mixed and solubilized in a solvent, with or without heating to form a solution. In some embodiments, the adsorbent is further added into the solution to form a homogeneous suspension and the solvent is removed to form the amorphous solid dispersion. In some embodiments, the solution is sprayed on to the adsorbent and the solvent is removed to form the amorphous solid dispersion. In some embodiments, the adsorbent is selected from silicon dioxide (also termed silica), magnesium aluminometasilicate (Neusilin), microcrystalline cellulose (MCC), silicified microcrystalline cellulose (SMCC), tale, crosslinked povidone, sodium carboxymethylcellulose, sodium carboxymethylstarch, sugars, and sugar alcohols. In some embodiments, sugars and sugar slcohold comprise sorbitol, mannitol, lactose, cyclodextrin, and maltodextrin. In some embodiments, the adsorbent is silicon dioxide.

### a) Cabozantinib

In some embodiments, the present invention discloses an ASD comprising cabozantinib free base a pharmaceutically acceptable salt thereof (such as cabozantinib malate), a surfactant, a hydrophilic polymer, optionally an adsorbent, and optionally an organic acid.

In some embodiments, the present invention discloses an ASD comprising cabozantinib free base or a pharmaceutically acceptable salt thereof (such as cabozantinib malate). In some embodiments, the ASD comprises a surfactant. In some embodiments, the surfactant comprises lecithin and TPGS, polyoxylglycerides, polyoxyl hydrogenated castor or a combination thereof. In some embodiments, the ASD comprises a hydrophilic polymer. In some embodiments, the hydrophilic polymer is HPMC, copovidone, polymethacrylates, polyvinyl acetate and polyvinylcaprolactame-based graft copolymer, HPMCAS, or a combination thereof. In some embodiments, the ASD comprises an adsorbent. In some embodiments, the adsorbent is silicone dioxide. In some embodiments, the ASD comprises an organic acid. In some embodiments, the organic acid is malic acid.

In some embodiments, the present invention discloses an ASD comprising cabozantinib free base or a pharmaceutically acceptable salt thereof (such as cabozantinib malate) in an amount of about 10% to about 30% by weight of the ASD. In some embodiments, the ASD comprises a surfactant in an amount of about 10% to about 55% by weight of the ASD. In some embodiments, the surfactant comprises lecithin and TPGS, polyoxylglycerides, polyoxyl hydrogenated castor, or a combination thereof. In some embodiments, the ASD comprises a hydrophilic polymer in an amount of about 5% to about 70% by weight of the ASD. In some embodiments, the hydrophilic polymer is HPMC, copovidone, polymethacrylates, polyvinyl acetate and polyvinylcaprolactame-based graft copolymer, HPMCAS, or a combination thereof. In some embodiments, the ASD comprises an adsorbent in an amount of about 5% to 40% by weight of the ASD. In some embodiments, the adsorbent is silicone dioxide. In some embodiments, the ASD comprises an organic acid in an amount of about 5% to 40% by weight of the ASD. In some embodiments, the organic acid is malic acid.

In some embodiments, the present invention discloses an ASD comprising cabozantinib free base or a pharmaceutically acceptable salt thereof (such as cabozantinib malate) in an amount of about 15% by weight of the ASD. In some embodiments, the ASD comprises a surfactant in an amount of about 45% by weight of the ASD. In some embodiments, the surfactant comprises lecithin and TPGS, polyoxylglycerides, polyoxyl hydrogenated castor, or a combination thereof. In some embodiments, the ASD comprises a hydrophilic polymer in an amount of about 15% by weight of the ASD. In some embodiments, the hydrophilic polymer is HPMC, copovidone, polymethacrylates, polyvinyl acetate and polyvinylcaprolactame-based graft copolymer, HPMCAS, or a combination thereof. In some embodiments, the ASD comprises an adsorbent in an amount of about 23% by weight of the ASD. In some embodiments, the adsorbent is silicone dioxide.

### b) Venetoclax

In some embodiments, the present invention discloses an ASD comprising venetoclax free base or a pharmaceutically acceptable salt thereof, a surfactant, a hydrophilic polymer, and an organic acid.

In some embodiments, the present invention discloses an ASD comprising venetoclax free base or a pharmaceutically acceptable salt thereof. In some embodiments, the ASD comprises a surfactant. In some embodiments, the surfactant is TPGS lecithin, polyoxyl hydrogenated castor oil, polyoxylglycerides, polysorbate, or a combination thereof. In some embodiments, the ASD comprises a hydrophilic polymer. In some embodiments, the hydrophilic polymer is copovidone, HPMCAS, polymethacrylates, polyvinyl acetate and polyvinylcaprolactame-based graft copolymer (PVAc-PVCap-PEG), or a combination thereof. In some embodiments, the ASD comprises an organic acid. In some embodiments, the organic acid is citric acid.

In some embodiments, the present invention discloses an ASD comprising venetoclax free base or a pharmaceutically acceptable salt thereof. In some embodiments, the venetoclax free base or a pharmaceutically acceptable salt thereof is in an amount of about 10% to about 50% by weight of the ASD. In some embodiments, the venetoclax free base or a pharmaceutically acceptable salt thereof is in an amount of about 10% to about 40% by weight of the ASD. In some embodiments, the ASD comprises a surfactant. In some embodiments, the surfactant is in an amount of about 10% to about 50% by weight of the ASD. In some embodiments, the surfactant comprises lecithin, TPGS lecithin, polyoxyl hydrogenated castor oil, polyoxylglycerides, polysorbate, or a combination thereof, or a combination thereof. In some embodiments, the ASD comprises a hydrophilic polymer. In some embodiments, the hydrophilic polymer is in an amount of about 10% to about 50% by weight of the ASD. In some embodiments, the hydrophilic polymer is copovidone, HPMCAS, polymethacrylates, polyvinyl acetate and polyvinylcaprolactame-based graft copolymer (PVAc-PVCap-PEG), or a combination thereof. In some embodiments, the ASD comprises an organic acid. In some embodiments, the organic acid is in an amount of about 1% to 20% by weight of the ASD. In some embodiments, the organic acid is citric acid. In some embodiments, the ASD comprises an adsorbent. In some embodiments, the adsorbent is in an amount of about 1% to 20% by weight of the ASD. In some embodiments, the adsorbent is silicone dioxide.

In some embodiments, the present invention discloses an ASD comprising venetoclax free base or a pharmaceutically acceptable salt thereof. in an amount of about 20% to about 40% by weight of the ASD. In some embodiments, the ASD comprises a surfactant. In some embodiments, the surfactant is in an amount of about 20% to about 40% by weight of the ASD. In some embodiments, the surfactant comprises lecithin, TPGS lecithin, polyoxyl hydrogenated castor oil, polyoxylglycerides, polysorbate, or a combination thereof, or a combination thereof. In some embodiments, the ASD comprises a hydrophilic polymer. In some embodiments, the hydrophilic polymer is in an amount of about 20% to about 40% by weight of the ASD. In some embodiments, the hydrophilic polymer is copovidone, HPMCAS, polymethacrylates, polyvinyl acetate and polyvinylcaprolactame-based graft copolymer (PVAc-PVCap-PEG), or a combination thereof. In some embodiments, the ASD comprises an organic acid. In some embodiments, the organic acid is in an amount of about 3% to 15% by weight of the ASD. In some embodiments, the organic acid is citric acid.

In some embodiments, the present invention discloses an ASD comprising venetoclax free base or a pharmaceutically acceptable salt thereof. in an amount of about 31% by weight of the ASD. In some embodiments, the ASD comprises a surfactant. In some embodiments, the surfactant is in an amount of about 31% by weight of the ASD. In some embodiments, the surfactant comprises lecithin, a block copolymer of polyethylene glycol and polypropylene glycol, polyoxyl hydrogenated castor oil, polyoxylglycerides, polysorbate, TPGS, or a combination thereof. In some embodiments, the ASD comprises a hydrophilic polymer. In some embodiments, the hydrophilic polymer is in an amount of about 31% by weight of the ASD. In some embodiments, the hydrophilic polymer is copovidone (such as VA64), HPMCAS, polymethacrylates, polyvinyl acetate and polyvinylcaprolactame-based graft copolymer (PVAc-PVCap-PEG). In some embodiments, the ASD comprises an organic acid. In some embodiments, the organic acid is in an amount of about 7% by weight of the ASD. In some embodiments, the organic acid is citric acid.

### c) Abiraterone Acetate

In some embodiments, the present invention discloses an ASD comprising abiraterone free base or abiraterone acetate, a hydrophilic polymer, optionally a surfactant, optionally an inorganic acid or organic acid, and optionally an adsorbent.

In some embodiments, the present invention discloses an ASD comprising abiraterone free base or abiraterone acetate. In some embodiments, the ASD comprises a hydrophilic polymer. In some embodiments, the hydrophilic polymer is HPMCAS, polymethacrylates, or copovidone or a combination thereof, In some embodiments, the ASD comprises a surfactant. In some embodiments, the surfactant is lecithin or TPGS or a combination thereof. In some embodiments, the ASD comprises an organic acid. In some embodiments, the organic acid is tartaric acid, oleic acid, or a combination thereof. In some embodiments, the ASD comprises an adsorbent. In some embodiments, the adsorbent is silicon dioxide or magnesium aluminum silicate or a combination thereof.

In some embodiments, the present invention discloses an ASD comprising abiraterone free base or abiraterone acetate in an amount of about 5% to about 40% by weight of the ASD. In some embodiments, the ASD comprises a hydrophilic polymer. In some embodiments, the hydrophilic polymer is in an amount of about 30% to about 95% by weight of the ASD. In some embodiments, the hydrophilic polymer is HPMCAS, polymethacrylates, or copovidone or a combination thereof. In some embodiments, the ASD comprises a surfactant. In some embodiments, the surfactant is in an amount of about 10% to about 40% by weight of the ASD. In some embodiments, the surfactant is lecithin or TPGS or a combination thereof. In some embodiments, the ASD comprises an organic acid. In some embodiments, the organic acid is tartaric acid, oleic acid, or a combination thereof. In some embodiments, the ASD comprises an adsorbent. In some embodiments, the adsorbent is silicon dioxide or magnesium aluminum silicate or a combination thereof.

In some embodiments, the present invention discloses an ASD comprising abiraterone free base or abiraterone acetate in an amount of about 8% to about 30% by weight of the ASD. In some embodiments, the ASD comprises a hydrophilic polymer. In some embodiments, the hydrophilic polymer is in an amount of about 50% to about 91% by weight of the ASD. In some embodiments, the hydrophilic polymer is HPMCAS, polymethacrylates, or copovidone or a combination thereof. In some embodiments, the ASD comprises a surfactant. In some embodiments, the surfactant is in an amount of about 15% to about 30% by weight of the ASD. In some embodiments, the surfactant is lecithin or TPGS or a combination thereof. In some embodiments, the ASD comprises an organic acid. In some embodiments, the organic acid is tartaric acid, oleic acid, or a combination thereof. In some embodiments, the ASD comprises an adsorbent. In some embodiments, the adsorbent is silicon dioxide or magnesium aluminum silicate or a combination thereof.

In some embodiments, the present invention discloses an ASD comprising abiraterone free base or abiraterone acetate in an amount of about 10% by weight of the ASD. In some embodiments, the ASD comprises a hydrophilic polymer. In some embodiments, the hydrophilic polymer is in an amount of about 90% by weight of the ASD. In some embodiments, the hydrophilic polymer is HPMCAS, polymethacrylates, or copovidone or a combination thereof. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof.

In some embodiments, the present invention discloses an ASD comprising abiraterone free base or abiraterone acetate in an amount of about 20% by weight of the ASD. In some embodiments, the ASD comprises a hydrophilic polymer. In some embodiments, the hydrophilic polymer is in an amount of about 60% by weight of the ASD. In some embodiments, the hydrophilic polymer is HPMCAS, polymethacrylates, or copovidone or a combination thereof. In some embodiments, the ASD comprises a surfactant. In some embodiments, the surfactant is in an amount of about 20% by weight of the ASD. In some embodiments, the surfactant is lecithin or TPGS or a combination thereof. In some embodiments, the ASD comprises an organic acid. In some embodiments, the ASD comprises an organic acid. In some embodiments, the organic acid is in an amount of about 5% to 30% by weight of the ASD. In some embodiments, the organic acid is tartaric acid, oleic acid, or a combination thereof. In some embodiments, the ASD comprises an adsorbent. In some embodiments, the adsorbent is in an amount of about 1% to 40% by weight of the ASD. In some embodiments, the adsorbent is silicon dioxide or magnesium aluminum silicate or a combination thereof.

### d) Alectinib Hydrochloride

In some embodiments, the present invention discloses an ASD comprising alectinib free base or a pharmaceutically acceptable salt thereof (such as alectinib hydrochloride), a hydrophilic polymer, a surfactant, optionally an organic acid, and optionally an adsorbent.

In some embodiments, the present invention discloses an ASD comprising alectinib free base or a pharmaceutically acceptable salt thereof. In some embodiments, the ASD comprises a hydrophilic polymer. In some embodiments, the hydrophilic polymer is HPMC, polymethacrylates, HPMCAS or Soluplus or a combination thereof. In some embodiments, the ASD comprises a surfactant. In some embodiments, the surfactant is TPGS, polyoxylglycerides, polysorbate, polyoxyl hydrogenated castor oil, or SLS or a combination thereof. In some embodiments, the ASD comprises an organic acid. In some embodiments, the organic acid is tartaric acid. In some embodiments, the ASD comprises an adsorbent. In some embodiments, the adsorbent is silicon dioxide

In some embodiments, the present invention discloses an ASD comprising alectinib free base or a pharmaceutically acceptable salt thereof in an amount of about 5% to about 60% by weight of the ASD. In some embodiments, the ASD comprises a hydrophilic polymer. In some embodiments, the hydrophilic polymer is in an amount of about 15% to about 80% by weight of the ASD. In some embodiments, the hydrophilic polymer is HPMC, polymethacrylates, HPMCAS or Soluplus or a combination thereof. In some embodiments, the ASD comprises a surfactant. In some embodiments, the surfactant is in an amount of about 10% to about 40% by weight of the ASD. In some embodiments, the surfactant is TPGS, polyoxylglycerides, polysorbate, polyoxyl hydrogenated castor oil, or SLS or a combination thereof. In some embodiments, the ASD comprises an organic acid. In some embodiments, the organic acid is in an amount of about 10% to about 50% by weight of the ASD. In some embodiments, the organic acid is tartaric acid. In some embodiments, the ASD comprises an adsorbent in an amount of about 1% to 40% by weight of the ASD. In some embodiments, the adsorbent is silicon dioxide. In some embodiments, the present invention discloses an ASD comprising alectinib free base or a pharmaceutically acceptable salt thereof in an amount of about 15% to about 55% by weight of the ASD. In some embodiments, the ASD comprises a hydrophilic polymer. In some embodiments, the hydrophilic polymer is in an amount of about 15% to about 70% by weight of the ASD. In some embodiments, the hydrophilic polymer is HPMC, polymethacrylates, HPMCAS or Soluplus or a combination thereof. In some embodiments, the ASD comprises a surfactant. In some embodiments, the surfactant is in an amount of about 15% to about 30% by weight of the ASD. In some embodiments, the surfactant is TPGS, polyoxylglycerides, polysorbate, polyoxyl hydrogenated castor oil, or SLS or a combination thereof. In some embodiments, the ASD comprises an organic acid. In some embodiments, the organic acid is in an amount of about 20% to about 40% by weight of the ASD. In some embodiments, the organic acid is tartaric acid. In some embodiments, the ASD comprises an adsorbent. In some embodiments, the adsorbent is in an amount of about 5% to 35% by weight of the ASD. In some embodiments, the adsorbent is silicon dioxide.

In some embodiments, the present invention discloses an ASD comprising alectinib free base or a pharmaceutically acceptable salt thereof in an amount of about 25% by weight of the ASD. In some embodiments, the ASD comprises a hydrophilic polymer. In some embodiments, the hydrophilic polymer is in an amount of about 50% by weight of the ASD. In some embodiments, the hydrophilic polymer is HPMCAS, polymethacrylates, or Soluplus or a combination thereof. In some embodiments, the ASD comprises a surfactant. In some embodiments, the surfactant is in an amount of about 25% by weight of the ASD. In some embodiments, the surfactant is SLS, polyoxylglycerides, polysorbate, polyoxyl hydrogenated castor oil, or a combination thereof. In some embodiments, the ASD comprises an adsorbent. In some embodiments, the adsorbent is in an amount of about 10% to 30% by weight of the ASD. In some embodiments, the adsorbent is silicon dioxide.

### e) Pazopanib Hydrochloride

In some embodiments, the present invention discloses an ASD comprising pazopanib free base or a pharmaceutically acceptable salt thereof (such as pazopanib hydrochloride), a hydrophilic polymer, a surfactant, and optionally an organic acid, and optionally an adsorbent.

In some embodiments, the present invention discloses an ASD comprising pazopanib free base or a pharmaceutically acceptable salt thereof (such as pazopanib hydrochloride). In some embodiments, the ASD comprises a hydrophilic polymer. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is TPGS or lecithin or a combination thereof. In some embodiments, the ASD comprises an organic acid. In some embodiments, the organic acid is tartaric acid. In some embodiments, the ASD comprises an adsorbent. In some embodiments, the adsorbent is SiO2.

In some embodiments, the present invention discloses an ASD comprising pazopanib free base or a pharmaceutically acceptable salt thereof (e.g., pazopanib hydrochloride). In some embodiments, pazopanib free base or a pharmaceutically acceptable salt thereof is in an amount of about 5% to about 40% by weight of the ASD. In some embodiments, the ASD comprises a hydrophilic polymer. In some embodiments, the hydrophilic polymer is in an amount of about 10% to about 70% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 1% to about 60% by weight of the ASD. In some embodiments, the surfactant is in an amount of about 10% to about 40% by weight of the ASD. In some embodiments, the surfactant is TPGS or Lecithin or a combination thereof. In some embodiments, the ASD comprises an organic acid. In some embodiments, the organic acid is in an amount of about 10% to about 40% by weight of the ASD. In some embodiments, the organic acid is tartaric acid. In some embodiments, the ASD comprises an adsorbent. In some embodiments, the adsorbent is in an amount of about 15% to about 40% by weight of the ASD. In some embodiments, the adsorbent is SiO₂.

In some embodiments, the present invention discloses an ASD comprising pazopanib free base or a pharmaceutically acceptable salt thereof. In some embodiments, the pazopanib free base or a pharmaceutically acceptable salt thereof is in an amount of about 15% to about 30% by weight of the ASD. In some embodiments, the ASD comprises a hydrophilic polymer. In some embodiments, the hydrophilic polymer is in an amount of about 15% to about 60% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 15% to about 30% by weight of the ASD. In some embodiments, the surfactant is in an amount of about 15% to about 50% by weight of the ASD. In some embodiments, the surfactant is TPGS or Lecithin or a combination thereof. In some embodiments, the ASD comprises an organic acid. In some embodiments, the organic acid is in an amount of about 15% to about 30% by weight of the ASD. In some embodiments, the organic acid is tartaric acid. In some embodiments, the ASD comprises an adsorbent. In some embodiments, the adsorbent is in an amount of about 20% to about 35% by weight of the ASD. In some embodiments, the adsorbent is SiO₂.

In some embodiments, the present invention discloses an ASD comprising pazopanib free base or a pharmaceutically acceptable salt thereof. In some embodiments, pazopanib free base or a pharmaceutically acceptable salt thereof is in an amount of about 15-20% by weight of the ASD. In some embodiments, the ASD comprises a hydrophilic polymer. In some embodiments, the hydrophilic polymer is in an amount of about 15-20% by weight of the ASD. In some embodiments, the hydrophilic polymer is HPMC, polymethacrylates, polyvinyl acetate and polyvinylcaprolactame-based graft copolymer, PVP, copovidone, or a combination thereof. In some embodiments, the ASD comprises a surfactant. In some embodiments, the surfactant is in an amount of about 15-20% by weight of the ASD. In some embodiments, the surfactant is TPGS. In some embodiments, the ASD comprises an organic acid. In some embodiments, the organic acid is in an amount of about 15-20% by weight of the ASD. In some embodiments, the organic acid is tartaric acid. In some embodiments, the ASD comprises an adsorbent in an amount of about 25-30% by weight of the ASD. In some embodiments, the adsorbent is SiO₂.

### f) Lurasidone Hydrochloride

In some embodiments, the present invention discloses an ASD comprising lurasidone free base or a pharmaceutically acceptable salt thereof (such as lurasidone hydrochloride), a hydrophilic polymer, a surfactant, and optionally an organic acid, and optionally an adsorbent.

In some embodiments, the present invention discloses an ASD comprising lurasidone free base or a pharmaceutically acceptable salt thereof. In some embodiments, the ASD comprises a hydrophilic polymer. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is tartaric acid or citric acid or a combination thereof. In some embodiments, the ASD comprises an adsorbent. In some embodiments, the adsorbent is SiO2.

In some embodiments, the present invention discloses an ASD comprising lurasidone free base or a pharmaceutically acceptable salt thereof. In some embodiments, the lurasidone free base or a pharmaceutically acceptable salt thereof is in an amount of about 5% to about 50% by weight of the ASD. In some embodiments, the ASD comprises a hydrophilic polymer. In some embodiments, the hydrophilic polymer is in an amount of about 10% to about 70% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 40% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 10% to about 40% by weight of the ASD. In some embodiments, the organic acid is tartaric acid or citric acid or a combination thereof. In some embodiments, the ASD comprises an adsorbent in an amount of about 15% to about 40% by weight of the ASD. In some embodiments, the adsorbent is SiO₂.

In some embodiments, the present invention discloses an ASD comprising lurasidone free base or a pharmaceutically acceptable salt thereof. In some embodiments, the lurasidone free base or a pharmaceutically acceptable salt thereof is in an amount of about 15% to about 40% by weight of the ASD. In some embodiments, the ASD comprises a hydrophilic polymer. In some embodiments, the hydrophilic polymer is in an amount of about 15% to about 60% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 30% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 15% to about 40% by weight of the ASD. In some embodiments, the organic acid is tartaric acid or citric acid or a combination thereof. In some embodiments, the ASD comprises an adsorbent in an amount of about 15% to about 30% by weight of the ASD. In some embodiments, the adsorbent is SiO₂.

In some embodiments, the present invention discloses an ASD comprising lurasidone free base or a pharmaceutically acceptable salt thereof. In some embodiments, the lurasidone free base or a pharmaceutically acceptable salt thereof is in an amount of about 10% to about 25% by weight of the ASD. In some embodiments, the ASD comprises a hydrophilic polymer. In some embodiments, the hydrophilic polymer is in an amount of about 10% to about 25% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 25% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 10% to about 25% by weight of the ASD. In some embodiments, the organic acid is tartaric acid.

In some embodiments, the present invention discloses an ASD comprising lurasidone free base or a pharmaceutically acceptable salt thereof. In some embodiments, the lurasidone free base or a pharmaceutically acceptable salt thereof is in an amount of about 25% to about 35% by weight of the ASD. In some embodiments, the ASD comprises a hydrophilic polymer. In some embodiments, the hydrophilic polymer is in an amount of about 25% to about 35% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 25% to about 35% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 25% to about 35% by weight of the ASD. In some embodiments, the organic acid is citric acid.

### g) Vilazodone Hydrochloride

In some embodiments, the present invention discloses an ASD comprising vilazodone free base or a pharmaceutically acceptable salt thereof such as vilazodone hydrochloride, a hydrophilic polymer, a surfactant, optionally an organic acid, and optionally an adsorbent.

In some embodiments, the present invention discloses an ASD comprising vilazodone free base or a pharmaceutically acceptable salt thereof such as vilazodone hydrochloride. In some embodiments, the ASD comprises a hydrophilic polymer. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is citric acid.

In some embodiments, the present invention discloses an ASD comprising vilazodone free base or a pharmaceutically acceptable salt thereof such as vilazodone hydrochloride. In some embodiments, the vilazodone free base or a pharmaceutically acceptable salt is in an amount of about 5% to about 40% by weight of the ASD. In some embodiments, the ASD comprises a hydrophilic polymer. In some embodiments, the hydrophilic polymer is in an amount of about 10% to about 50% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 10% to about 40% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 10% to about 40% by weight of the ASD. In some embodiments, the organic acid is citric acid.

In some embodiments, the present invention discloses an ASD comprising vilazodone free base or a pharmaceutically acceptable salt thereof. In some embodiments, the vilazodone free base or a pharmaceutically acceptable salt is in an amount of about 15% to about 30% by weight of the ASD. In some embodiments, the ASD comprises a hydrophilic polymer. In some embodiments, the hydrophilic polymer is in an amount of about 15% to about 30% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 15% to about 30% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 15% to about 30% by weight of the ASD. In some embodiments, the organic acid is citric acid.

In some embodiments, the present invention discloses an ASD comprising vilazodone free base or a pharmaceutically acceptable salt thereof. In some embodiments, the vilazodone free base or a pharmaceutically acceptable salt is in an amount of about 15% to about 25% by weight of the ASD. In some embodiments, the ASD comprises a hydrophilic polymer. In some embodiments, the hydrophilic polymer is in an amount of about 25% to about 40% by weight of the ASD. In some embodiments, the hydrophilic polymer is HPMC, polymethacrylates, PVP/VA, HPMC AS, PVP, or a combination thereof. In some embodiments, the ASD comprises a surfactant. In some embodiments, the surfactant is in an amount of about 15% to about 25% by weight of the ASD. In some embodiments, the surfactant is TPGS, PEG, block copolymer of polyethylene glycol and polypropylene glycol, polyoxyl hydrogenated castor oil, lecithin or a combination thereof. In some embodiments, the ASD comprises an organic acid. In some embodiments, the organic acid is in an amount of about 15% to about 25% by weight of the ASD. In some embodiments, the organic acid is citric acid.

### Hydrophilic Polymers

In one aspect, described herein are pharmaceutical compositions comprising anASD that comprises a lipophilic API, a hydrophilic polymer, optionally a surfactant, and optionally an adsorbent. In some embodiments, the API is an API of Table 1 or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone.

In some embodiments, the ASD comprises a hydrophilic polymer. In some embodiments, the hydrophilic polymer comprises at least one of polyvinylpyrrolidone (povidone or PVP), vinylpyrrolidone-vinyl acetate copolymer (copovidone), sulfobutylether-β-cyclodextrin, oligosaccharide, polysaccharide, HEC, HPC, PEO, cyclodextrin (CD) and its derivatives such as hydroxypropyl beta cyclodextrin (HP-β-CD), PEG, HPMCP, polymethacrylates (e.g., sold under trade name Eudragit), HPMC, PVP, polyvinylpolypyrrolidone (PVPP), Kollidon VA64 (VA64), PVA), and polyethylene glycol, polyvinyl acetate and polyvinylcaprolactame-based graft copolymer (PVAc-PVCap-PEG) or polyvinyl caprolactam-polyvinyl acetate-polyethylene glycol (PCL-PVAc-PEG, also termed Soluplus©). In some embodiments, PVP comprises PVP K30 and PVP K90. In some embodiments, the HMPC comprises HPMC-E5. In some embodiments, HMPC comprises HPMC E5 and HMPC E50. In some embodiments, the hydrophilic polymer comprises copovidone. In some embodiments, the hydrophilic polymer comprises povidone. In some embodiments, the copovidone is VA64. In some embodiments, the hydrophilic polymer comprises sulfobutylether-β-cyclodextrin. In some embodiments, the HPMCAS is HPMCAS-LF. In some embodiments, the hydrophilic polymer comprises vinylpyrrolidone-vinyl acetate copolymer or Kollidon VA64 (VA64). In some embodiments, the hydrophilic polymer is hydropropylmethylcellulose acetate succinate (HPMCAS), such as HPMCAS-LS. In some embodiments, the hydrophilic polymer is polymethacrylates, such as Eudragit. In some embodiments, the hydrophilic polymer is hypromellose phthalate (HPMCP).

In some embodiments, an amorphous solid dispersion described herein comprises a hydrophilic polymer. In some embodiments, the hydrophilic polymer is a non-ionic polymer. In some embodiments, the hydrophilic polymer is an ionic polymer. In some embodiments, the hydrophilic polymer is a cationic polymer. In some embodiments, the hydrophilic polymer is an anionic polymer. In some embodiments, the hydrophilic polymer comprises polyvinyl alcohol (PVA), oligosaccharide, polysaccharide, polyvinylpyrrolidone (PVP), hydroxypropyl methylcellulose (HPMC, or hypromellose), hydroxyethyl cellulose (HEC), hydroxypropyl cellulose (HPC), hydropropylmethylcellulose acetate succinate (HPMCAS), polyethylene glycol (PEG), polyvinyl acetate and polyvinylcaprolactame-based graft copolymer (PVAc-PVCap-PEG), or polyvinyl caprolactam-polyvinyl acetate-polyethylene glycol (PCL-PVAc-PEG also termed Soluplus®), polyethylene oxide, hydroxypropyl beta cyclodextrin (HP-β-CD), or a combination thereof. In some embodiments, the non-ionic hydrophilic polymer is HPMC, PVP, HP-β-CD, or PVA. In some embodiments, the hydrophilic polymer is a enteric polymer. In some embodiments, an enteric polymer comprises methacrylate copolymers, hydroxypropyl methylcellulose acetate succinates or cellulose acetate phthalate. In some embodiments, the enteric polymer remains unionized at low pH and remain insoluble. In some embodiments, the enteric polymer comprises polymethacrylates (e.g., Eudragit), Hypromellose Phthalate (HPMCP), HPMCAS, or Soluplus. In some embodiments, the polymethacrylates comprises Eudragit. In some embodiments, the polymethacrylates comprises Ammonio Methacrylate Copolymer (Type A), Ammonio Methacrylate Copolymer (Type B), Basic Butylated Methacrylate Copolymer, Methacrylic Acid-Ethyl Acrylate Copolymer (1:1), Methacrylic Acid-Ethyl Acrylate Copolymer (1:1), Dispersion 30 percent, Methacrylic Acid-Methyl Methacrylate Copolymer (1:1), Methacrylic Acid-Methyl Methacrylate Copolymer (1:2), Polyacrylate Dispersion (30 percent), Poly(butyl methacrylate, (2-dimethylaminoethyl) methacrylate, methyl methacrylate) 1:2:1, Poly(ethyl acrylate, methyl methacrylate) 2:1, Poly(methacrylic acid, methyl methacrylate) 1:1, Poly(methacrylic acid, ethyl acrylate) 1:1, Poly(methacrylic acid, methyl methacrylate) 1:2, Poly(methyl acrylate, methyl methacrylate, methacrylic acid) 7:3:1, Poly(ethyl acrylate, methyl methacrylate, trimethylammonioethyl methacrylate chloride) 1:2:0.2, or Poly(ethyl acrylate, methyl methacrylate, trimethylammonioethyl methacrylate chloride) 1:2:0.1. In some cases, the enteric polymer comprises Cellulose phthalate hydroxypropyl methyl ether; HPMCP; hydroxypropyl methylcellulose benzene-1,2-dicarboxylate; 2-hydroxypropyl methylcellulose phthalate; hypromellosi phthalas; Mantrocel HP-55; or methylhydroxypropylcellulose phthalate.

In some embodiments, the hydrophilic polymer is present in the ASD in a weight percent of about 5% to about 90%. In some embodiments, the hydrophilic polymer is present in the amorphous solid dispersion in an amount of about 5% to about 70%. In some embodiments, the hydrophilic polymer is present in the amorphous solid dispersion in an amount of about 15% to about 50%. In some embodiments, the hydrophilic polymer is present in the amorphous solid dispersion in an amount of about 20% to about 30%. In some embodiments, the hydrophilic polymer is present in the amorphous solid dispersion in an amount of about 25% to about 40%. In some embodiments, the hydrophilic polymer is present in an amorphous solid dispersion in a weight percent of about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some embodiments, the hydrophilic polymer is polyvinylpyrrolidone (PVP) or hydroxypropyl methylcellulose HMPC. In some embodiments, the hydrophilic polymer is polyvinylpyrrolidone. In some embodiments, the hydrophilic polymer is polyvinylpyrrolidone VA64. In some embodiments, the hydrophilic polymer is polyvinylpyrrolidone HPMCAS (such as HPMCAS-LF). In some embodiments, the hydrophilic polymer is polyvinylpyrrolidone HPMC E5. In some embodiments, the hydrophilic polymer is Soluplus. In some embodiments, the hydrophilic polymer comprises polymethacrylates (e.g., Eudragit). In some embodiments, the hydrophilic polymer comprises polyvinyl caprolactam-polyvinyl acetate-polyethylene glycol. In some embodiments, the ASD comprises two or more hydrophilic polymers. In some embodiments, the two or more hydrophilic polymers are selected from copovidone (such as VA64), HPMCAS, and HPMC E5. In some embodiments, the hydrophilic polymer is sulfobutylether-β-cyclodextrin.

In some embodiments, an ASD is formulated in a unit dosage form, such as a capsule or a tablet. In some embodiments, the hydrophilic polymer is present in the ASD in an amount of about 10 mg to about 6,000 mg. In some embodiments, the hydrophilic polymer is present in the amorphous solid dispersion in an amount of about 10 mg to about 50 mg, about 10 mg to about 100 mg, about 10 mg to about 150 mg, about 10 mg to about 200 mg, about 10 mg to about 300 mg, about 10 mg to about 500 mg, about 10 mg to about 800 mg, about 10 mg to about 1,000 mg, about 10 mg to about 2,000 mg, about 10 mg to about 4,000 mg, about 10 mg to about 6,000 mg, about 50 mg to about 100 mg, about 50 mg to about 150 mg, about 50 mg to about 200 mg, about 50 mg to about 300 mg, about 50 mg to about 500 mg, about 50 mg to about 800 mg, about 50 mg to about 1,000 mg, about 50 mg to about 2,000 mg, about 50 mg to about 4,000 mg, about 50 mg to about 6,000 mg, about 100 mg to about 150 mg, about 100 mg to about 200 mg, about 100 mg to about 300 mg, about 100 mg to about 500 mg, about 100 mg to about 800 mg, about 100 mg to about 1,000 mg, about 100 mg to about 2,000 mg, about 100 mg to about 4,000 mg, about 100 mg to about 6,000 mg, about 150 mg to about 200 mg, about 150 mg to about 300 mg, about 150 mg to about 500 mg, about 150 mg to about 800 mg, about 150 mg to about 1,000 mg, about 150 mg to about 2,000 mg, about 150 mg to about 4,000 mg, about 150 mg to about 6,000 mg, about 200 mg to about 300 mg, about 200 mg to about 500 mg, about 200 mg to about 800 mg, about 200 mg to about 1,000 mg, about 200 mg to about 2,000 mg, about 200 mg to about 4,000 mg, about 200 mg to about 6,000 mg, about 300 mg to about 500 mg, about 300 mg to about 800 mg, about 300 mg to about 1,000 mg, about 300 mg to about 2,000 mg, about 300 mg to about 4,000 mg, about 300 mg to about 6,000 mg, about 500 mg to about 800 mg, about 500 mg to about 1,000 mg, about 500 mg to about 2,000 mg, about 500 mg to about 4,000 mg, about 500 mg to about 6,000 mg, about 800 mg to about 1,000 mg, about 800 mg to about 2,000 mg, about 800 mg to about 4,000 mg, about 800 mg to about 6,000 mg, about 1,000 mg to about 2,000 mg, about 1,000 mg to about 4,000 mg, about 1,000 mg to about 6,000 mg, about 2,000 mg to about 4,000 mg, about 2,000 mg to about 6,000 mg, or about 4,000 mg to about 6,000 mg. In some embodiments, the hydrophilic polymer is present in the amorphous solid dispersion in an amount of about 10 mg, about 50 mg, about 100 mg, about 150 mg, about 200 mg, about 300 mg, about 500 mg, about 800 mg, about 1,000 mg, about 2,000 mg, about 4,000 mg, or about 6,000 mg. In some embodiments, the hydrophilic polymer is present in the amorphous solid dispersion in an amount of at least about 10 mg, about 50 mg, about 100 mg, about 150 mg, about 200 mg, about 300 mg, about 500 mg, about 800 mg, about 1,000 mg, about 2,000 mg, or about 4,000 mg. In some embodiments, the hydrophilic polymer is present in the amorphous solid dispersion in an amount of at most about 50 mg, about 100 mg, about 150 mg, about 200 mg, about 300 mg, about 500 mg, about 800 mg, about 1,000 mg, about 2,000 mg, about 4,000 mg, or about 6,000 mg. In some embodiments, the hydrophilic polymer is polyvinylpyrrolidone (PVP) or hydroxypropyl methylcellulose HMPC. In some embodiments, the hydrophilic polymer is polyvinylpyrrolidone. In some embodiments, the hydrophilic polymer is polyvinylpyrrolidone K30. In some embodiments, the hydrophilic polymer is polyvinylpyrrolidone VA64. In some embodiments, the hydrophilic polymer is polyvinylpyrrolidone HPMC. In some embodiments, the hydrophilic polymer is polyvinylpyrrolidone HPMC E5. In some embodiments, the hydrophilic polymer is polyvinylpyrrolidone HPMC E50.

In some embodiments, a pharmaceutical composition is provided that comprises from about 1 mg to about 500 mg of the hydrophilic polymer. In some embodiments, a pharmaceutical composition is provided that comprises from about 10 mg to about 400 mg of the hydrophilic polymer. In some embodiments, a pharmaceutical composition is provided that comprises from about 25 mg to about 200 mg of the hydrophilic polymer. In some embodiments, a pharmaceutical composition is provided that comprises from about 50 mg to about 150 mg of the hydrophilic polymer. In some embodiments, a pharmaceutical composition is provided that comprises from about 75 mg to about 125 mg of the hydrophilic polymer. In some embodiments, a pharmaceutical composition is provided that comprises from about 75 mg to about 100 mg of the hydrophilic polymer. In some embodiments, a pharmaceutical composition is provided that comprises from about 100 mg to about 125 mg of the hydrophilic polymer. In some embodiments, the hydrophilic polymer is polyvinylpyrrolidone (PVP) or hydroxypropyl methylcellulose HMPC. In some embodiments, the hydrophilic polymer is polyvinylpyrrolidone. In some embodiments, the hydrophilic polymer is polyvinylpyrrolidone VA64. In some embodiments, the hydrophilic polymer is polyvinylpyrrolidone HPMCAS. In some embodiments, the hydrophilic polymer is polyvinylpyrrolidone HPMC E5. In some embodiments, the hydrophilic polymer is Soluplus. In some embodiments, the ASD comprises two or more hydrophilic polymers. In some embodiments, the two or more hydrophilic polymers are selected from VA64, HPMCAS, and HPMC E5. In some embodiments, the pharmaceutical composition comprising the hydrophilic polymer is formulated in a unit dosage form, such as a capsule or a tablet.

In some embodiments, a pharmaceutical composition is provided that comprises from about 50 mg to about 150 mg of the hydrophilic polymer. In some embodiments, a pharmaceutical composition is provided that comprises from about 55 mg to about 150 mg of the hydrophilic polymer. In some embodiments, a pharmaceutical composition is provided that comprises from about 60 mg to about 150 mg of the hydrophilic polymer. In some embodiments, a pharmaceutical composition is provided that comprises from about 65 mg to about 150 mg of the hydrophilic polymer. In some embodiments, a pharmaceutical composition is provided that comprises from about 70 mg to about 150 mg of the hydrophilic polymer. In some embodiments, a pharmaceutical composition is provided that comprises from about 75 mg to about 150 mg of the hydrophilic polymer. In some embodiments, a pharmaceutical composition is provided that comprises from about 80 mg to about 150 mg of the hydrophilic polymer. In some embodiments, a pharmaceutical composition is provided that comprises from about 85 mg to about 150 mg of the hydrophilic polymer. In some embodiments, a pharmaceutical composition is provided that comprises from about 90 mg to about 150 mg of the hydrophilic polymer. In some embodiments, a pharmaceutical composition is provided that comprises from about 95 mg to about 150 mg of the hydrophilic polymer. In some embodiments, a pharmaceutical composition is provided that comprises from about 100 mg to about 150 mg of the hydrophilic polymer. In some embodiments, a pharmaceutical composition is provided that comprises from about 105 mg to about 150 mg of the hydrophilic polymer. In some embodiments, a pharmaceutical composition is provided that comprises from about 110 mg to about 150 mg of the hydrophilic polymer. In some embodiments, a pharmaceutical composition is provided that comprises from about 115 mg to about 150 mg of the hydrophilic polymer. In some embodiments, a pharmaceutical composition is provided that comprises from about 120 mg to about 150 mg of the hydrophilic polymer. In some embodiments, a pharmaceutical composition is provided that comprises from about 125 mg to about 150 mg of the hydrophilic polymer. In some embodiments, a pharmaceutical composition is provided that comprises from about 130 mg to about 150 mg of the hydrophilic polymer. In some embodiments, a pharmaceutical composition is provided that comprises from about 135 mg to about 150 mg of the hydrophilic polymer. In some embodiments, a pharmaceutical composition is provided that comprises from about 140 mg to about 150 mg of the hydrophilic polymer. In some embodiments, a pharmaceutical composition is provided that comprises from about 145 mg to about 150 mg of the hydrophilic polymer. In some embodiments, the hydrophilic polymer is polyvinylpyrrolidone (PVP) or hydroxypropyl methylcellulose HMPC. In some embodiments, the hydrophilic polymer is polyvinylpyrrolidone (PVP) or hydroxypropyl methylcellulose HMPC. In some embodiments, the hydrophilic polymer is polyvinylpyrrolidone. In some embodiments, the hydrophilic polymer is polyvinylpyrrolidone VA64. In some embodiments, the hydrophilic polymer is polyvinylpyrrolidone HPMCAS. In some embodiments, the hydrophilic polymer is polyvinylpyrrolidone HPMC E5. In some embodiments, the hydrophilic polymer is Soluplus. In some embodiments, the ASD comprises two or more hydrophilic polymers. In some embodiments, the two or more hydrophilic polymers are selected from VA64, HPMCAS (such as HPMCAS-LF), and HPMC (such as HPMC-E5). In some embodiments, the pharmaceutical composition comprising the hydrophilic polymer is formulated in a unit dosage form, such as a capsule or a tablet.

In some embodiments, the hydrophilic polymer comprises about 5% of the total weight of the composition. In some embodiments, the hydrophilic polymer comprises about 10% of the total weight of the composition. In some embodiments, the hydrophilic polymer comprises about 15% of the total weight of the composition. In some embodiments, the hydrophilic polymer comprises about 20% of the total weight of the composition. In some embodiments, the hydrophilic polymer comprises about 25% of the total weight of the composition. In some embodiments, the hydrophilic polymer comprises about 30% of the total weight of the composition. In some embodiments, the hydrophilic polymer comprises about 40% of the total weight of the composition. In some embodiments, the hydrophilic polymer comprises about 50% of the total weight of the composition. In some embodiments, the hydrophilic polymer is polyvinyl alcohol (PVA), oligosaccharide, polysaccharide, polyvinylpyrrolidone (PVP), hydroxypropyl methylcellulose (HPMC, or hypromellose), hydroxyethyl cellulose (HEC), hydroxypropyl cellulose (HPC), polyethylene oxide, cyclodextrin and its derivatives (CD), hydropropylmethylcellulose acetate succinate (HPMCAS), polymethacrylates, polyethylene glycol (PEG), polyvinyl acetate and polyvinylcaprolactame-based graft copolymer (PVAc-PVCap-PEG), polyvinyl caprolactam-polyvinyl acetate-polyethylene glycol (PCL-PVAc-PEG), or a combination thereof. In some embodiments, the hydrophilic polymer is polyvinylpyrrolidone (PVP) or hydroxypropyl methylcellulose (HMPC). In some embodiments, the hydrophilic polymer is polyvinylpyrrolidone. In some embodiments, the hydrophilic polymer is polyvinylpyrrolidone. In some embodiments, the hydrophilic polymer is polyvinylpyrrolidone HPMCAS. In some embodiments, the hydrophilic polymer is polyvinylpyrrolidone HPMC E5. In some embodiments, the hydrophilic polymer is Soluplus. In some embodiments, the ASD comprises two or more hydrophilic polymers. In some embodiments, the two or more hydrophilic polymers are selected from copovidone, HPMCAS, and HPMC E5.

In some embodiments, a weight ratio of the API selected from abiraterone acetate, alectinib, pazopanib, cabozantinib, venetoclax, lurasidone, and vilazodone, or a pharmaceutically acceptable salt thereof to the hydrophilic polymer is from about 1:1 to about 1:10. In some embodiments, a weight ratio of the API free base or a pharmaceutically acceptable salt thereof to the hydrophilic polymer is from about 10:1 to about 8:1, about 10:1 to about 6:1, about 10:1 to about 4:1, about 10:1 to about 2:1, about 10:1 to about 1:1, about 10:1 to about 1:2, about 10:1 to about 1:4, about 10:1 to about 1:6, about 10:1 to about 1:8, about 10:1 to about 1:10, about 8:1 to about 6:1, about 8:1 to about 4:1, about 8:1 to about 2:1, about 8:1 to about 1:1, about 8:1 to about 1:2, about 8:1 to about 1:4, about 8:1 to about 1:6, about 8:1 to about 1:8, about 8:1 to about 1:10, about 6:1 to about 4:1, about 6:1 to about 2:1, about 6:1 to about 1:1, about 6:1 to about 1:2, about 6:1 to about 1:4, about 6:1 to about 1:6, about 6:1 to about 1:8, about 6:1 to about 1:10, about 4:1 to about 2:1, about 4:1 to about 1:1, about 4:1 to about 1:2, about 4:1 to about 1:4, about 4:1 to about 1:6, about 4:1 to about 1:8, about 4:1 to about 1:10, about 2:1 to about 1:1, about 2:1 to about 1:2, about 2:1 to about 1:4, about 2:1 to about 1:6, about 2:1 to about 1:8, about 2:1 to about 1:10, about 1:1 to about 1:2, about 1:1 to about 1:4, about 1:1 to about 1:6, about 1:1 to about 1:8, about 1:1 to about 1:10, about 1:2 to about 1:4, about 1:2 to about 1:6, about 1:2 to about 1:8, about 1:2 to about 1:10, about 1:4 to about 1:6, about 1:4 to about 1:8, about 1:4 to about 1:10, about 1:6 to about 1:8, about 1:6 to about 1:10, or about 1:8 to about 1:10. In some embodiments, a weight ratio of the API free base or a pharmaceutically acceptable salt thereof to the hydrophilic polymer is from about 10:1, about 8:1, about 6:1, about 4:1, about 2:1, about 1:1, about 1:2, about 1:4, about 1:6, about 1:8, or about 1:10. In some embodiments, a weight ratio of the API free base or a pharmaceutically acceptable salt thereof to the hydrophilic polymer is from at least about 10:1, about 8:1, about 6:1, about 4:1, about 2:1, about 1:1, about 1:2, about 1:4, about 1:6, or about 1:8. In some embodiments, a weight ratio of the API free base or a pharmaceutically acceptable salt thereof to the hydrophilic polymer is from at most about 8:1, about 6:1, about 4:1, about 2:1, about 1:1, about 1:2, about 1:4, about 1:6, about 1:8, or about 1:10.

In some embodiments, a pharmaceutical composition is provided that comprises from about 0.1% to about 99% by weight of a hydrophilic polymer. In some embodiments, a pharmaceutical composition is provided that comprises from about 0.1% to about 80% by weight of the hydrophilic polymer. In some embodiments, a pharmaceutical composition is provided that comprises from about 0.1% to about 60% by weight of the hydrophilic polymer. In some embodiments, a pharmaceutical composition is provided that comprises from about 0.1% to about 40% by weight of the hydrophilic polymer. In some embodiments, a pharmaceutical composition is provided that comprises from about 0.1% to about 20% by weight of the hydrophilic polymer. In some embodiments, a pharmaceutical composition is provided that comprises from about 0.1% to about 10% by weight of the hydrophilic polymer. In some embodiments, a pharmaceutical composition is provided that comprises from about 0.1% to about 1% by weight of the hydrophilic polymer. In some embodiments, a pharmaceutical composition is provided that comprises from about 20% to about 99% by weight of the hydrophilic polymer. In some embodiments, a pharmaceutical composition is provided that comprises from about 20% to about 80% by weight of the hydrophilic polymer. In some embodiments, a pharmaceutical composition is provided that comprises from about 20% to about 60% by weight of the hydrophilic polymer. In some embodiments, a pharmaceutical composition is provided that comprises from about 20% to about 40% by weight of the hydrophilic polymer. In some embodiments, a pharmaceutical composition is provided that comprises from about 30% to about 99% by weight of the hydrophilic polymer. In some embodiments, a pharmaceutical composition is provided that comprises from about 30% to about 80% by weight of the hydrophilic polymer. In some embodiments, a pharmaceutical composition is provided that comprises from about 30% to about 60% by weight of the hydrophilic polymer. In some embodiments, a pharmaceutical composition is provided that comprises from about 30% to about 40% by weight of the hydrophilic polymer. In some embodiments, a pharmaceutical composition is provided that comprises from about 40% to about 99% by weight of the hydrophilic polymer. In some embodiments, a pharmaceutical composition is provided that comprises from about 40% to about 80% by weight of the hydrophilic polymer. In some embodiments, a pharmaceutical composition is provided that comprises from about 40% to about 60% by weight of the hydrophilic polymer. In some embodiments, the hydrophilic polymer is polyvinylpyrrolidone. In some embodiments, the composition is a herein described amorphous solid dispersion. In some embodiments, the composition is a herein described pharmaceutical composition.

In some embodiments, a pharmaceutical composition comprises an amorphous solid dispersion. In some embodiments, the amorphous solid dispersion comprises a hydrophilic polymer in a weight percent of about 1% to about 90%. In some embodiments, the amorphous solid dispersion comprises a hydrophilic polymer in a weight percent of about 1% to about 80%. In some embodiments, the amorphous solid dispersion comprises a hydrophilic polymer in a weight percent of about 10% to about 60%. In some embodiments, the amorphous solid dispersion comprises a hydrophilic polymer in a weight percent of about 20% to about 50%. In some embodiments, the amorphous solid dispersion comprises a hydrophilic polymer in a weight percent of about 1%, about 10%, about 20%, about 30%, about 35%, about 40%, about 45%, about 50%, about 55%, about 60%, about 80%, or about 90%. In some embodiments, the amorphous solid dispersion comprises a hydrophilic polymer in a weight percent of at least about 1%, about 10%, about 20%, about 30%, about 35%, about 40%, about 45%, about 50%, about 55%, about 60%, or about 80%. In some embodiments, the amorphous solid dispersion comprises a hydrophilic polymer in a weight percent of at most about 10%, about 20%, about 30%, about 35%, about 40%, about 45%, about 50%, about 55%, about 60%, about 80%, or about 90%. In some embodiments, the hydrophilic polymer is polyvinylpyrrolidone (PVP) or hydroxypropyl methylcellulose HMPC. In some embodiments, the hydrophilic polymer is polyvinylpyrrolidone. In some embodiments, the hydrophilic polymer is polyvinylpyrrolidone VA64. In some embodiments, the hydrophilic polymer is polyvinylpyrrolidone HPMCAS. In some embodiments, the hydrophilic polymer is polyvinylpyrrolidone HPMC. In some embodiments, the hydrophilic polymer is Soluplus. In some embodiments, the ASD comprises two or more hydrophilic polymers. In some embodiments, the two or more hydrophilic polymers are selected from VA64, HPMCAS, and HPMC.

### Surfactants

In one aspect, disclosed herein are pharmaceutical compositions comprising an amorphous solid dispersion that comprises a lipophilic API, a hydrophilic polymer, and optionally a surfactant. In some embodiments, the ASD comprises a lipophilic API, a hydrophilic polymer, and a surfactant. In some embodiments, the API is an API of Table 1 or a pharmaceutically acceptable salt thereof.

In some embodiments, the surfactants are compounds or mixture of compounds comprising a hydrophobic group (usually a hydrocarbon chain) and a hydrophilic group. They may perform one or more roles including solubility enhancer, bioavailability enhancer, stability enhancer, antioxidant and emulsifying agent. Other terms in the art for surfactants include emulsifier, emulsifying agent, surface-active agent, wetting agent, suspending agent and the like. Examples of surfactants include, but are not limited to phospholipids, lecithin, kolliphor series (rh40), sorbitan oleate, polyvinylcaprolactame-based graft copolymer (PVAc-PVCap-PEG), SDS (or SLS), Solutol, soluplus, sucrose esters of fatty acids, polyoxyl stearate, polyoxyethylene hydrogenated castor oil, polyoxyl 40 hydrogenated castor, macrogolglycerol hydroxystearate oil, peg-40 castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol arlasolve, a block copolymer of polyethylene glycol and polypropylene glycol (e.g., poloxamers or Pluronic F-68), labrafil, labrasol, tween 80, tocopherol polyethylene glycol succinate (i.e., TPGS or vitamin E TPGS) and the like. In some embodiments, SLS and SDS are used interchangeably. In some embodiments, Soluplus can be used as a surfactant. In some embodiments, polyvinylcaprolactame-based graft copolymer (PVAc-PVCap-PEG) can be used as a surfactant. In some embodiments, the surfactant is tocopherol polyethylene glycol 1000 succinate. In some embodiments, the surfactant is phospholipids or their derivatives such as lecithin. In some embodiments the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the lipophilic API has a calculated log P or log P of at least 2.0. In some embodiments, the lipophilic API is one listed in Table 1, or a pharmaceutically acceptable salt thereof. In some embodiments, the lipophilic API, hydrophilic polymer, and the surfactant is formulated as an amorphous solid dispersion.

The surfactant used in the present disclosure can be one or more non-ionic surfactant, one or more an ionic surfactant, or a mixture thereof. In some embodiments, a non-ionic surfactant has no charged groups in its head. Exemplary nonionic surfactants include, without limitation, fatty alcohols, cetyl alcohol, stearyl alcohol, cetostearyl alcohol, and oleyl alcohol. Exemplary nonionic surfactants include, but are not limited to, polyethylene glycol alkyl ethers (such as octaethylene glycol monododecyl ether, pentaethylene glycol monododecyl ether), polyoxylglycerides, (such as Caprylocaproyl macrogol-8 glycerides or PEG-8 caprylic/capric glycerides sold under the trade name Labrasol, or Lauroyl Polyoxyl-32 glycerides sold under the trade name Gelucire 44/14), polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate), polyoxyethylene glycol sorbitan alkyl esters (such as polysorbate), sorbitan alkyl esters (such as Spans), cocamide MEA, cocamide DEA, dodecyldimethylamine oxide, block copolymers of polyethylene glycol and polypropylene glycol (such as poloxamers), polyethoxylated tallow amine (POEA), and tocopherol polyethylene glycol succinate (TPGS or Vitamin E TPGS). In some embodiments, a non-ionic surfactant comprises one or more of fatty alcohols, cetyl alcohol, stearyl alcohol, cetostearyl alcohol, and oleyl alcohol. Exemplary nonionic surfactants include, but are not limited to, polyethylene glycol alkyl ethers (such as octaethylene glycol monododecyl ether, pentaethylene glycol monododecyl ether), polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate), polyoxyethylene glycol sorbitan alkyl esters (such as polysorbate), sorbitan alkyl esters (such as Spans), cocamide MEA, cocamide DEA, dodecyldimethylamine oxide, block copolymers of polyethylene glycol and polypropylene glycol (such as poloxamers), polyethoxylated tallow amine (POEA), and tocopherol polyethylene glycol succinate (TPGS or Vitamin E TPGS).

In some embodiments, a non-ionic surfactant comprises Vitamin E, a block copolymer of polyethylene glycol and polypropylene glycol, or any combination thereof. In some embodiments, the surfactant comprises two more repeating units, such as polyoxyalkylene units. In some embodiments, the surfactant is a non-ionic surfactant that comprises polyethylene glycol. In some embodiments, the surfactant comprises polyoxylglycerides (such as Caprylocaproyl macrogol-8 glycerides or PEG-8 caprylic/capric glycerides). In some embodiments, the surfactant is a block copolymer of polyethylene glycol and polypropylene glycol. In some embodiments, the ASD comprises two surfactants. In some embodiments, the ASD comprises two or more surfactants. In some embodiments, the ASD comprises a surfactant wherein the surfactant is lecithin. In some embodiments, the ASD comprises a surfactant wherein the surfactant is TPGS. In some embodiments, the ASD comprises a surfactant wherein the surfactant comprises lecithin and TPGS. In some embodiments, the ASD comprises a surfactant wherein the surfactant is polyvinylcaprolactame-based graft copolymer (PVAc-PVCap-PEG). In some embodiments, the ASD comprises a surfactant wherein the surfactant is polyoxyl hydrogenated castor oil. In some embodiments, the ASD comprises a surfactant wherein the surfactant is phospholipids or their derivatives such as lecithin.

In some embodiments, an ionic surfactant has a charged group in its head. In some embodiments, an ionic surfactant has an anionic head group or a cationic head group. In some embodiments, exemplary ionic surfactants include sodium lauryl sulfate (SLS), sodium dodecyl sulfate, calcium oleate, triethanolamine oleate, docusate sodium, benzalkonium chloride, and cetylpyridinium chloride. In some embodiments, the pharmaceutical composition or the amorphous solid dispersion comprises SLS. In some embodiments, the surfactant is a mixture of one or more non-ionic surfactants and one or more ionic surfactant. In some embodiments, the surfactant comprises SLS and TPGS.

In some embodiments, the non-ionic surfactant has a number average molecular weight of from about from about 1000 to about 100,000 Da, 2000 to about 20,000 Da, from about 4000 to about 15,000 Da, from about 6000 to about 12,000 Da, or from about 7000 to about 10,000 Da. In some embodiments, the non-ionic surfactant has a number average molecular weight of from about 7000 to about 10,000 Da. In some embodiments, the non-ionic surfactant has an ethylene glycol content of from about 30 wt % to about 99 wt %, from about 50 wt % to about 95 wt %, from about 60 wt % to about 95 wt %, from about 75 wt % to about 90 wt %, or from about 80 wt % to about 85 wt %. In some embodiments, the non-ionic surfactant has an ethylene glycol content of from about 80 wt % to about 85 wt %.

In some embodiments, the surfactants are selected from fatty acids, phospholipids, sphingolipids, saccharolipids, polyketides, sterol lipids, prenol lipids and the like. In some embodiments, phospholipids are made up of glycerol to which is attached a phosphate group and two fatty acids. Other terms in the art for phospholipids include glycerophospholipids, phosphoglycerides, diacylglycerides and the like. The phosphate group can be unmodified (i.e. in the structure below R═H) or modified by attachment (i.e. in the structure below R≠H) to simple organic molecules such as, but not limited to choline, ethanolamine or serine. Phospholipids may be further modified by substitution onto one or more for the hydrocarbon chains.

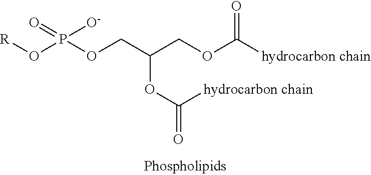

Pharmaceutical compositions described herein comprise an ASD that comprises a phospholipid. In some embodiments, phospholipids are selected from glycerophospholipid, sphingolipid, and/or phospholipid derivatives. In some embodiments, glycerophospholipids include, but are not limited to phosphatidylcholine, phosphatidyl ethanolamine, phosphatidyl ethanolamine, phosphatidyl serine, phosphatidyl glycerol, diphosphatidylglycerol, phosphatidylinositol, and mixtures thereof. Phospholipid derivatives according to the present invention include, but are not limited to dioleoylphosphatidylcholine, dimyristoylphosphatidylcholine, dipentadeanoylphosphatidylcholine, dilauroylphosphatidylchoine, dipalmitoylphosphatidylcholine (DPPC), distearoylphosphatidylcholine (DSPC), diarachidonyiphosphatidylcholine (DAPC), dioleoylphosphatidylethanolamine, dipalmitoylphosphatidylethanolamine (DPPE), and distearoylphosphatidylethanolamine (DSPE), disteraoylphosphatidylglycerol (DSPG), phosphatidylinositol, dipalmitoylphosphatidic acid (DPPA), distearoylphosphatidic acid (DSPA), and the like, and mixtures thereof. In some embodiments, the phospholipids comprise at least 40%, 50%, 60%, 70%, 80%, 90%, or 95% phosphatidylcholine by weight. In some embodiments, the phospholipids comprise greater than 80% phosphatidylcholine.

In some embodiments, the phospholipid is present in the pharmaceutical composition in an amount of about 25 mg to about 200 mg. In some embodiments, the phospholipid is present in an amount of about 50 mg to 150 mg. In some embodiments, the phospholipids comprise 2.5%-20% of the total weight of the pharmaceutical composition. In some embodiments, the phospholipids comprise 5%-17% of the total weight of the pharmaceutical composition. In some embodiments, the phospholipids comprise greater than 80% phosphatidylcholine.

In some embodiments, phosphatidylcholines are phospholipids wherein a choline group (Me₃N⁺—CH₂—CH₂—O—) is attached to the phosphate group.

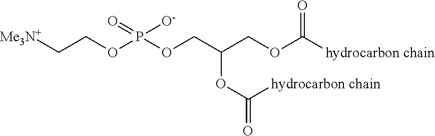

### Phosphatidylcholines

In some embodiments, the ASD comprise a phosphatidylcholine. A non-limiting example of a phosphatidylcholine is 1-oleoyl-2-palmitoyl-phosphatidyl choline, as shown below:

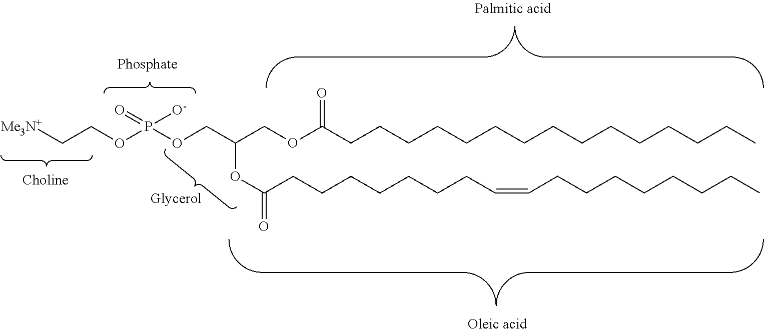

In some embodiments, the surfactant is lecithin. The USP 40 definition of lecithin is “a complex mixture of acetone-insoluble phosphatides, which consist chiefly of phosphatidylcholine, phosphatidylethanolamine, phosphatidylinositol, and phosphatidic acid, present in conjunction with various amounts of other substances such as triglycerides, fatty acids, and carbohydrates, as separated from the crude vegetable oil source.” In some embodiments, lecithin is a mixture of phospholipids. Lecithins can be isolated from various sources including, but not limited to eggs, soybeans, milk, marine sources, rapeseed, cottonseed and sunflower. In some embodiments, the lecithin used in the disclosed amorphous solid dispersions and/or pharmaceutical compositions is isolated from egg yolk. In some embodiments, lecithin comprises a mixture of phospholipids, including phosphatidylcholine, phosphatidylethanolamine, phosphatidylinositol, and phosphatidic acid. In some embodiments, lecithin comprises a mixture of phosphatidylcholine, phosphatidylethanolamine, phosphatidylinositol, phosphatidylserine, and phosphatidic acid. Lecithin can be isolated from various sources including, but not limited to eggs, soybeans, milk, marine sources, rapeseed, cottonseed and sunflower. In some embodiments, the lecithin used in the disclosed amorphous solid dispersions and/or pharmaceutical compositions is isolated from egg yolk. Lecithin can be E322. Lecithin can be egg lecithin. Lecithin can be LSC 5050. Lecithin can be LSC 6040. Lecithin can be mixed soybean phosphatides. Lecithin can be ovolecithin. Lecithin can be Phosal 53 MCT. Lecithin can be Phospholipon 100 H. Lecithin can be ProKote LSC. Lecithin can be soybean lecithin. Lecithin can be soybean phospholipids. Lecithin can be Sternpur. Lecithin can be vegetable lecithin. Lecithin can be 1,2-diacyl-sn-glycero-3-phosphocholine. In some embodiments, the lecithin contains more than 25% of phosphatidylcholine. In some embodiments, the lecithin contains more than 50% of phosphatidylcholine. In some embodiments, the lecithin contains more than 60% of phosphatidylcholine. In some embodiments, the lecithin contains more than 70% of phosphatidylcholine. In some embodiments, lecithin is from an extract of soybeans (e.g., CAS [8030-76-0]). In some embodiments, lecithin comprises egg yolk lecithin (e.g., CAS [93685-90-6]).

In some embodiments, the lecithin contains from about 10% to about 95% of phosphatidylcholine. In some embodiments, the lecithin contains from about 15% to about 80% of phosphatidylcholine In some embodiments, the lecithin contains from about 20% to about 75% of phosphatidylcholine. In some embodiments, the lecithin contains from about about 25% to about 70% of phosphatidylcholine. In some embodiments, the lecithin contains from about 30% to about 65% of phosphatidylcholine. In some embodiments, the lecithin contains from about 35% to about 60% of phosphatidylcholine. In some embodiments, the lecithin contains from about 40% to about 55% of phosphatidylcholine. In some embodiments, the lecithin contains about 10%, about 15%, about 20%, about 21%, about 25%, about 30%, about 35%, about 40%, about 45%, about 50%, about 55%, about 60%, about 65%, about 69%, about 70%, about 75%, about 85%, about 90% phosphatidylcholine. In some embodiments, the lecithin contains about 69% phosphatidylcholine. In some embodiments, the lecithin contains about 21% phosphatidylcholine. In some embodiments, the lecithin contains from about 1% to about 55%, about 1% to about 50%, from about 2% to about 40%, from about 3% to about 36%, from about 5% to about 35%, from about 10% to about 30%, from about 15% to about 25% of phosphatidylethanolamine. In some embodiments, the lecithin contains about 1%, about 2%, about 3%, about 4%, about 5%, about 10%, about 15%, about 20%, about 22%, about 24%, about 26%, about 29%, about 30%, about 35%, about 36%, about 40%, about 45%, about 50%, or about 55% phosphatidylethanolamine. In some embodiments, the lecithin contains about 21% phosphatidylcholine, about 22% phosphatidylethanolamine, and about 19% phosphatidylinositol. In some embodiments, the lecithin contains about 69% phosphatidylcholine and about 24% phosphatidylethanolamine. In some embodiments, lecithin is egg yolk lecithin. In some embodiments, In some embodiments, the phosphatidylcholine is from egg origin. In some embodiments, the phosphatidylcholine is from or soybean origin.

In some embodiments, the surfactant is a phospholipid. In some embodiments, the phospholipid is phosphatidylcholine. In some embodiments, the phospholipid is a mixture comprising phosphatidylcholine. In some embodiments, the surfactant is lecithin. In some embodiments, lecithin is a mixture of phospholipids. In some embodiments, the lecithin is comprised of phosphatidylcholine. In some embodiments, the lecithin contains more than 25% of phosphatidylcholine. In some embodiments, the lecithin contains more than 80% of phosphatidylcholine. In some embodiments, the phosphatidylcholine is from egg origin. In some embodiments, the phosphatidylcholine is from or soybean origin.

In some embodiments, the surfactant is present in an ASD in a weight percent of about 1% to about 70%. In some embodiments, the surfactant is present in an amorphous solid dispersion in a weight percent of about 10% to about 60%. In some embodiments, the surfactant is present in the ASD in a weight percent of about 1% to about 50%. In some embodiments, the surfactant is present in the ASD in a weight percent of about 15% to about 45%. In some embodiments, the surfactant is present in the ASD in a weight percent of about 20% to about 40%. In some embodiments, the surfactant is present in the ASD in a weight percent of about 20% to about 30%. In some embodiments, the surfactant is present in the ASD in a weight percent of about 30% to about 40%. In some embodiments, the surfactant is present in an amorphous solid dispersion in a weight percent of about 5% to about 30%. In some embodiments, the surfactant is present in an amorphous solid dispersion in a weight percent of about 10% to about 50%. In some embodiments, the surfactant is present in an amorphous solid dispersion in a weight percent of about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the surfactant is present in an amorphous solid dispersion in a weight percent of about 10%, about 15%, about 20%, about 25%, about 30%, about 35%, about 40%, about 45%, or about 50%. In some embodiments, the surfactant is present in an amorphous solid dispersion in a weight percent of at least about 10%, about 15%, about 20%, about 25%, about 30%, about 35%, about 40%, or about 45%. In some embodiments, the surfactant is present in an amorphous solid dispersion in a weight percent of at most about 15%, about 20%, about 25%, about 30%, about 35%, about 40%, about 45%, or about 50%. In some embodiments, the surfactant is present in an amorphous solid dispersion in a weight percent of about 20%. In some embodiments, the surfactant is present in an amorphous solid dispersion in a weight percent of about 25%. In some embodiments, the surfactant is TPGS. In some embodiments, the phospholipid is lecithin. In some embodiments, the surfactant is SLS. In some embodiment, surfactant is a combination of lecithin and TPGS. In some embodiment, surfactant is a combination of SLS and TPGS. In some embodiments, the surfactant is lecithin. In some embodiments, the surfactant comprises phospholipids or their derivatives such as lecithin, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof. In some embodiments, the surfactant comprises lecithin and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RH40). In some embodiments, the surfactant comprises polysorbate. In some embodiments, the surfactant comprises polyoxylglycerides (e.g., Labrasol or Gelucire). In some embodiments, the surfactant comprises TPGS.

In some embodiments, an ASD is formulated in a unit dosage form, such as a capsule or a tablet. In some embodiments, the surfactant is present in the ASD in an amount of about 5 mg to about 5,000 mg. In some embodiments, the surfactant is present in the ASD in an amount of about 5 mg to about 10 mg, about 5 mg to about 20 mg, about 5 mg to about 30 mg, about 5 mg to about 50 mg, about 5 mg to about 80 mg, about 5 mg to about 100 mg, about 5 mg to about 150 mg, about 5 mg to about 200 mg, about 5 mg to about 300 mg, about 5 mg to about 500 mg, about 5 mg to about 5,000 mg, about 10 mg to about 20 mg, about 10 mg to about 30 mg, about 10 mg to about 50 mg, about 10 mg to about 80 mg, about 10 mg to about 100 mg, about 10 mg to about 150 mg, about 10 mg to about 200 mg, about 10 mg to about 300 mg, about 10 mg to about 500 mg, about 10 mg to about 5,000 mg, about 20 mg to about 30 mg, about 20 mg to about 50 mg, about 20 mg to about 80 mg, about 20 mg to about 100 mg, about 20 mg to about 150 mg, about 20 mg to about 200 mg, about 20 mg to about 300 mg, about 20 mg to about 500 mg, about 20 mg to about 5,000 mg, about 30 mg to about 50 mg, about 30 mg to about 80 mg, about 30 mg to about 100 mg, about 30 mg to about 150 mg, about 30 mg to about 200 mg, about 30 mg to about 300 mg, about 30 mg to about 500 mg, about 30 mg to about 5,000 mg, about 50 mg to about 80 mg, about 50 mg to about 100 mg, about 50 mg to about 150 mg, about 50 mg to about 200 mg, about 50 mg to about 300 mg, about 50 mg to about 500 mg, about 50 mg to about 5,000 mg, about 80 mg to about 100 mg, about 80 mg to about 150 mg, about 80 mg to about 200 mg, about 80 mg to about 300 mg, about 80 mg to about 500 mg, about 80 mg to about 5,000 mg, about 100 mg to about 150 mg, about 100 mg to about 200 mg, about 100 mg to about 300 mg, about 100 mg to about 500 mg, about 100 mg to about 5,000 mg, about 150 mg to about 200 mg, about 150 mg to about 300 mg, about 150 mg to about 500 mg, about 150 mg to about 5,000 mg, about 200 mg to about 300 mg, about 200 mg to about 500 mg, about 200 mg to about 5,000 mg, about 300 mg to about 500 mg, about 300 mg to about 5,000 mg, or about 500 mg to about 5,000 mg. In some embodiments, the surfactant is present in the ASD in an amount of about 5 mg, about 10 mg, about 20 mg, about 30 mg, about 50 mg, about 80 mg, about 100 mg, about 150 mg, about 200 mg, about 300 mg, about 500 mg, or about 5,000 mg. In some embodiments, the surfactant is present in the ASD in an amount of at least about 5 mg, about 10 mg, about 20 mg, about 30 mg, about 50 mg, about 80 mg, about 100 mg, about 150 mg, about 200 mg, about 300 mg, or about 500 mg. In some embodiments, the surfactant is present in the ASD in an amount of at most about 10 mg, about 20 mg, about 30 mg, about 50 mg, about 80 mg, about 100 mg, about 150 mg, about 200 mg, about 300 mg, about 500 mg, or about 5,000 mg. In some embodiments, the surfactant is or comprises a phospholipid. In some embodiments, the phospholipid is or comprises lecithin. In some embodiments, the surfactant is TPGS. In some embodiments, the phospholipid is lecithin. In some embodiments, the surfactant is SLS. In some embodiment, surfactant is a combination of lecithin and TPGS. In some embodiment, surfactant is a combination of SLS and TPGS.

In some embodiments, an ASD is formulated in a unit dosage form, such as a capsule or a tablet. In some embodiments, the surfactant, such as TPGS, SLS, lecithin, polyethylene glycol (PEG), a block copolymer of polyethylene glycol and polypropylene glycol, polyvinyl acetate and polyvinylcaprolactame-based graft copolymer, polyoxyl hydrogenated castor oil, polyoxylglycerides, polysorbate, or a combination thereof, is present in an ASD in an amount of no less than 5 mg, 10 mg, 15 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 225 mg, or 250 mg. In some embodiments, the lecithin is present in an amorphous solid dispersion or in a pharmaceutical composition disclosed herein in an amount of about 5 mg, 10 mg, 15 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 225 mg, or 250 mg. In some embodiments the API is selected from abiraterone acetate, alectinib, pazopanib, cabozantinib, venetoclax, lurasidone, and vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the API is a lipophilic API. In some embodiments, the lipophilic API is one listed in Table 1, or a pharmaceutically acceptable salt thereof. In some embodiments, the lipophilic API has a log P 2.0 or higher. In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

In some embodiments, the ratio by weight of the API to the surfactant is from about 2:1 to about 1:10. In some embodiments, the ratio by weight of the API to the surfactant is from about 1:0.5 to about 1:6. In some embodiments, the ratio by weight of the API to the surfactant is from about 1:0.8 to about 1:5. In some embodiments, the ratio by weight of the API to the surfactant is from about 1:0.8 to about 1:3. In some embodiments, the ratio by weight of the API to the surfactant is from about 1:1 to about 1:3. In some embodiments, the ratio by weight of the API to the surfactant is from about 1:0.8 to about 1:2.8. In some embodiments, the ratio by weight of the API to the surfactant is from about 1:0.8 to about 1:2.5. In some embodiments, the ratio by weight of the API to the surfactant is from about 1:1 to about 1:2.5. In some embodiments, the ratio by weight of the API to the surfactant is from about 1:1 to about 1:2. In some embodiments, the ratio by weight of the API to the surfactant is from about 1:1 to about 1:1.5. In some embodiments, the ratio by weight of the API to the surfactant is from about 1:1 to about 1:4. In some embodiments, the ratio by weight of the API to the surfactant is from about 1:1 to about 1:3.5. In some embodiments, the surfactant is TPGS. In some embodiments, the phospholipid is lecithin. In some embodiments, the surfactant is SLS. In some embodiment, surfactant is a combination of lecithin and TPGS. In some embodiment, surfactant is a combination of SLS and TPGS. In some embodiments, the surfactant comprises lecithin. In some embodiments, the surfactant comprises PEG. In some embodiments, the surfactant comprises a block copolymer of polyethylene glycol and polypropylene glycol. In some embodiments, the surfactant comprises SLS. In some embodiments, the surfactant comprises polyvinyl acetate and polyvinylcaprolactame-based graft copolymer. In some embodiments, the surfactant comprises polyoxyl hydrogenated castor (e.g., sold under the trade name RIH40). In some embodiments, the surfactant comprises polysorbate. In some embodiments, the surfactant comprises polyoxylglycerides (e.g., Labrasol or Gelucire). In some embodiments, the surfactant comprises TPGS.

In some embodiments, the surfactant comprises 0.1%-50% of the total weight of a herein described pharmaceutical composition. In some embodiments, the pharmaceutical composition is an amorphous solid dispersion. In some embodiments, the surfactant comprises 1%-30% of the total weight of the composition. In some embodiments, the composition is a pharmaceutical composition. In some embodiments, the surfactant comprises 5%-20% of the total weight of the composition. In some embodiments, the surfactant comprises 10%-17% of the total weight of the composition. In some embodiments, the surfactant comprises about 15% of the total weight of the composition. In some embodiments, the surfactant comprises about 16% of the total weight of the composition. In some embodiments, the surfactant comprises about 17% of the total weight of the composition. In some embodiments, the ratio by weight of the hydrophilic polymer to lecithin is greater than 0.75. In some embodiments, the ratio by weight of the hydrophilic polymer to lecithin is greater than 1.0. In some embodiments, the ratio by weight of the hydrophilic polymer to lecithin is greater than 1.1. In some embodiments, the ratio by weight of the hydrophilic polymer to lecithin is greater 1.2. In some embodiments, the ratio by weight of the hydrophilic polymer to lecithin is greater 1.3. In some embodiments, the ratio by weight of the hydrophilic polymer to lecithin is greater 1.4. In some embodiments, the ratio by weight of the hydrophilic polymer to lecithin is greater than 1.5. In some embodiments, pharmaceutical compositions described herein include a lipophilic API, a hydrophilic polymer, and a surfactant. In some embodiments, pharmaceutical compositions described herein include a lipophilic API, a hydrophilic polymer, and a surfactant or poloxamer. In some embodiments, pharmaceutical compositions described herein include a lipophilic API, a hydrophilic polymer, and lecithin. In some embodiments, pharmaceutical compositions described herein include a lipophilic API, a hydrophilic polymer, and lecithin. In some embodiments the API is abiraterone acetate, alectinib, pazopanib, cabozantinib, venetoclax, lurasidone, vilazodone or a pharmaceutically acceptable salt thereof. In some embodiments, the API is abiraterone acetate, alectinib, pazopanib, cabozantinib, venetoclax, lurasidone, or vilazodone. In some embodiments, the API is a pharmaceutically acceptable salt of abiraterone acetate, alectinib, pazopanib, cabozantinib, venetoclax, lurasidone, or vilazodone. In some embodiments, the API is a lipophilic API. In some embodiments, the lipophilic API is one listed in Table 1, or a pharmaceutically acceptable salt thereof. In some embodiments, the lipophilic API has a log P 2.0 or higher.

In some embodiments, an amorphous solid dispersion includes a lipophilic API, a hydrophilic polymer, and a surfactant. In some embodiments, the amorphous solid dispersions described herein additionally comprise one or more adsorbents. In some embodiments, the amorphous solid dispersions described herein additionally comprise one or more other additives. In some embodiments, other additives comprise organic and inorganic acids. In some embodiments, other additives comprise antioxidants.

### Acids

In some embodiments, an amorphous solid dispersion comprises an API, and an inorganic acid or organic acid. In some embodiments, the organic acid is selected from the group consisting of tartaric acid, fumaric acid, succinic acid, citric acid, lactic acid, malic acid, methanesulfonic acid, ethanesulfonic acid, isethionic acid, benzenesulfonic acid, and p-toluenesulfonic acid. In some embodiments, the inorganic acid is selected from the group consisting of hydrochloric acid, sulfuric acid, and phosphoric acid. In some embodiments, the API is a compound of Table 1 or a pharmaceutically acceptable salt thereof.

In one aspect, described herein is an amorphous solid dispersion comprising an API and one or more acids. Also described herein is a pharmaceutical composition comprising an API and one or more acids. In some embodiments, the amorphous solid dispersion comprises an API, one or more acids, and a hydrophilic high-molecular weight material. In some embodiments, the API is at least partially protonated.

In some embodiments, an amorphous solid dispersion and/or a pharmaceutical composition disclosed herein comprises one or more organic acids. In some embodiments, the one or more organic acids comprise one or more of acetic acid, acrylic acid, adipic acid, alginic acid, amino acids, ascorbic acid, benzoic acid, benzenesulfonic acid, butyric acid, carbonic acid, citric acid, formic acid, fumaric acid, gluconic acid, isoascorbic acid, lactic acid, maleic acid, malic acid, methanesulfonic acid, fluorinated acids, trifluoromethanesulfonic acid, trifluoroacetic acid, oxalic acid, propionic acid, salicylic acid, stearic acid, succinic acid, tannic acid, tartaric acid, thioglycolic acid, aliphatic sulfonic acids (e.g., methanesulfonic acid, methanedisulfonic acid, triflic acid, ethanesulfonic acid, ethanedisulfonic acid, isethionic acid, 2-mercapto-1-ethansulfonic acid, propanesulfonic acid, butanesulfonic acid), aromatic sulfonic acids (e.g., benzenesulfonic acid, tolylsulfonic acid, or naphthalenesulfonic acid) and uric acid. In some embodiments, the one or more organic acids comprise methanesulfonic acid, tartaric acid, or both. In some embodiments, the one or more organic acids comprise methanesulfonic acid and tartaric acid. In some embodiments, the one or more organic acids excludes acetic acid.

In some embodiments, the organic acid or inorganic acid is present in the ASD by weight of about 1% to about 60%. In some embodiments, the organic acid or inorganic acid is present in the ASD by weight of about 5% to about 50%. In some embodiments, the organic acid or inorganic acid is present in the ASD by weight of about 10% to about 40%. In some embodiments, the organic acid or inorganic acid is present in the ASD by weight of about 15% to about 30%. In some embodiments, the organic acid or inorganic acid is present in the ASD by weight of about 15% to about 25%. In some embodiments, the organic acid or inorganic acid is present in the ASD by weight of about 20% to about 30%. In some embodiments, the organic acid or inorganic acid is present in the ASD by weight of about 1% to about 5%, about 1% to about 10%, about 1% to about 15%, about 1% to about 20%, about 1% to about 25%, about 1% to about 30%, about 1% to about 35%, about 1% to about 40%, about 1% to about 50%, about 1% to about 60%, about 5% to about 10%, about 5% to about 15%, about 5% to about 20%, about 5% to about 25%, about 5% to about 30%, about 5% to about 35%, about 5% to about 40%, about 5% to about 50%, about 5% to about 60%, about 10% to about 15%, about 10% to about 20%, about 10% to about 25%, about 10% to about 30%, about 10% to about 35%, about 10% to about 40%, about 10% to about 50%, about 10% to about 60%, about 15% to about 20%, about 15% to about 25%, about 15% to about 30%, about 15% to about 35%, about 15% to about 40%, about 15% to about 50%, about 15% to about 60%, about 20% to about 25%, about 20% to about 30%, about 20% to about 35%, about 20% to about 40%, about 20% to about 50%, about 20% to about 60%, about 25% to about 30%, about 25% to about 35%, about 25% to about 40%, about 25% to about 50%, about 25% to about 60%, about 30% to about 35%, about 30% to about 40%, about 30% to about 50%, about 30% to about 60%, about 35% to about 40%, about 35% to about 50%, about 35% to about 60%, about 40% to about 50%, about 40% to about 60%, or about 50% to about 60%. In some embodiments, the organic acid or inorganic acid is present in the ASD by weight of about 1%, about 5%, about 10%, about 15%, about 20%, about 25%, about 30%, about 35%, about 40%, about 50%, or about 60%. In some embodiments, the organic acid or inorganic acid is present in the ASD by weight of at least about 1%, about 5%, about 10%, about 15%, about 20%, about 25%, about 30%, about 35%, about 40%, or about 50%. In some embodiments, the organic acid or inorganic acid is present in the ASD by weight of at most about 5%, about 10%, about 15%, about 20%, about 25%, about 30%, about 35%, about 40%, about 50%, or about 60%. In some embodiments, the organic acid or inorganic acid is present in the pharmaceutical compositions and is not present in the ASD. In some embodiments, the organic acid is malic acid. In some embodiments, the organic acid is citric acid. In some embodiments, the organic acid is tartaric acid.

In some embodiments, the one or more organic acids are present in the amorphous solid dispersion and/or in the pharmaceutical composition in an amount of from about 0.1% to about 99% by weight of the total composition. In some embodiments, the one or more organic acids are present in the amorphous solid dispersion and/or in the pharmaceutical composition in an amount of from about 1% to about 80%, from about 1% to about 60%, from about 1% to about 50%, from about 1% to about 25%, from about 1% to about 10%, from about 1% to about 5%, from about 10% to about 80%, from about 10% to about 60%, from about 10% to about 50%, from about 20% to about 80%, from about 20% to about 60%, from about 20% to about 50%, from about 30% to about 80%, from about 30% to about 60%, from about 30% to about 50%, or from about 30% to about 40% by weight of the total composition. In some embodiments, the one or more organic acids are present in the amorphous solid dispersion and/or in the pharmaceutical composition in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight of the total composition. In some embodiments, the one or more organic acids comprise tartaric acid. In some embodiments, the one or more organic acids comprise methanesulfonic acid. In some embodiments, the one or more organic acids are present in the amorphous solid dispersion and/or in the pharmaceutical composition in an amount of about 1.0 mg to about 1000 mg, including but not limited to about 5.0 mg, 10.0 mg, 15.0 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg. In some embodiments, the one or more organic acids are present in the amorphous solid dispersion and/or in the pharmaceutical composition in an amount of 1 mg to 500 mg. In some embodiments, the one or more organic acids are present in an amount of from about from about 10 mg to about 400 mg, 20 mg to about 300 mg, from about 25 mg to about 200 mg, from about 50 mg to about 150 mg, from about 75 mg to about 125 mg, from about 75 mg to about 100 mg, from about 100 mg to about 125 mg, from about 1 mg to about 200 mg, or from about 50 mg to about 200 mg. In some embodiments, the one or more organic acids are present in an amount of 25 mg to 250 mg. In some embodiments, the one or more organic acids are present in an amount of 150 mg to 250 mg. In some embodiments, the one or more organic acids are present in an amount of 150 mg to 200 mg. In some embodiments, the one or more organic acids are present in an amount of 50 mg to 200 mg.

In some embodiments, an amorphous solid dispersion described herein comprises an API, one or more acids, and a hydrophilic high-molecular weight material. In some embodiments, one or more acids comprises a first acid and a second acid. In some embodiments, the molar ratio of the first acid to API is about 0.1:1 to about 10:1, about 0.5:1 to about 5:1, about 0.5:1 to about 3:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the molar ratio of the second acid to API is present in a molar ratio to the API of about 0.1:1 to about 10:1, about 1:1 to about 8:1, about 2:1 to about 7:1, about 4:1 to about 7:1, about 0.5:1 to about 3:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the mass ratio of the second acid to API is present in a molar ratio to the API of about 0.1:1 to about 10:1, about 0.2:1 to about 5:1, about 0.5:1 to about 3:1, about 0.2:1 to about 1.2:1, about 0.4:1 to about 1:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the molar ratio of the first acid to API is about 0.1:1 to 1.5:1. In some embodiments, the first acid is an inorganic acid. In some embodiments, the first acid is an organic acid.

In some embodiments, the mass ratio of the second acid to API is about 0.05:1 to about 20:1, about 0.5:1 to about 10:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the mass ratio of the second acid to API is about 0.5:1 to about 10:1. In some embodiments, the first acid is an inorganic acid. In some embodiments, the first acid is an organic acid.

In some embodiments, an amorphous solid dispersion and/or a pharmaceutical composition disclosed herein comprises one or more inorganic acids. In some embodiments, the one or more inorganic acids comprise one or more of hydrochloric acid, hydrobromic acid, hydriodic acid, sulfuric acid, nitric acid, boric acid, and phosphoric acid. In some embodiments, the one or more inorganic acids comprise hydrochloric acid. In some embodiments, the inorganic acid is completely ionized. In some embodiments, the inorganic acid is partially ionized. In some embodiments, partial ionization refers to an equilibrium in which 1% or more of the inorganic acid is ionized.

In some embodiments, the one or more inorganic acids are present in the amorphous solid dispersion and/or in the pharmaceutical composition in an amount of from about 0.1% to about 99% by weight of the total composition. In some embodiments, the one or more inorganic acids are present in the amorphous solid dispersion and/or in the pharmaceutical composition in an amount of from about 0.1% to about 80%, 1% to about 80%, from about 1% to about 60%, from about 1% to about 50%, from about 10% to about 80%, from about 10% to about 60%, from about 10% to about 50%, from about 20% to about 80%, from about 20% to about 60%, from about 20% to about 50%, from about 30% to about 80%, from about 30% to about 60%, from about 30% to about 50%, or from about 30% to about 40% by weight of the total composition. In some embodiments, the one or more inorganic acids are present in the amorphous solid dispersion and/or in the pharmaceutical composition in an amount of about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, or about 45% by weight of the total composition. In some embodiments, the one or more inorganic acids comprise hydrochloric acid. In some embodiments, the one or more inorganic acids are present in the amorphous solid dispersion and/or in the pharmaceutical composition in an amount of about 1.0 mg to about 1000 mg, including but not limited to about 1.0 mg, 2.0 mg, 3.0 mg, 4.0 mg, 5.0 mg, 10.0 mg, 15.0 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg. In some embodiments, the one or more inorganic acids are present in an amount of from about 0.1 mg to about 100 mg, from about 1 mg to about 50 mg, from about 2 mg to about 20 mg, from about 5 mg to about 15 mg, from about 7 mg to about 25 mg, from about 7 mg to about 20 mg, or from about 10 mg to about 18 mg.

In some embodiments, an amorphous solid dispersion disclosed herein comprises a first acid and a second acid. In some embodiments, a molar ratio of the second acid to the first acid is from about 0.05:1 to about 20:1. In some embodiments, the molar ratio of the second acid to the first acid is from about 0.5:1 to about 10:1. In some embodiments, the molar ratio of the second acid to the first acid is from about 1:1 to about 4:1. In some embodiments, the molar ratio of the second acid to the first acid is about 2:1. In some embodiments, a molar ratio of the API to the first acid is about 0.1:1 to about 10:1. In some embodiments, a molar ratio of the API to the first acid is from about 0.2:1 to about 5:1 or from about 0.5:1 to about 2:1. In some embodiments, a molar ratio of the API to the first acid is about 1:1. In some embodiments, a molar ratio of the API to the second acid is about 0.05:1 to about 20:1. In some embodiments, a molar ratio of the API to the second acid is from about 0.1:1 to about 5:1 or from about 0.2:1 to about 1:1. In some embodiments, a molar ratio of the API to the second acid is about 0.5:1.

### Adsorbents

In one aspect, the pharmaceutical compositions disclosed herein comprises an ASD comprising a lipophilic API, a hydrophilic polymer, optionally a surfactant, and optionally, an adsorbent. In some embodiments, the API is an API of Table 1 or a pharmaceutically acceptable salt thereof. In some embodiments, the ASD optionally comprises an adsorbent. In some embodiments, the ASD optionally comprises one or more adsorbents.

Many adsorbents are solid, porous or super porous adsorption materials. They comprise numerous micro- or nano-pores within their structures, resulting in very large surface areas, for example, greater than 500 m²/g. Exemplary adsorbents include, without limitation, silicon dioxide, active carbon, magnesium aluminum silicate, diatomite, microcrystalline cellulose (MCC), silicified microcrystalline cellulose (SMCC), tale, crosslinked povidone, sodium carboxymethylcellulose, sodium carboxymethyl starch, and also sugars or sugar alcohols such as sorbitol, mannitol, lactose, cyclodextrin, and maltodextrin. In some embodiments, the adsorbent is silicon dioxide.

In some embodiments, an adsorbent, such as silicon dioxide, is present in the ASD by weight of about 1% to about 70%. In some embodiments, an adsorbent is present in the ASD by weight of about 1% to about 60%. In some embodiments, an adsorbent is present in the ASD by weight of about 1% to about 50%. In some embodiments, an adsorbent is present in the ASD by weight of about 1% to about 40%. In some embodiments, an adsorbent is present in the ASD by weight of about 1% to about 30%. In some embodiments, an adsorbent is present in the ASD by weight of about 1% to about 20%. In some embodiments, an adsorbent is present in the ASD by weight of about 5% to about 15%. In some embodiments, an adsorbent is present in the ASD by weight of about 15% to about 30%. In In In some embodiments, an adsorbent is present in the ASD by weight of about 20% to about 30%. In some embodiments, an adsorbent is present in the ASD by weight of about 25% to about 40%. In some embodiments, the adsorbent is present in the ASD by weight of about 1% to about 5%, about 1% to about 10%, about 1% to about 20%, about 1% to about 30%, about 1% to about 40%, about 1% to about 50%, about 1% to about 60%, about 1% to about 70%, about 5% to about 10%, about 5% to about 20%, about 5% to about 30%, about 5% to about 40%, about 5% to about 50%, about 5% to about 60%, about 5% to about 70%, about 10% to about 20%, about 10% to about 30%, about 10% to about 40%, about 10% to about 50%, about 10% to about 60%, about 10% to about 70%, about 20% to about 30%, about 20% to about 40%, about 20% to about 50%, about 20% to about 60%, about 20% to about 70%, about 30% to about 40%, about 30% to about 50%, about 30% to about 60%, about 30% to about 70%, about 40% to about 50%, about 40% to about 60%, about 40% to about 70%, about 50% to about 60%, about 50% to about 70%, or about 60% to about 70%. In some embodiments, the adsorbent is present in the ASD by weight of about 1%, about 5%, about 10%, about 20%, about 30%, about 40%, about 50%, about 60%, or about 70%. In some embodiments, the adsorbent is present in the ASD by weight of at least about 1%, about 5%, about 10%, about 20%, about 30%, about 40%, about 50%, or about 60%. In some embodiments, the adsorbent is present in the ASD by weight of at most about 5%, about 10%, about 20%, about 30%, about 40%, about 50%, about 60%, or about 70%. In some embodiments, the adsorbent is SiO₂. In some embodiments, the adsorbent comprises silicon dioxide, active carbon, magnesium aluminum silicate, diatomite, microcrystalline cellulose (MCC), silicified microcrystalline cellulose (SMCC), tale, crosslinked povidone, sodium carboxymethylcellulose, sodium carboxymethyl starch, and also sugars or sugar alcohols such as sorbitol, mannitol, lactose, cyclodextrin, or maltodextrin, or a combination thereof. In some embodiments, the adsorbent comprises active carbon. In some embodiments, the adsorbent comprises magnesium aluminum silicate. In some embodiments, the adsorbent comprises diatomite. In some embodiments, the adsorbent comprises MCC. In some embodiments, the adsorbent comprises SMCC. In some embodiments, the adsorbent comprises tale. In some embodiments, the adsorbent comprises SMCC. In some embodiments, the adsorbent comprises crosslinked povidone. In some embodiments, the adsorbent comprises sodium carboxymethylcellulose. In some embodiments, the adsorbent comprises sodium carboxymethyl starch. In some embodiments, the adsorbent comprises sugars or sugar alcohols such as sorbitol, mannitol, lactose, cyclodextrin, or maltodextrin.

In some embodiments, an ASD is formulated in a unit dosage form, such as a capsule or a tablet. In some embodiments, the adsorbent is present in the ASD in an amount of about 1 mg to about 5,000 mg. In some embodiments, the adsorbent is present in the ASD in an amount of about 1 mg to about 5 mg, about 1 mg to about 10 mg, about 1 mg to about 20 mg, about 1 mg to about 30 mg, about 1 mg to about 50 mg, about 1 mg to about 100 mg, about 1 mg to about 200 mg, about 1 mg to about 500 mg, about 1 mg to about 1,000 mg, about 1 mg to about 3,000 mg, about 1 mg to about 5,000 mg, about 5 mg to about 10 mg, about 5 mg to about 20 mg, about 5 mg to about 30 mg, about 5 mg to about 50 mg, about 5 mg to about 100 mg, about 5 mg to about 200 mg, about 5 mg to about 500 mg, about 5 mg to about 1,000 mg, about 5 mg to about 3,000 mg, about 5 mg to about 5,000 mg, about 10 mg to about 20 mg, about 10 mg to about 30 mg, about 10 mg to about 50 mg, about 10 mg to about 100 mg, about 10 mg to about 200 mg, about 10 mg to about 500 mg, about 10 mg to about 1,000 mg, about 10 mg to about 3,000 mg, about 10 mg to about 5,000 mg, about 20 mg to about 30 mg, about 20 mg to about 50 mg, about 20 mg to about 100 mg, about 20 mg to about 200 mg, about 20 mg to about 500 mg, about 20 mg to about 1,000 mg, about 20 mg to about 3,000 mg, about 20 mg to about 5,000 mg, about 30 mg to about 50 mg, about 30 mg to about 100 mg, about 30 mg to about 200 mg, about 30 mg to about 500 mg, about 30 mg to about 1,000 mg, about 30 mg to about 3,000 mg, about 30 mg to about 5,000 mg, about 50 mg to about 100 mg, about 50 mg to about 200 mg, about 50 mg to about 500 mg, about 50 mg to about 1,000 mg, about 50 mg to about 3,000 mg, about 50 mg to about 5,000 mg, about 100 mg to about 200 mg, about 100 mg to about 500 mg, about 100 mg to about 1,000 mg, about 100 mg to about 3,000 mg, about 100 mg to about 5,000 mg, about 200 mg to about 500 mg, about 200 mg to about 1,000 mg, about 200 mg to about 3,000 mg, about 200 mg to about 5,000 mg, about 500 mg to about 1,000 mg, about 500 mg to about 3,000 mg, about 500 mg to about 5,000 mg, about 1,000 mg to about 3,000 mg, about 1,000 mg to about 5,000 mg, or about 3,000 mg to about 5,000 mg. In some embodiments, the adsorbent is present in the ASD in an amount of about 1 mg, about 5 mg, about 10 mg, about 20 mg, about 30 mg, about 50 mg, about 100 mg, about 200 mg, about 500 mg, about 1,000 mg, about 3,000 mg, or about 5,000 mg. In some embodiments, the adsorbent is present in the ASD in an amount of at least about 1 mg, about 5 mg, about 10 mg, about 20 mg, about 30 mg, about 50 mg, about 100 mg, about 200 mg, about 500 mg, about 1,000 mg, or about 3,000 mg. In some embodiments, the adsorbent is present in the ASD in an amount of at most about 5 mg, about 10 mg, about 20 mg, about 30 mg, about 50 mg, about 100 mg, about 200 mg, about 500 mg, about 1,000 mg, about 3,000 mg, or about 5,000 mg.

In some embodiments, an adsorbent is present in an ASD described herein. In some embodiments, an adsorbent powder described herein has an D50 value of 1-1000 n. In some embodiments, the D50 value of the adsorbent is from about 0.01 to 1000 nm. In some embodiments, the D50 value of the adsorbent is from about 0.01 nm to about 1,000 nm. In some embodiments, the D50 value of the adsorbent is from at least about 0.01 nm. In some embodiments, the D50 value of the adsorbent is from at most about 1,000 nm. In some embodiments, the D50 value of the adsorbent is from about 1 nm to about 500 nm. In some embodiments, the D50 value of the adsorbent is from at least about 1 nm. In some embodiments, the D50 value of the adsorbent is from at most about 500 nm. In some embodiments, the D50 value of the adsorbent is from about 1 nm to about 300 nm, about 1 nm to about 700 nm, about 1 nm to about 100 nm, about 1 nm to about 130 nm, about 1 nm to about 170 nm, about 1 nm to about 200 nm, about 1 nm to about 230 n, about 1 nm to about 270 nm, about 1 nm to about 30 n, about 1 nm to about 400 n, about 1 nm to about 500 nm, about 10 nm to about 130 nm, about 10 nm to about 170 nm, about 100 nm to about 200 nm, about 100 nm to about 230 nm, about 100 nm to about 270 nm, about 100 nm to about 300 nm, about 100 nm to about 400 nm, about 100 nm to about 500 nm, about 200 nm to about 270 nm, about 200 nm to about 300 nm, about 200 nm to about 400 nm, about 200 nm to about 500 nm, about 300 nm to about 400 nm, about 300 nm to about 500 nm, or about 400 nm to about 500 nm. In some embodiments, the D50 value of the adsorbent is from about 1 nm to about 100 nm. In some embodiments, the D50 value of the adsorbent is from at least about 1 nm. In some embodiments, the D50 value of the adsorbent is about 0.1, 1, 30, 50, 70, 100, 130, 170, 200, 230, 250, 270, 300, 330, 350, 370, 400, 430, 450, 470, 500, 600, 700, 800, 900, or 1000 nm or less. In some embodiments, the adsorbent is silicon dioxide powder with an average diameter of 1-1000 n. In some embodiments, the D50 value of the silicon dioxide is from about 0.01 to 1000 nm. In some embodiments, the D50 value of the silicon dioxide is from about 0.01 nm to about 1,000 nm. In some embodiments, the D50 value of the silicon dioxide is from about 1 nm to about 100 n. In some embodiments, the D50 value of the silicon dioxide is from at least about 1 nm. In some embodiments, the D50 value of the silicon dioxide is about 0.1, 1, 30, 50, 70, 100, 130, 170, 200, 230, 250, 270, 300, 330, 350, 370, 400, 430, 450, 470, 500, 600, 700, 800, 900, or 1000 nm or less.

In some embodiments, the ASD comprises an adsorbent, wherein the adsorbent is silicon dioxide. In some embodiments, the silicon dioxide is present in the amorphous solid dispersion. In some embodiments, the amorphous solid dispersion is granulated and incorporated into a pharmaceutical composition with extra granular additives. In some embodiments, the silicon dioxide is present outside of the amorphous solid dispersion as an extra-granular additive. In some embodiments, silicon dioxide is present in the amorphous solid dispersion as well as being an extra-granular additive.

### Other Additives

In one aspect, pharmaceutical compositions described herein comprise an ASD comprising a lipophilic API, a hydrophilic polymer, optionally a surfactant, optionally an acid, optionally an adsorbent, and optionally an additional additive or additives. In some embodiments, the API is an API of Table 1 or a pharmaceutically acceptable salt thereof. In some embodiments, a pharmaceutically acceptable organic or inorganic acid or acid is included as an internal additive and thus as part of a solid dispersion. In some embodiments, a pharmaceutically acceptable organic or inorganic acid or acids are included as an external acid that is not part of the ASD. In some embodiments, a pharmaceutically acceptable organic or inorganic acid or acids are included in the pharmaceutical compositions. The pharmaceutically acceptable organic acid is selected from the group consisting of tartaric acid, fumaric acid, succinic acid, citric acid, lactic acid, malic acid, aliphatic sulfonic acids (e.g., methanesulfonic acid, ethanesulfonic acid, isethionic acid, etc.) and aromatic sulfonic acids (e.g., benzenesulfonic acid, p-toluenesulfonic acid, etc.), and the pharmaceutically acceptable inorganic acid is selected from the group consisting of hydrochloric acid, sulfuric acid, phosphoric acid and the like.

In some embodiments, the external acid in an amount of from about 1 mg to about 5,000 mg. In some embodiments, the external acid is present in the pharmaceutical composition in an amount of from about 1 mg to about 5 mg, about 1 mg to about 50 mg, about 1 mg to about 100 mg, about 1 mg to about 500 mg, about 1 mg to about 1,000 mg, about 1 mg to about 3,000 mg, about 1 mg to about 5,000 mg, about 10 mg to about 50 mg, about 10 mg to about 100 mg, about 10 mg to about 1,000 mg, about 10 mg to about 3,000 mg, about 10 mg to about 5,000 mg, about 20 mg to about 50 mg, about 20 mg to about 100 mg, about 20 mg to about 200 mg, about 50 mg to about 100 mg, about 50 mg to about 200 mg, about 50 mg to about 500 mg, or about 100 mg to about 200 mg. In some embodiments, the external acid is present in the pharmaceutical compositions and is not present in the ASD. In some embodiments, the external acid is malic acid. In some embodiments, the external acid is citric acid. In some embodiments, the external acid is tartaric acid.

In some embodiments, the external acid is present in a pharmaceutical composition by weight of about 1% to about 60%. In some embodiments, the external acid is present in a pharmaceutical composition in amount of about 1% to about 5%, about 1% to about 10%, about 1% to about 15%, about 1% to about 20%, about 1% to about 25%, about 1% to about 30%, about 1% to about 35%, about 1% to about 40%, about 1% to about 50%, about 1% to about 60%, about 5% to about 10%, about 5% to about 15%, about 5% to about 20%, about 5% to about 25%, about 5% to about 30%, about 5% to about 35%, about 5% to about 40%, about 5% to about 50%, about 5% to about 60%, about 10% to about 15%, about 10% to about 20%, about 10% to about 25%, about 10% to about 30%, about 10% to about 35%, about 10% to about 40%, about 10% to about 50%, about 10% to about 60%, about 15% to about 20%, about 15% to about 25%, about 15% to about 30%, about 15% to about 35%, about 15% to about 40%, about 15% to about 50%, about 15% to about 60%, about 20% to about 25%, about 20% to about 30%, about 20% to about 35%, about 20% to about 40%, about 20% to about 50%, about 20% to about 60%, about 25% to about 30%, about 25% to about 35%, about 25% to about 40%, about 25% to about 50%, about 25% to about 60%, about 30% to about 35%, about 30% to about 40%, about 30% to about 50%, about 30% to about 60%, about 35% to about 40%, about 35% to about 50%, about 35% to about 60%, about 40% to about 50%, about 40% to about 60%, or about 50% to about 60% by weight. In some embodiments, the external acid is present in a pharmaceutical composition in an amount of about 1%, about 5%, about 10%, about 15%, about 20%, about 25%, about 30%, about 35%, about 40%, about 50%, or about 60% by weight. In some embodiments, the external acid is present in a pharmaceutical composition in an amount of at least about 1%, about 5%, about 10%, about 15%, about 20%, about 25%, about 30%, about 35%, about 40%, or about 50% by weight. In some embodiments, the external acid is present in a pharmaceutical composition in an amount of at most about 5%, about 10%, about 15%, about 20%, about 25%, about 30%, about 35%, about 40%, about 50%, or about 60% by weight. In some embodiments, the external acid is present in the pharmaceutical compositions and is not present in the ASD. In some embodiments, the external acid is malic acid. In some embodiments, the external acid is citric acid. In some embodiments, the external acid is tartaric acid.

A pharmaceutical composition described herein comprises one or more preservatives. The ASD comprises one or more preservatives. The preservatives can include anti-microbials, antioxidants, and agents that enhance sterility. Exemplary preservatives include ascorbic acid, ascorbyl palmitate, butylatedhydroxyanisole (BHA), Butylatedhydroxytoulene (BHT), propyl gallate, citric acid, EDTA and its salts, erythorbic acid, fumaric acid, malic acid, propyl gallate, sodium ascorbate, sodium bisulfate, sodium metabisulfite, sodium sulfite, parabens (such as methylparaben, ethylparaben, propylparaben, butylparaben and their salts), benzoic acid, sodium benzoate, potassium sorbate, vanillin, and the like. In some embodiments, an amorphous solid dispersion composition or a pharmaceutical composition described herein comprises an antioxidant. In some embodiments, the antioxidant comprises a-tocopherol acetate, acetone sodium bisulfite, acetylcysteine, ascorbic acid, vitamin E, ascorbyl palmitate, BHA, BHT, cysteine, cysteine hydrochloride, d-a-tocopherol (natural or synthetic), dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, or tocopherols.

In some embodiments, pharmaceutical compositions described herein comprise an ASD comprising a lipophilic API, a hydrophilic polymer, optionally a surfactant, optionally an adsorbent, and optionally an additional additive or additives. In some embodiments, an antioxidant or mixture of antioxidants are included as the internal additive thus as part of a solid dispersion. In some embodiments, an antioxidant or mixture of antioxidants are included as an external additive. The exemplary antioxidants include but are not limited to BHT, BHA, gallic acid, propyl gallate, ascorbic acid, ascorbyl palmitate, 4hydroxymethyl-2,6-di-tert-butyl phenol, and tocopherol.

In some embodiments, the antioxidant is present in the ASD by weight of about 0.0001% to about 15%. In some embodiments, the antioxidant is present in the ASD by weight of about 0.0001% to about 0.1%, about 0.0001% to about 1%, about 0.0001% to about 2%, about 0.01% to about 1%, about 0.01% to about 2%, about 0.01% to about 10%, about 1% to about 2%, about 1% to about 5%, about 1% to about 7%, about 1% to about 10%, about 1% to about 15%, about 2% to about 10%, about 2% to about 15%, about 5% to about 10%, about 5% to about 15%, or about 10% to about 15%. In some embodiments, the antioxidant is present in the ASD by weight of at least about 0.0001%, about 0.001%, about 0.01%, about 0.1%, about 1%, about 2%, about 5%, about 7%, or about 10%. In some embodiments, the antioxidant is present in the ASD by weight of at most about 0.001%, about 0.01%, about 0.1%, about 1%, about 2%, about 5%, about 7%, about 10%, or about 15%.

In some embodiments, the antioxidant is present in a pharmaceutical composition described herein by weight of about 0.0001% to about 15%. In some embodiments, the antioxidant is present in a pharmaceutical composition described herein by weight of about 0.0001% to about 0.1%, about 0.0001% to about 1%, about 0.001% to about 0.1%, about 0.001% to about 1%, about 0.01% to about 10%, about 0.1% to about 1%, about 0.1% to about 2%, about 0.1% to about 5%, about 0.1% to about 10%, about 1% to about 2%, about 1% to about 5%, about 1% to about 10%, about 1% to about 15%, about 2% to about 5%, about 2% to about 10%, about 5% to about 7%, about 5% to about 10%, or about 1% to about 15%. In some embodiments, the antioxidant is present in a pharmaceutical composition described herein in an amount of at least about 0.0001%, about 0.001%, about 0.01%, about 0.1%, about 1%, about 2%, or about 5% by weight. In some embodiments, the antioxidant is present in a pharmaceutical composition described herein in an amount of at most about 0.001%, about 0.01%, about 0.1%, about 1%, about 2%, about 5%, about 7%, about 10%, or about 15% by weight. In some embodiments, the antioxidant is present in the amorphous solid dispersion. In some embodiments, the antioxidant is present in the pharmaceutical composition but not present in the ASD.

The above different additives can be used alone or together.

In some embodiments, a pharmaceutical composition described herein comprises a glidants. In some embodiments, the glidant is silicon dioxide powder. In some embodiments, the silicon dioxide is present in the amorphous solid dispersion. In some embodiments, silicon dioxide is not present in the amorphous solid dispersion, but is included in the pharmaceutical composition. In some embodiments, the silicon dioxide is present in the amorphous solid dispersion as well as being a component of the pharmaceutical composition outside of the amorphous solid dispersion.

In some embodiments, other additives conventionally mixed with the ASD is included. In some embodiments, other additives conventionally mixed with pharmaceutical compositions is included but are not present in the ASD. Such additives are well known in the art. The additives include, but are not limited to, anti-adherents (anti-sticking agents, glidants, flow promoters, lubricants) (e.g., tale, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, silicone dioxide, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding, (e.g., matrix binders (dry starch, dry sugars), film binders (starch paste, celluloses, bentonite, sucrose)), chemical binders (e.g., polymeric cellulose derivatives, such as carboxy methyl cellulose, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PEG, pregelatinized starch, sorbitol, glucose, etc.), bufferants, where the acid is a pharmaceutically acceptable acid, (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc) and where the base is a pharmaceutically acceptable base, (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a pharmaceutically acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants, (e.g., titanium dioxide, food dyes, lakes, natural vegetable colorants, iron oxides, silicates, sulfates, magnesium hydroxide and aluminum hydroxide), coolants, (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers, (e.g., lactose, mannitol, tale, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, sodium starch glycolate and microcrystalline cellulose), hydrogen bonding agents, (e.g., magnesium oxide), flavorants or desensitizers, (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents), (e.g., sugars, cellulosics, polymers and alginates).

In some embodiments, other additives are used in the pharmaceutical composition described herein but not as a part of an ASD. In some embodiments, other additives are used for inner granulation that is not part of an ASD. In some embodiments, other additives are used for outer granulation, such as for forming a tablet. In some embodiments, other additives are used as outer diluent. In some embodiments, the other additives comprise microcrystalline cellulose (MCC), an organic acid (e.g., tartaric acid), magnesium stearate, povidone, copovidone, crospovidone (e.g., PVPP-XL), or a combination thereof. In some embodiments, the other additives comprise MCC (e.g., PH102). In some embodiments, the other additives comprise magnesium stearate. In some embodiments, the other additives comprise crospovidone (e.g., PVPP-XL). In some embodiments, the other additives comprise an organic acid, such as tartaric acid. In some embodiments, the other additives are present in the ASD by weight of about 1% to about 60%. In some embodiments, the other additives are present in the ASD by weight of about 1% to about 50%. In some embodiments, the other additives are present in the ASD by weight of about 1% to about 40%. In some embodiments, the other additives are present in the ASD by weight of about 1% to about 30%. In some embodiments, the other additives are present in the ASD by weight of about 1% to about 20%. In some embodiments, the other additives are present in the ASD by weight of about 5% to about 15%. In some embodiments, the other additives are present in the ASD by weight of about 15% to about 30%. In some embodiments, the other additives are present in the ASD by weight of about 20% to about 30%. In some embodiments, the other additives are present in the ASD by weight of about 25% to about 40%.

Additives can also be materials such as proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, (e.g., dicalcium phosphate, hydroxyapitite, tricalcium phosphate, tale and titania), polyols (e.g., mannitol, xylitol and sorbitol polyethylene glycol esters) and polymers (e.g., alginates, poly(lactide coglycolide), gelatin, crosslinked gelatin and agar-agar). In some embodiments, the proteins comprise amino acids such as glutamic acid, aspartic acid, or acidic salts of glycine, alanine or serine.

In some embodiments, the pharmaceutical compositions described herein comprises a pharmaceutically acceptable carrier or excipient, wherein the pharmaceutically acceptable carrier or excipient is not part of the ASD. In some embodiments, the pharmaceutically acceptable carrier or excipient is free of organic acid. In some embodiments, the pharmaceutically acceptable carrier or excipient is free of inorganic acid. In some embodiments, the pharmaceutically acceptable carrier or excipient is free of acid. In some embodiments, the pharmaceutically acceptable carrier or excipient comprises an external acid that is not present in the amorphous solid dispersion.

In some embodiments, the external acid is a surface modified acid. In some embodiments, a surface modified acid comprises a powdered or granulated acid with a neutral salt layer at least partially coating the exterior of the powdered or granulated acid. In some embodiments, the surface modified acid comprises a powdered or granulated acid selected from tartaric acid, fumaric acid, succinic acid, citric acid, lactic acid, malic acid, maleic acid, benzenesulfonic acid, p-toluenesulfonic acid, glutamic acid, aspartic acid, and acidic salts of glycine, alanine or serine. In some embodiments, the surface modified acid comprises a powdered or granulated acid selected from tartaric acid, fumaric acid, succinic acid, citric acid, lactic acid, and malic acid. In some embodiments, the surface modified acid comprises a powdered or granulated acid selected from tartaric acid.

In some embodiments, the neutral salt layer decreases the reactivity of the acid with other components of the composition, such as the active ingredient. In some embodiments, an API shows improved solubility in the presence of acids. In some embodiments, pharmaceutical compositions with surface modified acids provide increased solubility while preserving the stability of the active ingredient.

In some embodiments, the surface modified acid is prepared by reacting a basic solution with the acid particle (e.g., powdered or granulated acid to form a neutral salt layer on the surface of the acid. In some embodiments, the neutral salt layer comprises an anion from the acid and a cation from the base. In some embodiments, the basic solution comprises a pharmaceutically acceptable base. In some embodiments, the pharmaceutically acceptable base is selected from sodium carbonate, potassium carbonate, sodium acetate, potassium acetate, sodium stearate, potassium stearate, lysine, arginine and histidine. In some embodiments, the pharmaceutically acceptable base is a carbonate base.

In some embodiments, the surface modified acid is prepared by reacting a basic solution with the powdered or granulated acid to form a neutral salt layer on the surface of the acid. In some embodiments, the concentration of the basic solution in the pharmaceutical composition by weight is about 1% to about 30%. In some embodiments, the concentration of the basic solution by weight in the pharmaceutical composition is about 5% to about 15%. In some embodiments, the concentration of the basic solution in the pharmaceutical composition by weight is about 1% to about 5%, about 1% to about 10%, about 1% to about 12.5%, about 1% to about 15%, about 1% to about 20%, about 1% to about 25%, about 1% to about 30%, about 2.5% to about 10%, about 2.5% to about 15%, about 2.5% to about 20%, about 2.5% to about 30%, about 5% to about 7.5%, about 5% to about 10%, about 5% to about 20%, about 5% to about 25%, about 10% to about 15%, about 10% to about 20%, about 10% to about 25%, about 10% to about 30%, about 12.5% to about 15%, about 12.5% to about 20%, about 12.5% to about 25%, about 12.5% to about 30%, about 15% to about 20%, about 15% to about 25%, about 15% to about 30%, or about 25% to about 30%. In some embodiments, the concentration of the basic solution in the pharmaceutical composition by weight is at most about 2.5%, about 5%, about 7.5%, about 10%, about 12.5%, about 15%, about 20%, about 25%, or about 30%. In some embodiments, the weight ratio of the base to acid is about 1% to about 20%. In some embodiments, the weight ratio of the base to acid is about 1% to about 5%, about 1% to about 10%, about 1% to about 15%, about 1% to about 20%, about 2% to about 20%, about 5% to about 10%, about 5% to about 15%, about 5% to about 20%, about 10% to about 15%, about 10% to about 20%, or about 15% to about 20%. In some embodiments, the weight ratio of the base to acid in the pharmaceutical composition is at least about 1%, about 2%, about 3%, about 4%, about 5%, about 6%, about 7%, about 8%, about 9%, about 10%, or about 15%. In some embodiments, the weight ratio of the base to acid is at most about 2%, about 3%, about 4%, about 5%, about 6%, about 7%, about 8%, about 9%, about 10%, about 15%, or about 20%.

In an exemplary embodiment, surface modified tartaric acid is prepared by reacting a basic solution of sodium carbonate with powdered tartaric acid to form a neutral salt layer on the tartaric acid. In some embodiments, the sodium carbonate aqueous solution is formulated at a concentration of about 5-30%. The amount of sodium carbonate is about 1-10% in a ratio by weight to the tartaric acid Second, the formulated sodium carbonate aqueous solution is added to tartaric acid powder particles having a particle size of about 40 to 60 mesh. After stirring, the tartaric acid powder particles are dried in a drying oven or a fluidized bed to yield the modified tartaric acid powder particles.

### Oral Dosage Forms

The present pharmaceutical compositions can take the form of solutions, suspensions, emulsion, tablets, pills, pellets, capsules, capsules containing liquids, powders, suppositories, emulsions, suspensions, or any other form suitable for use. Preferred pharmaceutical compositions are formulated for oral delivery. In some embodiments, the pharmaceutically acceptable vehicle is a capsule. Capsules may be hard capsules or soft capsules, push-fit capsules made of gelatin, as well as soft, sealed capsules made of gelatin and a plasticizer (such as glycerol or sorbitol). In some embodiments, the capsule contains about 1000 mg of the pharmaceutical composition. In some embodiments, the capsule contains less than 1000 mg of the pharmaceutical composition. Capsules can be of any size. Examples of standard sizes include, but are not limited to those listed in Table 2, (#000, #00, #0, #1, #2, #3, #4, and #5). In some embodiments, the pharmaceutical composition is in the dosage form of a liquid filled into a hard capsule. In some embodiments, the pharmaceutical composition is in the dosage form of a liquid filled into a soft capsule. In some embodiments, the pharmaceutical composition is in the dosage form of a tablet. In some embodiments, the pharmaceutical composition comprises an amorphous solid dispersion. In some embodiments, the pharmaceutical composition comprises an amorphous solid dispersion in the dosage form of a tablet. In some embodiments, the pharmaceutical composition is in the dosage form of a multilayer tablet. In some embodiments, the tablet has one, two, three, four or more layers. In some embodiments, the tablet has an inner core and an outer core.

TABLE 2

| | | Locked | External |
|---|---|---|---|
| | Volume | length | diameter |
| Size | (mL) | (mm) | (mm) |
| 000 | 1.37 | 26.1 | 9.9 |
| 00 | 0.91 | 23.3 | 8.5 |
| 0 | 0.68 | 21.7 | 7.6 |
| 1 | 0.50 | 19.4 | 6.9 |
| 2 | 0.37 | 18.0 | 6.3 |
| 3 | 0.30 | 15.9 | 5.8 |
| 4 | 0.21 | 14.3 | 5.3 |

See, e.g., Remington's Pharmaceutical Sciences, page 1658-1659 (Alfonso Gennaro ed., Mack Publishing Company, Easton Pa., 18^{th} ed., 1990), which is incorporated by reference. In some embodiments, the capsules used herein are of size #00 or #0.

### Methods of Administration

Pharmaceutical compositions described herein are administered for the treatment or prevention of diseases. When used to treat or prevent diseases or disorders, pharmaceutical compositions are administered or applied singly, or in combination with other agents. Pharmaceutical compositions may also be administered or applied singly, in combination with other pharmaceutically active agents. Provided herein are methods of treatment and prophylaxis by administration to a subject in need of such treatment of a therapeutically effective amount of a pharmaceutical composition of the invention. In some embodiments, the subject is an animal, e.g., a mammal such as a human. In some embodiments, pharmaceutical compositions described herein include an ASD comprising lipophilic API, a hydrophilic polymer, optionally a surfactant, optionally an adsorbent and optionally an organic or inorganic acid. In some embodiments, the API is a lipophilic API. In some embodiments, the lipophilic API is one listed in Table 1, or a pharmaceutically acceptable salt thereof. In some embodiments, the lipophilic API, hydrophilic polymer, and the surfactant is formulated as an amorphous solid dispersion.

In some embodiments, the pharmaceutical compositions are administered orally. In some embodiments, the pharmaceutical compositions are administered in an oral liquid, semi-liquid or semisolid dosage form. In some embodiments, the pharmaceutical compositions are administered as a solid oral dosage form. In some embodiments, the pharmaceutical compositions are administered as a liquid oral dosage form. In some embodiments, the pharmaceutical compositions are administered as a pill, tablet, chewable tablet, specialty tablet, buccal tablet, sub-lingual tablet, orally-disintegrating tablet, capsule, gel capsule, soft gel capsule, hard gel capsule, specialty capsule, buccal capsule, sub-lingual capsule, orally-disintegrating capsule, powder, granule, crystal or orally dispersible film. In some embodiments, the pharmaceutical compositions are administered as a liquid or a capsule. In some embodiments, the pharmaceutical compositions are administered as a soft gel capsule. In some embodiments, the pharmaceutical compositions are administered as a hard gel capsule.

In some embodiments, the pharmaceutical composition is formulated for oral administration. In some embodiments, the pharmaceutical composition is formulated for once daily dosing. In some embodiments, the pharmaceutical composition is formulated for twice daily dosing.

In some embodiments, a herein described pharmaceutical composition is storage stable for a period of at least 1 month, 2 months, 3 months, 6 months, 9 months, 12 months, or 24 months at 5±3° C., wherein a storage stable pharmaceutical composition retains at least 90 wt % of the API compound or the pharmaceutically acceptable salt thereof at the end of the period. In some embodiments, a herein described pharmaceutical composition is storage stable for a period of at least 2 weeks, 1 month, 2 months, 3 months, 6 months, 9 months, 12 months, or 24 months at 25±2° C., wherein a storage stable pharmaceutical composition retains at least 90 wt % of the API compound or the pharmaceutically acceptable salt thereof at the end of the period. In some embodiments, a herein described pharmaceutical composition is storage stable for a period of at least 12 months at 25±2° C., wherein a storage stable pharmaceutical composition retains at least 90 wt % of the API compound or the pharmaceutically acceptable salt thereof at the end of the period. In some embodiments, a herein described pharmaceutical composition is storage stable for a period of at least 2 weeks, 1 month, 2 months, 3 months, 6 months, 12 months, or 24 months at 40±2° C., wherein a storage stable pharmaceutical composition retains at least 90 wt % of the API compound or the pharmaceutically acceptable salt thereof at the end of the period. In some embodiments, a herein described pharmaceutical composition is storage stable for a period of at least 1 month, 2 months, 3 months, 6 months, 9 months, 12 months, or 24 months at 5±3° C., wherein a storage stable pharmaceutical composition contains at most 0.5 wt % total impurity at the end of the period. In some embodiments, a herein described pharmaceutical composition is storage stable for a period of at least 2 weeks, 1 month, 2 months, 3 months, 6 months, 9 months, 12 months, or 24 months at 25±2° C., wherein a storage stable pharmaceutical composition contains at most 0.5 wt % total impurity at the end of the period. In some embodiments, a herein described pharmaceutical composition is storage stable for a period of at least 12 months at 25±2° C., wherein a storage stable pharmaceutical composition contains at most 0.5 wt % total impurity at the end of the period. In some embodiments, a herein described pharmaceutical composition is storage stable for a period of at least 2 weeks, 1 month, 2 months, 3 months, 6 months, 12 months, or 24 months at 40±2° C., wherein a storage stable pharmaceutical composition contains at most 0.5 wt % total impurity at the end of the period.

### Conditions

In one aspect, described herein is a method of treating a disease or condition by administering to a subject in need thereof the pharmaceutical compositions or the ASDs described herein. In one aspect, the pharmaceutical compositions or ASDs described herein is used to treat a disease or condition by administering to a subject in need thereof the pharmaceutical compositions or the ASDs. In some embodiments, the disease or condition is cancer. In some embodiments, the disease or condition is a mental disorder.

In some embodiments, the pharmaceutical compositions may be used to inhibit one or more tyrosine kinases in a subject in need of inhibiting such tyrosine kinases. In some embodiments, the subject has a disease or condition associated with tyrosine kinase. In some embodiments, pharmaceutical compositions described herein include a lipophilic API, a hydrophilic polymer, and a surfactant. In some embodiments, pharmaceutical compositions described herein include a lipophilic API, a hydrophilic polymer, and a surfactant. In some embodiments, the API is a lipophilic API. In some embodiments, the lipophilic API is one listed in Table 1, or a pharmaceutically acceptable salt thereof. In some embodiments, the lipophilic API, hydrophilic polymer, and the surfactant is formulated as an amorphous solid dispersion.

In one aspect, described herein is a method of treating a disease or condition in a subject. The disease or condition can be a cancer. In some embodiments, a pharmaceutical composition described herein may be used to treat or prevent cancer. In some embodiments, the pharmaceutical compositions may be used to treat or prevent prostate cancer, breast cancer, ovarian cancer, endometrial cancer, bladder cancer, pancreatic cancer, hepatocellular cancer, kidney cancer, liver cancer, salivary gland carcinoma, hair loss, acne, hirsutism, ovarian cysts, polycystic ovary disease, precocious puberty, spinal and bulbar muscular atrophy, or age-related macular degeneration. In some embodiments, the pharmaceutical compositions may be used to treat or prevent prostate cancer. In some embodiments, the pharmaceutical compositions can be used to treat or prevent one or more of leukemia, Philadelphia chromosome (Ph+)-positive chronic myelogenous leukemia, gastrointestinal stromal tumor, Parkinson's disease, castration-resistant prostate cancer, metastatic castration-resistant prostate cancer, castration-recurrent prostate cancer, high-risk castration-sensitive prostate cancer, metastatic high-risk castration-sensitive prostate cancer, hormone-resistant prostate cancer, hormone-refractory prostate cancer, androgen-independent prostate cancer, androgen deprivation resistant prostate cancer, androgen ablation resistant prostate cancer, androgen depletion-independent prostate cancer, anti-androgen-recurrent prostate cancer, metastatic castration-resistant prostate cancer in patients who have already received prior chemotherapy containing docetaxel, newly diagnosed high risk metastatic hormone sensitive prostate cancer (mHSPC), metastatic castration resistant prostate cancer in patients who are asymptomatic, mildly symptomatic after failure of androgen deprivation therapy in whom chemotherapy is not yet clinically indicated, metastatic castration resistant prostate cancer in patients whose disease has progressed on or after a docetaxel-based chemotherapy regimen. In some embodiments, the pharmaceutical compositions are used to treat newly diagnosed adult patients with Philadelphia chromosome positive chronic myeloid leukemia (Ph+ CML) in chronic phase. In some embodiments, the pharmaceutical compositions are used to treat children with newly diagnosed Philadelphia chromosome-positive (Ph+) chronic myeloid leukemia (CML) in the chronic phase (CP). In some embodiments, the pharmaceutical compositions are used to treat chronic phase (CP) and accelerated phase (AP) Ph+ CML in adult patients resistant to or intolerant to prior therapy that included imatinib. In some embodiments, pharmaceutical compositions described herein include a lipophilic API, a hydrophilic polymer, and a surfactant. In some embodiments, pharmaceutical compositions described herein include a lipophilic API, a hydrophilic polymer, and a phospholipid or poloxamer. In some embodiments, pharmaceutical compositions described herein include a lipophilic API, a hydrophilic polymer, and lecithin. In some embodiments, pharmaceutical compositions described herein include a lipophilic API, a hydrophilic polymer, and lecithin. In some embodiments, the API is a lipophilic API. In some embodiments, the lipophilic API is one listed in Table 1, or a pharmaceutically acceptable salt thereof. In some embodiments, the lipophilic API, hydrophilic polymer, and the surfactant is formulated as an amorphous solid dispersion.

In some embodiments, the subject is an adult. In some embodiments, the subject is a child. In some embodiments, the subject is at least one year old. In some embodiments, the subject is less than one year old. In some embodiments, the subject is 1 to 12 years old. In some embodiments, the subject is 1 to 18 years old. In some embodiments, the subject is 12 to 18 years old. In some embodiments, the subject is at least 18 years old. In some embodiments, the subject is at least 24 years old. In some embodiments, the subject is 1 to 90 years old. In one aspect, described herein is a method of inhibiting one or more tyrosine kinases.

In some embodiments, the pharmaceutical composition is used to treat a cancer selected from the group consisting of breast cancer, cancer is selected from the group consisting of acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor. In some embodiments, the disease or condition is associated with tyrosine kinase.

In some embodiments, pharmaceutical compositions described herein can be used in combination therapy with at least one other therapeutic agent. The pharmaceutical composition and the therapeutic agent can act additively or, more preferably, synergistically. In some embodiments, the pharmaceutical composition is administered concurrently with the administration of another therapeutic agent. In some embodiments, a pharmaceutical composition is administered prior or subsequent to administration of another therapeutic agent. In some embodiments, pharmaceutical compositions described herein include a lipophilic API, a hydrophilic polymer, and a surfactant. In some embodiments, pharmaceutical compositions described herein include a lipophilic API, a hydrophilic polymer, and a phospholipid or poloxamer. In some embodiments, pharmaceutical compositions described herein include a lipophilic API, a hydrophilic polymer, and lecithin. In some embodiments, pharmaceutical compositions described herein include a lipophilic API, a hydrophilic polymer, and lecithin. In some embodiments, the API is a lipophilic API. In some embodiments, the lipophilic API is one listed in Table 1, or a pharmaceutically acceptable salt thereof. In some embodiments, the lipophilic API, hydrophilic polymer, and the surfactant is formulated as an amorphous solid dispersion.

### a) Cabozantinib

In some embodiments, the pharmaceutical compositions or ASD described herein comprising cabozantinib, cabozantinib malate, or a pharmaceutically acceptable salt thereof is used to treat cancer. In some embodiments, the cancer comprises kidney cancer, liver cancer, and thyroid cancer. In some embodiments, the kidney cancer is advanced renal cell carcinoma. In some embodiments, the liver cancer is hepatocellular carcinoma. In some embodiments, the thyroid cancer is locally advanced or metastatic differentiated thyroid cancer or medullary thyroid cancer.

In some embodiments, the pharmaceutical compositions or ASD described herein comprising cabozantinib or a pharmaceutically acceptable salt thereof (such as cabozantinib malate) is used to inhibit multiple tyrosine-kinases, comprising administering to a subject in need thereof the pharmaceutical compositions or the ASD. In some embodiments, the multiple tyrosine-kinases comprises VEGFR2. In some embodiments, the multiple tyrosine-kinases comprises MET, RET, AXL, VEGFR2, FLT3, and c-KIT. In some embodiments, the multiple tyrosine-kinases comprises MET. In some embodiments, the multiple tyrosine-kinases comprises RET. In some embodiments, the multiple tyrosine-kinases comprises AXL. In some embodiments, the multiple tyrosine-kinases comprises VEGFR2. In some embodiments, the multiple tyrosine-kinases comprises FLT3. In some embodiments, the multiple tyrosine-kinases comprises c-KIT.

In some embodiments, the pharmaceutical compositions or ASD described herein comprising cabozantinib or a pharmaceutically acceptable salt thereof (such as cabozantinib malate) is administered to a subject in combination of an immunotherapeutic agent. In some embodiments, the immunotherapeutic agent is nivolumab. In some embodiments, the subject was previously treated with sorafenib. In some embodiments, the subject is 12 years of age or older. In some embodiments, the subject progressed following prior VEGFR-targeted therapy. In some embodiments, the subject is radioactive iodine-refractory or ineligible. In some embodiments, the subject is 12 years of age or older, progressed following prior VEGFR-targeted therapy, and is radioactive iodine-refractory or ineligible.

### b) Venetoclax

In some embodiments, the pharmaceutical compositions or ASD described herein comprising venetoclax or a pharmaceutically acceptable salt thereof is used to treat cancer. In some embodiments, the cancer comprises blood cancer. In some embodiments, the blood cancer is chronic lymphocytic leukemia. In some embodiments, the blood cancer is acute myeloid leukemia. In some embodiments, the cancer comprises a solid tumor. In some embodiments, the solid tumor is lymphoma. In some embodiments, the lymphoma is small lymphocytic lymphoma.

In some embodiments, the pharmaceutical compositions or ASD described herein comprising venetoclax or a pharmaceutically acceptable salt thereof is used to inhibit B-cell lymphoma-2 (Bel-2) protein, comprising administering to a subject in need thereof the pharmaceutical compositions or the ASD.

In some embodiments, the pharmaceutical compositions or ASD described herein comprising venetoclax or a pharmaceutically acceptable salt thereof is administered to a subject in combination of an immunotherapeutic agent. In some embodiments, the immunotherapeutic agent is obinutuzumab or rituximab. In some embodiments, the immunotherapeutic agent is Obinutuzumab. In some embodiments, the immunotherapeutic agent is rituximab.

In some embodiments, the pharmaceutical compositions or ASD described herein comprising venetoclax or a pharmaceutically acceptable salt thereof is administered to a subject in combination of a chemotherapeutic agent. In some embodiments, the chemotherapeutic agent is azacitidine, or decitabine, or low-dose cytarabine. In some embodiments, the chemotherapeutic agent is azacitidine. In some embodiments, the chemotherapeutic agent is decitabine. In some embodiments, the chemotherapeutic agent is low-dose cytarabine.

In some embodiments, the subject was previously untreated. In some embodiments, the subject was previously treated. In some embodiments, the subject is 12 years of age or older. In some embodiments, the subject is newly diagnosed of acute myeloid leukemia, and is 75 years of age or older or has other medical conditions that prevent the use of standard chemotherapy. In some embodiments, the subject is an adult.

### c) Abiraterone Acetate

In some embodiments, the pharmaceutical compositions or ASD described herein comprising abiraterone free base, abiraterone acetate, or a pharmaceutically acceptable salt thereof is used to treat cancer. In some embodiments, the cancer comprises a solid tumor. In some embodiments, the solid tumor is prostate cancer. In some embodiments, the prostate cancer is metastatic castration-resistant prostate cancer. In some embodiments, the prostate cancer is metastatic high-risk castration-sensitive prostate cancer.

In some embodiments, the pharmaceutical compositions or ASD described herein comprising abiraterone free base, abiraterone acetate, or a pharmaceutically acceptable salt thereof is used to inhibit 17 alpha-hydroxylase/C17, 20-lyase (CYP17), comprising administering to a subject in need thereof the pharmaceutical compositions or the ASD.

In some embodiments, the pharmaceutical compositions or ASD described herein comprising abiraterone, abiraterone acetate, or a pharmaceutically acceptable salt thereof is administered to a subject in combination of a corticosteroid. In some embodiments, the corticosteroid is prednisone or methylprednisolone. In some embodiments, the corticosteroid is prednisone. In some embodiments, the corticosteroid is methylprednisolone. In some embodiments, the subject was previously treated with sorafenib. In some embodiments, the subject is a male adult.

### d) Alectinib Hydrochloride

In some embodiments, the pharmaceutical compositions or ASD described herein comprising alectinib free base, alectinib hydrochloride, or a pharmaceutically acceptable salt thereof is used to treat cancer. In some embodiments, the cancer comprises a solid tumor. In some embodiments, the solid tumor is lung cancer. In some embodiments, the lung cancer is non-small cell lung cancer. In some embodiments, the non-small cell lung cancer is anaplastic lymphoma kinase (ALK)-positive.

In some embodiments, the pharmaceutical compositions or ASD described herein comprising alectinib free base, alectinib hydrochloride, or a pharmaceutically acceptable salt thereof is used to inhibit a tyrosine kinase, comprising administering to a subject in need thereof the pharmaceutical compositions or the ASD. In some embodiments, the tyrosine kinase comprises ALK. In some embodiments, the tyrosine kinase comprises RET. In some embodiments, the tyrosine kinase comprises ALK and RET.

In some embodiments, the pharmaceutical compositions or ASD described herein comprising alectinib free base, alectinib hydrochloride, or a pharmaceutically acceptable salt thereof is administered to a subject in need thereof. In some embodiments, the subject is intolerant to crizotinib. In some embodiments, the subject is an adult.

### e) Pazopanib Hydrochloride

In some embodiments, the pharmaceutical compositions or ASD described herein comprising pazopanib free base, pazopanib hydrochloride, or a pharmaceutically acceptable salt thereof is used to treat cancer. In some embodiments, the cancer comprises a solid tumor. In some embodiments, the solid tumor is soft tissue sarcoma. In some embodiments, the soft tissue sarcoma is advanced soft tissue sarcoma. In some embodiments, the cancer is kidney cancer. In some embodiments, the kidney cancer is advanced renal cell cancer.

In some embodiments, the pharmaceutical compositions or ASD described herein comprising pazopanib free base, pazopanib hydrochloride, or a pharmaceutically acceptable salt thereof is used to inhibit a tyrosine kinase, comprising administering to a subject in need thereof the pharmaceutical compositions or the ASD. In some embodiments, the tyrosine kinase comprises VEGF receptor (VEGFR). In some embodiments, the tyrosine kinase comprises PDGF receptor (PDGFR). In some embodiments, the tyrosine kinase comprises VEGFR and PDGFR.

In some embodiments, the pharmaceutical compositions or ASD described herein comprising pazopanib free base, pazopanib hydrochloride, or a pharmaceutically acceptable salt thereof is administered to a subject in need thereof. In some embodiments, the subject previously received chemotherapy. In some embodiments, the subject is an adult.

### f) Lurasidone Hydrochloride

In some embodiments, the pharmaceutical compositions or ASD described herein comprising lurasidone free base, lurasidone hydrochloride, or a pharmaceutically acceptable salt thereof is used to treat a mental disorder. In some embodiments, the mental disorder comprises depression. In some embodiments, the depression is associated with bipolar I disorder. In some embodiments, the depression is bipolar depression.

In some embodiments, the pharmaceutical compositions or ASD described herein comprising lurasidone free base, lurasidone hydrochloride, or a pharmaceutically acceptable salt thereof is used to inhibit one or more receptors, comprising administering to a subject in need thereof the pharmaceutical compositions or the ASD. In some embodiments, the one or more receptors comprise central dopamine D2 and serotonin Type 2 (5HT2A) receptor. In some embodiments, the one or more receptors comprise central dopamine D2. In some embodiments, the one or more receptors comprise 5HT2A.

In some embodiments, the pharmaceutical compositions or ASD described herein comprising lurasidone free base, lurasidone hydrochloride, or a pharmaceutically acceptable salt thereof is administered to a subject in need thereof in combination of an anticonvulsant. In some embodiments, the anticonvulsant is lithium or valproate. In some embodiments, the anticonvulsant is lithium. In some embodiments, the anticonvulsant is valproate. In some embodiments, the subject previously received chemotherapy. In some embodiments, the subject is an adult. In some embodiments, the subject is an adolescent. In some embodiments, the adolescent is 13 to 17 years old.

### g) Vilazodone Hydrochloride

In some embodiments, the pharmaceutical compositions or ASD described herein comprising vilazodone free base, vilazodone hydrochloride, or a pharmaceutically acceptable salt thereof is used to treat a mental disorder. In some embodiments, the mental disorder comprises major depressive disorder.

In some embodiments, the pharmaceutical compositions or ASD described herein comprising vilazodone free base, vilazodone hydrochloride, or a pharmaceutically acceptable salt thereof is used to inhibit one or more receptors, comprising administering to a subject in need thereof the pharmaceutical compositions or the ASD. In some embodiments, the one or more receptors comprise serotonin Type 2 (5HT2A) receptor.

In some embodiments, the pharmaceutical compositions or ASD described herein comprising vilazodone free base, vilazodone hydrochloride, or a pharmaceutically acceptable salt thereof is used to stimulate one or more transporters, comprising administering to a subject in need thereof the pharmaceutical compositions or the ASD. In some embodiments, the one or more transporters comprise serotonin transporter. In some embodiment, the stimulation is via partial agonism.

In some embodiments, the pharmaceutical compositions or ASD described herein comprising vilazodone free base, vilazodone hydrochloride, or a pharmaceutically acceptable salt thereof is administered to a subject in need thereof. In some embodiments, the subject is an adult.

### Methods Of Manufacture

Disclosed herein is a method for preparing an amorphous solid dispersion, comprising the steps of (a) combining (i) an active pharmaceutical ingredient or a pharmaceutically acceptable salt thereof, (ii) a surfactant (e.g., polymeric non-ionic surfactants and phospholipids), (iii) a hydrophilic polymer (e.g., non-ionic hydrophilic polymer), (iv) optionally an adsorbent or adsorbents, and (v) optionally other additional additives such as an acid. In some embodiments, the API, surfactants, and hydrophilic polymer can be combined by any suitable methods in the art. In some embodiments, the API, surfactants, and hydrophilic polymer are combined by melt extrusion (such as hot melt extrusion or HME). Disclosed herein is a method for preparing an amorphous solid dispersion, comprising the steps of (a) combining (i) an active pharmaceutical ingredient or a pharmaceutically acceptable salt thereof, (ii) a surfactant (e.g., polymeric non-ionic surfactants and phospholipids), (iii) a hydrophilic polymer (e.g., non-ionic hydrophilic polymer), (iv) optionally an adsorbent or adsorbents, (v) optionally other additional additives such as an acid, and (vi) a solvent or solvent mixture, thereby producing a liquid mixture (a solution or suspension), and (b) removing all or a part of the solvent from said mixture, thereby producing an amorphous solid dispersion.

In some embodiments, the solvent is selected from an organic solvent and water. In some embodiments, the organic solvent is ethyl acetate, ethanol, isopropanol, or methanol, n-butanol, n-propanol, isopropanol, formic acid, nitromethane, ethanol, acetic acid, N-methylpyrrolidone, tetrahydrofuran (THF), methyl acetate, dimethylformamide, acetonitrile, dimethyl sulfoxide, dichloromethane (DCM), acetone, and any combination thereof. In some embodiments, the solvent is an alcohol. In some embodiments, the alcohol is ethanol. In some embodiments, the solvent is selected from dichloromethane, methanol, tetrahydrofuran, and acetone. In some embodiments, the solvent is selected from a mixture of these solvents. In some embodiments, combining comprises dissolving the active pharmaceutical ingredient or a pharmaceutically acceptable salt thereof, the surfactant, the hydrophilic polymer, and optionally an adsorbent and/or additional additives in the solvent. In some embodiments, the adsorbent is suspended in the solvent. In some embodiments, removing of the solvent comprises spray-drying, rotor evaporation or fluid bed drying. In some embodiments, the API is an API of Table 1 or a pharmaceutically acceptable salt thereof.

In some embodiments, the method for preparing an amorphous solid dispersion, comprises the steps of (a) combining (i) an active pharmaceutical ingredient or a pharmaceutically acceptable salt thereof, (ii) a surfactant (e.g., polymeric non-ionic surfactants and phospholipids), (iii) a hydrophilic polymer (e.g., non-ionic hydrophilic polymer), (iv) optionally other additional additives and (v) a solvent or solvent mixture, to produce a liquid mixture or solution; (b) spraying the liquid mixture or solution onto an adsorbent or adsorbents; and (c) removing all or a part of the solvent from the liquid mixture or solution to produce an amorphous solid dispersion. In some embodiments, the solvent is selected from an organic solvent and water. In some embodiments, the organic solvent is ethyl acetate, ethanol, isopropanol, or methanol, n-butanol, n-propanol, isopropanol, formic acid, nitromethane, ethanol, acetic acid, N-methylpyrrolidone, tetrahydrofuran, methyl acetate, dimethylformamide, acetonitrile, dimethyl sulfoxide, dichloromethane (DCM), acetone, and any combination thereof. In some embodiments, the solvent is an alcohol. In some embodiments, the alcohol is ethanol. In some embodiments, the solvent is selected from dichloromethane, tetrahydrofuran, methanol and acetone. In some embodiments, the solvent is selected from a mixture of these solvents. In some embodiments, combining comprises dissolving the active pharmaceutical ingredient or a pharmaceutically acceptable salt thereof the surfactant, the hydrophilic polymer, and optionally additional additives in the solvent. In some embodiments, an amorphous solid dispersion is produced by a fluid-bed spraying and drying process. In some embodiments, an amorphous solid dispersion is produced by rotary evaporation. In some embodiments, the adsorbent is suspended in the solvent. In some embodiments, removing of the solvent comprises vacuum-drying, spray-drying, rotary evaporation or fluid bed drying. In some embodiments, the solvent or solvent mixture is one selected from Table 10.

TABLE 10
Exemplary solvents or solvent mixture
suitable for manufacturing an ASD.

| Solvent or solvent mixture | Ratio, volume by volume (V/V) |
|---|---|
| Methanol | — |
| Water | — |
| DCM | — |
| Ethyl acetate | — |
| Dioxane | — |
| Methyl ethyl ketone | — |
| Tetrahydrofuran | — |
| Chloroform | — |
| Acetonitrile | — |
| Ethanol | — |
| Acetone | — |
| Water/Methanol | 5/5 to 4/6 |
| Methanol/Methyl ethyl ketone | 7/3 to 6/4 |
| Ethanol/Acetonitrile | 3/7 to 4/6 |
| Isopropanol/DCM | 3/7 to 5/5 |
| Dichloromethane (DCM)/Methanol | 7/3 to 5/5 |
| Acetone/Water | 5/5 to 8/2 |
| Methyl ethyl ketone/chloroform | 5/5 to 4/6 |
| Dioxane/Methanol | 7/3 to 5/5 |
| Methanol/Chloroform | 7/3 to 6/4 |
| Acetonitrile/DCM | 3/7 to 4/6 |
| Tetrahydrofuran/Acetonitrile | 2/8 to 3/7 |
| Ethyl acetate/DCM | 5/5 to 4/6 |
| Chloroform/Water | 3/7 to 4/6 |
| Acetonitrile/Methanol | 3/7 to 5/5 |

In an exemplary manufacturing workflow, an amorphous solid dispersion is formed by first the API, hydrophilic polymer, surfactant, optionally an acid, optionally an adsorbent, and optionally an additive or additives in a solvent (e.g., a solvent or solvent mixture selected from Table 10) or water at a room temperature or heated to form a clear solution. The clear solution is then spray dried or vacuum dried to form an amorphous solid dispersion. Additionally, an amorphous solid dispersion is formed by first the API, hydrophilic polymer, surfactant, optionally an acid, optionally an adsorbent, and optionally an additive or additives in a solvent or water in a room temperature or heated to form a clear solution. An adsorbent or a mixture of adsorbents is further added at a room temperature or heated to form a homogenous suspension. The homogenous suspension is then spray dried or vacuum dried to form an amorphous solid dispersion. Following the formation of an amorphous solid dispersion, the amorphous solid dispersion is mixed with other additives and excipients used in the composition. The mixture is then pressed into tablets or loaded into capsules. In some embodiments, dry granulation is performed by slugging, milling and screening to form dry granules. Additional excipients can be mixed with the dry granules and then filled into capsules. Additional excipients can be mixed with the dry granules and then compressed into tablets.

In an exemplary manufacturing workflow, an amorphous solid dispersion is formed by first dispersing an API (e.g., an API listed in Table 1) in a solvent (e.g., a solvent or solvent mixture selected from Table 6) or water, optionally with stirring, at a room temperature or heated to form a clear solution. Then, a hydrophilic polymer, a surfactant, and optionally an additive or additives are added in the solution. Optionally, an adsorbent or a mixture of adsorbents is further added to the solution at a room temperature or heated to form a homogenous suspension. The solution or suspension is then spray dried or vacuum dried to form an amorphous solid dispersion. The homogenous suspension is then spray dried or vacuum dried to form an amorphous solid dispersion. Following the formation of an amorphous solid dispersion, the amorphous solid dispersion is mixed with other additives and excipients used in the formulation. The mixture is then pressed into tablets or loaded into capsules.

A typical spray dryer comprises three chambers, a drying chamber, a cyclone chamber and a sample collection chamber. During the spray drying process, the spray dried dispersion solid is collected in the sample collection chamber. However, the solid may also reside on the surfaces of the drying chamber and cyclone chamber, thus causing a low production yield (a low amount of solid in the sample collection chamber). In some embodiments, the amorphous solid dispersions comprising of an API, a hydrophilic polymer and a surfactant have low production yields. When an adsorbent is incorporated into these amorphous solid dispersions, the production yield can be significantly increased. In some embodiments, the production yield of an amorphous solid dispersion with an adsorbent is increased by at least 10%, in comparison to the solid dispersion without an adsorbent. For clarity, the term of percentage means to be the absolute difference of the yields. For example, if the production yield of an amorphous solid dispersion without an adsorbent is 10% and the production yield of an amorphous solid dispersion with an adsorbent is 20%, the increase of the yield is the difference of these two yields, i.e., 10%. In some embodiments, the production yield of an amorphous solid dispersion with an adsorbent is increased by at least 20%, in comparison to the solid dispersion without an adsorbent. In some embodiments, the production yield of an amorphous solid dispersion with an adsorbent is increased by at least 30%, in comparison to the solid dispersion without an adsorbent. In some embodiments, the production yield of an amorphous solid dispersion with an adsorbent is increased by at least 40%, in comparison to the solid dispersion without an adsorbent. In some embodiments, the production yield of an amorphous solid dispersion with an adsorbent is increased by at least 50%, in comparison to the solid dispersion without an adsorbent. In some embodiments, the production yield of an amorphous solid dispersion with an adsorbent is increased by at least 60%, in comparison to the solid dispersion without an adsorbent. In some embodiments, the production yield of an amorphous solid dispersion with an adsorbent is increased by at least 70%, in comparison to the solid dispersion without an adsorbent. In some embodiments, the production yield of an amorphous solid dispersion with an adsorbent is increased by at least 80%, in comparison to the solid dispersion without an adsorbent. In some embodiments, the production yield of an amorphous solid dispersion with an adsorbent is increased by at least 90%, in comparison to the solid dispersion without an adsorbent. In some

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1. Abiraterone Acetate Compositions and PK Study in Beagle Dogs

This example illustrates the process of improving the oral absorption of and reducing or removing the food-effect of abiraterone acetate, according to some embodiments of the present disclosure.

Abiraterone acetate is a CYP17 inhibitor in combination with prednisone for the treatment of patients with metastatic castration-resistant prostate cancer. It was developed by J&J and approved by FDA in 2011 under the brand name of Zytiga. Abiraterone acetate is very poorly water soluble and has lower oral bioavailability. The prescribing information for Zytiga® tablets recommends 1,000 mg (4×250 mg tablets) administered orally once daily in combination with prednisone (5 mg) administered orally twice daily.

The label states that ZYTIGA must be taken on an empty stomach, which means no food should be consumed for at least two hours before the dose is taken and for at least one hour after the dose is taken. Food sometimes increases systemic exposure of abiraterone and increases the risk for adverse effects.

Two compositions for abiraterone acetate as provided in Table 3A were prepared.

TABLE 3A

| | Batch No. | | |
|---|---|---|---|
| | P211115-1 | P211115-2 | |
| | Unit weight | Unit weight | |
| Components | (mg) | (mg) | Description |
| Abiraterone acetate | 50.0 | 50.0 | API |
| HPMCAS (grade LF) | 500.0 | 150.0 | polymer |
| Lecithin | / | 50.0 | surfactant |

Amorphous solid dispersions were prepared by spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 3B. After collection, the particles were dried in a convection tray dryer to remove the residual solvent before further use.

TABLE 3B

| | |
|---|---|
| Feed rate (rpm) | 6.0 |
| Inlet temperature (C. °) | 70 |
| Outlet temperature (C. °) | 48 |
| Atomization pressure (bar) | 2.6 |

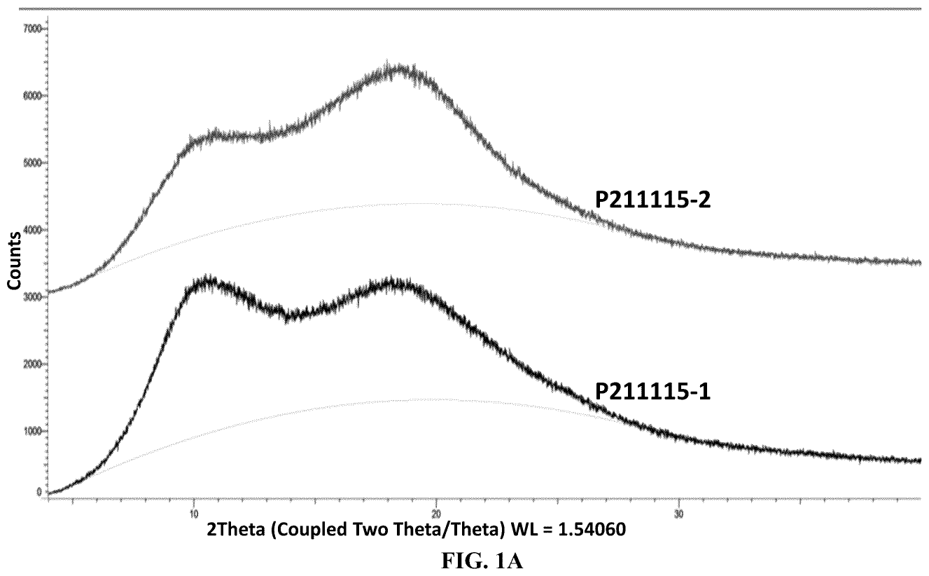

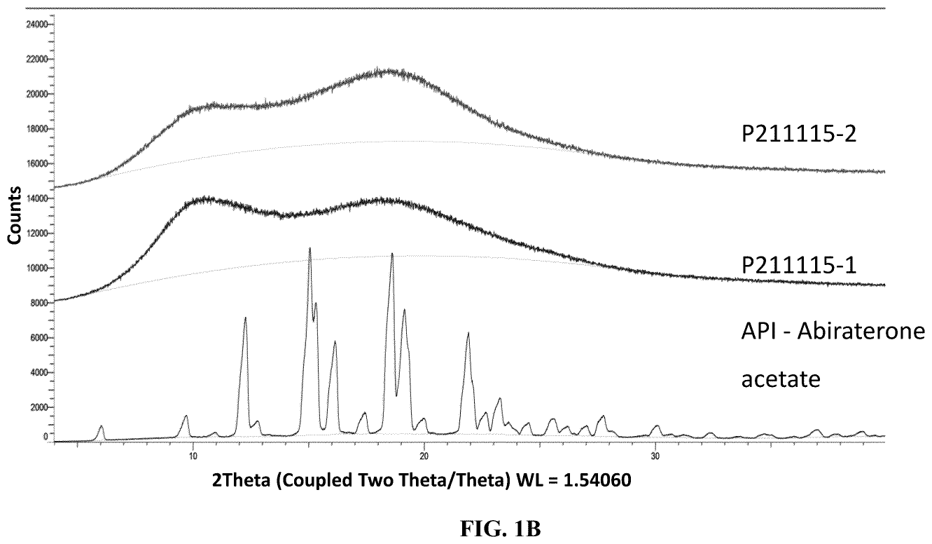

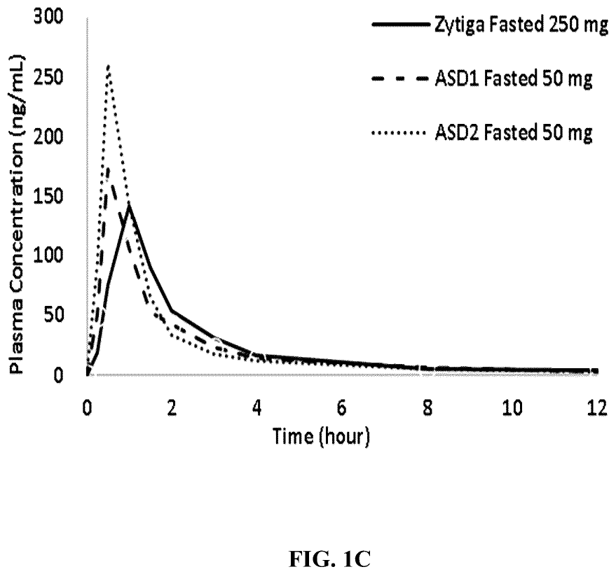

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step sze, 0.01 degree; time/step, 0.5 s; sample stage rotation, 15 r/min. The results (FIGS. 1A-1B) for both batches show that the particles were in amorphous state.

The ASD capsules (P211115-1 and P211115-2) and the reference product, ZYTIGA® tablet, were tested orally in six beagle dogs under fasted condition. Six dogs were classified into three groups using a three-way crossover design, each group were fed with P211115-1, P211115-2 and ZYTIGA. The dose is 50 mg per dog in ASD capsule groups and 250 mg per dog in ZYTIGA group. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.25, 0.5, 1, 1.5, 2, 3, 4, 8, 12 and 24 h after the drug administration. Abiraterone plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 3C include the geometric mean values as well as coefficient of variation (CV) of the pharmacokinetic parameters of the three dosing groups. FIG. 1C also shows a comparison of plasma concentrations of API (Abiraterone) in dog model when reference product ZYTIGA (250 mg) and two ASD compositions of Abiraterone (Batch No. P211115-1 and P211115-2) were given orally at the dose of 50 mg API in fasted condition. The two test capsules increased the absorption significantly compared to Zytiga in fasted condition, given that the dosing of abiraterone in ASD composition is 50 mg, only ⅕ of that in the reference product ZYTIGA (250 mg). High mass ratio of HPMCAS to API (5:1) showed a 5-fold increase in absorption compared to Zytiga. However, the addition of lecithin, lower mass ratio of HPMCAS to API (3:1) indicated the same absorption increasement but higher Cmax.

TABLE 3C

| | | Cₘₐₓ | AUCₗₐₛₜ | AUC_{inf} |
|---|---|---|---|---|
| Study design | | ng/mL | h*ng/mL | h*ng/mL |
| P211115-1 (50 mg) in | Average | 175.6 | 266.2 | 282.3 |
| fasted condition | CV(%) | 39.8% | 30.1% | 27.9% |
| P211115-2 (50 mg) in | Average | 281.4 | 310.4 | 328.7 |
| fasted condition | CV(%) | 66.1% | 71.8% | 71.4% |
| ZYTIGA (250 mg) in | Average | 163.3 | 286.4 | 307.8 |
| fasted condition | CV(%) | 77.0% | 82.4% | 81.5% |

Additional ASD compositions for abiraterone acetate as provided in Table 3D were prepared by spray drying. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 4.0-8.0 rpm, inlet temperature 50-100° C., outlet temperature 30-80° C., and atomization pressure 0-4.0 bar. The particles were collected and filled into the capsules.

TABLE 3D

| | | Batch No. | | | |
|---|---|---|---|---|---|
| Description | Ingredients | ABI-A | ABI-B | ABI-C | ABI-D |
| API | Abiraterone | 20.00 | 16.67 | 12.50 | 18.18 |
| | acetate (% wt) | | | | |
| Acid | Tartaric acid | — | — | — | 18.18 |
| | (% wt) | | | | |
| | Oleic acid (% wt) | — | — | 12.50 | — |
| Polymer | VA64 (% wt) | — | — | — | 18.18 |
| | HPMCAS-LF | 60.00 | 50.00 | 37.50 | — |
| | (% wt) | | | | |
| Surfactant | VE-TPGS (% wt) | — | — | — | 9.09 |
| | Lecithin (% wt) | 20.00 | 16.67 | — | 9.09 |
| Adsorbent | SiO₂ (% wt) | — | — | — | 27.27 |
| | Magnesium | — | — | 37.50 | — |
| | aluminum | | | | |
| | silicate (% wt) | | | | |

| | | | | |
|---|---|---|---|---|
| Total unit weight for each | 500.00 | 300.00 | 400.00 | 550.00 |
| batch (mg) | | | | |

### Example 2. Alectinib Hydrochloride Compositions and PK Study in Beagle Dogs

This example illustrates the process of improving the oral absorption of and reducing or removing the food-effect of alectinib hydrochloride, according to some embodiments of the present disclosure.

Alectinib is a kinase inhibitor indicated for the treatment of patients with anaplastic lymphoma kinase (ALK)-positive metastatic non-small cell lung cancer (NSCLC). It was developed by by Roche and approved by FDA in 2015 under the brand name ALECENSA. ALECENSA is administrated at 600 mg orally per day, which means four capsules once, taken with food. Alectinib is very hard to dissolve in aqueous solution and food helps increase the oral absorption. A high-fat, high-calorie meal increased the combined exposure of alectinib plus M4 (alectinib metabolite) by 3.1-fold following oral administration of a single 600 mg dose of Alecense. However, the absolute bioavailability of alectinib was 37% under fed conditions.

Alectinib compositions as described in Table 4A-1 were prepared.

TABLE 4A-1

| | Batch No. | | | | | | | |
|---|---|---|---|---|---|---|---|---|
| | I-M211221-1 | I-M211221-3 | I-M211221-4 | I-M211221-5 | I-M211202-3 | I-M211229-1 | M211021-2-I | |
| | Unit | Unit | Unit | Unit | Unit | Unit | Unit | |
| | weight | weight | weight | weight | weight | weight | weight | |
| Component | (mg) | (mg) | (mg) | (mg) | (mg) | (mg) | (mg) | Description |
| Alectinib | 150 | 150 | 150 | 150 | 150 | 150 | 150 | API |
| hydrochloride | | | | | | | | |
| HPMC-E5 | 150 | 150 | 150 | 150 | — | 150 | 150 | polymer |
| HPMCAS-LF | — | — | — | — | 300 | — | — | |
| TPGS | — | — | — | — | — | 150 | 150 | surfactant |
| SLS | — | — | — | — | 150 | — | — | |
| Tartaric acid | — | 150 | — | — | — | 150 | — | acid |
| Citric acid | — | — | 150 | — | — | — | — | excipient |
| Malic acid | — | — | — | 150 | — | — | — | |

Amorphous solid dispersions were prepared by Spray drying method. Briefly, API was dispersed in (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 4B. After collection, the particles were dried in a convection tray dryer to remove the residual solvent before further use.

TABLE 4B

| | |
|---|---|
| Feed rate (rpm) | 3 |
| Inlet temperature (C. °) | 90 |
| Outlet temperature (C. °) | 60 |
| Atomization pressure (bar) | 2.6 |

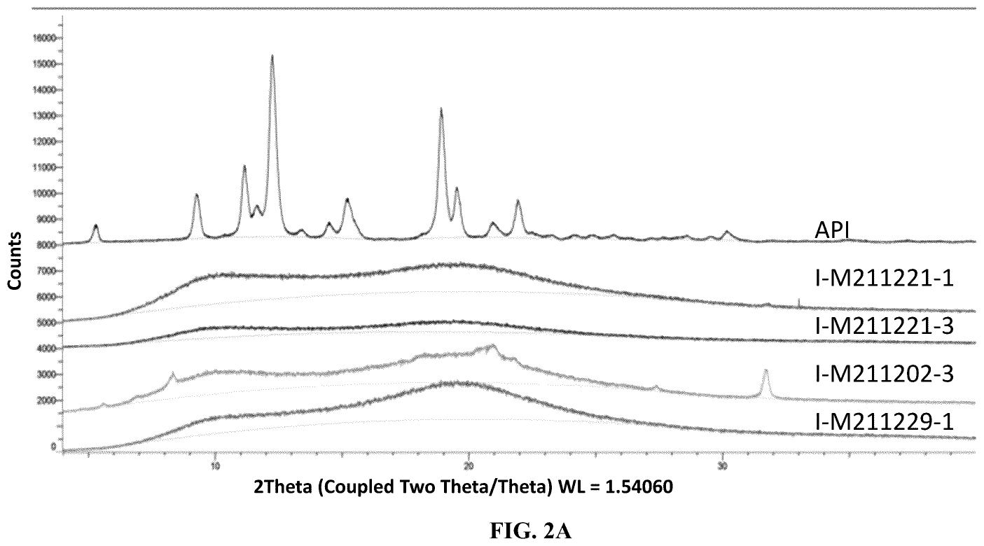

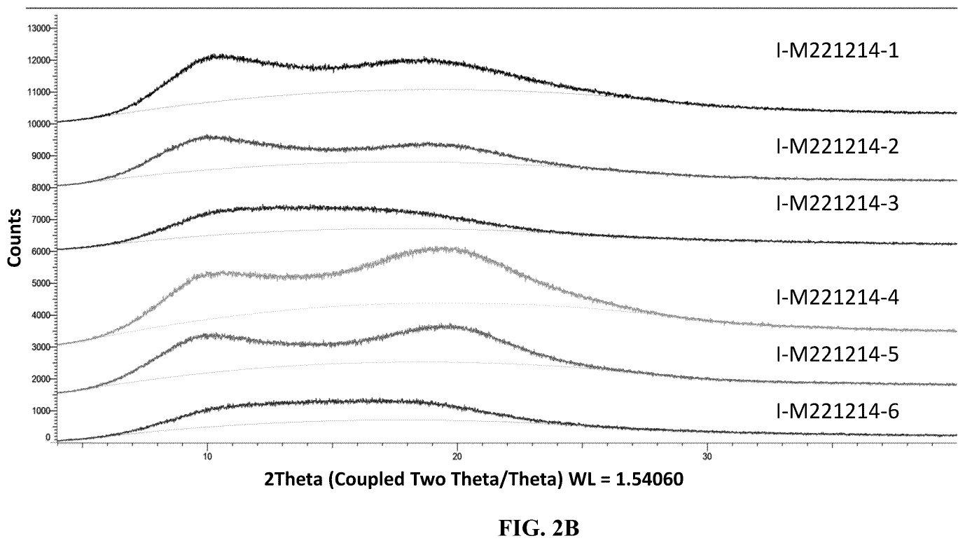

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The results (FIG. 2A) show that batch No. I-M211202-3 is mostly amorphous but with very small level of crystalline SLS and the other batches are in amorphous state. The results indicate that alectinib can be formulated to be an amorphous solid dispersion when SLS is not in the ASD or mostly amorphous when SLS is in ASD.

Kinetic solubility was also measured. Excess of SDD (spray-dried dispersions) powder was added into 40 ml FaSSIF medium at 37° C. under 400 rpm rotation. 1 ml medium was withdrawn into a centrifugal tube at specified time point (5, 15, 30, 60, 90, and 120 min) and then centrifuged for 10 min under 10000 rpm. The upper solution was collected for assay measure by HPLC.

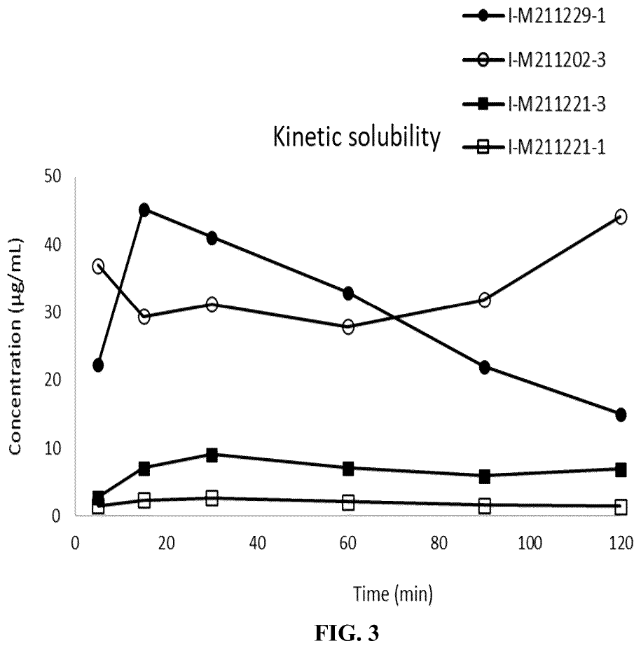

The results (Table 4C and FIG. 3) suggest that alectinib is very hard to dissolve in aqueous solution. When it was prepared as amorphous solid dispersion with HPMC E5 or HPMC E5 plus tartaric acid, it showed lower dissolution profile. Surfactant was found to help increase the dissolution even when alectinib was in crystalline form (batch No. J-M21 1202-3). When the ASD composition contains polymer, surfactant, and acid combination, the dissolution could be significantly increased and maintained for 2 hours or more.

TABLE 4C-1

| Time | Kinetic solubility (μg/mL) | | | | | | |
|---|---|---|---|---|---|---|---|
| (min) | I-M211221-1 | I-M211221-4 | I-M211221-5 | M211021-2-I | I-M211221-3 | I-M211202-3 | I-M211229-1 |
| 5 | 1.52 | 2.19 | 0.47 | 16.94 | 2.77 | 36.95 | 22.33 |
| 15 | 2.35 | 6.21 | 2.02 | 16.38 | 7.06 | 29.41 | 45.21 |
| 30 | 2.63 | 10.1 | 6.19 | 18.21 | 9.06 | 31.16 | 41.06 |
| 60 | 2.10 | 7.65 | 11.41 | 7.21 | 7.09 | 27.91 | 32.95 |
| 90 | 1.57 | 2.19 | 0.47 | 16.94 | 5.86 | 31.83 | 21.96 |
| 120 | 1.40 | 6.21 | 2.02 | 16.38 | 6.90 | 44.17 | 15.00 |

Six alectinib compositions as described in Table 4A-2 were prepared to evaluate the effect of polymers used in the composition.

TABLE 4A-2

| | | Batch No. | | | | | |
|---|---|---|---|---|---|---|---|
| | | I-M221214-1 | I-M221214-2 | I-M221214-3 | I-M221214-4 | I-M221214-5 | I-M221214-6 |
| Description | Ingredient | Unit weight (mg) | | | | | |
| API | Alectinib | 150 | 150 | 150 | 150 | 150 | 150 |
| | Hydrochloride | | | | | | |
| Polymer | HPMC-E5 | — | 450 | — | — | 450 | — |
| | HPMCAS-MF | 450 | — | — | 450 | — | — |
| | Eudragit | — | — | 450 | — | — | 450 |
| | (L100-55) | | | | | | |
| Surfactant | TPGS | — | — | — | 150 | 150 | 150 |

Amorphous solid dispersions were prepared by Spray drying method. Briefly, API was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 3.0-8.0 rpm, inlet temperature 50-100° C., outlet temperature 30-80° C., and atomization pressure 0-4.0 bar. The particles were collected and used for test of crystalline phase and dissolution in FaSSIF medium.

XRPD studies were conducted using the same method as described above. The results (FIG. 2B) show that all six batches are in amorphous state.

Excess of SDD (spray-dried dispersions) powder was added into 40 ml FaSSIF medium at 37° C. under 75 rpm rotation. 1 ml medium was withdrawn into a centrifugal tube at specified time point (5, 15, 30, 60, 90, and 120 min) and then centrifuged for 10 min under 10000 rpm. The upper solution was collected for assay measure by HPLC. Kinetic solubility results are shown in Table 4C-2. The results suggest that an ASD of API (Alectinib Hydrochloride), polymer, and surfactant results in a higher solubility than an ASD of API and polymer alone, especially in enteric polymer based formulation.

TABLE 4C-2

| Time | Kinetic solubility (μg/mL) | | | | | |
|---|---|---|---|---|---|---|
| (min) | I-M221214-1 | I-M221214-2 | I-M221214-3 | I-M221214-4 | I-M221214-5 | I-M221214-6 |
| 5 | 34.94 | 27.89 | 13.47 | 14.85 | 26.04 | 32.28 |
| 15 | 21.31 | 8.20 | 15.35 | 33.93 | 16.61 | 36.53 |
| 30 | 15.13 | 5.05 | 16.27 | 19.13 | 9.96 | 39.83 |
| 60 | 13.61 | 3.36 | 17.12 | 20.94 | 7.61 | 43.82 |
| 90 | 15.61 | 3.09 | 18.33 | 23.66 | 6.70 | 47.58 |
| 120 | 17.49 | 2.96 | 19.82 | 29.38 | 6.35 | 49.44 |

Four alectinib compositions as described in Table 4A-3 were prepared for evaluating the quantity of polymer in ASD, using the same spray dry method described above.

TABLE 4A-3

| | | Batch No. | | | |
|---|---|---|---|---|---|
| Description | Ingredient | M211021-1-I | I-M211202-3 | I-M211202-1 | M211101-2-I |
| API | Alectinib | 33.33 | 25.00 | 16.67 | 12.50 |
| | Hydrochloride | | | | |
| | (% wt) | | | | |
| Polymer | HPMCAS-LF (% wt) | 33.33 | 50.00 | 66.67 | 75.00 |
| Surfactant | TPGS (% wt) | 33.33 | — | — | — |
| | SDS (% wt) | — | 25.00 | 16.67 | 12.50 |

| | | | | |
|---|---|---|---|---|
| Total unit weight (mg) | 450 | 600 | 900 | 1200 |

Kinetic solubility was measured using the same method as described above, and the results are shown in Table 4C-3. The increase of polymer in ASD, which includes API, Polymer, and surfactant, results in a much better dissolution profile and higher kinetic solubility.

TABLE 4C-3

| Time | Kinetic solubility (μg/mL) | | | |
|---|---|---|---|---|
| (min) | M211021-1-I | I-M211202-3 | I-M211202-1 | M211101-2-I |
| 5 | 42.61 | 36.95 | 40.41 | 54.43 |
| 15 | 29.58 | 29.41 | 41.36 | 56.23 |
| 30 | 28.96 | 31.16 | 43.16 | 58.00 |
| 60 | 25.31 | 27.91 | 45.38 | 60.04 |
| 90 | 21.79 | 31.83 | 48.91 | 72.31 |
| 120 | 19.26 | 44.17 | 52.34 | 68.48 |

Four alectinib compositions as described in Table 4A-4 were prepared for evaluating the quantity of polymer in ASD, using the same spray dry method described above.

TABLE 4A-4

| | | Batch No. | | | |
|---|---|---|---|---|---|
| Description | Ingredient | M211021-5-I | I-M211202-4 | M211101-3-I | I-M211202-2 |
| API | Alectinib | 33.33 | 25.00 | 20.00 | 16.67 |
| | Hydrochloride | | | | |
| | (% wt) | | | | |
| Polymer | Soluplus (% wt) | 33.33 | 50.00 | 60.00 | 66.67 |
| Surfactant | TPGS (% wt) | 33.33 | — | — | — |
| | SDS (% wt) | — | 25.00 | 20.00 | 16.67 |

| | | | | |
|---|---|---|---|---|
| Total unit weight (mg) | 450 | 600 | 750 | 900 |

Kinetic solubility was measured using the same method as described above, and the results are shown in Table 4C-4. The increase of polymer in ASD, which includes API, Polymer, and surfactant, results in higher solubility.

TABLE 4C-4

| Time | Kinetic solubility (μg/mL) | | | |
|---|---|---|---|---|
| (min) | M211021-5-I | I-M211202-4 | M211101-3-I | I-M211202-2 |
| 5 | 46.04 | 236.36 | 251.12 | 223.22 |
| 15 | 66.33 | 371.13 | 389.54 | 370.68 |
| 30 | 72.83 | 340.5 | 422.73 | 449.55 |
| 60 | 83.69 | 321.19 | 396.88 | 541.26 |
| 90 | 79.56 | 314.93 | 367.43 | 600.74 |
| 120 | 65.62 | 311.28 | 372.74 | 603.07 |

Six alectinib compositions as described in Table 4A-5 were prepared to evaluate the effect of surfactants used in the composition, using the same spray dry method described above.

TABLE 4A-5

| | | Batch No. | | | | | |
|---|---|---|---|---|---|---|---|
| | | M210803-1-I | M210803-2-I | M210804-1-I | M210804-2-I | M210722-1-I | M210818-2-I |
| Description | Ingredient | Unit weight (mg) | | | | | |
| API | Alectinib | 150 | 150 | 150 | 150 | 150 | 150 |
| | Hydrochloride | | | | | | |
| Polymer | HPMCAS-LF | 150 | 150 | 150 | 150 | 150 | 150 |
| Acid | Tartaric acid | 150 | 150 | 150 | 150 | 150 | 150 |
| Surfactant | Tween-20 | 150 | — | — | — | — | — |
| | Gelucire44/14 | — | 150 | — | — | — | — |
| | RH40 | — | — | 150 | — | — | — |
| | SDS | — | — | — | 150 | — | 75 |
| | TPGS | — | — | — | — | 150 | 75 |
| Adsorbent | SiO2 | 150 | 150 | 150 | — | — | 150 |

Kinetic solubility was measured using the same method as described above, and the results are shown in Table 4C-5. The results suggest that surfactants can enhance the solubility of alectinib, especially TPGS and SDS.

TABLE 4C-5

| Time | Kinetic solubility (μg/mL) | | | | | |
|---|---|---|---|---|---|---|
| (min) | M210803-1-I | M210803-2-I | M210804-1-I | M210804-2-I | M210722-1-I | M210818-2-I |
| 5 | 30.91 | 18.64 | 27.60 | 53.73 | 40.13 | 38.30 |
| 15 | 42.33 | 29.09 | 33.68 | 51.26 | 46.54 | 53.01 |
| 30 | 45.29 | 30.34 | 34.60 | 63.84 | 53.82 | 60.02 |
| 60 | 44.80 | 29.73 | 34.30 | 62.66 | 57.06 | 64.22 |
| 90 | 42.25 | 27.35 | 34.16 | 63.14 | 58.81 | 66.90 |
| 120 | 40.11 | 25.20 | 34.30 | 63.78 | 59.18 | 69.76 |

Three alectinib compositions as described in Table 4A-6 were prepared to evaluate the quantity of surfactants in the composition, using the same spray dry method described above.

TABLE 4A-6

| | | Batch No. | | |
|---|---|---|---|---|
| | | M210823-3-I | M210804-2-I | M210927-1-I |
| Description | Ingredient | Unit weight (mg) | | |
| API | Alectinib | 150 | 150 | 150 |
| | Hydrochloride | | | |
| Polymer | HPMCAS-LF | 150 | 150 | 150 |
| Acid | Tartaric acid | 150 | 150 | 150 |
| Surfactant | SDS | 300 | 150 | 75 |

Kinetic solubility was measured using the same method as described above, and the results are shown in Table 4C-6. The results suggest that increase in surfactants quantity can enhance the solubility of alectinib.

TABLE 4C-6

| Time | Kinetic solubility (μg/mL) | | |
|---|---|---|---|
| (min) | M210803-1-I | M210803-2-I | M210804-1-I |
| 5 | 56.9 | 53.73 | 6.87 |
| 15 | 76.35 | 51.26 | 11.25 |
| 30 | 94.53 | 63.84 | 17.6 |
| 60 | 110.62 | 62.66 | 33.2 |
| 90 | 118.3 | 63.14 | 33.98 |
| 120 | 128.31 | 63.78 | 38.99 |

Two alectinib free base compositions as described in Table 4A-7 were prepared to evaluate the effect of API free base in the composition, using the same spray dry method described above.

TABLE 4A-6

| | | Batch No. | |
|---|---|---|---|
| | | M210714-2-I | M210907-1-I |
| Description | Ingredient | Unit weight (mg) | |
| API | Alectinib free base | 150 | 150 |
| Polymer | HPMCAS-LF | — | 150 |
| | Eudragit L100 | 150 | — |
| Acid | Tartaric acid | 150 | 150 |
| Surfactant | TPGS | 150 | 150 |

Kinetic solubility was measured using the same method as described above, and the results are shown in Table 4C-6. The results suggest that increase in surfactants quantity can enhance the solubility of alectinib.

TABLE 4C-6

| | |
|---|---|
| Time | Kinetic solubility (μg/mL) |

| (min) | M210714-2-I | M210907-1-I |
|---|---|---|
| 5 | 34.56 | 11.85 |
| 15 | 46.38 | 43.96 |
| 30 | 49.82 | 58.7 |
| 60 | 52.08 | 72.86 |
| 90 | 51.87 | 76.12 |
| 120 | 53.72 | 74.26 |

A separate alectinib composition as described in Table 4D was prepared.

TABLE 4D

| | Batch No. | |
|---|---|---|
| | M211207 | |
| Component | Unit weight (mg) | Description |
| Alectinib hydrochloride | 75 | API |
| Soluplus | 225 | polymer |
| SLS | 75 | surfactant |

Amorphous solid dispersions were prepared by spray drying method. Briefly, API was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were shown in Table 4E. After collection, the particles were dried in a convection tray dryer to remove the residual solvent before further use. The particles were filled into gel capsule, each capsule contains 75 mg alectinib hydrochloride.

TABLE 4E

| | |
|---|---|
| Feed rate (rpm) | 3 |
| Inlet temperature (C. °) | 90 |
| Outlet temperature (C. °) | 60 |
| Atomization pressure (bar) | 2.6 |

The reference product, ALECENSA® capsule (150 mg), was tested orally in six beagle dogs under two different dosing pretreatments (with or without high-fat food), using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 10, 12, 24, 48, 72 and 96 h after the drug administration. Alectinib plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different dosing pretreatments.

The results shown in Table 4F include the geometric mean values as well as the coefficient of variation (CV) of the pharmacokinetic parameters of two dosing groups. The absorption of ALECENSA under fed situation is about 2 times higher than that under fasted condition, which indicates a great food effect.

TABLE 4F

| | | Cₘₐₓ | AUCₗₐₛₜ | AUC_{inf} |
|---|---|---|---|---|
| Study design | | ng/mL | h*ng/mL | h*ng/mL |
| ALECENSA (150 mg) | Average | 819.0 | 33623.3 | 36208.0 |
| in fasted condition | CV(%) | 58.7 | 48.4 | 51.6 |
| ALECENSA (150 mg) | Average | 1311.3 | 60333.3 | 73500.0 |
| in fed condition | CV(%) | 26.6 | 26.0 | 23.5 |

The ASD capsules, batch No. M211207, was tested orally in six beagle dogs under two different dosing pretreatments (with or without high-fat food), using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 10, 12, 24, 48, 72 and 96 h after the drug administration. Alectinib plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 4G include the geometric mean values as well as coefficient of variation (CV) of the pharmacokinetic parameters of two dosing groups. The absorption of the ASD capsules (batch No. M211207) prepared was increased compared to ALECENSA (Table 4F) in fasted condition given that the dosing of alectinib in ASD is 75 mg, only half of that in the reference product ALECENSA (150 mg). In addition, the food effect of the ASD capsules was significantly reduced, which indicates that the ASD formulation enhances the bioavailability of Alectinib significantly

TABLE 4G

| | | Cₘₐₓ | AUCₗₐₛₜ | AUC_{inf} |
|---|---|---|---|---|
| Study design | | ng/mL | h*ng/mL | h*ng/mL |
| ASD capsules batch No. | Average | 947.4 | 39936.7 | 47740.0 |
| M211207 (75 mg) in | CV(%) | 21.5 | 29.1 | 34.8 |
| fasted condition | | | | |
| ASD capsules batch No. | Average | 1073.1 | 49371.7 | 60033.3 |
| M211207 (75 mg) in | CV(%) | 15.2 | 17.8 | 27.3 |
| fed condition | | | | |

A separate alectinib composition as described in Table 4H was prepared.

TABLE 4H

| Batch No. | Alectinib T2 formulation | |
|---|---|---|
| Component | Unit weight (mg) | Description |
| Alectinib Hydrochloride | 75 | ASD Particles |
| HPMCAS-LF | 75 | |
| SDS | 75 | |
| Tartaric acid | 75 | |
| Tartaric acid | 75 | Granulation |
| MCC (102) | 72.5 | |
| PVPP-XL | 50 | |
| MgSt | 2.5 | |
| Total weight (mg) | 500 | |

Briefly, Alectinib Hydrochloride was dispersed in a solvent e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then HPMCAS-LF, SDS and tartaric acid were dissolved into the solution completely. The solution was introduced into spray dryer (SD-06AG, Labplant, UK) via flash atomization. The parameters were the following: feed rate 3.0 rpm, inlet temperature 90° C., outlet temperature 60° C., and atomization pressure 2.6 bar. The SDD particles were collected and used for granulating. The SDD particles, tartaric acid, MCC(102), PVPP-XL and Magnesium stearate were blended evenly, then used for dry granulation. Finally, the granules were filled into HPMC capsules. Each capsule contains 75 mg alectinib hydrochloride.

The formulation Alectinib T2 capsule 75 mg was tested orally in six beagle dogs under fasted and fed condition. Six dogs were classified into two groups using a two-way crossover design. one group were fasted for 12 hours before administration, and then given food 4 hours after drug administration, the other group were fed with high-fat food according to FDA guidance (Assessing the effects of food on drug in INDs and NDAs-Clinical pharmacology considerations, 2019) 30 mins before administration. During the study, the dogs were allowed to drink water freely and each dog was administered with 50 ml water in total.

Blood samples were taken from each group of animals at the following time points: 0 h (before test drug administration), and 0.25, 0.5, 1, 1.5, 2, 3, 4, 8, 12 and 24 h after the drug administration. Alectinib plasma concentrations were analyzed by an LC-MS/MS method. The non-compartment model of software WinNonlin was used to calculate the pharmacokinetic parameters of each dog, Cₘₐₓ, AUCₗₐₛₜ and AUCinf. The geometric averages of each parameters were used for comparisons of the in vivo absorption under different doing pretreatments.

Table 41 shows the geometric mean values as well as coefficient of variation (CV) of the pharmacokinetic parameters of two groups. The results showed that the alectinib T2 formulation completely removed the food effect.

TABLE 4I

| | | Cₘₐₓ | AUCₗₐₛₜ | AUC_{inf} |
|---|---|---|---|---|
| Study design | | ng/mL | h*ng/mL | h*ng/mL |
| Alectinib T2 (75 mg) | Average | 549.8 | 25547.3 | 26492.6 |
| in fasted condition | CV(%) | 42.9 | 50.5 | 52.3 |
| Alectinib T2 (75 mg) | Average | 459.9 | 24334.2 | 29177.5 |
| in fed condition | CV(%) | 49.5 | 51.3 | 37.8 |

A separate alectinib composition as described in Table 4J was prepared.

TABLE 4J
Batch No. Alectinib T3 formulation

| Description | Ingredient | Unit weight (mg) |
|---|---|---|
| ASD particles | Alectinib Hydrochloride | 75 |
| | VA64 | 75 |
| | TPGS | 75 |
| Dry granulation | PVPP-XL | 47.5 |
| | Magnesium stearate | 2.5 |
| Outer diluents | Tartaric acid | 225 |

| | |
|---|---|
| Total weight (mg) | 500 |

Briefly, Alectinib Hydrochloride was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then VA64, and TPGS were dissolved into the solution completely. The solution was introduced into spray dryer (SD-06AG, Labplant, UK) via flash atomization. The parameters were the following: feed rate 3.0 rpm, inlet temperature 90° C., outlet temperature 60° C., and atomization pressure 2.6 bar. The SDD particles were collected and used for granulating. The SDD particles, PVPP-XL and Magnesium stearate were blended evenly, then used for dry granulation. Finally, the granules and tartaric acid were filled into HPMC capsules. Each capsule contains 75 mg alectinib hydrochloride.

The ASD capsule (T3) and the reference product (RLD), ALECENSA® capsule, were tested orally in six beagle dogs under fasted and fed condition. Six dogs were classified into three groups using a three-way crossover design, two groups were administrated with T3 and ALECENSA capsule in fed condition, the other group were administrated with T3 in fasted condition. The dose is 75 mg per dog in ASD capsule groups and 150 mg per dog in ALECENSA group. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Abiraterone plasma concentrations were analyzed by an LC-MS/MS method. The non-compartment model of software WinNonlin was used to calculate the pharmacokinetic parameters of each dog, Cₘₐₓ, AUCt and AUCinf. The geometric averages of each parameters were used for comparisons of the in vivo absorption under different doing pretreatments.

Table 4K shows the geometric mean values as well as coefficient of variation (CV) of the pharmacokinetic parameters of two groups. The results showed that the absorption of T3 (75 mg) is comparable to RLD (150 mg) under fed condition. It reveals that the ASD capsule enhances the absorption significantly.

TABLE 4K

| | | Cₘₐₓ | AUCₗₐₛₜ | AUC_{inf} |
|---|---|---|---|---|
| Study design | | ng/mL | h*ng/mL | h*ng/mL |
| Alectinib T3 (75 mg) in | Average | 650.0 | 23166.7 | 26675.0 |
| fasted condition | CV(%) | 53.9 | 42.4 | 40.1 |
| Alectinib T3 (75 mg) in | Average | 1371.7 | 55116.7 | 62120.0 |
| fed condition | CV(%) | 20.2 | 20.5 | 26.2 |
| RLD (150 mg) in fed | Average | 1161.2 | 50916.7 | 57840.0 |
| condition | CV(%) | 35.5 | 37.1 | 39.9 |

### Example 3. Pazopanib Hydrochloride Compositions and PK Study in Beagle Dogs

This example illustrates the process of improving the oral absorption of and reducing or removing the food-effect of pazopanib hydrochloride, according to some embodiments of the present disclosure.

Pazopanib is a multi-tyrosine kinase inhibitor indicated for the treatment of patients with advanced renal cell carcinoma. It was developed by Novartis and approved by FDA under the brand name VOTRIENT in 2009. Pazopanib is very slightly soluble at pH 1 and practically insoluble above pH 4 in aqueous media. Systemic exposure to pazopanib is increased when administered with food. Administration of pazopanib with a high-fat or low-fat meal results in an approximately 2-fold increase in AUC and Cₘₐₓ. Therefore, pazopanib should be administered at least 1 hour before or 2 hours after a meal.

Three pazopanib compositions as described in Table 5A were prepared.

TABLE 5A

| | M210706-1 | M210706-2 | M211214-2 | |
|---|---|---|---|---|
| Batch No. | Unit weight | Unit weight | Unit weight | |
| Component | (mg) | (mg) | (mg) | Description |
| Pazopanib | 100 | 100 | 100 | API |
| hydrochloride | | | | |
| HPMC-E5 | 200 | 200 | 100 | Polymer |
| Tartaric acid | 100 | 100 | — | Acid |
| | | | | excipient |
| TPGS | — | 100 | 100 | Surfactant |
| lecithin | — | — | 100 | |

Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 5B. After collection, the particles were dried in a convection tray dryer to remove the residual solvent before further use. The particles were filled into gel capsule.

TABLE 5B

| | |
|---|---|
| Feed rate (rpm) | 5 |
| Inlet temperature (C. °) | 60 |
| Outlet temperature (C. °) | 40 |
| Atomization pressure (bar) | 2 |

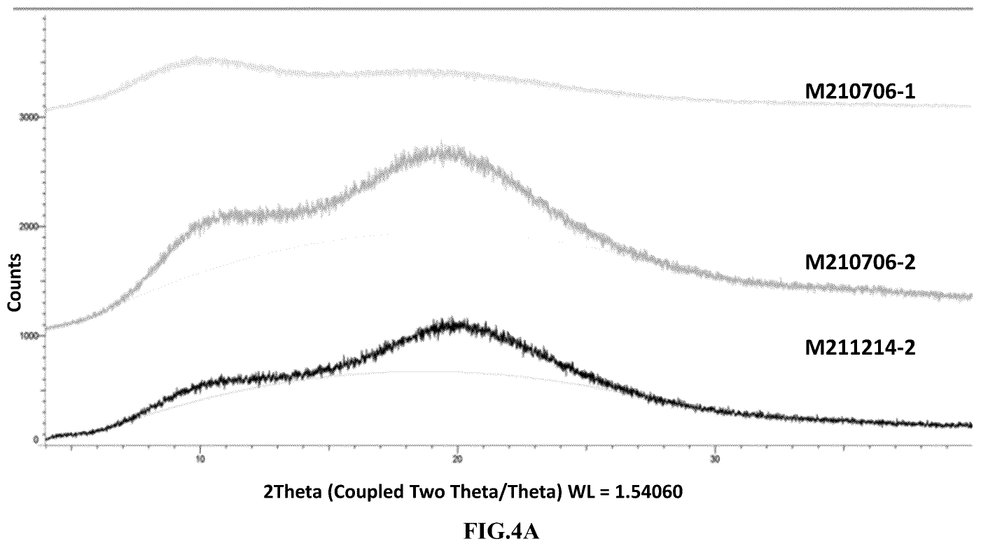

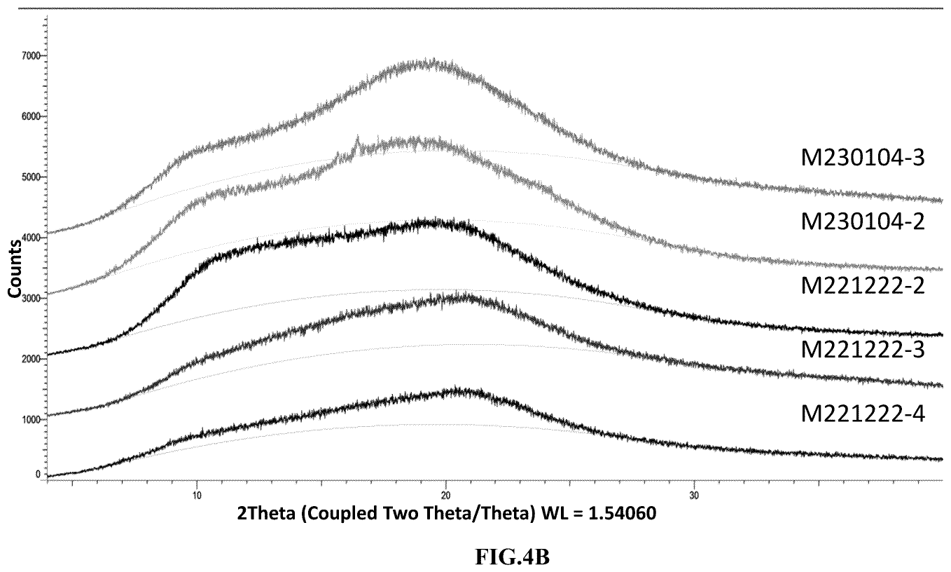

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The results (FIG. 4A) show that all three batches are in amorphous state.

A separate pazopanib composition as described in Table 5C was prepared.

TABLE 5C

| Batch No. | M211101 | |
|---|---|---|
| Component | Unit weight (mg) | Description |
| Pazopanib hydrochloride | 50 | API |
| HPMC-E5 | 50 | polymer |
| TPGS | 50 | surfactant |
| Tartaric acid | 50 | acid excipient |
| SiO2 | 75 | adsorbent |

Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were shown in Table 5D. After collection, the particles were dried in a convection tray dryer to remove the residual solvent before further use. The particles were filled into gel capsule. Each capsule contains 50 mg pazopanib hydrochloride.

TABLE 5D

| | |
|---|---|
| Feed rate (rpm) | 5 |
| Inlet temperature (C. °) | 60 |
| Outlet temperature (C. °) | 40 |
| Atomization pressure (bar) | 2 |

The reference product, VOTRIENT® tablet (200 mg), was tested orally in six beagle dogs under two different dosing pretreatments (with or without high-fat food), using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 5E include the geometric mean values as well as the coefficient of variation (CV) of the pharmacokinetic parameters of two dosing groups. The absorption of VOTRIENT® under fed situation is about 2 times higher than that under fasted, which indicates a great food effect.

TABLE 5E

| | | Cₘₐₓ | AUCₗₐₛₜ | AUC_{inf} |
|---|---|---|---|---|
| Study design | | ng/mL | h*ng/mL | h*ng/mL |
| VOTRIENT (200 mg) in | Average | 5282.3 | 19395.0 | 21130.0 |
| fasted condition | CV(%) | 98.8% | 103.9% | 125.2% |
| VOTRIENT (200 mg) in | Average | 15283.3 | 43166.7 | 46620.0 |
| fed condition | CV(%) | 77.2% | 78.5% | 78.9% |

The capsules batch No. M211101 was tested orally in six beagle dogs under two different dosing pretreatments (with or without high-fat food), using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 5F include the geometric mean values as well as coefficient of variation (CV) of the pharmacokinetic parameters of two dosing groups. The absorption of pazopanib in batch No. M211101 under fasted situation is comparable to that under fed situation, which indicates no food effect.

TABLE 5F

| | | Cₘₐₓ | AUCₗₐₛₜ | AUC_{inf} |
|---|---|---|---|---|
| Study design | | ng/mL | h*ng/mL | h*ng/mL |
| Batch No. M211101 | Average | 3884.4 | 11248.0 | 11330.0 |
| (50 mg) capsules in | CV(%) | 77.0% | 65.7% | 65.4% |
| fasted condition | | | | |
| Batch No. M211101 | Average | 3730.0 | 10184.0 | 10266.0 |
| (50 mg) capsules in | CV(%) | 77.6% | 46.4% | 46.0% |
| fed condition | | | | |

Five pazopanib compositions as described in Table 5G were prepared to evaluate the effect of polymers. Briefly, API was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 3.0-8.0 rpm, inlet temperature 50-100° C., outlet temperature 30-80° C., and atomization pressure 0-4.0 bar. The particles were collected and used for test of crystalline phase and dissolution in FaSSIF medium.

TABLE 5G

| | | Batch No. | | | | |
|---|---|---|---|---|---|---|
| | | M221222-2 | M221222-3 | M221222-4 | M230104-2 | M230104-3 |
| Description | Component | Unit weight (mg) | | | | |
| API | Pazopanib | 200 | 200 | 200 | 200 | 200 |
| | hydrochloride | | | | | |
| Polymer | Soluplus | — | — | — | 400 | 400 |
| | PVP-K30 | 400 | 400 | 400 | — | — |
| Surfactant | TPGS | 200 | — | 200 | 200 | 200 |
| Acid | Tartaric acid | — | 200 | 200 | — | 200 |
| excipient | | | | | | |

XRPD studies were performed using the same method as described above. The results (FIG. 4B) show that all five batches are in amorphous state.

Kinetic solubility was measured. Excess of SDD powder was added into 40 ml FaSSIF medium at 37° C. under 400 rpm rotation. 1 ml medium was withdrawn into a centrifugal tube at specified time point and then centrifuged for 10 mi under 0000 rpm. The upper solution was collected for assay measure by HPLC. The results are shown in Table 5H, suggesting that concurrent use of surfactant and acid excipient in formulation does not increase the solubility significantly in vitro, but unexpectedly do enhance the availability significantly in vivo.

TABLE 5H

| | Kinetic solubility (μg/mL) | | | | |
|---|---|---|---|---|---|
| Time | M221222- | M221222- | M221222- | M230104- | M230104- |
| (min) | 2 | 3 | 4 | 2 | 3 |
| 5 | 14.56 | 20 | 4.57 | 9.58 | 10.43 |
| 15 | 1.88 | 4.89 | 3.1 | 9.23 | 11.48 |
| 30 | 1.23 | 2.09 | 1.32 | 9.94 | 11.17 |
| 60 | 1.34 | 2.21 | 1.36 | 10.48 | 9.03 |
| 90 | 1 | 1.27 | 1.34 | 6.33 | 6.86 |
| 120 | 0.94 | 1.42 | 1.22 | 5.42 | 5.66 |

Two pazopanib compositions as described in Table 51 were prepared using the same method as described above to evaluate the effect of polymers.

TABLE 5I

| | Batch No. | M210630-1 | M210630-3 |
|---|---|---|---|
| Description | Component | Unit weight (mg) | |
| API | Pazopanib hydrochloride | 200 | 200 |
| Polymer | PVP-K30 | 600 | 600 |
| Surfactant | TPGS | — | 50 |
| Acid excipient | Tartaric acid | — | 600 |

Kinetic solubility was measured using the same method as described above. The results are shown in Table 5J suggesting that concurrent use of surfactant and acid excipient under high dose in the formulation does increase the solubility significantly.

TABLE 5J

Kinetic solubility (μg/mL)

| Time (min) | M210630-1 | M210630-3 |
|---|---|---|
| 5 | 6.9 | 57.01 |
| 15 | 39.08 | 72.05 |
| 30 | 21.36 | 42.83 |

A pazopanib hydrochloride composition as described in Table 5K was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, TPGS and Tartaric acid were dissolved into the solution completely. Finally, the SiO2 was added into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The particles were collected and filled into gel capsule. Each capsule contains 50 mg pazopanib hydrochloride.

TABLE 5K
Batch No: pazopanib hydrochloride T1 formulation

| Description | Ingredient | Unit weight (mg) |
|---|---|---|
| API | Pazopanib Hydrochloride | 50 |
| Polymer | HPMC-E5 | 50 |
| Surfactant | TPGS | 50 |
| Acid excipient | Tartaric acid | 50 |
| Adsorbent | SiO2 | 75 |

The pazopanib hydrochloride formulation T1 capsule 50 mg in API, was tested orally in six beagle dogs under two different dosing pretreatments (with or without high-fat food), using a two-way crossover design. During the study, the dogs were allowed to drink water freely and were fasted for 12 hours before administration, all dogs are given food 4 hours after drug administration. Each dog was administered with 50 ml water in total. Blood samples were taken from each group of animals at the following time points: 0 h (before test drug administration), and 0.25, 0.5, 1, 1.5, 2, 3, 4, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCₗₐₛₜ and AUCinf. The geometric averages of each parameters were used for comparisons of the in vivo absorption under different doing pretreatments.

Table 5L shows as below the geometric mean values as well as coefficient of variation (CV) of the pharmacokinetic parameters of two groups. The result show that the absorption of pazopanib under fasted situation is comparable to that under fed situation, which indicates pazopanib hydrochloride formulation T1 completely removed the food effect.

TABLE 5L

| | | Cₘₐₓ | AUCₗₐₛₜ | AUC_{inf} |
|---|---|---|---|---|
| Study design | | ng/mL | h*ng/mL | h*ng/mL |
| Pazopanib hydrochloride T1 | Average | 3884.4 | 11248 | 11330 |
| (50 mg) in fasted condition | CV(%) | 77 | 65.7 | 65.4 |
| Pazopanib hydrochloride T1 | Average | 3730 | 10184 | 10266 |
| (50 mg) in fed condition | CV(%) | 77.6 | 46.4 | 46 |

| | | | |
|---|---|---|---|
| Pazopanib hydrochloride T1 Fed/Fasted | 110.2 | 112.1 | 128.7 |
| Mean Ratio(%) | | | |
| Pazopanib hydrochloride T1 Fed/Fasted | 350.3 | 275.7 | 347.4 |
| UP 90(%) | | | |
| Pazopanib hydrochloride T1 Fed/Fasted | 34.6 | 45.6 | 47.7 |
| Low 90(%) | | | |

A pazopanib hydrochloride composition as described in Table 5M was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, and TPGS were dissolved into the solution completely. Finally, the SiO2 was added into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The particles were collected and used for dry granulation with MCC and Magnesium stearate. The dry granules, tartaric acid, MCC(102), PVPP-XL and Magnesium stearate were blended evenly, then compressed into a tablet. Each tablet contains 75 mg pazopanib free base.

TABLE 5M

| Batch No.: | | | Unit |
|---|---|---|---|
| Pazopanib T2 | Description | Ingredient | weight (mg) |
| Inner | ASD | Pazopanib Hydrochloride | 81.25 |
| granulation | particles | HPMC-E5 | 75 |
| | | TPGS | 75 |
| | | SiO2 | 75 |
| | | MCC(PH102) | 75 |
| | | Magnesium stearate | 4 |

| Outer granulation | MCC(PH102) | 135 |
|---|---|---|
| | Tartaric acid | 150 |
| | PVPP-XL | 25.75 |
| | Magnesium stearate | 4 |

| | |
|---|---|
| Total unit weight (mg) | 700 mg |

The Pazopanib Hydrochloride ASD tablet (Pazopanib T2) and the reference product (RLD), VOTRIENT® tablet, were tested orally in six beagle dogs under fasted and fed condition. Six dogs were classified into three groups using a three-way crossover design, two groups were administrated with T3 and VOTRIENT® tablet in fast condition, the other group were administrated with T3 in fed condition. The dose is 75 mg of pazopanib free base per dog in ASD tablet groups and 200 mg per dog in VOTRIENT® group. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Abiraterone plasma concentrations were analyzed by an LC-MS/MS method. The non-compartment model of software WinNonlin was used to calculate the pharmacokinetic parameters of each dog, Cₘₐₓ, AUCₗₐₛₜ and AUCinf. The geometric averages of each parameters were used for comparisons of the in vivo absorption under different doing pretreatments.

Table 5N shows the geometric mean values as well as coefficient of variation (CV) of the pharmacokinetic parameters of three dosing groups. The results in these two tables show that the absorption of Pazopanib T2 formulation (75 mg) is comparable to RLD (200 mg) under fast condition and the food effect is completely removed in Pazopanib T2 formulation. It reveals that the ASD tablet enhances the absorption of pazopanib significantly.

TABLE 5N

| | | Cₘₐₓ | AUCₗₐₛₜ | AUC_{inf} |
|---|---|---|---|---|
| Study design | | ng/mL | h*ng/mL | h*ng/mL |
| Pazopanib T2 (75 mg) | Average | 7996.0 | 29427.4 | 29522.0 |
| in fasted condition | CV(%) | 92.5 | 97.0 | 96.8 |
| Pazopanib T2 (75 mg) | Average | 6578.0 | 27198.4 | 27445.2 |
| in fed condition | CV(%) | 53.4 | 57.0 | 57.1 |
| VOTRIENT (200 mg) | Average | 8220.9 | 26490.2 | 26567.4 |
| in fasted condition | CV(%) | 124.6 | 123.1 | 122.8 |

A pazopanib hydrochloride composition as described in Table 50 was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, Lecithin and TPGS were dissolved into the solution completely. Finally, the SiO2 was added into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The ASD particles were collected and used for dry granulation blended with PVPP-XL and Magnesium stearate. The dry granules, tartaric acid, MCC(102), PVPP-XL and Magnesium stearate were blended evenly, then compressed into a tablet. Each tablet contains 75 mg pazopanib free base.

TABLE 5O

| Batch No.: | | | Unit weight |
|---|---|---|---|
| Pazopanib T3 | Description | Ingredient | (mg) |
| Inner | ASD | Pazopanib Hydrochloride | 81.25 |
| granulation | particles | HPMC-E5 | 75 |
| | | TPGS | 75 |
| | | Lecithin | 75 |
| | | SiO2 | 112.5 |
| | | PVPP-XL | 75 |
| | | Magnesium stearate | 4 |

| Outer granulation | MCC(PH102) | 75 |
|---|---|---|
| | Tartaric acid | 75 |
| | PVPP-XL | 48.25 |
| | Magnesium stearate | 4 |

| | |
|---|---|
| Total unit weight (mg) | 700 |

The ASD tablet (Pazopanib T3) and the reference product (RLD), VOTRIENT® tablet, were tested orally in six beagle dogs under fasted and fed condition. Six dogs were classified into three groups using a three-way crossover design, two groups were fast administrated with Pazopanib T3 and VOTRIENT® tablet, the other group were fed administrated with Pazopanib T3. The dose is 75 mg of pazopanib free base per dog in ASD tablet groups and 200 mg per dog in VOTRIENT® group. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Abiraterone plasma concentrations were analyzed by an LC-MS/MS method. The non-compartment model of software WinNonlin was used to calculate the pharmacokinetic parameters of each dog, Cₘₐₓ, AUCt and AUCinf. The geometric averages of each parameters were used for comparisons of the in vivo absorption under different doing pretreatments.

Table 5P shows as below the geometric mean values as well as coefficient of variation (CV) of the pharmacokinetic parameters of three dosing groups. The results in these two tables show that the absorption of Pazopanib T3 formulation (75 mg in pazopanib freebase) is comparable to RLD (200 mg in pazopanib freebase) under fast condition. The food effect is mostly removed in Pazopanib T3 group. It reveals that the ASD tablet enhances the absorption of pazopanib significantly.

TABLE 5P

| | | Cₘₐₓ | AUCₗₐₛₜ | AUC_{inf} |
|---|---|---|---|---|
| Study design | | ng/mL | h*ng/mL | h*ng/mL |
| Pazopanib T3 (75 mg) | Average | 7395.7 | 23590.0 | 23681.7 |
| in fasted condition | CV(%) | 28.8 | 33.2 | 33.1 |
| Pazopanib T3 (75 mg) | Average | 4723.2 | 18650.3 | 18818.3 |
| in fed condition | CV(%) | 34.1 | 40.1 | 39.7 |
| VOTRIENT (200 mg) | Average | 6545.7 | 19024.5 | 19178.6 |
| in fasted condition | CV(%) | 87.5 | 96.7 | 95.9 |

A pazopanib hydrochloride composition as described in Table 5Q was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, Lecithin, tartaric acid and TPGS were dissolved into the solution completely. Finally, the SiO2 was dispersed into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The ASD particles were collected and used for dry granulation blended with PVPP-XL and Magnesium stearate. The dry granules, MCC(102), PVPP-XL and Magnesium stearate were blended evenly, then compressed into a tablet. Each tablet contains 75 mg pazopanib free base.

TABLE 5Q

| Batch No.: | | | Unit |
|---|---|---|---|
| Pazopanib T4 | Description | Ingredient | weight (mg) |
| Inner | ASD | Pazopanib Hydrochloride | 81.25 |
| granulation | particles | HPMC-E5 | 75 |
| | | TPGS | 75 |
| | | Lecithin | 75 |
| | | Tartaric acid | 75 |
| | | SiO2 | 112.5 |
| | | PVPP-XL | 75 |
| | | Magnesium stearate | 4 |

| Outer granulation | MCC(PH102) | 38.25 |
|---|---|---|
| | PVPP-XL | 85 |
| | Magnesium stearate | 4 |

| | |
|---|---|
| Total unit weight (mg) | 700 |

The ASD tablet (Pazopanib T4) and the reference product (RLD), VOTRIENT® tablet, were tested orally in six beagle dogs under fasted and fed condition. Six dogs were classified into three groups using a three-way crossover design, two groups were administrated with T4 and VOTRIENT® tablet in fast condition, the other group were administrated with T4 in fed condition. The dose is 75 mg of pazopanib free base per dog in ASD tablet groups and 200 mg per dog in VOTRIENT® group. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCₗₐₛₜ and AUCinf. The geometric averages of each parameters were used for comparisons of the in vivo absorption under different doing pretreatments.

Table 5R shows as below the geometric mean values as well as coefficient of variation (CV) of the pharmacokinetic parameters of three dosing groups. The results in these two tables show that the absorption of pazopanib T4 formulation (75 mg in pazopanib freebase) is comparable to RLD (200 mg in pazopanib freebase) under fast condition and the food effect is mainly removed in pazopanib T4 formulation. It reveals that the ASD tablet enhances the absorption of pazopanib significantly.

TABLE 5R

| | | Cmax | AUClast | AUCinf |
|---|---|---|---|---|
| Study design | | ng/mL | h*ng/mL | h*ng/mL |
| Pazopanib T4 (75 mg) | Average | 9032.2 | 31140.0 | 31224.7 |
| in fasted condition | CV(%) | 69.6 | 66.5 | 66.3 |
| Pazopanib T4 (75 mg) | Average | 4591.0 | 17048.2 | 17268.0 |
| in fed condition | CV(%) | 65.9 | 51.2 | 50.6 |
| VOTRIENT (200 mg) | Average | 10768.0 | 32862.3 | 32951.0 |
| in fasted condition | CV(%) | 87.7 | 80.0 | 79.8 |

A pazopanib hydrochloride composition as described in Table 5S was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the VA64, Lecithin, tartaric acid and TPGS were dissolved into the solution completely. Finally, the SiO2 was dispersed into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The ASD particles were collected and used for dry granulation blended with PVPP-XL and Magnesium stearate. The dry granules, MCC(102), PVPP-XL and Magnesium stearate were blended evenly, then compressed into a tablet. Each tablet contains 75 mg pazopanib free base.

TABLE 5S

| Batch No.: | | | Unit |
|---|---|---|---|
| Pazopanib T5 | Description | Ingredient | weight (mg) |
| Inner | ASD | Pazopanib Hydrochloride | 81.25 |
| granulation | particles | VA64 | 75 |
| | | TPGS | 75 |
| | | Lecithin | 75 |
| | | Tartaric acid | 75 |
| | | SiO2 | 112.5 |
| | | PVPP-XL | 75 |
| | | Magnesium stearate | 4 |

| Outer granulation | MCC(PH102) | 75 |
|---|---|---|
| | PVPP-XL | 48.25 |
| | Magnesium stearate | 4 |

| | |
|---|---|
| Total unit weight (mg) | 700 |

The ASD tablet (Pazopanib T5) and the reference product (RLD), VOTRIENT® tablet, were tested orally in six beagle dogs under fasted and fed condition. Six dogs were classified into three groups using a three-way crossover design, two groups were administrated with Pazopanib T5 and VOTRIENT® tablet in fast condition, the other group were administrated with Pazopanib T5 in fed condition. The dose is 75 mg of pazopanib free base per dog in ASD tablet groups and 200 mg per dog in VOTRIENT® group. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCt and AUCinf. The geometric averages of each parameters were used for comparisons of the in vivo absorption under different doing pretreatments.

Table 5T shows the geometric mean values as well as coefficient of variation (CV) of the pharmacokinetic parameters of three dosing groups. The results in these two tables show that the absorption of Pazopanib T5 (75 mg in pazopanib freebase) is higher than RLD (200 mg in pazopanib freebase) under fasted condition and the food effect is markedly decreased in T5 group. It reveals that the ASD tablet enhances the absorption of pazopanib significantly.

TABLE 5T

| | | Cmax | AUClast | AUCinf |
|---|---|---|---|---|
| Study design | | ng/mL | h*ng/mL | h*ng/mL |
| Pazopanib T5 (75 mg) | Average | 9280.2 | 28534.0 | 28692.0 |
| in fasted condition | CV(%) | 93.7 | 94.3 | 93.7 |
| Pazopanib T5 (75 mg) | Average | 3554.2 | 15245.5 | 15523.7 |
| in fed condition | CV(%) | 34.0 | 38.8 | 37.6 |
| VOTRIENT (200 mg) | Average | 11448.4 | 34668.5 | 41804.0 |
| in fasted condition | CV(%) | 59.8 | 71.8 | 48.8 |

A pazopanib hydrochloride composition as described in Table 5U was prepared. Briefly, Pazopanib Hydrochloride was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the PVP-K90, Lecithin, and TPGS were dissolved into the solution completely. Finally, the SiO2 was dispersed into the solution to form a suspension under stirring. The suspension was introduced into spray dryer (SD-06AG, Labplant, UK) via flash atomization. The parameters were the following: feed rate 5.0 rpm, inlet temperature 60° C., outlet temperature 40° C., and atomization pressure 2.0 bar. The ASD particles were collected and used for dry granulation blended with PVPP-XL and Magnesium stearate. The dry granules, tartaric acid, MCC(102), PVPP-XL and Magnesium stearate were blended evenly, then compressed into a tablet. Each tablet contains 75 mg pazopanib free base.

TABLE 5U

| Batch No.: | | | Unit |
|---|---|---|---|
| Pazopanib T6 | Description | Ingredient | weight (mg) |
| Inner | ASD | Pazopanib Hydrochloride | 81.25 |
| granulation | particles | PVP-K90 | 75 |
| | | TPGS | 75 |
| | | Lecithin | 75 |
| | | SiO2 | 150 |
| | | PVPP-XL | 75 |
| | | Magnesium stearate | 4 |

| Outer granulation | MCC(PH102) | 75 |
|---|---|---|
| | Tartaric acid | 75 |
| | PVPP-XL | 10.75 |
| | Magnesium stearate | 4 |

| | |
|---|---|
| Total unit weight (mg) | 700 |

The ASD tablet (Pazopanib T6 and Pazopanib T3) and the reference product (RLD), VOTRIENT® tablet, were tested orally in six Beagle dogs under fasted condition. Six dogs were classified into three groups using a three-way crossover design. The dose is 75 mg of pazopanib free base per dog in ASD tablet groups and 200 mg per dog in VOTRIENT® group. In Fasted condition, the dogs were allowed to drink water freely and were fasted for 12 hours before administration, and then given food 4 hours after drug administration.

Blood samples were taken from each group of animals at the following time points: 0 h (before test drug administration), and 0.25, 0.5, 1, 1.5, 2, 3, 4, 8, 12 and 24 h after the drug administration. Pazopanib plasma concentrations were analyzed by an LC-MS/MS method. The non-compartment model of software WinNonlin was used to calculate the pharmacokinetic parameters of each dog, Cₘₐₓ, AUCₗₐₛₜ and AUCinf. The geometric averages of each parameters were used for comparisons of the in vivo absorption under different doing pretreatments.

Table 5V shows as below the geometric mean values as well as coefficient of variation (CV) of the pharmacokinetic parameters of three dosing groups. The results in these two tables show that the absorption of T3 and T6 (75 mg in pazopanib freebase) is comparable to RLD (200 mg in pazopanib freebase) under fasted condition which reveals that the ASD tablet enhances the absorption of pazopanib significantly.

TABLE 5V

| | | Cmax | AUClast | AUCinf |
|---|---|---|---|---|
| Study design | | ng/mL | h*ng/mL | h*ng/mL |
| Pazopanib T3 (75 mg) | Average | 3701.8 | 13286.6 | 13512.0 |
| in fastedcondition | CV(%) | 21.1 | 62.6 | 63.4 |
| Pazopanib T6 (75 mg) | Average | 4154.4 | 14552.8 | 14766.8 |
| in fasted condition | CV(%) | 93.7 | 80.6 | 79.2 |
| VOTRIENT (200 mg) | Average | 4823.2 | 12412.2 | 12531.0 |
| in fasted condition | CV(%) | 78.4 | 65.5 | 64.6 |

Finally, a randomized, single-dose, open-label, 3-sequence, 3-period cross-over human study was performed in healthy volunteers. Twelve subjects each received a single dose of pazopanib formulation T4 (as described in Table 5Q) and RLD, VOTRIENT® tablet, under 6 different conditions or treatments (overnight fasted [A]/T, high-fat meal predose [B]/T, high-fat meal predose [C]/T, overnight fasted [D]/T, overnight fasted [E]/R, overnight fasted [F]/R). Each group has 2 subjects. The treatment sequences used for Period 1 through 3 are presented in Table 5W.

TABLE 5W

| Sequence | Group A | Group B | Group C | Group D | Group E | Group F |
|---|---|---|---|---|---|---|
| 1 | Fasted/T | Fed/T | Fed/T | Fasted/T | Fasted/R | Fasted/R |
| 2 | Fed/T | Fasted/T | Fasted/R | Fasted/R | Fed/T | Fasted/T |
| 3 | Fasted/R | Fasted/R | Fasted/T | Fed/T | Fasted/T | Fed/T |

There was a washout period of 14 days between study periods. Following treatment administration, subjects underwent PK sampling for 72 hours. The pazopanib formulation T4 (75 mg in API freebase) exhibited higher oral absorption compared to RLD (200 mg in API freebase). The relative bioavailability of pazopanib formulation T4 is 199% compared to RLD (200 mg in API freebase). The AUC and Cₘₐₓ of pazopanib formulation T4 in fed condition is about 125% and about 150%, respectively, of that in fasted condition. The AUC of RLD in fed condition is about 200% of that in fasted condition (as illustrated in Table 5E). This results as shown in Table 5X indicated that the pazopanib formulation T4 reduced the food-effect and increased the oral absorption.

TABLE 5X

| | | Cmax | AUClast | AUCINF_obs |
|---|---|---|---|---|
| | | μg/mL | μg*h/mL | μg*h/mL |
| T Fasted | Average | 9.3 | 230.3 | 302.9 |
| | CV(%) | 37.4% | 37.2% | 41.8% |
| T Fed | Average | 11.0 | 330.3 | 437.1 |
| | CV(%) | 12.7% | 18.5% | 22.0% |
| R fasted | Average | 16.0 | 432.1 | 575.3 |
| | CV(%) | 61.9% | 65.0% | 69.3% |

### Example 4. Cabozantinib Compositions

This example illustrates the process of improving the oral absorption of and reducing or removing the food-effect of cabozantinib malate, according to some embodiments of the present disclosure.

Cabozantinib is developed by COMETRIQ and approved by FDA in 2012 under the brand name COMETRIQ. It is a kinase inhibitor indicated for the treatment of patients with progressive, metastatic medullary thyroid cancer (MTC). Cabozantinib is practically insoluble in aqueous media. It has significant food-effect. A high-fat meal increased Cₘₐₓ and AUC values by 41% and 57%, respectively, relative to fasted conditions in healthy subjects. Thus, the patients should have no food for at least 2 hours before and at least 1 hour after taking COMETRIQ.

Three cabozantinib compositions as described in Table 6A were prepared.

TABLE 6A

| | M210702-1-I | M210702-2-I | M210702-3-I | |
|---|---|---|---|---|
| Batch No. | Unit weight | Unit weight | Unit weight | |
| Component | (mg) | (mg) | (mg) | Description |
| Cabozantinib | 60 | 60 | 60 | API |
| malte | | | | |
| HPMC-E5 | 60 | 120 | 60 | Polymer |
| lecithin | 120 | — | — | Surfactant |
| TPGS | 60 | — | 60 | Surfactant |
| Malic acid | — | 60 | 60 | Acid excipient |
| SiO2 | 90 | 60 | 60 | Absorbent |

Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 6B. After collection, the particles were dried in a convection tray dryer to remove the residual solvent before further use. The particles were filled into gel capsule.

TABLE 6B

| | |
|---|---|
| Feed rate (rpm) | 3 |
| Inlet temperature (C. °) | 55 |
| Outlet temperature (C. °) | 45 |
| Atomization pressure (bar) | 2.2 |

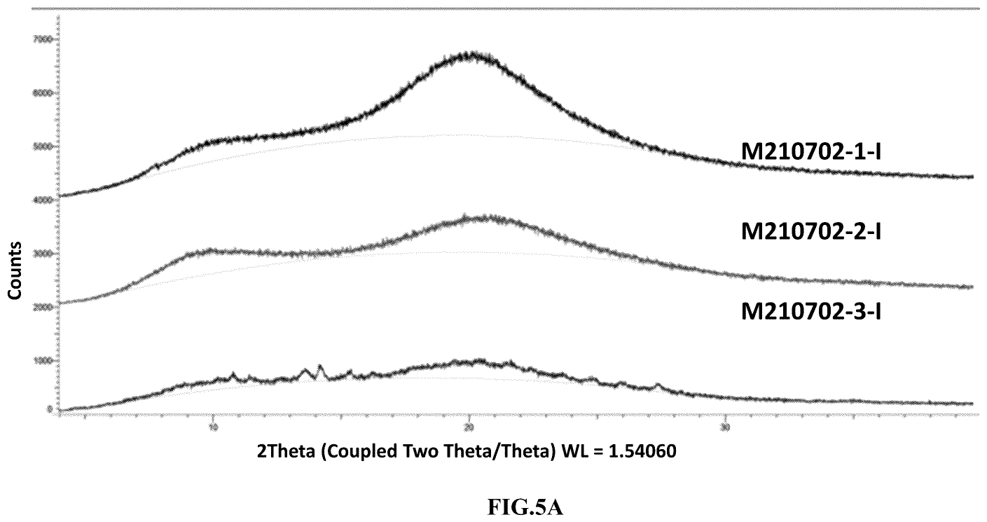

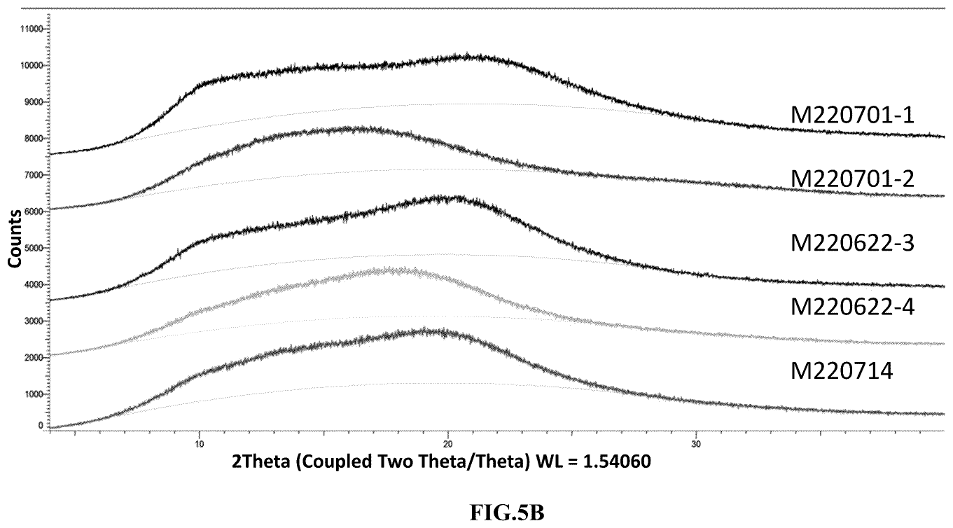

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The results (FIG. 5A) show that all three batches are in amorphous state.

Kinetic solubility of the three cabozantinib compositions was measured. Excess of SDD powder was added into 40 ml FeSSIF medium at 37° C. under 400 rpm rotation. 1 ml medium was withdrawn into a centrifugal tube at specified time point (5, 15, 30, 45, 60, and 90 min) and then centrifuged for 10 min under 10000 rpm. The upper solution was collected for assay measure by HPLC.

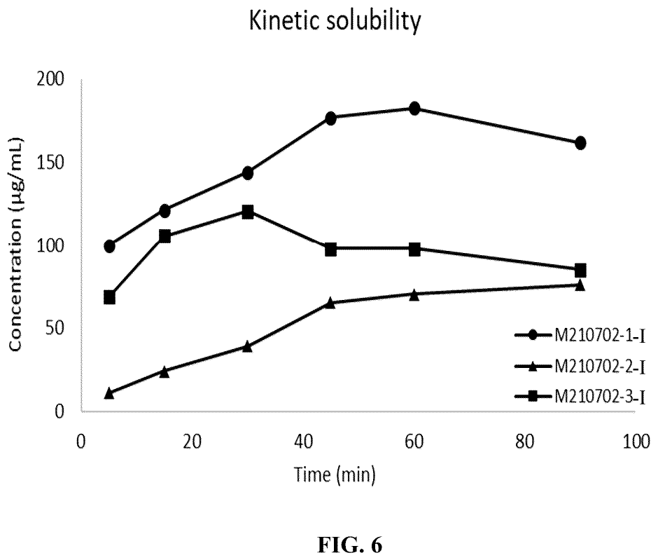

The results (Table 6C and FIG. 6) show that ASD composition with polymer and surfactant (M210702-1-I) has better dissolution profile than the composition with polymer, surfactant, and acid combination (M210702-3-I), and the composition with polymer and acid combination (M210702-2-I) has the worst dissolution profile among the three.

TABLE 6C

| | Kinetic solubility (μg/mL) | | |
|---|---|---|---|
| Time (min) | M210702-1-I | M210702-2-I | M210702-3-I |
| 5 | 99.8 | 11.3 | 68.8 |
| 15 | 121.0 | 24.3 | 105.6 |
| 30 | 144.0 | 39.3 | 120.6 |
| 45 | 176.7 | 65.5 | 97.7 |
| 60 | 182.2 | 70.2 | 97.7 |
| 90 | 161.6 | 75.9 | 85.1 |

Five cabozantinib compositions as described in Table 6D were prepared. Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 3.0-8.0 rpm, inlet temperature 50-100° C., outlet temperature 30-80° C., and atomization pressure 0-4.0 bar. The particles were collected and used for test of crystalline phase and dissolution in FaSSIF medium.

TABLE 6D

| | | Batch No | | | | |
|---|---|---|---|---|---|---|
| | | M220701-1 | M220701-2 | M220622-3 | M220622-4 | M220714 |
| Description | Component | Unit weight (mg) | | | | |
| API | Cabozantinib | 76 | 76 | 76 | 76 | 76 |
| | malate | | | | | |
| Polymer | VA64 | 60 | — | 60 | — | 30 |
| | Eudragit L100-55 | — | 60 | — | 60 | 30 |
| Surfactant | TPGS | — | — | 60 | 60 | 60 |

XRPD studies were performed using the same method described above. The results (FIG. 5B) show that all five batches are in amorphous state.

Kinetic solubility was measured. Excess of SDD powder was added into 40 ml FaSSIF medium at 37° C. under 400 rpm rotation. 1 ml medium was withdrawn into a centrifugal tube at specified time point and then centrifuged for 10 min under 10000 rpm. The upper solution was collected for assay measure by HPLC. The results are shown in Table 6E, suggesting that the addition of surfactant to formulation markedly improves the solubility of API.

TABLE 6E

| | | Kinetic solubility (μg/mL) | | | | |
|---|---|---|---|---|---|---|
| | | M220701- | M220701- | M220622- | M220622- | |
| Time | | 1 | 2 | 3 | 4 | M220714 |
| 5 | min | 3.75 | 15.8 | 44.55 | 27.99 | 36.13 |
| 10 | min | 3.67 | 17.03 | 48.46 | 29.62 | 41.22 |
| 20 | min | 4.5 | 15.32 | 48.99 | 31.88 | 49.24 |
| 30 | min | 6.13 | 14.76 | 46.42 | 33.16 | 52.38 |
| 45 | min | 8.74 | 16.23 | 24.61 | 33.98 | 53.07 |
| 60 | min | 10.41 | 16.65 | 16.02 | 35.93 | 54.63 |
| 90 | min | 11.76 | 16.72 | 15.3 | 36.51 | 55.01 |
| 120 | min | 11.06 | 16.54 | 11.76 | 37.6 | 56.06 |

Three cabozantinib compositions as described in Table 6F were prepared using the same method described above for evaluating the quantity of polymer.

TABLE 6F

| | | Batch No | | |
|---|---|---|---|---|
| | | M220623- | M220822- | M220801- |
| | | 2 | 2 | 1 |
| Description | Component | Unit weight (mg) | | |
| API | Cabozantinib malate | 76 | 76 | 76 |
| Polymer | HPMCAS-LF | 60 | 180 | — |
| | Soluplus | — | — | 120 |
| Surfactant | TPGS | 60 | 60 | 60 |

Kinetic solubility was measured using the same method described above. The results are shown in Table 6G, suggesting that the increase of polymer quantity in formulation results in a higher solubility of API.

TABLE 6G

Kinetic solubility (μg/mL)

| | | | |
|---|---|---|---|
| Time | M220623-2 | M220822-1 | M220801-1 |

| | | | | |
|---|---|---|---|---|
| 5 | min | 19.08 | 49.34 | 31.85 |
| 10 | min | 24.02 | 58.78 | 31.55 |
| 20 | min | 28.15 | 55.47 | 33.76 |
| 30 | min | 31.65 | 60.27 | 33.21 |
| 45 | min | 36.34 | 48.34 | 33.76 |
| 60 | min | 37.62 | 50 | 34.27 |
| 90 | min | 34.94 | 43.59 | 35.59 |
| 120 | min | 34.37 | 42.32 | 35.78 |

Four cabozantinib compositions as described in Table 6H were prepared using the same method described above for evaluating the effect of various surfactants.

TABLE 6H

| | Batch No | M220930-1 | M221010-1 | M221010-3 | M221011-2 |
|---|---|---|---|---|---|
| Description | Component | Unit weight (mg) | | | |
| API | Cabozantinib | 76 | 76 | 76 | 76 |
| | malate | | | | |
| Polymer | VA64 | 30 | 30 | 30 | 30 |
| | Eudragit L100-55 | 30 | 30 | 30 | 30 |
| Surfactant | TPGS | 60 | — | — | — |
| | Gelucire44/14 | — | 60 | — | — |
| | Poloxamer 407 | — | — | 60 | — |
| | RH40 | — | — | — | 60 |

Kinetic solubility was measured using the same method described above. The results are shown in Table 61, suggesting that various surfactants enhance the solubility of API, especially TPGS.

TABLE 6I

| | | Kinetic solubility (μg/mL) | | | |
|---|---|---|---|---|---|
| Time | | M220930-1 | M221010-1 | M221010-3 | M221011-2 |
| 5 | min | 42.3 | 19.71 | 22.5 | 32.71 |
| 10 | min | 43.2 | 22.08 | 25.86 | 31.73 |
| 20 | min | 47.4 | 18.28 | 25.06 | 32.35 |
| 30 | min | 46.62 | 18.4 | 24.31 | 33.45 |
| 45 | min | 47.43 | 15.17 | 23.2 | 33.04 |
| 60 | min | 48.16 | 14.86 | 26.09 | 33.14 |
| 90 | min | 46.06 | 13.72 | 27.27 | 34.87 |
| 120 | min | 43.51 | 13.15 | 28.45 | 35.91 |

Four cabozantinib compositions as described in Table 6J were prepared using the same method described above for evaluating the quantity of surfactant.

TABLE 6J

| | | Batch No | | | |
|---|---|---|---|---|---|
| | | M221130- | M220930- | M221130- | M221130- |
| Descrip- | | 1 | 1 | 2 | 3 |
| tion | Component | Unit weight (mg) | | | |
| API | Cabozantinib | 76 | 76 | 76 | 76 |
| | malate | | | | |
| Polymer | VA64 | 30 | 30 | 30 | 30 |
| | L100-55 | 30 | 30 | 30 | 30 |
| Surfactant | TPGS | 30 | 60 | 90 | 120 |
| Adsorbent | SiO2 | 60 | — | 60 | 60 |

Kinetic solubility was measured using the same method described above. The results are shown in Table 6K, suggesting that the increase of surfactant quantity results in a higher solubility of API.

TABLE 6K

| | | Kinetic solubility (μg/mL) | | | |
|---|---|---|---|---|---|
| Time | | M221130-1 | M220930-1 | M221130-2 | M221130-3 |
| 5 | min | 28.22 | 42.3 | 51.26 | 53.95 |
| 10 | min | 28.26 | 43.2 | 56.35 | 60.88 |
| 20 | min | 29.22 | 47.4 | 57.08 | 66.31 |
| 30 | min | 29.49 | 46.62 | 57.07 | 69.52 |
| 45 | min | 29.85 | 47.43 | 57.8 | 70.58 |
| 60 | min | 29.85 | 48.16 | 58.92 | 70.4 |
| 90 | min | 31.1 | 46.06 | 58.14 | 73.2 |
| 120 | min | 31.56 | 43.51 | 57.8 | 69.03 |

Two cabozantinib compositions as described in Table 6L were prepared using the same method described above for evaluating the effect of lecithin.

TABLE 6L

| | Batch No | M221019-2 | M221019-3 |
|---|---|---|---|
| Description | Component | Unit weight (mg) | |
| API | Cabozantinib malate | 76 | 76 |
| Polymer | VA64 | 30 | 30 |
| | L100-55 | 30 | 30 |
| Surfactant | TPGS | 60 | 60 |
| | Lecithin | — | 60 |
| Adsorbent | SiO2 | — | 60 |

Kinetic solubility was measured using the same method described above. The results are shown in Table 6M, suggesting that the addition of lecithin into the formulation improves the solubility in vitro, and especially enhances the bioavailability in vivo.

TABLE 6M

Kinetic solubility (μg/mL)

| | | |
|---|---|---|
| Time | M221019-2 | M221019-3 |

| | | | |
|---|---|---|---|
| 5 | min | 37.14 | 49.69 |
| 10 | min | 35.89 | 43.82 |
| 20 | min | 37.27 | 47.82 |
| 30 | min | 46.14 | 47.3 |
| 45 | min | 37.87 | 45.93 |
| 60 | min | 39.73 | 46.51 |
| 90 | min | 40.26 | 47.35 |
| 120 | min | 39.63 | 50.42 |

Two cabozantinib compositions as described in Table 6N were prepared using the same method described above for evaluating the effect of lecithin.

TABLE 6N

| | Batch No | M210702-2 | M210702-3 |
|---|---|---|---|
| Description | Component | Weight (g) | |
| API | Cabozantinib malate | 3.1675 | 3.1675 |
| Polymer | HPMC-E5 | 5.00 | 2.50 |
| Surfactant | Lecithin | — | — |
| | TPGS | — | 2.50 |
| Adsorbent | SiO2 | 2.6669 | 2.6669 |
| Acid excipient | Malic acid | 2.50 | 2.50 |

Kinetic solubility was measured using the same method described above. The results are shown in Table 6O, suggesting that concurrent use of surfactant and acid excipient in formulation does increase the solubility significantly.

TABLE 6O

Kinetic solubility (μg/mL)

| | | |
|---|---|---|
| Time | M210702-2-I | M210702-3-I |

| | | | |
|---|---|---|---|
| 5 | min | 11.3 | 68.8 |
| 15 | min | 24.3 | 105.6 |
| 30 | min | 39.3 | 120.6 |
| 60 | min | 65.5 | 97.7 |
| 90 | min | 70.2 | 97.7 |
| 120 | min | 75.9 | 85.1 |

The PK studies were performed to evaluate the food effect of the reference product. The reference product (RLD), COMETRIQ® tablet (60 mg), was tested orally in six beagle dogs with a two-way crossover design. Blood samples were taken from each group of animals at the following time points: 0 h (before test drug administration), and 0.25, 0.5, 0.75, 1, 1.5, 2, 3, 4, 6, 8, 12 and 24 h after the drug administration. Cabozantinib plasma concentrations were analyzed by an LC-MS/MS method. The non-compartment model of software WinNonlin was used to calculate the pharmacokinetic parameters of each dog, Cmax, AUClast and AUCinf. The geometric averages of each parameters were used for comparisons of the in vivo absorption under different doing pretreatments.

Table 6P shows the geometric mean values as well as coefficient of variation (CV) of the pharmacokinetic parameters of two dosing groups. The result shows that the absorption of RLD under fed situation is much higher than that under fasted, which indicates a great food effect.

TABLE 6P

| | | Cₘₐₓ | AUCₗₐₛₜ | AUC_{inf} |
|---|---|---|---|---|
| Study design | | ng/mL | h*ng/mL | h*ng/mL |
| COMETRIQ (60 mg) in | Average | 1163.1 | 7241.3 | 7947.6 |
| fasted condition | CV(%) | 55.0 | 63.5 | 66.4 |
| COMETRIQ (60 mg) in | Average | 1937.7 | 17481.5 | 18682.0 |
| fed condition | CV(%) | 46.8 | 28.1 | 29.2 |

A cabozantinib compositions as described in Table 6Q was prepared. Briefly, Cabozantinib malate was dispersed a (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10). And then VA64, L100-55 and TPGS were dissolved into the solution completely. Finally, the SiO2 was dispersed into the solution to form a suspension under stirring. The suspension was introduced into spray dryer (SD-06AG, Labplant, UK) via flash atomization. The parameters were the following: feed rate 3.0 rpm, inlet temperature 55° C., outlet temperature 45° C., and atomization pressure 2.2 bar. The SDD particles were collected and used for granulating. The SDD particles, PVPP-XL and Magnesium stearate were blended evenly, then used for dry granulation. Finally, the granules were filled into enteric capsule. Each capsule contains 60 mg cabozantinib free base.

TABLE 6Q
Batch No: Cabozantinib T1 formulation

| Description | Component | Unit weight (mg) |
|---|---|---|
| ASD particles | Cabozantinib malate | 76 |
| | VA64 | 30 |
| | L100-55 | 30 |
| | TPGS | 60 |
| | SiO2 | 60 |
| Granulation | PVPP-XL | 60 |
| | MgSt | 4 |

| | |
|---|---|
| Total weight (mg) | 380 |

The Cabozantinib T1 formulation capsules were tested orally in six beagle dogs under fasted and fed condition. Six dogs were classified into two groups using a two-way crossover design. one group were fasted for 12 hours before administration, and then given food 4 hours after drug administration, the other group were fed with high-fat food according to FDA guidance (Assessing the effects of food on drug in INDs and NDAs-Clinical pharmacology considerations, 2019) 30 mins before administration. During the study, the dogs were allowed to drink water freely and each dog was administered with 50 ml water in total. Blood samples were taken from each group of animals at the following time points: 0 h (before test drug administration), and 0.25, 0.5, 1, 1.5, 2, 3, 4, 8, 12 and 24 h after the drug administration. Cabozantinib plasma concentrations were analyzed by an LC-MS/MS method. The non-compartment model of software WinNonlin was used to calculate the pharmacokinetic parameters of each dog, Cmax, AUCt and AUCinf. The geometric averages of each parameters were used for comparisons of the in vivo absorption under different doing pretreatments.

Table 6R shows the geometric mean values as well as coefficient of variation (CV) of the pharmacokinetic parameters of two groups. The result shows that the absorption of Cabozantinib in Cabozantinib T1 ASD under fast situation is comparable to that under fed situation, which indicates a reduced food effect as compared to RLD. It reveals that Cabozantinib T1 formulation enhances the absorption of cabozantinib under fasted situation.

TABLE 6R

| | | Cₘₐₓ | AUCₗₐₛₜ | AUC_{inf} |
|---|---|---|---|---|
| Study design | | ng/mL | h*ng/mL | h*ng/mL |
| Cabozantinib T1 (60 mg) | Average | 1535.2 | 11239.2 | 11960.8 |
| in fasted condition | CV(%) | 59.6 | 37.3 | 37.0 |
| Cabozantinib T1 (60 mg) | Average | 938.9 | 10463.3 | 11201.8 |
| in fed condition | CV(%) | 51.2 | 28.3 | 28.3 |

### Example 5. Venetoclax Compositions

This example illustrates the process of improving the oral absorption of and reducing or removing the food-effect of venetoclax, according to some embodiments of the present disclosure.

Venetoclax was developed by ABBVIE and approved by FDA in 2016 under the brand name VENCLEXTA. It is a BCL-2 inhibitor indicated for the treatment of patients with chronic lymphocytic leukemia (CLL) with 17p deletion. Venetoclax is considered to have poor water solubility. VENCLEXTA tablets should be taken orally once daily with a meal and water to help improve the absorption.

Two venetoclax compositions as described in Table 7A were prepared.

TABLE 7A

| | Batch No. | | |
|---|---|---|---|
| | M210618-1-I | M210618-2-I | |
| Component | Unit weight (mg) | Unit weight (mg) | Description |
| Venetoclax | 100 | 100 | API |
| VA64 | 100 | — | Polymer |
| HPMCAS-LF | — | 100 | Polymer |
| TPGS | 100 | 100 | Surfactant |
| Citric acid | 22.5 | 22.5 | Acid excipient |

Amorphous solid dispersions were prepared by Spray drying method. Briefly, API was dispersed a mixed organic solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 7B. After collection, the particles were dried in a convection tray dryer to remove the residual solvent before further use.

TABLE 7B

| | |
|---|---|
| Feed rate (rpm) | 3 |
| Inlet temperature (C. °) | 60 |
| Outlet temperature (C. °) | 45 |
| Atomization pressure (bar) | 2.6 |

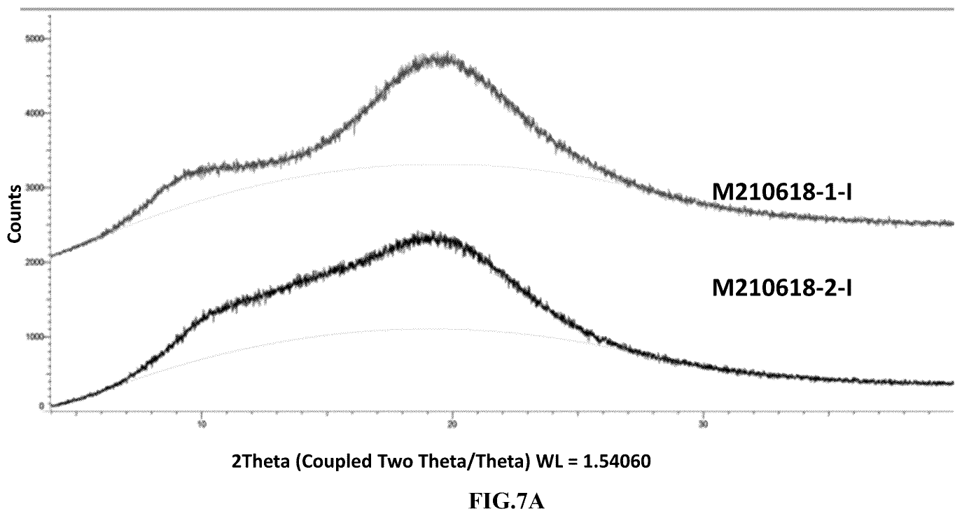

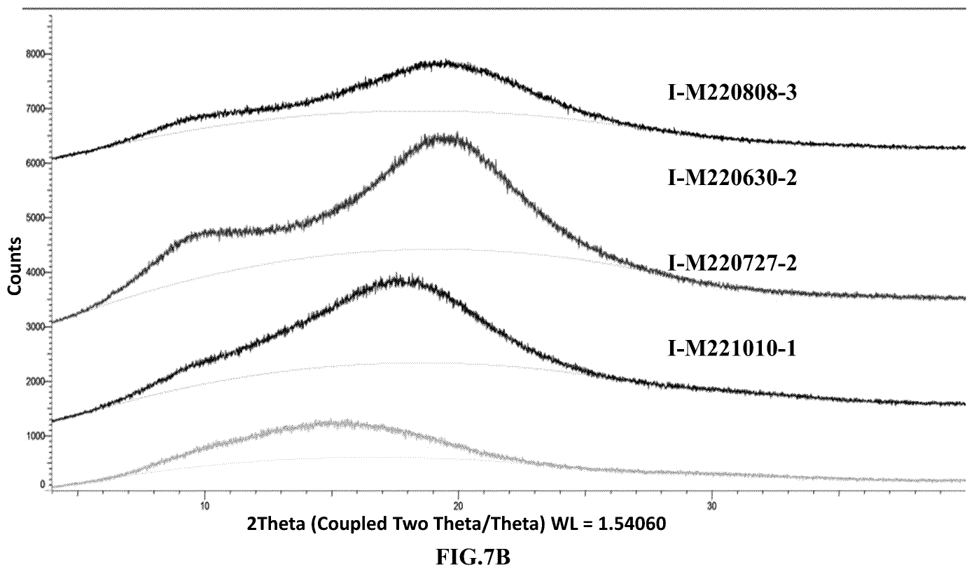

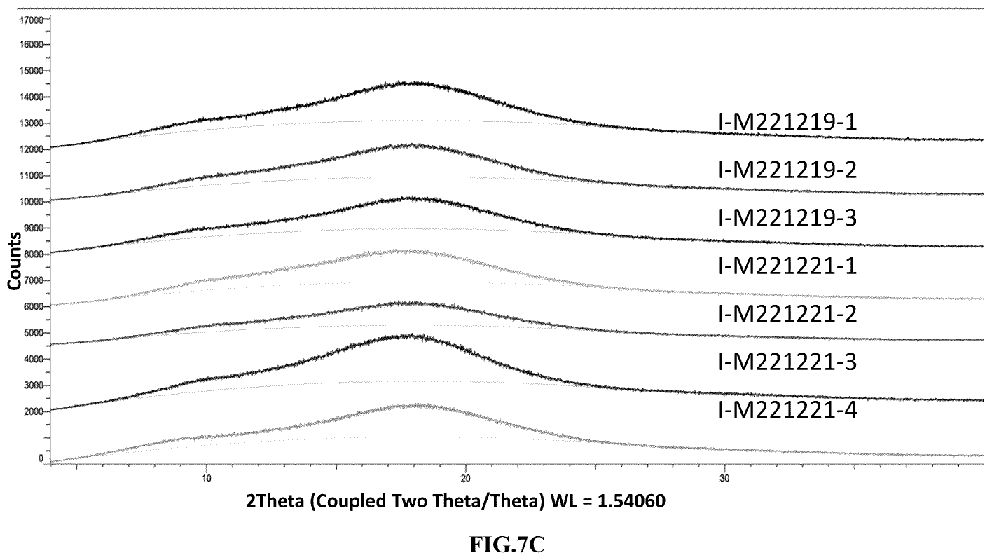

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The results (FIG. 7A) show that the two batches are in amorphous state.

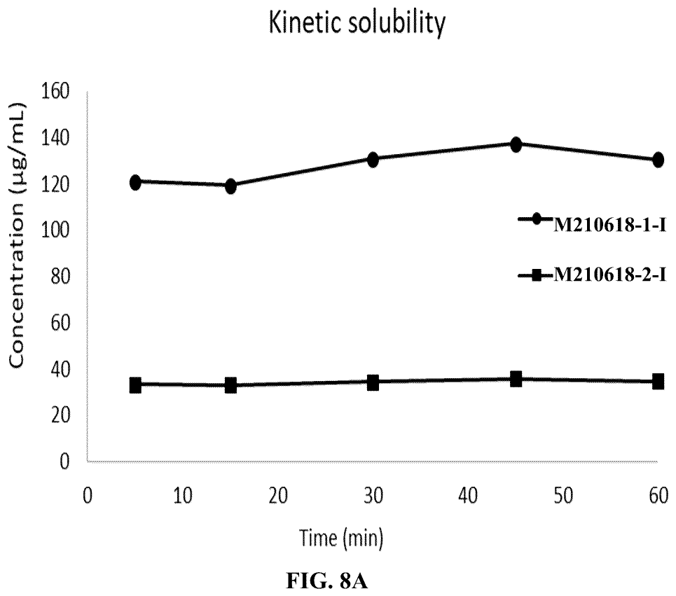

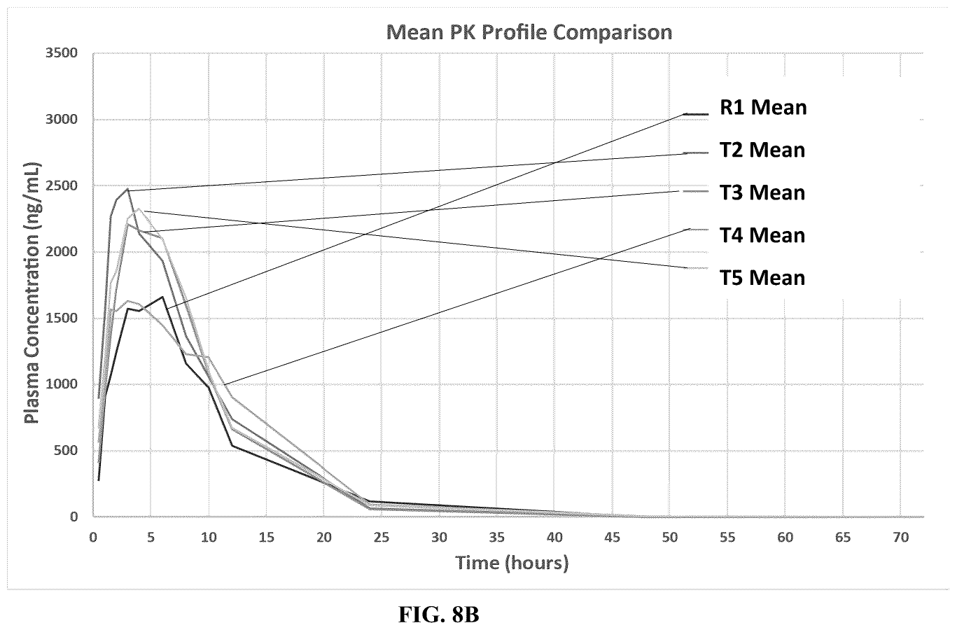

Kinetic solubility of the two venetoclax compositions was measured. Excess of SDD powder was added into 40 ml FeSSIF medium at 37° C. under 400 rpm rotation. 1 ml medium was withdrawn into a centrifugal tube at specified time point (5, 15, 30, 45, and 60 min) and then centrifuged for 10 min under 10000 rpm. The upper solution was collected for assay measure by HPLC. The results as shown in Table 7C and FIG. 8A suggest that HPMCAS is more effective than VA64 to increase kinetic solubility for the ASD formulation.

TABLE 7C

Kinetic solubility (μg/mL)

| Time (min) | M210618-1-I | M210618-2-I |
|---|---|---|
| 5 | 120.88 | 33.29 |
| 15 | 119.22 | 33.10 |
| 30 | 130.63 | 34.32 |
| 45 | 137.09 | 35.83 |
| 60 | 130.42 | 34.77 |

Four venetoclax compositions as described in Table 7D were prepared to evaluate the effect of various polymers.

TABLE 7D

| | | Batch No | | | |
|---|---|---|---|---|---|
| | | I-M220808-3 | I-M220630-2 | I-M220727-2 | I-M221010-1 |
| Description | Component | Unit weight (mg) | | | |
| API | Venetoclax | 100 | 100 | 100 | 100 |
| Polymer | Soluplus | 100 | — | — | — |
| | HPMC-E5 | — | 100 | — | — |
| | Eudragit L100 | — | — | 100 | 100 |
| Surfactant | TPGS | 100 | 100 | 100 | — |

Amorphous solid dispersions were prepared by spray drying method. Briefly, API was dispersed a mixed organic solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 3.0-8.0 rpm, inlet temperature 50-100° C., outlet temperature 30-80° C., and atomization pressure 0-4.0 bar. The particles were collected and used for test of crystalline phase and dissolution in FaSSIF medium.

XRPD studies were performed using the same method described above. The results (FIG. 7B) show that all four batches are in amorphous state.

Kinetic solubility was measured. Excess of SDD powder was added into 250 ml medium (Ph 6.5) at 37° C. under 75 rpm rotation. 1 ml medium was withdrawn into a centrifugal tube at specified time point and then centrifuged for 10 min under 10000 rpm. The upper solution was collected for assay measure by HPLC. The results are shown in Table 7E, suggesting that the addition of various polymers to formulation markedly improves the solubility of API, especially enteric polymers (Eudragit L100).

TABLE 7E

| | | Kinetic solubility (μg/mL) | | | |
|---|---|---|---|---|---|
| Time | | I-M220808-3 | I-M220630-2 | I-M220727-2 | I-M221010-1 |
| 10 | min | 13.4 | 9.4 | 139.72 | 0.36 |
| 30 | min | 22.09 | 10.83 | 281.33 | 0.36 |
| 60 | min | 23.05 | 10.85 | 382.92 | 0.36 |
| 90 | min | 25.41 | 8.53 | 431.48 | 0.36 |
| 120 | min | 22.92 | 7.43 | 430.31 | 1.13 |

Three venetoclax compositions as described in Table 7F were prepared using the same method described above for evaluating the quantity of polymer.

TABLE 7F

| | | Batch No | | |
|---|---|---|---|---|
| | | I-M220727-2 | I-M220816-2 | I-M220816-3 |
| Description | Component | Unit weight (mg) | | |
| API | Venetoclax | 100 | 100 | 100 |
| Polymer | Eudragit | 100 | 300 | 500 |
| | L100 (L100) | | | |
| Surfactant | TPGS | 100 | 100 | 100 |

Kinetic solubility was measured using the same method described above. The results are shown in Table 7G, suggesting that the increase of polymer quantity in formulation results in a higher solubility of API.

TABLE 7G

Kinetic solubility (μg/mL)

| | | | |
|---|---|---|---|
| Time | I-M220727-2 | I-M220816-2 | I-M220816-3 |

| | | | | |
|---|---|---|---|---|
| 10 | min | 139.72 | 201.96 | 204.87 |
| 30 | min | 281.33 | 361.34 | 312.32 |
| 60 | min | 382.92 | 450.98 | 366.96 |
| 90 | min | 431.48 | 518.95 | 406.76 |
| 120 | min | 430.31 | 543.56 | 417.15 |

Six venetoclax compositions as described in Table 7H were prepared using the same method described above for evaluating the effect of various surfactants.

TABLE 7H

| | | Batch No | | | | | |
|---|---|---|---|---|---|---|---|
| | | I-M220727-2 | I-M221219-1 | I-M221219-2 | I-M221219-3 | I-M221221-1 | I-M221221-4 |
| Description | Component | Unit weight (mg) | | | | | |
| API | Venetoclax | 100 | 100 | 100 | 100 | 100 | 100 |
| Polymer | L100 | 100 | 100 | 100 | 100 | 100 | 100 |
| Surfactant | TPGS | 100 | — | — | — | — | — |
| | Tween-80 | — | 100 | — | — | — | — |
| | RH40 | — | — | 100 | — | — | — |
| | Gelucire44/14 | — | — | — | 100 | — | — |
| | Poloxamer 407 | — | — | — | — | 100 | — |
| | Labrasol | — | — | — | — | — | 100 |

Kinetic solubility was measured using the same method described above. The results are shown in Table 71, suggesting that various surfactants enhance the solubility of API, especially TPGS, RH40, Tween-80 and Poloxamer.

TABLE 7I

| | Kinetic solubility (μg/mL) | | | | | |
|---|---|---|---|---|---|---|
| Time | I-M220727-2 | I-M221219-1 | I-M221219-2 | I-M221219-3 | I-M221221-1 | I-M221221-4 |
| 10 min | 609.82 | 930.24 | 915.37 | 853.16 | 81.73 | 532.55 |
| 30 min | 878.97 | 931.4 | 946.52 | 817.29 | 190.37 | 615.57 |
| 60 min | 922.62 | 932.48 | 937.13 | 650.31 | 573.12 | 596.32 |
| 90 min | 927.52 | 938.69 | 927.16 | 423.12 | 756.35 | 265.26 |
| 120 min | 934.82 | 950.19 | 931.34 | 409.63 | 823.4 | 350.1 |

XRPD studies were performed using the same method described above. The results (FIG. 7C) show that all six batches are in amorphous state.

Six venetoclax compositions as described in Table 7J were prepared using the same method described above for evaluating the effect of various surfactants.

TABLE 7J

| | | Batch No | | | | |
|---|---|---|---|---|---|---|
| | | I-M220727-2 | I-M221201-2 | I-M221208-4 | I-M221208-3 | I-M221010-1 |
| Description | Component | Unit weight (mg) | | | | |
| API | Venetoclax | 100 | 100 | 100 | 100 | 100 |
| Polymer | L100 | 100 | 100 | 100 | 100 | 100 |
| Surfactant | TPGS | 100 | 75 | 50 | 25 | — |

Kinetic solubility was measured using the same method described above. The results are shown in Table 7K, suggesting that increase in surfactant quantity results in a higher API solubility.

TABLE 7K

| | | Kinetic solubility (μg/mL) | | | | |
|---|---|---|---|---|---|---|
| Time | | I-M220727-2 | I-M221201-2 | I-M221208-4 | I-M221208-3 | I-M221010-1 |
| 10 | min | 609.82 | 59.28 | 4.48 | 1.09 | 0.36 |
| 30 | min | 878.97 | 137.56 | 26.66 | 5.56 | 0.36 |
| 60 | min | 922.62 | 206.57 | 108.41 | 19.91 | 0.36 |
| 90 | min | 927.52 | 263.86 | 209.72 | 35.83 | 0.36 |
| 120 | min | 934.82 | 326.61 | 274.27 | 52.97 | 1.13 |

Three venetoclax compositions as described in Table 7L were prepared using the same method described above for evaluating the effect of acid excipient.

TABLE 7L

| | | Batch No | | |
|---|---|---|---|---|
| | | I-M220630-1 | I-M220531-2 | I-M220531-3 |
| Description | Component | Unit weight (mg) | | |
| API | Venetoclax | 100 | 100 | 100 |
| Polymer | VA64 | 100 | 100 | 100 |
| Surfactant | TPGS | 100 | 100 | 100 |
| Acid excipient | Citric acid | — | 50 | 100 |
| Adsorbent | SiO2 | — | 150 | 150 |

Kinetic solubility was measured using the same method described above. The results are shown in Table 7M, suggesting that adding both surfactant and acid into formulation results in a higher solubility of API.

TABLE 7M

Kinetic solubility (μg/mL)

| | | | |
|---|---|---|---|
| Time | I-M220630-1 | I-M220531-2 | I-M220531-3 |

| | | | | |
|---|---|---|---|---|
| 10 | min | 21.32 | 55.74 | 62.11 |
| 30 | min | 36.85 | 61.79 | 63.77 |
| 60 | min | 41.22 | 65.23 | 69.14 |
| 90 | min | 29.43 | 68.4 | 67.99 |
| 120 | min | 33.22 | 70.12 | 68.4 |

Five venetoclax compositions as described in Table 7N were prepared for PK studies. VENCLEXTA tablet (RLD) was milled into powder, and then suspended in 0.5% CMC-Na solution. Amorphous solid dispersions were prepared by spray drying method. Briefly, API was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution. The solution was introduced into spray dryer (SD-06AG, Labplant, UK) via flash atomization. The parameters were the following: feed rate 3.0 rpm, inlet temperature 60° C., outlet temperature 45° C., and atomization pressure 2.6bar. The particles were collected and suspended in 0.5% CMC-Na solution for PK study.

TABLE 7N

| | | Batch No. | | | |
|---|---|---|---|---|---|
| | | I-M221201-2 | I-M221118 | I-M221219-1 | I-M221219-2 |
| Component | | Unit weight (mg) | | | |
| Venetoclax | RLD | 100 | 100 | 100 | 100 |
| L100 | batch | 100 | 100 | 100 | 100 |
| TPGS | 1153165 | 75 | 75 | — | — |
| T-80 | | — | — | 100 | — |
| RH40 | | — | — | — | 100 |
| SiO2 | | — | 150 | — | — |

A parallel PK study using rat model was conducted to evaluate the oral bioavailability of Venetoclax RLD and ASD batches in Table 7N. Rats were divided into give study groups, each with three rats. Rats were administered orally under fasted condition with either RLD or an ASD composition at the dose of 36 mg/kg of Venetoclax.

The results shown in Table 70 include the geometric mean values as well as the coefficient of variation (CV) of the pharmacokinetic parameters of the five study groups. Table 70 and FIG. 8B suggest that ASD formulation exhibit a higher absorption than RLD.

TABLE 7O

| | | Cmax | AUClast | AUCinf |
|---|---|---|---|---|
| Study design | | ng/mL | h*ng/mL | h*ng/mL |
| RLD (R1) | Average | 1811.7 | 17104.3 | 17919 |
| | CV(%) | 61 | 74 | 71.5 |
| I-M221201-2 (T2) | Average | 2491 | 22570 | 21506.7 |
| | CV(%) | 14.2 | 4.1 | 4.1 |
| I-M221118 (T3) | Average | 2290 | 21210 | 21506.7 |
| | CV(%) | 12.4 | 22.4 | 22 |
| I-M221219-1 (T4) | Average | 1725.7 | 20016.7 | 20323.3 |
| | CV(%) | 14 | 27.4 | 27.1 |
| I-M221219-2 (T5) | Average | 2346.3 | 23160 | 23373.3 |
| | CV(%) | 14.6 | 32.8 | 32.5 |

### Example 6. Lurasidone Compositions and PK Study in Beagle Dogs

This example illustrates the process of improving the oral absorption of and reducing or removing the food-effect of lurasidone, according to some embodiments of the present disclosure.

Lurasidone is an atypical antipsychotic indicated for the treatment schizophrenia and depressive. It is developed by SUNOVION PHARMS and approved by FDA in 2010 under the brand name LATUDA. Lurasidone is very slightly soluble in water, so it must be administered with food (at least 350 calories), which substantially increases the absorption. Thus, there is a need for developing pharmaceutical lurasidone compositions and methods of treatment, which address or eliminate the food effect and/or provide improved bioavailability.

Three lurasidonecompositions as described in Table 8A-1 were prepared.

TABLE 8A-1

| | Batch No. | | | |
|---|---|---|---|---|
| | I-M211207-2 | I-M211209-4 | M211028-1 | |
| | Unit weight | Unit weight | Unit weight | |
| Component | (mg) | (mg) | (mg) | Description |
| Lurasidone | 40 | 40 | 40 | API |
| hydrochloride | | | | |
| HPMC-E5 | 120 | 40 | 40 | polymer |
| TPGS | 40 | — | 40 | surfactant |
| TA | — | 40 | 40 | acid |
| | | | | excipient |
| SiO2 | 60 | — | — | adsorbent |

Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 8B. After collection, the particles were dried in a convection tray dryer to remove the residual solvent before further use.

TABLE 8B

| | |
|---|---|
| Feed rate (rpm) | 3 |
| Inlet temperature (C. °) | 70 |
| Outlet temperature (C. °) | 50 |
| Atomization pressure (bar) | 2.6 |

Two lurasidonecompositions as described in Table 8A-2 were prepared by spray drying method. Briefly, API was dispersed a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were the following: feed rate 3.0-8.0 rpm, inlet temperature 50-100° C., outlet temperature 30-80° C., and atomization pressure 0-4.0 bar. The particles were collected and used for test of crystalline phase and dissolution in FaSSIF medium.

TABLE 8A-2

Batch No

| | |
|---|---|
| M211101-1 | M211101-2 |

| | | |
|---|---|---|
| Description | Component | Unit weight (mg) |

| | | | |
|---|---|---|---|
| API | Lurasidone | 40 | 40 |
| | hydrochloride | | |
| Polymer | VA64 | 40 | — |
| | PVP-K30 | — | 40 |
| Surfactant | TPGS | 40 | 40 |
| Acid excipient | Tartaric acid | 40 | 40 |

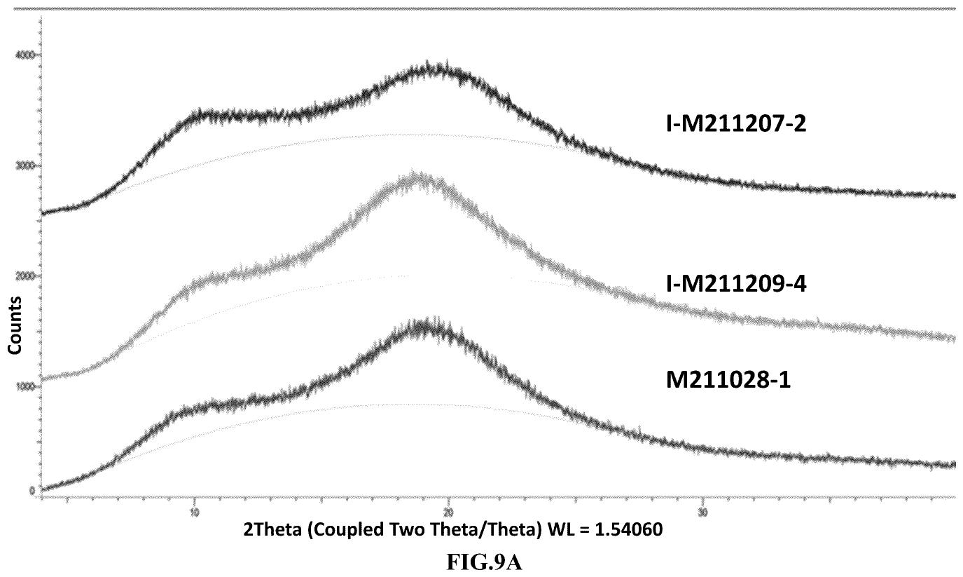

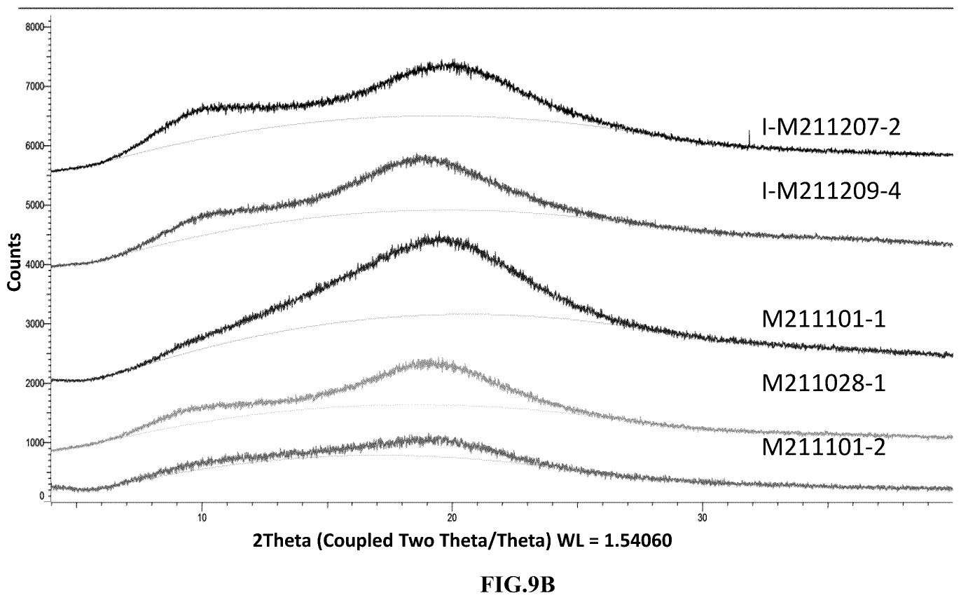

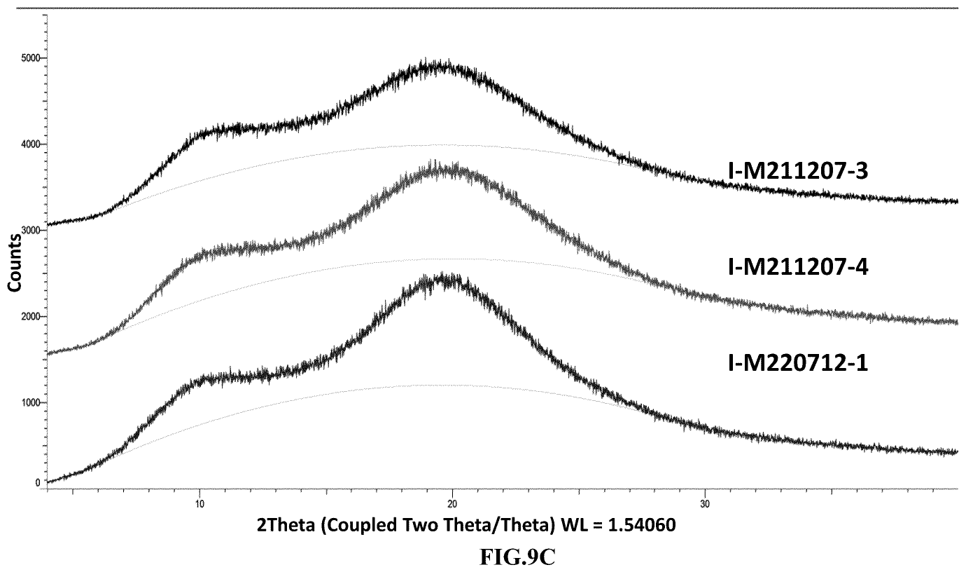

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The results (FIGS. 9A-9B) show all five batches were in amorphous state.

Kinetic solubility of the three pazopanib compositions was measured. Excess of SDD powder was added into 40 ml FaSSIF medium at 37° C. under 400 rpm rotation. 1 ml medium was withdrawn into a centrifugal tube at specified time point (5, 15, 30, 60, 90, and 120 min) and then centrifuged for 10 min under 10000 rpm. The upper solution was collected for assay measure by HPLC.

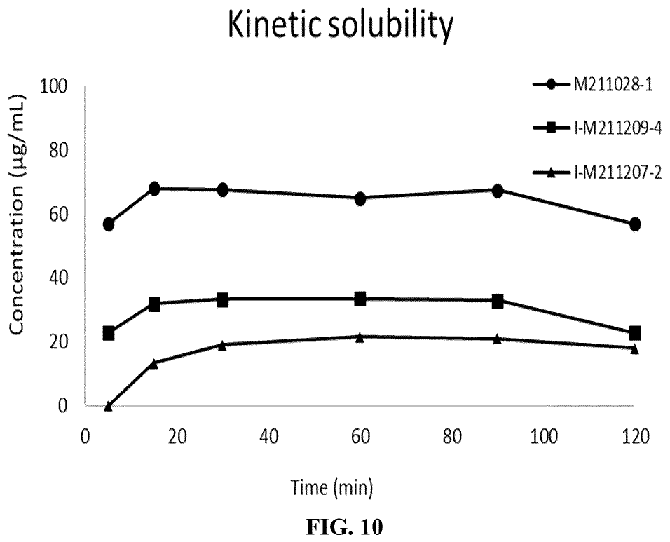

The results (Table 8C-1, Table 8C-2 and FIG. 10) showed that lurasidone has poor dissolution profile if it exists in crystalline form in the composition. However, when lurasidone is prepared in amorphous state, the dissolution rate increased significantly. The addition of surfactant into formulation improves the solubility of lurasidone. Especially, when concurrent use of surfactant and acid excipient in the formulation do enhance the solubility significantly.

TABLE 8C-1

| Time | Kinetic solubility (μg/mL) | | |
|---|---|---|---|
| (min) | I-M211207-2 | I-M211209-4 | M211028-1 |
| 5 | — | 22.78 | 56.93 |
| 15 | 13.28 | 31.92 | 67.95 |
| 30 | 19.01 | 33.30 | 67.70 |
| 60 | 21.46 | 33.39 | 65.06 |
| 90 | 21.07 | 33.01 | 67.53 |
| 120 | 17.92 | 22.78 | 56.93 |

TABLE 8C-2

| | |
|---|---|
| Time | Kinetic solubility (μg/mL) |

| (min) | M211101-1 | M211101-2 |
|---|---|---|
| 5 | 54.29 | 57.12 |
| 15 | 67.11 | 71.4 |
| 30 | 59.41 | 65.14 |
| 60 | 63.37 | 64.05 |
| 90 | 57.44 | 52.01 |
| 120 | 54.29 | 57.12 |

Six lurasidone compositions as described in Table 8A-3 and Table 8A-4 were prepared using the same method described above for evaluating the polymer quantity.

TABLE 8A-3

| | | Batch No | | |
|---|---|---|---|---|
| | | M211028-1 | M211104-1 | M211104-2 |
| Description | Component | Unit weight (mg) | | |
| API | Lurasidone | 40 | 40 | 40 |
| | hydrochloride | | | |
| Polymer | HPMC-E5 | 40 | 80 | 200 |
| Surfactant | TPGS | 40 | 40 | 40 |
| Acid | Tartaric | 40 | 40 | 40 |
| excipient | acid | | | |

TABLE 8A-4

| | | Batch No | | |
|---|---|---|---|---|
| | | I-M211207-3 | I-M211207-4 | I-M220712-1 |
| Description | Component | Unit weight (mg) | | |
| API | Lurasidone | 40 | 40 | 40 |
| | hydrochloride | | | |
| Polymer | HPMCAS-LF | 40 | 120 | 120 |
| Surfactant | TPGS | 40 | 40 | 40 |
| | Lecithin | — | — | 40 |
| Adsorbent | SiO2 | 60 | 60 | 60 |

Kinetic solubility was measured using the same method described above. The results shown in Table 8C-3 suggests that the increase of HPMC-E5 quantity has a slight influence in solubility in vitro, but it may have the preparation process more feasibility. The results shown in Table 8C-4 suggests that increase in polymer quantity results in a higher solubility.

TABLE 8C-3

Kinetic solubility (μg/mL)

| | | | |
|---|---|---|---|
| Time | M211028-1 | M211104-1 | M211104-2 |

| | | | | |
|---|---|---|---|---|
| 10 | min | 56.93 | 55.97 | 43.55 |
| 30 | min | 67.95 | 67.38 | 56.15 |
| 60 | min | 67.7 | 70.42 | 70.74 |
| 90 | min | 65.06 | 71.03 | 73.66 |
| 120 | min | 67.53 | 70.89 | 72.86 |

TABLE 8C-4

Kinetic solubility (μg/mL)

| | | | |
|---|---|---|---|
| Time | I-M211207-3 | I-M211207-4 | I-M220712-1 |

| | | | | |
|---|---|---|---|---|
| 10 | min | 11.58 | 21.85 | 45.86 |
| 30 | min | 5.75 | 24.23 | 55.17 |
| 60 | min | 4.51 | 26.6 | 160.64 |
| 90 | min | 2.45 | 30.36 | 113.4 |
| 120 | min | 4.6 | 30.67 | 209.55 |

XRPD studies were performed using the same method described above. The results (FIG. 9C) show that the three batches from Table 8A-4 are in amorphous state.

Four lurasidone compositions as described in Table 8A-5 were prepared using the same method described above for evaluating the effect of surfactant.

TABLE 8A-5

| | | Batch No | | | |
|---|---|---|---|---|---|
| | | I-M211118-1 | I-M211123-1 | I-M211123-3 | I-M211125-2 |
| Description | Component | Unit weight (mg) | | | |
| API | Lurasidone | 40 | 40 | 40 | 40 |
| | hydrochloride | | | | |
| Polymer | HPMC-E5 | 40 | 40 | 40 | 40 |
| Acid | Tartaric | 40 | 40 | 40 | 40 |
| | acid | | | | |
| Surfactant | TPGS | 40 | — | — | — |
| | RH40 | — | 40 | — | — |
| | Pluronic F-68 | — | — | 40 | — |
| | PEG6000 | — | — | — | 40 |
| Adsorbent | SiO2 | 60 | 60 | 60 | 60 |

Kinetic solubility was measured using the same method described above. The results shown in Table 8C-5 suggests that various surfactants enhance the solubility of lurasidone, especially TPGS.

TABLE 8C-5

| | | Kinetic solubility (μg/mL) | | | |
|---|---|---|---|---|---|
| Time | | I-M211118-1 | I-M211123-1 | I-M211123-3 | I-M211125-2 |
| 10 | min | 43.54 | 34.15 | 15.6 | 17.84 |
| 30 | min | 53.98 | 37.76 | 20.57 | 21.29 |
| 60 | min | 53.68 | 22.62 | 22.28 | 22.98 |
| 90 | min | 53.24 | 13.94 | 23.46 | 22.53 |
| 120 | min | 49.2 | 6.52 | 23.45 | 21.81 |

Three lurasidone compositions as described in Table 8A-6 were prepared using the same method described above for evaluating the surfactant quantity.

TABLE 8A-6

| | | Batch No | | |
|---|---|---|---|---|
| | | M211028-4 | M211028-3 | M211028-1 |
| Description | Component | Unit weight (mg) | | |
| API | Lurasidone | 40 | 40 | 40 |
| | hydrochloride | | | |
| Polymer | HPMC-E5 | 40 | 40 | 40 |
| Surfactant | TPGS | — | 20 | 40 |
| Acid | Tartaric | 40 | 40 | 40 |
| excipient | acid (TA) | | | |

Kinetic solubility was measured using the same method described above. The results shown in Table 8C-6 suggests that increase in surfactant quantity results in a higher solubility.

TABLE 8C-6

Kinetic solubility (μg/mL)

| | | | |
|---|---|---|---|
| Time | M211028-4 | M211028-3 | M211028-1 |

| | | | | |
|---|---|---|---|---|
| 10 | min | 5.98 | 43.94 | 56.93 |
| 30 | min | 14.46 | 44.16 | 67.95 |
| 60 | min | 10.71 | 48.66 | 67.7 |
| 90 | min | 29.41 | 49.29 | 65.06 |
| 120 | min | 23.82 | 47.64 | 67.53 |

Five lurasidone compositions as described in Table 8A-7 were prepared using the same method described above for evaluating the ratio of TPGS to lecithin.

TABLE 8A-7

| | | Batch No | | | | |
|---|---|---|---|---|---|---|
| | | I-M220801-1 | I-M220801-2 | I-M220801-3 | I-M220801-4 | I-M220801-5 |
| Description | Component | Unit weight (mg) | | | | |
| API | Lurasidone | 40 | 40 | 40 | 40 | 40 |
| | hydrochloride | | | | | |
| Polymer | HPMCAS-LF | 40 | 120 | 120 | 120 | 120 |
| Surfactant | TPGS | 40 | — | 20 | 40 | 20 |
| | Lecithin | 40 | 40 | 40 | 20 | 20 |

Kinetic solubility was measured using the same method described above. The results shown in Table 8C-7 suggests that various ratio of TPGS/Lecithin in formulation all result in a higher solubility.

TABLE 8C-7

| | | Kinetic solubility (μg/mL) | | | | |
|---|---|---|---|---|---|---|
| Time | | I-M220801-1 | I-M220801-2 | I-M220801-3 | I-M220801-4 | I-M220801-5 |
| 10 | min | 67.04 | 9.38 | 49.9 | 52.11 | 44.22 |
| 30 | min | 88.06 | 37.62 | 54.46 | 64.68 | 39.48 |
| 60 | min | 143.35 | 35.92 | 68.11 | 104.49 | 38.72 |
| 90 | min | 169.9 | 50.26 | 121.14 | 77.09 | 45.38 |
| 120 | min | 183.97 | 51.42 | 86.67 | 72.22 | 26.93 |

Three lurasidone compositions as described in Table 8A-8 were prepared using the same method described above for evaluating the adsorbent quantity.

TABLE 8A-8

| | | Batch No | | |
|---|---|---|---|---|
| | | I-M211028-1 | I-M211104-3 | I-M211104-4 |
| Description | Component | Unit weight (mg) | | |
| API | Lurasidone | 40 | 40 | 40 |
| | hydrochloride | | | |
| Polymer | HPMC-E5 | 40 | 40 | 60 |
| Surfactant | TPGS | 40 | 40 | 40 |
| Acid | Tartaric acid | 40 | 40 | 40 |
| excipient | | | | |
| Adsorbent | SiO2 | — | 40 | 20 |

| | | | |
|---|---|---|---|
| Yield (%) | <10 | 32 | 40 |

Kinetic solubility was measured using the same method described above. The results shown in Table 8C-8 suggests that increase in adsorbent quantity results a higher yield.

TABLE 8C-8

Kinetic solubility (μg/mL)

| | | | |
|---|---|---|---|
| Time | I-M211028-1 | I-M211104-3 | I-M211104-4 |

| | | | | |
|---|---|---|---|---|
| 10 | min | 56.93 | 47.92 | 50.53 |
| 30 | min | 67.95 | 60.72 | 63.95 |
| 60 | min | 67.7 | 57.3 | 63.2 |
| 90 | min | 65.06 | 55.88 | 64.8 |
| 120 | min | 67.53 | 56.46 | 68.56 |

Three lurasidone compositions as described in Table 8A-9 were prepared using the same method described above for evaluating the acid excipient.

TABLE 8A-9

| | | Batch No | | |
|---|---|---|---|---|
| | | M211027-2 | M211028-2 | M211028-1 |
| Description | Component | Unit weight (mg) | | |
| API | Lurasidone | 40 | 40 | 40 |
| | hydrochloride | | | |
| Polymer | HPMC-E5 | 40 | 40 | 40 |
| Surfactant | TPGS | 40 | 40 | 40 |
| Acid | Tartaric | | 20 | 40 |
| excipient | acid | | | |

Kinetic solubility was measured using the same method described above. The results shown in Table 8C-9 suggests that increase in acid quantity results a higher solubility.

TABLE 8C-9

Kinetic solubility (μg/mL)

| | | | |
|---|---|---|---|
| Time | M211027-2 | M211028-2 | M211028-1 |

| | | | | |
|---|---|---|---|---|
| 10 | min | 6.31 | 42.45 | 56.93 |
| 30 | min | 11.57 | 45.48 | 67.95 |
| 60 | min | 16.07 | 47.81 | 67.7 |
| 90 | min | 16.7 | 46.8 | 65.06 |
| 120 | min | 3.77 | 46.24 | 67.53 |

A separate lurasidone composition as described in Table 8D was prepared.

TABLE 8D

| Batch No. | P210324-1 | Description |
|---|---|---|
| Lurasidone hydrochloride | 4 g | API |
| HPMC-E5 | 4 g | polymer |
| TPGS | 4 g | surfactant |
| Citric acid | 4 g | acid excipient |

Amorphous solid dispersions were prepared by Spray drying method. Briefly, API was dispersed a mixed organic solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters were shown in Table 8E. After collection, the particles were dried in a convection tray dryer to remove the residual solvent before further use. The particles were filled into gel capsule. Each capsule contains 40 mg lurasidone hydrochloride.

TABLE 8E

| | |
|---|---|
| Feed rate (rpm) | 9 |
| Inlet temperature (C.°) | 75 |
| Outlet temperature (C.°) | 49 |
| Atomization pressure (bar) | 2.2 |

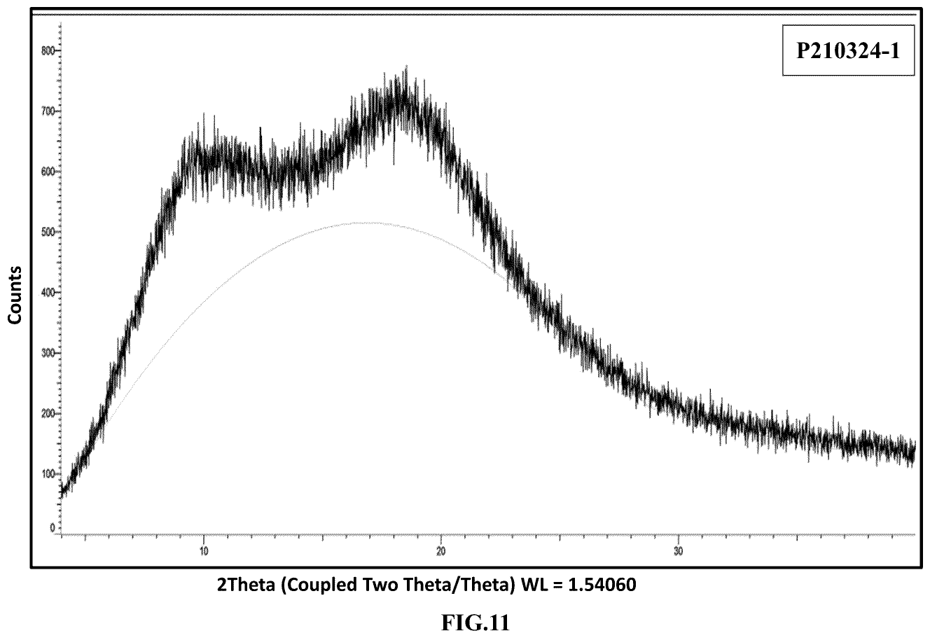

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The result (FIG. 11) shows that batch P210324-1 was in amorphous state.

Kinetic solubility of the lurasidone composition (batch P210324-1) was measured. Excess of SDD powder was added into 40 ml FeSSIF medium at 37° C. under 400 rpm rotation. 1 ml medium was withdrawn into a centrifugal tube at specified time point (5, 30, 60, and 120 min) and then centrifuged for 10 min under 10000 rpm. The upper solution was collected for assay measure by HPLC.

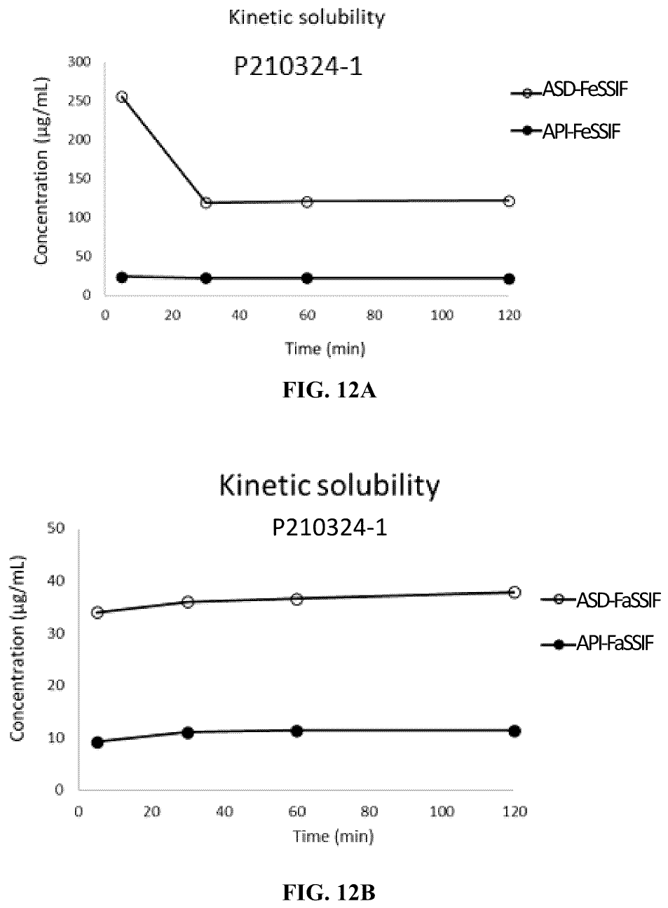

The results (Table 8F and FIGS. 12A-B) show that crystalline form of lurasidone has limited solubility in FaSSIF and FeSSIF medium. When it is prepared to ASD, which contains polymer, acid and surfactant, its kinetic solubility increases in FaSSIF and increases significantly in FeSSIF, suggesting that ASD composition improves the absorption of lurasidone.

TABLE 8F

| | Kinetic solubility (μg/mL) | |
|---|---|---|
| Time | API | P210324-1 |

| (min) | FaSSIF | FeSSIF | FaSSIF | FeSSIF |
|---|---|---|---|---|
| 5 | 9.2 | 23.58 | 34.02 | 255.33 |
| 30 | 11.05 | 22.49 | 36.01 | 118.92 |
| 60 | 11.44 | 22.33 | 36.61 | 120.61 |
| 120 | 11.4 | 21.86 | 37.89 | 121.74 |

The reference product, LATUDA® tablet (40 mg), was tested orally in six beagle dogs under two different dosing pretreatments, using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.25, 0.5, 0.75, 1, 1.5, 2, 3, 4, 6, 8, 12 and 24 h after the drug administration. Lurasidone plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 8G include the geometric mean values as well as the coefficient of variation (CV) of the pharmacokinetic parameters of two dosing groups. The absorption of LATUDA® under fed situation is about 6 times higher than that under fasted, which indicates a great food effect.

TABLE 8G

| | | Cₘₐₓ | AUCₗₐₛₜ | AUC_{inf} |
|---|---|---|---|---|
| Study design | | ng/mL | h*ng/mL | h*ng/mL |
| LATUDA (40 mg) in | Average | 30.8 | 90.1 | 112.3 |
| fasted condition | CV(%) | 69.9% | 51.6% | 37.9% |
| LATUDA (40 mg) in | Average | 213.5 | 495.0 | 531.1 |
| fed condition | CV(%) | 32.9% | 28.6% | 29.0% |

| | | | |
|---|---|---|---|
| LATUDA (40 mg) -Fed/Fasted | 1163.1 | 696.0 | 502.2 |
| Mean Ratio(%) | | | |
| LATUDA (40 mg) -Fed/Fasted | 2342.9 | 1056.2 | 697.2 |
| UP 90% | | | |
| LATUDA (40 mg) -Fed/Fasted | 577.4 | 458.6 | 361.7 |
| Low90% | | | |

The ASD capsules (40 mg), batch No. P210324-1 was tested orally in six beagle dogs under two different dosing pretreatments, using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.25, 0.5, 0.75, 1, 1.5, 2, 3, 4, 6, 8, 12 and 24 h after the drug administration. Lurasidone plasma concentrations were analyzed by an LC-MS/MS method. The non-compartment model of software WinNonlin was used to calculate the pharmacokinetic parameters of each dog, Cmax, AUCₗₐₛₜ and AUCinf. The geometric averages of each parameters were used for comparisons of the in vivo absorption under different doing pretreatments.

The results shown in Table 8H include the geometric mean values as well as coefficient of variation (CV) of the pharmacokinetic parameters of two dosing groups. The absorption of lurasidone in ASD capsules under fasted condition is about six times higher than the reference product LATUDA under fasted condition when the API dosing is the same in both compositions. Additionally, the absorption of lurasidone in ASD capsules under fasted condition is comparable to that under fed situation, which indicates a reduced food effect than RLD.

TABLE 8H

| | | Cₘₐₓ | AUCₗₐₛₜ | AUC_{inf} |
|---|---|---|---|---|
| Study design | | ng/mL | h*ng/mL | h*ng/mL |
| ASD capsules batch | Average | 355.7 | 628.8 | 670.1 |
| No. P210324-1 (40 mg) | CV(%) | 41.0 | 38.6 | 38.2 |
| in fasted condition | | | | |
| ASD capsules batch | Average | 191.5 | 519.6 | 589.3 |
| No. P210324-1 (40 mg) | CV(%) | 64.1 | 50.0 | 60.2 |
| in fed condition | | | | |

| | | | |
|---|---|---|---|
| No. P210324-1 (40 mg) -Fed/Fasted | 54.9 | 87.0 | 95.7 |
| Mean Ratio(%) | | | |
| No. P210324-1 (40 mg) -Fed/Fasted | 83.7 | 134.8 | 166.9 |
| UP 90% | | | |
| No. P210324-1 (40 mg) -Fed/Fasted | 36.0 | 56.1 | 54.8 |
| Low90% | | | |

A lurasidone composition as described in Table 81 was prepared. Briefly, Lurasidone hydrochloride was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, Citric acid and TPGS were dissolved into the solution completed. Finally, the SiO2 was dispersed into the solution to form a suspension under stirring. The suspension was introduced into spray dryer (SD-06AG, Labplant, UK) via flash atomization. The parameters were the following: feed rate 9.0 rpm, inlet temperature 75° C., outlet temperature 49° C., and atomization pressure 2.2bar. The particles were collected and used for dry granulation with MCC and Magnesium stearate. The dry granules, PVPP-XL and Magnesium stearate were blended evenly, then compressed into a tablet. Each tablet contains 40 mg Lurasidone hydrochloride.

TABLE 8I
Batch No: Lurasidone T2 formulation

| | | | Unit weight |
|---|---|---|---|
| | Description | Component | (mg) |
| Inner granulation | ASD particles | Lurasidone | 40 |
| | | Hydrochloride | |
| | | HPMC-E5 | 40 |
| | | TPGS | 40 |
| | | Citric acid | 40 |
| | | SiO2 | 60 |
| | | MCC(PH102) | 30 |
| | | Magnesium stearate | 1.15 |

| Outer granulation | PVPP-XL | 57.7 |
|---|---|---|
| | Magnesium stearate | 1.15 |

| | |
|---|---|
| Total weight (mg) | 310 |

The Lurasidone T2 formulation tablets were tested orally in six beagle dogs under fasted and fed condition. Six dogs were classified into two groups using a two-way crossover design. One group were fasted for 12 hours before administration, and then given food 4 hours after drug administration. The other group were fed with high-fat food according to FDA guidance (Assessing the effects of food on drug in INDs and NDAs-Clinical pharmacology considerations, 2019) 30 mins before administration. During the study, the dogs were allowed to drink water freely and each dog was administered with 50 ml water in total.

Blood samples were taken from each group of animals at the following time points: 0 h (before test drug administration), and 0.25, 0.5, 1, 1.5, 2, 3, 4, 8, 12 and 24 h after the drug administration. Lurasidone plasma concentrations were analyzed by an LC-MS/MS method. The non-compartment model of software WinNonlin was used to calculate the pharmacokinetic parameters of each dog, Cmax, AUClast and AUCinf. The geometric averages of each parameters were used for comparisons of the in vivo absorption under different doing pretreatments.

Table 8J shows the geometric mean values as well as coefficient of variation (CV) of the pharmacokinetic parameters of two groups. The result shows that the absorption of lurasidone under fast situation is comparable to that under fed situation, which indicates a reduced food effect than RLD.

TABLE 8J

| | | Cₘₐₓ | AUCₗₐₛₜ | AUC_{inf} |
|---|---|---|---|---|
| Study design | | ng/mL | h*ng/mL | h*ng/mL |
| Lurasidone T2 | Average | 540.4 | 795.8 | 873.6 |
| formulation (40 mg) in | CV(%) | 39.5 | 24.3 | 22.3 |
| fasted condition | | | | |
| Lurasidone T2 | Average | 309.5 | 735.0 | 800.3 |
| formulation (40 mg) in | CV(%) | 54.9 | 33.4 | 34.9 |
| fed condition | | | | |

| | | | |
|---|---|---|---|
| T2 formulation-Fed/Fasted | 52.6 | 90.0 | 80.5 |
| Mean Ratio(%) | | | |
| T2 formulation -Fed/Fasted | 80.2 | 121.5 | 104.2 |
| UP 90% | | | |
| T2 formulation -Fed/Fasted | 34.5 | 66.7 | 62.1 |
| Low90% | | | |

A lurasidone composition as described in Table 8K was prepared. Briefly, Lurasidone hydrochloride was dispersed in a solvent (e.g., ethanol and acetonitrile (3/7, V/V), 60% tetrahydrofuran solution, methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. Then, the HPMC-E5, Citric acid, Lecithin and TPGS were dissolved into the solution completely. Finally, the SiO2 was dispersed into the solution to form a suspension under stirring. The suspension was introduced into spray dryer (SD-06AG, Labplant, UK) via flash atomization. The parameters were the following: feed rate 9.0 rpm, inlet temperature 75° C., outlet temperature 49° C., and atomization pressure 2.2bar. The particles were collected and used for dry granulation with PVPP-XL and Magnesium stearate. The dry granules, PVPP-XL, MCC and Magnesium stearate were blended evenly, then compressed into a tablet. Each tablet contains 40 mg Lurasidone hydrochloride.

TABLE 8K
Batch No: Lurasidone T3 formulation

| | | | Unit weight |
|---|---|---|---|
| | Description | Ingredient | (mg) |
| Inner granulation | ASD particles | Lurasidone | 40 |
| | | Hydrochloride | |
| | | HPMC-E5 | 40 |
| | | TPGS | 40 |
| | | Lecithin | 40 |
| | | Citric acid | 40 |
| | | SiO2 | 60 |
| | | PVPP-XL | 57.7 |
| | | Magnesium stearate | 1.15 |

| Outer granulation | PVPP-XL | 30 |
|---|---|---|
| | MCC | 10 |
| | Magnesium stearate | 1.15 |

| | |
|---|---|
| Total weight (mg) | 360 |

The ASD tablet (Lurasidone T3 formulation, or T3) and the reference product (RLD), LATUDA® tablet, were tested orally in six beagle dogs under fasted and fed condition. Six dogs were classified into three groups using a three-way crossover design, two groups were administrated with T3 and LATUDA® tablet in fast condition, the other group were administrated with T3 in fed condition. The dose is 40 mg per in all groups. In fasted condition, the dogs were allowed to drink water freely and were fasted for 12 hours before administration, and then given food 4 hours after drug administration. In fed condition, the dogs were fed with high-fat food according to FDA guidance (Assessing the effects of food on drug in INDs and NDAs-Clinical pharmacology considerations, 2019) 30 mins before administration.

Blood samples were taken from each group of animals at the following time points: 0 h (before test drug administration), and 0.25, 0.5, 1, 1.5, 2, 3, 4, 8, 12 and 24 h after the drug administration. Lurasidone plasma concentrations were analyzed by an LC-MS/MS method. The non-compartment model of software WinNonlin was used to calculate the pharmacokinetic parameters of each dog, Cₘₐₓ, AUCt and AUCinf. The geometric averages of each parameters were used for comparisons of the in vivo absorption under different doing pretreatments.

Table 8K shows the geometric mean values as well as coefficient of variation (CV) of the pharmacokinetic parameters of three dosing groups. The results suggest that the food effect in T3 group is completely removed and the absorption of T3 under fast condition is comparable to that in RLD group under fed condition, which reveals that the ASD tablet enhances the absorption of lurasidone significantly.

TABLE 8K

| | | Cmax | AUClast | AUCinf |
|---|---|---|---|---|
| Study design | | ng/mL | h*ng/mL | h*ng/mL |
| Lurasidone T3 (40 mg) in | Average | 241.7 | 556.0 | 625.9 |
| fasted condition | CV(%) | 33.7 | 29.9 | 34.4 |
| Lurasidone T3 (40 mg) in | Average | 220.7 | 591.5 | 631.9 |
| fed condition | CV(%) | 42.7 | 23.6 | 24.1 |
| LATUDA ® (40 mg) in | Average | 278.6 | 652.0 | 703.8 |
| fasted condition | CV(%) | 48.1 | 25.7 | 25.9 |

| | | | |
|---|---|---|---|
| Lurasidone T3 Fed/Fasted | 89.9 | 107.8 | 102.0 |
| Mean ratio (%) | | | |
| Lurasidone T3 Fed/Fasted | 118.5 | 116.1 | 113.7 |
| UP 90% ratio (%) | | | |
| Lurasidone T3 Fed/Fasted | 68.1 | 100.1 | 91.4 |
| Low 90% ratio (%) | | | |
| Lurasidone T3 Fasted/RLD-Fasted | 89.3 | 84.1 | 86.8 |
| Mean ratio (%) | | | |
| Lurasidone T3 Fasted/RLD-Fasted | 145.9 | 98.4 | 103.9 |
| UP 90% ratio (%) | | | |
| Lurasidone T3 Fasted/RLD-Fasted | 54.6 | 71.8 | 72.5 |
| Low 90% ratio (%) | | | |

### Example 7. Vilazodone Compositions and PK Study in Beagle Dogs

This example illustrates the process of improving the oral absorption of and reducing or removing the food-effect of vilazodone hydrochloride, according to some embodiments of the present disclosure.

Vilazodone is indicated for the major depressive disorder. It is developed by ALLERGAN and approved by FDA in 2011 under the brand name VIIBRYD. Vilazodone is very slightly soluble in water, so it must be administered with food to help increases the absorption. Administration without food sometimes results in inadequate drug concentrations and reduces effectiveness. Thus, there is a need for developing pharmaceutical vilazodone compositions and methods of treatment to address or eliminate the food effect and/or provide improved bioavailability.

Vilazodone compositions as described in Table 9A was prepared.

TABLE 9A

| | | Batch No | | | | | | |
|---|---|---|---|---|---|---|---|---|
| | | P210324-1 | | | | | | |
| | | Vilazodone | P220215-1 | I-M230109-1 | I-M230109-2 | I-M230109-3 | I-M230109-6 | I-M230109-7 |
| Description | Component | Weight (g) | Unit weight (mg) | | | | | |
| API | Vilazodone | 4 | 10 | 40 | 40 | 40 | 40 | 40 |
| | hydrochloride | | | | | | | |
| Acid | Citric acid | 4 | 20 | — | — | — | — | — |
| Polymer | HPMC E5 | 4 | 20 | | 80 | | | |
| | PVP/VA | — | — | 80 | — | — | — | — |
| | PVP K30 | — | — | — | — | 80 | — | — |
| | Soluplus | — | — | — | — | — | 80 | — |
| | HPMCAS-LF | — | — | — | — | — | — | 80 |
| Surfactant | VE-TPGS | 4 | — | 40 | 40 | 40 | 40 | 40 |
| | Lecithin | — | 20 | — | — | — | — | — |

Amorphous solid dispersions were prepared by Spray drying method. Briefly, API was dispersed a mixed organic solvent (e.g., ethanol and acetonitrile (3/7, V/V), methyl ethyl ketone and chloroform (5/5, V/V), dichloromethane and methanol (7/3, V/V), ethyl acetate and DCM (4/6, V/V), tetrahydrofuran and acetonitrile (2/8, V/V) or any solvent or mixed organic solvent listed in Table 10) with stirring to form a clear solution. And then the other ingredients were dissolved into the solution completely. The solution was introduced into spray dryer (SD-06AG, Labplant, UK) via flash atomization. The parameters used are provided Table 9B. Other parameters used include the following: feed rate 4.0-10.0 rpm, inlet temperature 50-100° C., outlet temperature 30-80° C., and atomization pressure 0-4.0 bar. The particles were collected and filled into the capsules. After collection, the particles were dried in a convection tray dryer to remove the residual solvent before further use. The particles were filled into gel capsule. Each capsule contains 10 mg vilazodone hydrochloride.

TABLE 9B

| | |
|---|---|
| Feed rate (rpm) | 9 |
| Inlet temperature (C.°) | 75 |
| Outlet temperature (C.°) | 49 |
| Atomization pressure (bar) | 2.2 |

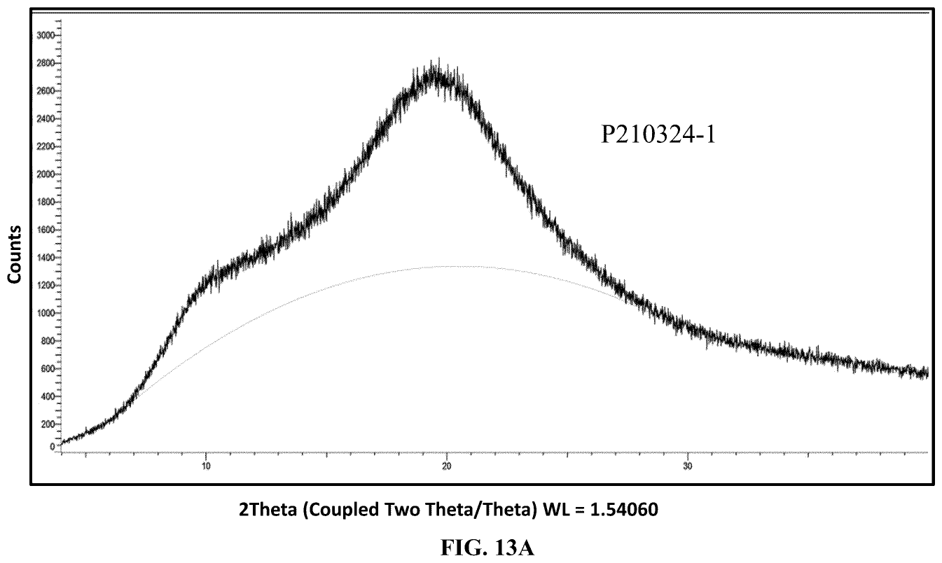

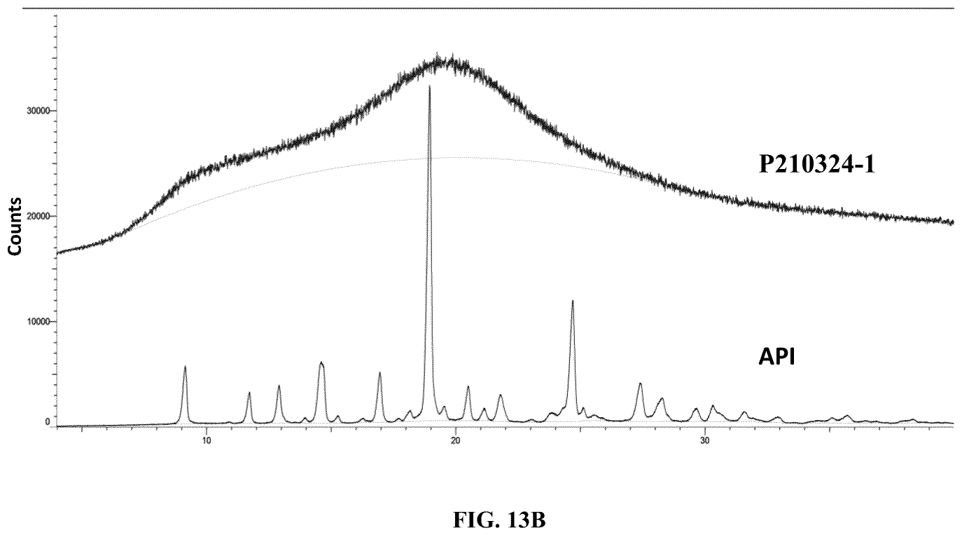

The XRPD testing was performed using D2-Phasher (Bruker) equipment, and with the following parameters: generator, 30.0 kV/10.0 mA; detector, Lynxeye; wavelength, Cu Ka1(1.54060); scan mode, continuous PSD fast; scan range, 4-40 degree; step Size, 0.01 degree; time/step, 0.5 s; sample stage rotation, 15 r/min. The result (FIGS. 13A-13B) suggests that the particles of this ASD composition for vilazodone were in amorphous state.

Kinetic solubility of the vilazodone compositions was measured. Excess of SDD powder was added into 40 ml FaSSIF medium at 37° C. under 400 rpm rotation. One mL medium was withdrawn into a centrifugal tube at specified time point (5, 30, 60, and 120 min) and then centrifuged for 10 min under 10000 rpm. The upper solution was collected for assay measure by HPLC.

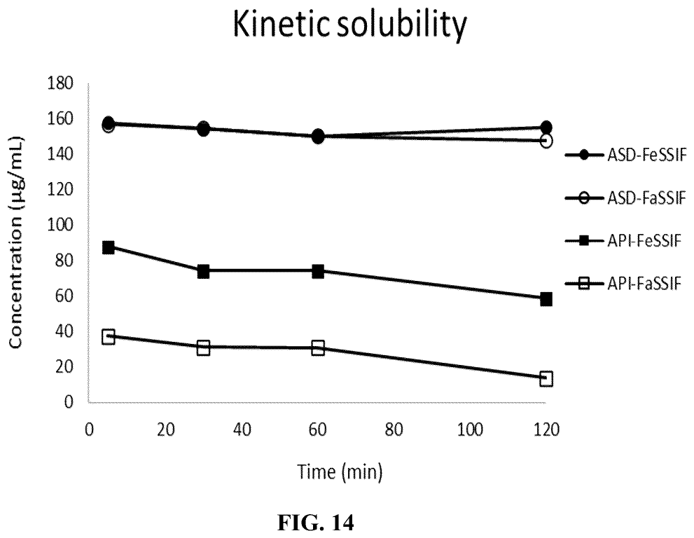

The results (Table 9C and FIG. 14) showed that the crystalline form of vilazodone has limited solubility in FaSSIF and FeSSIF medium, and the solubility in FaSSIF medium is lower than that in FaSSIF. When it is prepared in the form of ASD composition containing polymer, acid and surfactant, the kinetic solubility increases significantly in both FaSSIF and FeSSIF medium and the solubility in the two media are about the same.

TABLE 9C

| | Kinetic solubility (μg/mL) | |
|---|---|---|
| Time | API | P210324-1 Vilazodone |

| (min) | FaSSIF | FeSSIF | FaSSIF | FeSSIF |
|---|---|---|---|---|
| 5 | 37.4 | 87.63 | 156.71 | 157.42 |
| 30 | 30.93 | 73.99 | 154.46 | 154.28 |
| 60 | 30.76 | 74.08 | 149.98 | 150.12 |
| 120 | 13.63 | 58.54 | 147.6 | 155.11 |

The reference product, VIIBRYD® tablet (10 mg), was tested orally in six beagle dogs under two different dosing pretreatments, using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 12 and 24 h after the drug administration. Vilazodone plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 9D include the geometric mean values as well as the coefficient of variation (CV) of the pharmacokinetic parameters of two dosing groups. The absorption of VIIBRYD® under fed situation is about 2 times higher than that under fasted, which indicates a great food effect.

TABLE 9D

| | | Cₘₐₓ | AUCₗₐₛₜ | AUC_{inf} |
|---|---|---|---|---|
| Study design | | ng/mL | h*ng/mL | h*ng/mL |
| VIIBRYD (10 mg) in | Average | 20.3 | 105.5 | 115.3 |
| fasted condition | CV(%) | 75.1 | 108.0 | 103.9 |
| VIIBRYD (10 mg) in | Average | 39.1 | 192.4 | 211.0 |
| fed condition | CV(%) | 42.9 | 60.5 | 64.0 |

The capsules batch No. P210624-1 was tested orally in six beagle dogs under two different dosing pretreatments (with or without high-fat food), using a two-way crossover design. During the study, the dogs were allowed to drink water freely and were fasted for 12 hours before administration, all dogs were given food 4 hours after drug administration. Each dog was administered together with 50 ml water.

Blood samples were taken from each group of animals at the following time points: 0 h (before test drug administration), and 0.5, 1, 1.5, 2, 3, 4, 6, 8, 12 and 24 h after the drug administration. Vilazodone plasma concentrations were analyzed by an LC-MS/MS method. The non-compartment model of software WinNonlin was used to calculate the pharmacokinetic parameters of each dog, Cₘₐₓ, AUCₗₐₛₜ and AUC_{inf}. The geometric averages of each parameters were used for comparisons of the in vivo absorption under different doing pretreatments.

The results shown in Table 9E include the geometric mean values as well as coefficient of variation (CV) of the pharmacokinetic parameters of two dosing groups. The absorption of vilazodone in batch No. P210624-1 under fasted situation is about three times higher than that of reference product VIIBRYD under fasted condition, when the dose of vilazodone is the same in both compositions (10 mg). The absorption of vilazodone in batch No. P210624-1 under fasted situation is comparable to that under fed situation, which indicates no food effect.

TABLE 9E

| | | Cₘₐₓ | AUCₗₐₛₜ | AUC_{inf} |
|---|---|---|---|---|
| Study design | | ng/mL | h*ng/mL | h*ng/mL |
| ASD capsules batch | Average | 71.8 | 317.4 | 332.4 |
| No. P210624-1 | CV(%) | 55.3 | 70.5 | 70.8 |
| Vilazodone (10 mg) in | | | | |
| fasted condition | | | | |
| ASD capsules batch | Average | 49.6 | 287.7 | 304.5 |
| No. P210624-1 | CV(%) | 49.6 | 69.7 | 66.5 |
| Vilazodone (10 mg) in | | | | |
| fed condition | | | | |

The ASD capsules (batch No. P220215-1) and the reference product, VIIBRYD® tablet (10 mg), was tested orally in six beagle dogs with a three-way crossover design. Blood samples were taken from each group of animals at the following time points: 0 h (before test drug administration), and 0.5, 1, 1.5, 2, 3, 4, 6, 8, 12 and 24 h after the drug administration. Vilazodone plasma concentrations were analyzed by an LC-MS/MS method. The non-compartment model of software WinNonlin was used to calculate the pharmacokinetic parameters of each dog, Cmax, AUCt and AUCinf. The geometric averages of each parameters were used for comparisons of the in vivo absorption under different doing pretreatments. Table 9F shows the geometric mean values as well as coefficient of variation (CV) of the pharmacokinetic parameters of two dosing groups. The result shows that vilazodone ASD has nearly removed food-effect, and the oral absorption of ASD is comparable to Viibryd in fed condition.

TABLE 9F

| | | Cₘₐₓ | AUCₗₐₛₜ | AUC_{inf}_{_{—}}_{obs} |
|---|---|---|---|---|
| Study design | | ng/mL | h*ng/mL | h*ng/mL |
| VIIBRYD in Fed | Average | 44.83 | 216.8 | 227.6 |
| condition | CV(%) | 61.4 | 67.5 | 66.4 |
| ASD (P220215-1) | Average | 55.88 | 212.4 | 236.9 |
| in Fasted | CV(%) | 73.7 | 80.8 | 82.7 |
| condition | | | | |
| ASD (P220215-1) | Average | 52.16 | 232.9 | 279.8 |
| in Fed | CV(%) | 63.0 | 78.9 | 67.8 |
| condition | | | | |

It should be appreciated that there is considerable overlap between the above listed components in common usage, since a given component is often classified differently by different practitioners in the field, or is commonly used for any of several different functions, or may have differing functions depending on the levels in the composition. Thus, the above-listed components should be taken as merely exemplary, and not limiting, of the types of components that can be included in compositions of the present invention.

### Enumerated Embodiments

1. A pharmaceutical composition, wherein the pharmaceutical composition comprises an amorphous solid dispersion (ASD) that comprises:
   a) an active pharmaceutical ingredient (API) in an amount of about 5% to about 35% by weight of the ASD, wherein the API is cabozantinib or a pharmaceutically acceptable salt thereof;
   b) a surfactant in an amount of about 5% to about 60% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof;
   c) a hydrophilic polymer in an amount of about 1% to about 80% by weight of the ASD; and
   d) optionally an adsorbent in an amount of about 1% to 40% by weight of the ASD, wherein the adsorbent is silicone dioxide.
2. A pharmaceutical composition, wherein the pharmaceutical composition comprises an amorphous solid dispersion (ASD) that comprises:
   a) an active pharmaceutical ingredient (API) in an amount of about 5% to about 45% by weight of the ASD, wherein the API is venetoclax or a pharmaceutically acceptable salt thereof;
   b) a surfactant in an amount of about 5% to about 50% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof;
   c) a non-ionic hydrophilic polymer in an amount of about 1% to about 80% by weight of the ASD;
   d) optionally an inorganic acid or organic acid in an amount of about 1% to 20% by weight of the ASD; and
   e) optionally an adsorbent in an amount of about 1% to 40% by weight of the ASD, wherein the adsorbent is silicone dioxide.
3. A pharmaceutical composition, wherein the pharmaceutical composition comprises an amorphous solid dispersion (ASD) that comprises:
   a) an active pharmaceutical ingredient (API) in amount of about 5% to about 60% by weight of the ASD, wherein the API is selected from abiraterone acetate, alectinib hydrochloride, pazopanib hydrochloride, lurasidone, and vilazodone;
   b) optionally, a surfactant in an amount of about 5% to about 60% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof;
   c) a hydrophilic polymer in an amount of about 1% to about 90% by weight of the ASD;
   d) optionally an inorganic acid or organic acid in an amount of about 5% to 40% by weight of the ASD; and
   e) optionally an adsorbent in an amount of about 1% to 40% by weight of the ASD.
4. The pharmaceutical composition of any one of embodiments 1 to 3, wherein the hydrophilic polymer is polyvinyl alcohol (PVA), oligosaccharide, polysaccharide, polyvinylpyrrolidone (PVP), hydroxypropyl methylcellulose (HPMC, or hypromellose), hydroxyethyl cellulose (HEC), hydroxypropyl cellulose (HPC), polyethylene oxide, cyclodextrin and its derivatives (CD), hydropropylmethylcellulose acetate succinate (HPMCAS), polyethylene glycol (PEG), polyvinyl acetate and polyvinylcaprolactame-based graft copolymer (PVAc-PVCap-PEG), polyvinyl caprolactam-polyvinyl acetate-polyethylene glycol (PCL-PVAc-PEG), or a combination thereof
5. The pharmaceutical composition of any one of embodiments 1 to 3, wherein the hydrophilic polymer is polyvinyl alcohol (PVA), hydroxypropyl methylcellulose (HPMC), hydropropylmethylcellulose acetate succinate (HPMCAS), polyvinylpyrrolidone (povidone or PVP), vinylpyrrolidone-vinyl acetate copolymer (copovidone) or a combination thereof.
6. The pharmaceutical composition of any one of embodiments 1 to 3, wherein the hydrophilic polymer comprises polyvinyl alcohol (PVA), polyvinylpyrrolidone (PVP), hydroxypropyl methylcellulose (HPMC), hydroxypropyl beta cyclodextrin (HP-β-CD), sulfobutylether-β-cyclodextrin, or a combination thereof.
7. The pharmaceutical composition of any one of embodiments 1 to 6, wherein the pharmaceutical composition is in a form of a tablet or capsule.
8. The pharmaceutical composition of any one of embodiments 1 to 7, wherein the API is present in the pharmaceutical composition in an amount of about 5% to 30% by weight.
9. The pharmaceutical composition of any one of embodiments 1 to 7, wherein the API is present in the pharmaceutical composition in an amount of about 10 mg to about 500 mg.
10. The pharmaceutical composition of any one of embodiments 1 to 7, wherein the API is present in the pharmaceutical composition in an amount of about 20 mg to about 200 mg.
11. The pharmaceutical composition of any one of embodiments 1 to 7, wherein the API is present in the pharmaceutical composition in an amount of about 40 mg to about 150 mg.
12. The pharmaceutical composition of any one of embodiments 1 to 7, wherein the API is present in the pharmaceutical composition in an amount of about 50 mg to about 125 mg.
13. The pharmaceutical composition of any one of embodiments 1 to 12, wherein the surfactant comprises phospholipids or their derivatives such as lecithin, polyethylene glycol, polypropylene glycol, polysorbate, tocopherol polyethylene glycol succinate (TPGS), polyethylene glycol castor oil, sorbitan oleate, polyvinylcaprolactame-based graft copolymer (PVAc-PVCap-PEG), or any combination thereof.
14. The pharmaceutical composition of any one of embodiments 1 to 12, wherein the surfactant comprises TPGS.
15. The pharmaceutical composition of any one of embodiments 1 to 12, wherein the surfactant comprises phospholipids or their derivatives such as lecithin.
16. The pharmaceutical composition of any one of embodiments 1 to 12, wherein the surfactant comprises a block copolymer of polyethylene glycol and polypropylene glycol.
17. The pharmaceutical composition of any one of embodiments 1 to 12, wherein the surfactant comprises polysorbate, tocopherol polyethylene glycol succinate (TPGS), polyethylene glycol castor oil, sorbitan oleate, polyvinylcaprolactame-based graft copolymer (PVAc-PVCap-PEG), or a combination thereof.
18. The pharmaceutical composition of any one of embodiments 1 to 17, wherein the surfactant is present in the pharmaceutical composition in an amount of about 1% to 50% by weight.
19. The pharmaceutical composition of any one of embodiments 1 to 17, wherein the surfactant is present in the pharmaceutical composition in an amount of about 5% to 30% by weight.
20. The pharmaceutical composition of any one of embodiments 1 to 17, wherein the surfactant is present in the pharmaceutical composition in an amount of about 10 mg to about 500 mg.
21. The pharmaceutical composition of any one of embodiments 1 to 17, wherein the surfactant is present in the pharmaceutical composition in an amount of about 20 mg to about 200 mg.
22. The pharmaceutical composition of any one of embodiments 1 to 17, wherein the surfactant is present in the pharmaceutical composition in an amount of about 40 mg to about 150 mg.
23. The pharmaceutical composition of any one of embodiments 1 to 22, wherein the hydrophilic polymer is present in the amorphous solid dispersion in an amount of about 1% to about 75% by weight.
24. The pharmaceutical composition of any one of embodiments 1 to 22, wherein the hydrophilic polymer is present in the amorphous solid dispersion in an amount of about 20% to about 60% by weight.
25. The pharmaceutical composition of any one of embodiments 1 to 22, wherein the hydrophilic polymer is present in the pharmaceutical composition in an amount of about 10 mg to about 500 mg.
26. The pharmaceutical composition of any one of embodiments 1 to 25, wherein the adsorbent is selected from silicon dioxide, active carbon, magnesium aluminum silicate, diatomite, microcrystalline cellulose (MCC), silicified microcrystalline cellulose (SMCC), tale, crosslinked povidone, sodium carboxymethylcellulose, sodium carboxymethyl starch, and also sugars or sugar alcohols such as sorbitol, mannitol, lactose, cyclodextrin, and maltodextrin.
27. The pharmaceutical composition of any one of embodiments 1 to 25, wherein the adsorbent is silicon dioxide.
28. The pharmaceutical composition of any one of embodiments 1 to 27, wherein the adsorbent is present in the amorphous solid dispersion in an amount of about at least 20% by weight (such as 20-30% or 20% to 50%).
29. The pharmaceutical composition of any one of embodiments 1 to 27, wherein the adsorbent is present in the pharmaceutical composition in an amount of about 30 mg to about 200 mg.
30. The pharmaceutical composition of any one of embodiments 1 to 27, wherein the adsorbent is present in the pharmaceutical composition in an amount of about 50 mg to about 100 mg.
31. The pharmaceutical composition of any one of embodiments 1 to 30, wherein the pharmaceutical composition comprises an inorganic acid or organic acid.
32. The pharmaceutical composition of embodiment 31, wherein the organic acid or inorganic acid is selected from tartaric acid, fumaric acid, succinic acid, citric acid, lactic acid, malic acid, methanesulfonic acid, ethanesulfonic acid, isethionic acid, benzenesulfonic acid, p-toluenesulfonic acid, hydrochloric acid, sulfuric acid, and phosphoric acid.
33. The pharmaceutical composition of embodiment 31, wherein the pharmaceutical composition comprises an organic acid.
34. The pharmaceutical composition of embodiment 33, wherein the organic acid is selected from tartaric acid, succinic acid, citric acid, malic acid, and methanesulfonic acid.
35. The pharmaceutical composition of any one of embodiments 31 to 34, wherein the organic acid or inorganic acid is present in the amorphous solid dispersion in an amount of about 5% to about 40% by weight.
36. The pharmaceutical composition of any one of embodiments 31 to 34, wherein the organic acid or inorganic acid is present in the amorphous solid dispersion in an amount of about 20% to about 30% by weight.
37. The pharmaceutical composition of any one of embodiments 31 to 34, wherein the organic acid or inorganic acid is present in the pharmaceutical composition in an amount of about 10 mg to about 500 mg.
38. The pharmaceutical composition of any one of embodiments 31 to 34, wherein the organic acid or inorganic acid is present in the pharmaceutical composition in an amount of about 10 mg to about 200 mg.
39. The pharmaceutical composition of any one of embodiments 31 to 34, wherein the organic acid or inorganic acid is present in the pharmaceutical composition in an amount of about 30 mg to about 100 mg.
40. The pharmaceutical composition of any one of embodiments 1 to 39, wherein a D50 value of the amorphous solid dispersion is from 1 μm to 1000 μm.
41. The pharmaceutical composition of embodiment 40, wherein the D50 value is from about 1 μm to about 50 μm.
42. The pharmaceutical composition of embodiment 1, wherein the ASD comprises:
   a) cabozantinib free base or a pharmaceutical acceptable salt thereof in an amount of about 10% to about 30% by weight of the ASD;
   b) a surfactant in an amount of about 10% to about 55% by weight of the ASD, wherein the surfactant comprises lecithin, a block copolymer of polyethylene glycol and polypropylene glycol, TPGS, or a combination thereof;
   c) a hydrophilic polymer in an amount of about 5% to about 70% by weight of the ASD, wherein the hydrophilic polymer is HPMC;
   d) optionally an adsorbent in an amount of about 5% to 40% by weight of the ASD, wherein the adsorbent is silicone dioxide; and
   e) optionally an organic acid in an amount of about 5% to 40% by weight of the ASD, wherein the organic acid is malic acid.
43. The pharmaceutical composition of embodiment 42, wherein the ASD comprises:
   a) cabozantinib free base or a pharmaceutical acceptable salt thereof in an amount of about 12% to about 25% by weight of the ASD;
   b) a surfactant in an amount of about 15% to about 50% by weight of the ASD, wherein the surfactant comprises lecithin, TPGS, or a combination thereof;
   c) a hydrophilic polymer in an amount of about 10% to about 50% by weight of the ASD, wherein the hydrophilic polymer is HPMC (such as HPMC-E5);
   d) optionally an adsorbent in an amount of about 15% to 30% by weight of the ASD, wherein the adsorbent is silicone dioxide; and
   e) optionally an organic acid in an amount of about 10% to 30% by weight of the ASD, wherein the organic acid is malic acid.
44. The pharmaceutical composition of embodiment 1, wherein the ASD comprises:
   a) cabozantinib free base or cabozantinib malate in an amount of about 20 mg to about 80 mg; b) a surfactant in an amount of about 20 mg to about 220 mg, wherein the surfactant comprises lecithin, a block copolymer of polyethylene glycol and polypropylene glycol, TPGS, or a combination thereof;
   c) a hydrophilic polymer in an amount of about 40 mg to about 150 mg, wherein the hydrophilic polymer is HPMC (such as HPMC-E5);
   d) optionally an adsorbent in an amount of about 40 mg to about 120 mg, wherein the adsorbent is silicone dioxide; and
   e) optionally an organic acid in an amount of about 20 mg to about 80 mg, wherein the organic acid is malic acid.
45. The pharmaceutical composition of embodiment 44, wherein the ASD comprises:
   a) cabozantinib free base or cabozantinib malate in an amount of about 20 mg to about 80 mg;
   b) a surfactant in an amount of about 20 mg to about 200 mg, wherein the surfactant comprises lecithin, TPGS, or a combination thereof;
   c) a hydrophilic polymer in an amount of about 20 mg to about 150 mg, wherein the hydrophilic polymer is HPMC (such as HPMC-E5);
   d) optionally an adsorbent in an amount of about 30 mg to about 100 mg, wherein the adsorbent is silicone dioxide; and
   e) optionally an organic acid in an amount of about 20 mg to about 70 mg, wherein the organic acid is malic acid.
46. An amorphous solid dispersion (ASD), wherein the amorphous solid dispersion comprises:
   a) cabozantinib free base or cabozantinib malate;
   b) a surfactant;
   c) a hydrophilic polymer;
   d) optionally an adsorbent; and
   e) optionally an organic acid.
47. The ASD of embodiment 46, wherein the ASD comprises:
   a) cabozantinib free base or cabozantinib malate;
   b) a surfactant, wherein the surfactant comprises lecithin, a block copolymer of polyethylene glycol and polypropylene glycol, TPGS, or a combination thereof;
   c) a non-ionic hydrophilic polymer, wherein the non-ionic hydrophilic polymer is HPMC (such as HPMC-E5);
   d) optionally an adsorbent, wherein the adsorbent is silicone dioxide; and
   e) optionally an organic acid, wherein the organic acid is malic acid.
48. The ASD of embodiment 47, wherein the ASD comprises:
   a) cabozantinib free base or cabozantinib malate in an amount of about 10% to about 30% by weight of the ASD;
   b) a surfactant in an amount of about 10% to about 55% by weight of the ASD, wherein the surfactant comprises lecithin, a block copolymer of polyethylene glycol and polypropylene glycol, TPGS, or a combination thereof;
   c) a hydrophilic polymer in an amount of about 5% to about 70% by weight of the ASD, wherein the non-ionic hydrophilic polymer is HPMC (such as HPMC-E5);
   d) optionally an adsorbent in an amount of about 5% to 40% by weight of the ASD, wherein the adsorbent is silicone dioxide; and
   e) optionally an organic acid in an amount of about 5% to 40% by weight of the ASD, wherein the organic acid is malic acid.
49. A method of treating a disease or condition, comprising administering to a subject in need thereof the pharmaceutical composition or the amorphous solid dispersion of any one of embodiments 1 to 48.
50. A method of treating cancer, comprising administering to a subject in need thereof the pharmaceutical composition or the amorphous solid dispersion of any one of embodiments 1, 42 to 48.
51. The method of embodiment 50, wherein the cancer comprises kidney cancer, liver cancer, and thyroid cancer.
52. The method of embodiment 50, wherein the kidney cancer is advanced renal cell carcinoma.
53. The method of embodiment 50, wherein the liver cancer is hepatocellular carcinoma.
54. The method of embodiment 50, wherein the thyroid cancer is locally advanced or metastatic differentiated thyroid cancer or medullary thyroid cancer.
55. A method of inhibiting multiple tyrosine-kinases, comprising administering to a subject in need thereof the pharmaceutical composition or the amorphous solid dispersion of any one of embodiments 1, or 42 to 48.
56. The method of embodiment 55, wherein the multiple tyrosine-kinases comprise VEGFR2, c-MET or RET.
57. The method of any one of embodiments 49 to 56, further comprising administering an immunotherapeutic agent.
58. The method of embodiment 57, wherein the immunotherapeutic agent is nivolumab.
59. The method of any one of embodiments 49 to 56 wherein the subject was previously treated with sorafenib.
60. The method of any one of embodiments 49 to 56, wherein the subject
   a) is 12 years of age or older,
   b) progressed following prior VEGFR-targeted therapy, and
   c) is radioactive iodine-refractory or ineligible.
61. The pharmaceutical composition of embodiment 2, wherein the ASD comprises:
   a) venetoclax or a pharmaceutically acceptable salt thereof in an amount of about 10% to about 40% by weight of the ASD;
   b) a surfactant in an amount of about 10% to about 50% by weight of the ASD, wherein the surfactant comprises lecithin, a block copolymer of polyethylene glycol and polypropylene glycol, TPGS, or a combination thereof;
   c) a hydrophilic polymer in an amount of about 10% to about 50% by weight of the ASD, wherein the non-ionic hydrophilic polymer is copovidone, HPMCAS, or a combination of both; and
   d) an organic acid in an amount of about 1% to 20% by weight of the ASD, wherein the organic acid is citric acid.
62. The pharmaceutical composition of embodiment 61, wherein the ASD comprises:
   a) venetoclax or a pharmaceutically acceptable salt thereof in an amount of about 20% to about 40% by weight of the ASD;
   b) a surfactant in an amount of about 20% to about 40% by weight of the ASD, wherein the surfactant is TPGS;
   c) a non-ionic hydrophilic polymer in an amount of about 20% to about 40% by weight of the ASD, wherein the non-ionic hydrophilic polymer is copovidone, HPMCAS, or a combination of both; and
   d) an organic acid in an amount of about 3% to 15% by weight of the ASD, wherein the organic acid is citric acid.
63. The pharmaceutical composition of embodiment 2, wherein the ASD comprises:
   a) venetoclax or a pharmaceutically acceptable salt thereof in an amount of about 60 mg to about 140 mg;
   b) a surfactant in an amount of about 50 mg to about 150 mg, wherein the surfactant is TPGS;
   c) a non-ionic hydrophilic polymer in an amount of about 50 mg to about 150 mg, wherein the non-ionic hydrophilic polymer is copovidone, HPMCAS, or a combination of both; and
   d) an organic acid in an amount of about 5 mg to about 50 mg, wherein the organic acid is citric acid.
64. The pharmaceutical composition of embodiment 63, wherein the ASD comprises:
   a) venetoclax or a pharmaceutically acceptable salt thereof in an amount of about 80 mg to about 120 mg;
   b) a surfactant in an amount of about 70 mg to about 130 mg, wherein the surfactant is TPGS;
   c) a non-ionic hydrophilic polymer in an amount of about 70 mg to about 130 mg, wherein the non-ionic hydrophilic polymer is copovidone, HPMCAS, or a combination of both; and
   d) an organic acid in an amount of about 10 mg to about 40 mg, wherein the organic acid is citric acid.
65. An amorphous solid dispersion (ASD), wherein the amorphous solid dispersion comprises:
   a) venetoclax or a pharmaceutically acceptable salt thereof;
   b) a surfactant;
   c) a non-ionic hydrophilic polymer; and
   d) an organic acid.
66. The ASD of embodiment 65, wherein the ASD comprises:
   a) venetoclax or a pharmaceutically acceptable salt thereof;
   b) a surfactant, wherein the surfactant is TPGS;
   c) a non-ionic hydrophilic polymer, wherein the non-ionic hydrophilic polymer is copovidone, HPMCAS, or a combination of both; and
   d) an organic acid, wherein the organic acid is citric acid.
67. The ASD of embodiment 66, wherein the ASD comprises:
   a) venetoclax or a pharmaceutically acceptable salt thereof in an amount of about 10% to about 40% by weight of the ASD;
   b) a surfactant in an amount of about 10% to about 50% by weight of the ASD, wherein the surfactant comprises lecithin, a block copolymer of polyethylene glycol and polypropylene glycol, TPGS, or a combination thereof;
   c) a non-ionic hydrophilic polymer in an amount of about 10% to about 50% by weight of the ASD, wherein the non-ionic hydrophilic polymer is copovidone (such as VA64); and
d) an organic acid in an amount of about 1% to 20% by weight of the ASD, wherein the organic acid is citric acid.
68. A method of treating a disease or condition, comprising administering to a subject in need thereof the pharmaceutical composition or the amorphous solid dispersion of any one of embodiments 2, or 61 to 67.
69. A method of treating cancer, comprising administering to a subject in need thereof the pharmaceutical composition or the amorphous solid dispersion of any one of embodiments 2, or 61 to 67.
70. The method of embodiment 69, wherein the caner is a blood cancer.
71. The method of embodiment 70, wherein the blood cancer is chronic lymphocytic leukemia.
72. The method of embodiment 70, wherein the blood cancer is acute myeloid leukemia.
73. The method of embodiment 69, wherein the caner is a solid tumor.
74. The method of embodiment 73, wherein the solid tumor is lymphoma.
75. The method of embodiment 74, wherein the lymphoma is small lymphocytic lymphoma.
76. A method of inhibiting B-cell lymphoma-2 (Bel-2) protein, comprising administering to a subject in need thereof the pharmaceutical composition or the amorphous solid dispersion of any one of embodiments 2, or 61 to 67.
77. The method of any one of embodiments 69 to 71, or 73 to 76, further comprising administering an immunotherapeutic agent.
78. The method of embodiment 77, wherein the immunotherapeutic agent is obinutuzumab or rituximab.
79. The method of any one of embodiments 69 to 70, or 72, further comprising administering a chemotherapeutic agent.
80. The method of embodiment 79, wherein the chemotherapeutic agent is azacitidine, or decitabine, or low-dose cytarabine.
81. The method of any one of embodiments 69 to 78, wherein the subject is previously untreated.
82. The method of any one of embodiments 69 to 71, or 73 to 78, wherein the subject is previously treated.
83. The method of any one of embodiments 69 to 70, 72, or 79 to 80, wherein the subject
   a) is newly diagnosed of acute myeloid leukemia; and
   b) is 75 years of age or older; or
   c) has other medical conditions that prevent the use of standard chemotherapy.
84. The method of any one of embodiments 69 to 78, wherein the subject is an adult.
85. The pharmaceutical composition of embodiment 3, wherein the ASD comprises:
   a) abiraterone free base or abiraterone acetate in an amount of about 5% to about 40% by weight of the ASD;
   b) a non-ionic hydrophilic polymer in an amount of about 30% to about 95% by weight of the ASD, wherein the non-ionic hydrophilic polymer is HPMCAS; and
   c) optionally a surfactant in an amount of about 10% to about 40% by weight of the ASD, wherein the surfactant is lecithin.
86. The pharmaceutical composition of embodiment 85, wherein the ASD comprises:
   a) abiraterone free base or abiraterone acetate in an amount of about 8% to about 30% by weight of the ASD;
   b) a non-ionic hydrophilic polymer in an amount of about 50% to about 91% by weight of the ASD, wherein the non-ionic hydrophilic polymer is HPMCAS; and
   c) optionally a surfactant in an amount of about 15% to about 30% by weight of the ASD, wherein the surfactant is lecithin.
87. The pharmaceutical composition of embodiment 3, wherein the ASD comprises:
   a) abiraterone free base or abiraterone acetate in an amount of about 20 mg to about 80 mg;
   b) a non-ionic hydrophilic polymer in an amount of about 80 mg to about 700 mg, wherein the non-ionic hydrophilic polymer is HPMCAS; and
   c) optionally a surfactant in an amount of about 10 mg to about 80 mg, wherein the surfactant is lecithin.
88. The pharmaceutical composition of embodiment 87, wherein the ASD comprises:
   a) abiraterone free base or abiraterone acetate in an amount of about 30 mg to about 60 mg;
   b) a non-ionic hydrophilic polymer in an amount of about 100 mg to about 600 mg, wherein the non-ionic hydrophilic polymer is HPMCAS; and
   c) optionally a surfactant in an amount of about 20 mg to about 60 mg, wherein the surfactant is lecithin.
89. An amorphous solid dispersion (ASD), wherein the amorphous solid dispersion comprises:
   a) abiraterone free base or abiraterone acetate;
   b) a non-ionic hydrophilic polymer; and
   c) optionally a surfactant.
90. The ASD of embodiment 89, wherein the ASD comprises:
   a) abiraterone free base or abiraterone acetate;
   b) a non-ionic hydrophilic polymer, wherein the non-ionic hydrophilic polymer is HPMCAS; and
   c) optionally a surfactant, wherein the surfactant is lecithin.
91. The ASD of embodiment 90, wherein the ASD comprises:
   a) abiraterone free base or abiraterone acetate in an amount of about 5% to about 40% by weight of the ASD;
   b) a non-ionic hydrophilic polymer in an amount of about 30% to about 95% by weight of the ASD, wherein the non-ionic hydrophilic polymer is HPMCAS; and
   c) optionally a surfactant in an amount of about 10% to about 40% by weight of the ASD, wherein the surfactant is lecithin.
92. The pharmaceutical composition of any one of embodiments 85 to 91, wherein the pharmaceutical composition exhibits a bioavailability that is from about 75% to about 200% of a bioavailability of a corresponding reference composition comprising abiraterone acetate, when measured as AUCₗₐₛₜ after oral administration, wherein the reference pharmaceutical composition does not comprise an amorphous solid dispersion, and wherein the reference formulation is at least 3 times the dosage of the pharmaceutical composition.
93. The pharmaceutical composition of any one of embodiments 85 to 91, wherein the pharmaceutical composition exhibits a bioavailability that is from about 100% to 200% of a bioavailability of a corresponding reference composition comprising abiraterone acetate, when measured as Cₘₐₓ after oral administration, wherein the reference pharmaceutical composition does not comprise an amorphous solid dispersion, and wherein the reference composition is at least 3 times the dosage of the pharmaceutical composition.
94. A method of treating a disease or condition, comprising administering to a subject in need thereof the pharmaceutical composition or the amorphous solid dispersion of any one of embodiments 3, or 85 to 93.
95. A method of treating cancer, comprising administering to a subject in need thereof the pharmaceutical composition or the amorphous solid dispersion of any one of embodiments 3, or 85 to 93.
96. The method of embodiment 95, wherein the caner is a solid tumor.
97. The method of embodiment 96, wherein the solid tumor is prostate cancer.
98. The method of embodiment 97, wherein the prostate cancer is metastatic castration-resistant prostate cancer.
99. The method of embodiment 97, wherein the prostate cancer is metastatic high-risk castration-sensitive prostate cancer.
100. A method of inhibiting 17 alpha-hydroxylase/C17, 20-lyase (CYP17), comprising administering to a subject in need thereof the pharmaceutical composition or the amorphous solid dispersion of any one of embodiments 3, or 85 to 93.
101. The method of any one of embodiments 94 to 98, or 100, further comprising administering a corticosteroid.
102. The method of embodiment 101, wherein the corticosteroid is prednisone or methylprednisolone.
103. The method of any one of embodiments 94 to 97, or 99 to 100, further comprising administering a corticosteroid.
104. The method of embodiment 10377, wherein the corticosteroid is methylprednisolone.
105. The method of any one of embodiments 94 to 104, wherein the subject is a male adult.
106. The pharmaceutical composition of embodiment 3, wherein the ASD comprises:
   a) alectinib free base or a pharmaceutically acceptable salt thereof in an amount of about 5% to about 60% by weight of the ASD;
   b) a non-ionic hydrophilic polymer in an amount of about 15% to about 80% by weight of the ASD, wherein the non-ionic hydrophilic polymer is HPMC (such as HPMC-E5), HPMCAS (such as HPMCAS-LF) or PCL-PVAc-PEG (or Soluplus) or a combination thereof;
   c) a surfactant in an amount of about 10% to about 40% by weight of the ASD, wherein the surfactant is TPGS or SLS or a combination thereof; and
   d) optionally an organic acid in an amount of about 10% to about 50% by weight of the ASD, wherein the organic acid is tartaric acid.
107. The pharmaceutical composition of embodiment 106, wherein the ASD comprises:
   a) alectinib free base or a pharmaceutically acceptable salt thereof in an amount of about 15% to about 55% by weight of the ASD;
   b) a non-ionic hydrophilic polymer in an amount of about 15% to about 70% by weight of the ASD, wherein the non-ionic hydrophilic polymer is HPMC (such as HPMC-E5), HPMCAS or Soluplus or a combination thereof;
   c) a surfactant in an amount of about 15% to about 30% by weight of the ASD, wherein the surfactant is TPGS or SLS or a combination thereof; and
   d) optionally an organic acid in an amount of about 20% to about 40% by weight of the ASD, wherein the organic acid is tartaric acid.
108. The pharmaceutical composition of embodiment 3, wherein the ASD comprises:
   a) alectinib free base or a pharmaceutically acceptable salt thereof in an amount of about 50 mg to about 200 mg;
   b) a non-ionic hydrophilic polymer in an amount of about 100 mg to about 500 mg, wherein the non-ionic hydrophilic polymer is HPMC (such as HPMC-E5), HPMCAS or Soluplus or a combination thereof;
   c) a surfactant in an amount of about 40 mg to about 250 mg, wherein the surfactant is TPGS or SLS or a combination thereof; and
   d) optionally an organic acid in an amount of about 70 mg to 250 mg, wherein the organic acid is tartaric acid.
109. The pharmaceutical composition of embodiment 108, wherein the ASD comprises:
   a) alectinib free base or a pharmaceutically acceptable salt thereof in an amount of about 60 mg to about 180 mg;
   b) a non-ionic hydrophilic polymer in an amount of about 100 mg to about 400 mg, wherein the non-ionic hydrophilic polymer is HPMC (such as HPMC-E5), HPMCAS (such as HPMCAS-LF) or Soluplus or a combination thereof;
   c) a surfactant in an amount of about 50 mg to about 200 mg, wherein the surfactant is TPGS or SLS or a combination thereof; and
   d) optionally an organic acid in an amount of about 130 mg to 180 mg, wherein the organic acid is tartaric acid.
110. An amorphous solid dispersion (ASD), wherein the amorphous solid dispersion comprises:
   a) alectinib free base or a pharmaceutically acceptable salt thereof;
   b) a non-ionic hydrophilic polymer;
   c) a surfactant and
   d) optionally an organic acid.
111. The ASD of embodiment 89, wherein the ASD comprises:
   a) alectinib free base or a pharmaceutically acceptable salt thereof;
   b) a non-ionic hydrophilic polymer, wherein the non-ionic hydrophilic polymer is HPMC, HPMCAS or Soluplus or a combination thereof;
   c) a surfactant, wherein the surfactant is TPGS or SLS or a combination thereof; and
   d) optionally an organic acid, wherein the organic acid is tartaric acid.
112. The ASD of embodiment 90, wherein the ASD comprises:
   a) alectinib free base or a pharmaceutically acceptable salt thereof in an amount of about 5% to about 60% by weight of the ASD;
   b) a non-ionic hydrophilic polymer in an amount of about 15% to about 80% by weight of the ASD, wherein the non-ionic hydrophilic polymer is HPMC, HPMCAS or Soluplus or a combination thereof;
   c) a surfactant in an amount of about 10% to about 40% by weight of the ASD, wherein the surfactant is TPGS or SLS or a combination thereof; and
   d) optionally an organic acid in an amount of about 10% to 50% by weight of the ASD, wherein the organic acid is tartaric acid.
113. The pharmaceutical composition of any one of embodiments 106 to 112, wherein the pharmaceutical composition exhibits a bioavailability that is from about 75% to about 200% of a bioavailability of a corresponding reference formulation comprising alectinib hydrochloride, when measured as AUCₗₐₛₜ after oral administration, wherein the reference pharmaceutical composition does not comprise an amorphous solid dispersion, and wherein the reference formulation is at least 1.75 times the dosage of the pharmaceutical composition.
114. The pharmaceutical composition of any one of embodiments 106 to 112, wherein the pharmaceutical composition exhibits a bioavailability that is from about 75% to about 200% of a bioavailability of a corresponding reference formulation comprising alectinib hydrochloride, when measured as Cₘₐₓ after oral administration, wherein the reference pharmaceutical composition does not comprise an amorphous solid dispersion, and wherein the reference formulation is at least 1.75 times the dosage of the pharmaceutical composition.
115. A method of treating a disease or condition, comprising administering to a subject in need thereof the pharmaceutical composition or the amorphous solid dispersion of any one of embodiments 3, or 106 to 115.
116. A method of treating cancer, comprising administering to a subject in need thereof the pharmaceutical composition or the amorphous solid dispersion of any one of embodiments 3, or 106 to 115.
117. The method of embodiment 95, wherein the caner is a solid tumor.
118. The method of embodiment 96, wherein the solid tumor is lung cancer.
119. The method of embodiment 97, wherein the lung cancer is non-small cell lung cancer.
120. The method of embodiment 97, wherein the non-small cell lung cancer is anaplastic lymphoma kinase (ALK)-positive.
121. A method of inhibiting ALK and/or RET tyrosine kinases, comprising administering to a subject in need thereof the pharmaceutical composition or the amorphous solid dispersion of any one of embodiments 3, or 106 to 115.
122. The method of any one of embodiments 115 to 121, wherein the subject is an adult.
123. The method of any one of embodiments 115 to 122, wherein the subject is intolerant to crizotinib.
124. The pharmaceutical composition of embodiment 3, wherein the ASD comprises:
   a) pazopanib free base or a pharmaceutically acceptable salt thereof in an amount of about 5% to about 40% by weight of the ASD;
   b) a non-ionic hydrophilic polymer in an amount of about 10% to about 70% by weight of the ASD, wherein the non-ionic hydrophilic polymer is HPMC (such as HPMC-E5);
   c) a surfactant in an amount of about 10% to about 40% by weight of the ASD, wherein the surfactant is TPGS or lecithin or a combination thereof;
   d) optionally an organic acid in an amount of about 10% to about 40% by weight of the ASD, wherein the organic acid is tartaric acid; and
   e) optionally an adsorbent in an amount of about 15% to about 40% by weight of the ASD, wherein the adsorbent is SiO₂.
125. The pharmaceutical composition of embodiment 124, wherein the ASD comprises:
   a) pazopanib free base or a pharmaceutically acceptable salt thereof in an amount of about 15% to about 30% by weight of the ASD;
   b) a non-ionic hydrophilic polymer in an amount of about 15% to about 60% by weight of the ASD, wherein the non-ionic hydrophilic polymer is HPMC;
   c) a surfactant in an amount of about 15% to about 30% by weight of the ASD, wherein the surfactant is TPGS or lecithin or a combination thereof;
   d) optionally an organic acid in an amount of about 15% to about 30% by weight of the ASD, wherein the organic acid is tartaric acid; and
   e) optionally an adsorbent in an amount of about 20% to about 35% by weight of the ASD, wherein the adsorbent is SiO₂.
126. The pharmaceutical composition of embodiment 3, wherein the ASD comprises:
   a) pazopanib free base or a pharmaceutically acceptable salt thereof in an amount of about 30 mg to about 200 mg;
   b) a non-ionic hydrophilic polymer in an amount of about 30 mg to about 400 mg by weight of the ASD, wherein the non-ionic hydrophilic polymer is HPMC;
   c) a surfactant in an amount of about 30 mg to about 200 mg, wherein the surfactant is TPGS or lecithin or a combination thereof;
   d) optionally an organic acid in an amount of about 30 mg to about 200 mg, wherein the organic acid is tartaric acid; and
   e) optionally an adsorbent in an amount of about 30 mg to about 150 mg, wherein the adsorbent is SiO₂.
127. The pharmaceutical composition of embodiment 127, wherein the ASD comprises:
   a) pazopanib free base or a pharmaceutically acceptable salt thereof in an amount of about 30 mg to about 150 mg;
   b) a non-ionic hydrophilic polymer in an amount of about 30 mg to about 300 mg by weight of the ASD, wherein the non-ionic hydrophilic polymer is HPMC;
   c) a surfactant in an amount of about 30 mg to about 150 mg, wherein the surfactant is TPGS or lecithin or a combination thereof;
   d) optionally an organic acid in an amount of about 30 mg to about 150 mg, wherein the organic acid is tartaric acid; and
   e) optionally an adsorbent in an amount of about 40 mg to about 100 mg, wherein the adsorbent is SiO₂.
128. An amorphous solid dispersion (ASD), wherein the amorphous solid dispersion comprises:
   a) pazopanib free base or a pharmaceutically acceptable salt thereof;
   b) a non-ionic hydrophilic polymer;
   c) a surfactant;
   d) optionally an organic acid; and
   e) optionally an adsorbent.
129. The ASD of embodiment 128, wherein the ASD comprises:
   a) pazopanib free base or a pharmaceutically acceptable salt thereof;
   b) a non-ionic hydrophilic polymer, wherein the non-ionic hydrophilic polymer is HPMC;
   c) a surfactant, wherein the surfactant is TPGS or lecithin or a combination thereof;
   d) optionally an organic acid, wherein the organic acid is tartaric acid; and
   e) optionally an adsorbent, wherein the adsorbent is SiO₂.
130. The ASD of embodiment 129, wherein the ASD comprises:
   a) pazopanib free base or a pharmaceutically acceptable salt thereof in an amount of about 5% to about 40% by weight of the ASD;
   b) a non-ionic hydrophilic polymer in an amount of about 10% to about 70% by weight of the ASD, wherein the non-ionic hydrophilic polymer is HPMC;
   c) a surfactant in an amount of about 10% to about 40% by weight of the ASD, wherein the surfactant is TPGS or lecithin or a combination thereof;
   d) optionally an organic acid in an amount of about 10% to about 40% by weight of the ASD, wherein the organic acid is tartaric acid; and
   e) optionally an adsorbent in an amount of about 15% to about 40% by weight of the ASD, wherein the adsorbent is SiO₂.
131. The pharmaceutical composition of any one of embodiments 124 to 130, wherein the pharmaceutical composition exhibits a bioavailability that is from about 40% to about 100% of a bioavailability of a corresponding reference formulation comprising pazopanib hydrochloride, when measured as AUCₗₐₛₜ after oral administration under fasted condition, wherein the reference pharmaceutical composition does not comprise an amorphous solid dispersion, and wherein the reference formulation is at least 3 times the dosage of the pharmaceutical composition.
132. The pharmaceutical composition of any one of embodiments 124 to 130, wherein the pharmaceutical composition exhibits a bioavailability that is from about 50% to about 120% of a bioavailability of a corresponding reference formulation comprising pazopanib hydrochloride, when measured as Cₘₐₓ after oral administration under fasted condition, wherein the reference pharmaceutical composition does not comprise an amorphous solid dispersion, and wherein the reference formulation is at least 3 times the dosage of the pharmaceutical composition.
133. A method of treating a disease or condition, comprising administering to a subject in need thereof the pharmaceutical composition or the amorphous solid dispersion of any one of embodiments 3, or 124 to 132.
134. A method of treating cancer, comprising administering to a subject in need thereof the pharmaceutical composition or the amorphous solid dispersion of any one of embodiments 3, or 124 to 130.
135. The method of embodiment 134, wherein the caner is a solid tumor.
136. The method of embodiment 135, wherein the solid tumor is soft tissue sarcoma.
137. The method of embodiment 136, wherein the soft tissue sarcoma is advanced soft tissue sarcoma.
138. The method of embodiment 134, wherein the cancer is kidney cancer.
139. The method of embodiment 138, wherein the kidney cancer is advanced renal cell cancer.
140. A method of inhibiting tyrosine kinases of VEGF receptor (VEGFR) and/or PDGF receptor (PDGFR), comprising administering to a subject in need thereof the pharmaceutical composition or the amorphous solid dispersion of any one of embodiments 3, or 124 to 132.
141. The method of any one of embodiments 133 to 140, wherein the subject is an adult.
142. The method of any one of embodiments 133 to 137, wherein the subject previously received chemotherapy.
143. The pharmaceutical composition of embodiment 3, wherein the ASD comprises:
   a) lurasidone free base or a pharmaceutically acceptable salt thereof in an amount of about 5% to about 50% by weight of the ASD;
   b) a hydrophilic polymer in an amount of about 10% to about 70% by weight of the ASD, wherein the hydrophilic polymer is HPMC, PVP/VA, HPMC AS, PVP, or a combination thereof;
   c) a surfactant in an amount of about 10% to about 40% by weight of the ASD, wherein the surfactant is TPGS, lecithin or a combination thereof;
   d) an organic acid in an amount of about 10% to about 40% by weight of the ASD, wherein the organic acid is tartaric acid or citric acid or a combination thereof; and
   e) optionally an adsorbent in an amount of about 15% to about 40% by weight of the ASD, wherein the adsorbent is SiO₂.
144. The pharmaceutical composition of embodiment 143, wherein the ASD comprises:
   a) lurasidone free base or a pharmaceutically acceptable salt thereof in an amount of about 10% to about 40% by weight of the ASD;
   b) a non-ionic hydrophilic polymer in an amount of about 15% to about 60% by weight of the ASD, wherein the non-ionic hydrophilic polymer is HPMC;
   c) a surfactant in an amount of about 10% to about 30% by weight of the ASD, wherein the surfactant is TPGS;
   d) an organic acid in an amount of about 15% to about 40% by weight of the ASD, wherein the organic acid is tartaric acid or citric acid or a combination thereof; and
   e) optionally an adsorbent in an amount of about 15% to about 30% by weight of the ASD, wherein the adsorbent is SiO₂. 145. The pharmaceutical composition of embodiment 3, wherein the ASD comprises:
a) lurasidone free base or a pharmaceutically acceptable salt thereof in an amount of about 20 mg to about 120 mg (e.g., 20, 40, 60, 80, or 120 mg);
   b) a hydrophilic polymer in an amount of about 20 mg to about 240 mg, wherein the hydrophilic polymer is HPMC (such as HPMC-E5);
   c) a surfactant in an amount of about 20 mg to about 120 mg, wherein the surfactant is TPGS;
   d) an organic acid in an amount of about 20 mg to about 120 mg, wherein the organic acid is tartaric acid or citric acid or a combination thereof; and
e) optionally an adsorbent in an amount of about 30 mg to about 180 mg, wherein the adsorbent is SiO₂.
146. The pharmaceutical composition of embodiment 145, wherein the ASD comprises:
   a) lurasidone free base or a pharmaceutically acceptable salt thereof in an amount of about 20 mg to about 60 mg;
   b) a non-ionic hydrophilic polymer in an amount of about 20 mg to about 160 mg, wherein the non-ionic hydrophilic polymer is HPMC;
   c) a surfactant in an amount of about 20 mg to about 60 mg, wherein the surfactant is TPGS;
   d) optionally an organic acid in an amount of about 20 mg to about 60 mg, wherein the organic acid is tartaric acid or citric acid or a combination thereof; and
   e) optionally an adsorbent in an amount of about 30 mg to about 80 mg, wherein the adsorbent is SiO₂.
147. An amorphous solid dispersion (ASD), wherein the amorphous solid dispersion comprises:
   a) lurasidone free base or a pharmaceutically acceptable salt thereof;
   b) a non-ionic hydrophilic polymer;
   c) a surfactant;
   d) optionally an organic acid; and
   e) optionally an adsorbent.
148. The ASD of embodiment 147 wherein the ASD comprises:
   a) lurasidone free base or a pharmaceutically acceptable salt thereof;
   b) a non-ionic hydrophilic polymer, wherein the non-ionic hydrophilic polymer is HPMC;
   c) a surfactant, wherein the surfactant is TPGS;
   d) optionally an organic acid, wherein the organic acid is tartaric acid or citric acid or a combination thereof; and
   e) optionally an adsorbent, wherein the adsorbent is SiO₂.
149. The ASD of embodiment 148, wherein the ASD comprises:
   a) lurasidone free base or a pharmaceutically acceptable salt thereof in an amount of about 5% to about 50% by weight of the ASD;
   b) a non-ionic hydrophilic polymer in an amount of about 10% to about 70% by weight of the ASD, wherein the non-ionic hydrophilic polymer is HPMC;
   c) a surfactant in an amount of about 10% to about 40% by weight of the ASD, wherein the surfactant is TPGS;
   d) optionally an organic acid in an amount of about 10% to about 40% by weight of the ASD, wherein the organic acid is tartaric acid or citric acid or a combination thereof; and
   e) optionally an adsorbent in an amount of about 15% to about 40% by weight of the ASD, wherein the adsorbent is SiO₂.
150. The pharmaceutical composition of any one of embodiments 143 to 149, wherein the pharmaceutical composition exhibits a bioavailability that is from about 300% to about 1000% of a bioavailability of a corresponding reference formulation comprising lurasidone hydrochloride, when measured as AUCₗₐₛₜ after oral administration under fasted condition, wherein the reference pharmaceutical composition does not comprise an amorphous solid dispersion.
151. The pharmaceutical composition of any one of embodiments 143 to 149, wherein the pharmaceutical composition exhibits a bioavailability that is from about 500% to about 1500% of a bioavailability of a corresponding reference formulation comprising lurasidone hydrochloride, when measured as Cₘₐₓ after oral administration under fasted condition, wherein the reference pharmaceutical composition does not comprise an amorphous solid dispersion.
152. A method of treating a disease or condition, comprising administering to a subject in need thereof the pharmaceutical composition or the amorphous solid dispersion of any one of embodiments 3, or 143 to 151.
153. A method of treating a mental disorder, comprising administering to a subject in need thereof the pharmaceutical composition or the amorphous solid dispersion of any one of embodiments 3, or 143 to 151.
154. The method of embodiment 153, wherein the mental disorder is schizophrenia.
155. The method of embodiment 153, wherein the mental disorder is depression.
156. The method of embodiment 155, wherein the depression is associated with bipolar I disorder.
157. The method of embodiment 155, wherein the depression is bipolar depression.
158. A method of inhibiting central dopamine D2 and serotonin Type 2 (5HT2A) receptor, comprising administering to a subject in need thereof the pharmaceutical composition or the amorphous solid dispersion of any one of embodiments 3, or 143 to 151.
159. The method of any one of embodiments 152 to 158, wherein the subject is an adult.
160. The method of any one of embodiments 152 to 154, wherein the subject is an adolescent, wherein the adolescent is 13 to 17 years old.
161. The method of any one of embodiments 152 to 153, or 155 to 158, further comprising administering an anticonvulsant.
162. The method of embodiment 161, wherein the anticonvulsant is lithium or valproate.
163. The pharmaceutical composition of embodiment 3, wherein the ASD comprises:
   a) vilazodone free base or a pharmaceutically acceptable salt thereof in an amount of about 5% to about 40% by weight of the ASD;
   b) a non-ionic hydrophilic polymer in an amount of about 10% to about 50% by weight of the ASD, wherein the non-ionic hydrophilic polymer is HPMC;
   c) a surfactant in an amount of about 10% to about 40% by weight of the ASD, wherein the surfactant is TPGS; and
   d) an organic acid in an amount of about 10% to about 40% by weight of the ASD, wherein the organic acid is citric acid.
164. The pharmaceutical composition of embodiment 163, wherein the ASD comprises:
   a) vilazodone free base or a pharmaceutically acceptable salt thereof in an amount of about 15% to about 30% by weight of the ASD;
   b) a non-ionic hydrophilic polymer in an amount of about 15% to about 30% by weight of the ASD, wherein the non-ionic hydrophilic polymer is HPMC;
   c) a surfactant in an amount of about 15% to about 30% by weight of the ASD, wherein the surfactant is TPGS; and
   d) an organic acid in an amount of about 15% to about 30% by weight of the ASD, wherein the organic acid is citric acid.
165. An amorphous solid dispersion (ASD), wherein the amorphous solid dispersion comprises:
   a) vilazodone free base or a pharmaceutically acceptable salt thereof;
   b) a non-ionic hydrophilic;
   c) a surfactant; and
   d) an organic acid.
166. The ASD of embodiment 165, wherein the ASD comprises:
   a) vilazodone free base or a pharmaceutically acceptable salt thereof;
   b) a non-ionic hydrophilic polymer, wherein the non-ionic hydrophilic polymer is HPMC (such as HPMC-E5);
   c) a surfactant, wherein the surfactant is TPGS; and
   d) an organic acid, wherein the organic acid is citric acid.
167. The ASD of embodiment 166, wherein the ASD comprises:
   a) vilazodone free base or a pharmaceutically acceptable salt thereof in an amount of about 5% to about 40% by weight of the ASD;
   b) a non-ionic hydrophilic polymer in an amount of about 10% to about 50% by weight of the ASD, wherein the non-ionic hydrophilic polymer is HPMC;
   c) a surfactant in an amount of about 10% to about 40% by weight of the ASD, wherein the surfactant is TPGS; and
   d) an organic acid in an amount of about 10% to about 40% by weight of the ASD, wherein the organic acid is citric acid.
168. The pharmaceutical composition of any one of embodiments 163 to 167, wherein the pharmaceutical composition exhibits a bioavailability that is from about 130% to about 500% of a bioavailability of a corresponding reference formulation comprising vilazodone hydrochloride, when measured as AUCₗₐₛₜ after oral administration, wherein the reference pharmaceutical composition does not comprise an amorphous solid dispersion.
169. The pharmaceutical composition of any one of embodiments 163 to 167, wherein the pharmaceutical composition exhibits a bioavailability that is from about 115% to about 600% of a bioavailability of a corresponding reference formulation comprising vilazodone hydrochloride, when measured as Cₘₐₓ after oral administration, wherein the reference pharmaceutical composition does not comprise an amorphous solid dispersion.
170. A method of treating a disease or condition, comprising administering to a subject in need thereof the pharmaceutical composition or the amorphous solid dispersion of any one of embodiments 3, or 163 to 169.
171. A method of treating a mental disorder, comprising administering to a subject in need thereof the pharmaceutical composition or the amorphous solid dispersion of any one of embodiments 3, or 163 to 169.
172. The method of embodiment 171, wherein the mental disorder is major depressive disorder.
173. A method of inhibiting serotonin (5-HT1A) receptors, comprising administering to a subject in need thereof the pharmaceutical composition or the amorphous solid dispersion of any one of embodiments 3, or 170 to 172.
174. A method of stimulating serotonin transporter, comprising administering to a subject in need thereof the pharmaceutical composition or the amorphous solid dispersion of any one of embodiments 3, or 170 to 172.
175. The method of embodiment 174, wherein the stimulation is via partial agonism.
176. The method of any one of embodiments 170 to 175, wherein the subject is an adult.

1-202. (canceled)

203. A pharmaceutical composition comprising an amorphous solid dispersion (ASD), wherein the ASD comprises:
a) an active pharmaceutical ingredient (API) in an amount of from about 5% to about 60% by weight of the ASD, wherein the API comprises abiraterone, alectinib, pazopanib, lurasidone, or vilazodone, or a pharmaceutically acceptable salt thereof;
b) a surfactant in an amount of from about 5% to about 60% by weight of the ASD, wherein the surfactant comprises phospholipids or their derivatives, a block copolymer of polyethylene glycol and polypropylene glycol, TPGS, polyvinylcaprolactame-based graft copolymer (PVAc-PVCap-PEG), polyoxyl hydrogenated castor oil, or a combination thereof; and
c) a hydrophilic polymer in an amount of from about 1% to about 90% by weight of the ASD.

204. The pharmaceutical composition of claim 203, wherein the ASD comprises:
a) alectinib free base or a pharmaceutically acceptable salt thereof;
b) the surfactant;
c) the hydrophilic polymer in an amount of about 1% to about 80% by weight of the ASD;
d) optionally an inorganic acid or organic acid; and
e) optionally an adsorbent.

205. The pharmaceutical composition of claim 204, wherein the hydrophilic polymer comprises polyvinyl alcohol (PVA), hydroxypropyl methylcellulose (HPMC), polymethacrylates, hydropropylmethylcellulose acetate succinate (HPMCAS), polyvinylpyrrolidone (povidone or PVP), vinylpyrrolidone-vinyl acetate copolymer (copovidone) or a combination thereof.

206. The pharmaceutical composition of claim 204, wherein the API is present in the pharmaceutical composition in an amount of from about 10% to about 50% by weight.

207. The pharmaceutical composition of claim 204, wherein the surfactant comprise lecithin, and wherein the surfactant is present in the pharmaceutical composition in an amount of from about 10% to 50% by weight.

208. The pharmaceutical composition of claim 204, wherein the ASD comprises the adsorbent, wherein the adsorbent is selected from the group consisting of: silicon dioxide, active carbon, magnesium aluminum silicate, diatomite, microcrystalline cellulose (MCC), silicified microcrystalline cellulose (SMCC), talc, crosslinked povidone, sodium carboxymethylcellulose, sodium carboxymethyl starch, sugar, and sugar alcohol, and wherein the adsorbent is present in the amorphous solid dispersion in an amount of from about 5% to about 30%.

209. The pharmaceutical composition of claim 204, wherein the ASD comprises the inorganic acid or organic acid, wherein the inorganic acid or organic acid is selected from the group consisting of: fatty acids, tartaric acid, fumaric acid, succinic acid, citric acid, lactic acid, malic acid, methanesulfonic acid, ethanesulfonic acid, isethionic acid, benzenesulfonic acid, p-toluenesulfonic acid, hydrochloric acid, sulfuric acid, and phosphoric acid, and wherein the organic acid or inorganic acid is present in the amorphous solid dispersion in an amount of from about 20% to about 30% by weight.

210. The pharmaceutical composition of claim 204, wherein the ASD comprises:
a) alectinib free base or a pharmaceutically acceptable salt thereof in an amount of from about 5% to about 50% by weight of the ASD;
b) the hydrophilic polymer in an amount of from about 1% to about 80% by weight of the ASD;
c) the surfactant in an amount of from about 10% to about 40% by weight of the ASD, wherein the surfactant comprises phospholipids or their derivatives, polyethylene glycol (PEG), a block copolymer of polyethylene glycol and polypropylene glycol, sodium lauryl sulfate (SLS), TPGS, polyvinyl acetate and polyvinylcaprolactame-based graft copolymer, polyoxyl hydrogenated castor oil, polyoxylglycerides, polysorbate, or a combination thereof;
d) the organic acid in an amount of from about 10% to about 35% by weight of the ASD; and
e) the adsorbent in an amount of from about 15% to about 40% by weight of the ASD.

211. The pharmaceutical composition of claim 204, wherein the ASD comprises:
a) alectinib free base or a pharmaceutically acceptable salt thereof in an amount of from about 5% to about 50% by weight of the ASD;
b) the hydrophilic polymer in an amount of from about 10% to about 40% by weight of the ASD, wherein the hydrophilic polymer comprises HPMC, HPMCAS, polyvinyl acetate and polyvinylcaprolactame-based graft copolymer, PVP, copovidone, polymethacrylates, or a combination thereof;
c) the surfactant in an amount of from about 10% to about 40% by weight of the ASD, wherein the surfactant comprises lecithin, polyethylene glycol (PEG), TPGS, polyvinyl acetate and polyvinylcaprolactame-based graft copolymer, polyoxylglycerides, polysorbate, or a combination thereof;
d) the organic acid in an amount of from about 10% to about 35% by weight of the ASD, wherein the organic acid is tartaric acid, citric acid, or a combination thereof; and
e) the adsorbent in an amount of from about 15% to about 40% by weight of the ASD, wherein the adsorbent is silicon dioxide.

212. The pharmaceutical composition of claim 204, wherein the ASD comprises:
a) alectinib free base or a pharmaceutically acceptable salt thereof in an amount of from about 5% to about 60% by weight of the ASD;
b) the hydrophilic polymer in an amount of from about 15% to about 80% by weight of the ASD, wherein the hydrophilic polymer comprises HPMC, polymethacrylates, HPMCAS, or PCL-PVAc-PEG or a combination thereof;
c) the surfactant in an amount of from about 10% to about 40% by weight of the ASD, wherein the surfactant comprises TPGS, polyoxylglycerides, polysorbate, polyoxyl hydrogenated castor oil, or SLS or a combination thereof;
d) the organic acid in an amount of from about 10% to about 50% by weight of the ASD, wherein the organic acid is tartaric acid, citric acid, or malic acid, or a combination thereof;
e) the adsorbent in an amount of from about 1% to 40% by weight of the ASD, wherein the adsorbent is silicon dioxide.

213. The pharmaceutical composition of claim 204, wherein the ASD comprises:
a) alectinib free base or a pharmaceutically acceptable salt thereof in an amount of from about 15% to about 55% by weight of the ASD;
b) the hydrophilic polymer in an amount of from about 15% to about 70% by weight of the ASD, wherein the hydrophilic polymer comprises HPMC, polymethacrylates, HPMCAS, or Soluplus or a combination thereof;
c) the surfactant in an amount of from about 15% to about 30% by weight of the ASD, wherein the surfactant comprises TPGS, polyoxylglycerides, polysorbate, polyoxyl hydrogenated castor oil, or SLS or a combination thereof;
d) the organic acid in an amount of from about 20% to about 40% by weight of the ASD, wherein the organic acid is tartaric acid, citric acid, or malic acid, or a combination thereof; and
e) the adsorbent in an amount of from about 1% to 30% by weight of the ASD, wherein the adsorbent is silicon dioxide.

214. The pharmaceutical composition of claim 204, wherein the pharmaceutical composition is formulated in a unit dosage form, wherein the ASD comprises:
a) alectinib free base or a pharmaceutically acceptable salt thereof in an amount of from about 50 mg to about 400 mg;
b) the hydrophilic polymer in an amount of from about 100 mg to about 1000 mg, wherein the hydrophilic polymer comprises HPMC, polymethacrylates, HPMCAS or Soluplus or a combination thereof;
c) the surfactant in an amount of from about 40 mg to about 250 mg, wherein the surfactant comprises TPGS, polyoxylglycerides, polysorbate, polyoxyl hydrogenated castor oil, or SLS or a combination thereof;
d) optionally the organic acid in an amount of from about 70 mg to 250 mg, wherein the organic acid comprises tartaric acid, citric acid, or malic acid, or a combination thereof; and
e) optionally the adsorbent in an amount of from about 20 mg to 300 mg by weight of the ASD, wherein the adsorbent comprises silicon dioxide.

215. The pharmaceutical composition of claim 204, wherein the pharmaceutical composition is formulated in a unit dosage form, wherein the ASD comprises:
a) alectinib free base or a pharmaceutically acceptable salt thereof in an amount of from about 60 mg to about 180 mg;
b) the hydrophilic polymer in an amount of from about 100 mg to about 900 mg, wherein the hydrophilic polymer comprises HPMC, polymethacrylates, HPMCAS or Soluplus or a combination thereof;
c) the surfactant in an amount of from about 50 mg to about 200 mg, wherein the surfactant comprises TPGS, polyoxylglycerides, polysorbate, polyoxyl hydrogenated castor oil, or SLS or a combination thereof;
d) the organic acid in an amount of from about 130 mg to 180 mg, wherein the organic acid comprises tartaric acid, citric acid, or malic acid, or a combination thereof; and
e) the adsorbent in an amount of from about 50 mg to 200 mg by weight of the ASD, wherein the adsorbent comprises silicon dioxide.

216. The pharmaceutical composition of claim 204, wherein the pharmaceutical composition exhibits a bioavailability that is from about 75% to about 200% relative to a bioavailability of a corresponding reference formulation comprising alectinib hydrochloride, when measured as AUCₗₐₛₜ or Cₘₐₓ after oral administration, wherein the corresponding reference pharmaceutical composition does not comprise an amorphous solid dispersion, and wherein the corresponding reference formulation is at least 1.75 times the dosage of the pharmaceutical composition.

217. A pharmaceutical composition comprising an amorphous solid dispersion (ASD), wherein the ASD comprises:
a) an active pharmaceutical ingredient (API) in an amount of from about 5% to about 35% by weight of the ASD, wherein the API is cabozantinib or a pharmaceutically acceptable salt thereof;
b) a surfactant in an amount of from about 5% to about 60% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof;
c) a hydrophilic polymer in an amount of from about 1% to about 80% by weight of the ASD; and
d) optionally an adsorbent in an amount of from about 1% to 40% by weight of the ASD, wherein the adsorbent is silicon dioxide.

218. A pharmaceutical composition comprising an amorphous solid dispersion (ASD), wherein the ASD comprises:
a) an active pharmaceutical ingredient (API) in an amount of from about 5% to about 45% by weight of the ASD, wherein the API is venetoclax or a pharmaceutically acceptable salt thereof;
b) a surfactant in an amount of from about 5% to about 50% by weight of the ASD, wherein the surfactant comprises phospholipids or their derivatives such as lecithin, a block copolymer of polyethylene glycol and polypropylene glycol, TPGS, polyvinylcaprolactame-based graft copolymer (PVAc-PVCap-PEG), polyoxyl hydrogenated castor oil, or a combination thereof;
c) a non-ionic hydrophilic polymer in an amount of from about 1% to about 80% by weight of the ASD;
d) optionally an inorganic acid or organic acid in an amount of from about 1% to 20% by weight of the ASD; and
e) optionally an adsorbent in an amount of from about 1% to 40% by weight of the ASD, wherein the adsorbent is silicon dioxide.

219. A method of treating a disease or condition, comprising administering to a subject in need thereof the pharmaceutical composition or the ASD of claim 203.

220. A method of treating a disease or condition, comprising administering to a subject in need thereof the pharmaceutical composition or the ASD of claim 204.

221. A method of treating a disease or condition, comprising administering to a subject in need thereof the pharmaceutical composition or the ASD of claim 217.

222. A method of treating a disease or condition, comprising administering to a subject in need thereof the pharmaceutical composition or the ASD of claim 218.
